(19) United States
(12) Patent Application Publication
(10) Pub. No.: US 2011/0002866 A1
(43) Pub. Date: Jan. 6, 2011

(54) METHODS TO PREVENT A HAIR-RELATED SIDE EFFECT OF TREATMENT WITH A CHEMOTHERAPEUTIC AGENT

(76) Inventors: Beverly W. Lubit, Kinnelon, NJ (US); Pamela R. Lipkin, New York, NY (US)

Correspondence Address: GREENBERG TRAURIG, LLP, 200 PARK AVE., P.O. BOX 677, FLORHAM PARK, NJ, 07932, US

(21) Appl. No.: 12/881,945

(22) Filed: Sep. 14, 2010

Related U.S. Application Data
(63) Continuation-in-part of application No. 12/235,664, filed on Sep. 23, 2008, now Pat. No. 7,550,508.
Application No. 12/235,664 is a continuation-in-part of application No. 12/235,683, filed on Sep. 23, 2008, now Pat. No. 7,645,800.
Application No. 12/235,683 is a continuation-in-part of application No. 12/235,704, filed on Sep. 23, 2008, now Pat. No. 7,514,474.
Application No. 12/235,704 is a continuation-in-part of application No. 12/235,736, filed on Sep. 23, 2008, now Pat. No. 7,632,867.
Application No. 12/235,736 is a continuation-in-part of application No. 12/235,747, filed on Sep. 23, 2008, now Pat. No. 7,553,874.
Application No. 12/235,747 is a continuation-in-part of application No. 12/235,762, filed on Sep. 23, 2008, now Pat. No. 7,649,021.
Application No. 12/235,762 is a continuation-in-part of application No. 12/235,776, filed on Sep. 23, 2008, now Pat. No. 7,517,912.
Application No. 12/235,776 is a continuation-in-part of application No. 12/235,791, filed on Sep. 23, 2008, now Pat. No. 7,638,557.
Application No. 12/235,791 is a continuation-in-part of application No. 12/235,807, filed on Sep. 23, 2008, now Pat. No. 7,553,875.
Application No. 12/235,807 is a continuation-in-part of application No. 12/235,887, filed on Sep. 23, 2008, now Pat. No. 7,635,720.
Application No. 12/235,887 is a continuation-in-part of application No. 12/235,926, filed on Sep. 23, 2008, now Pat. No. 7,541,382.
Application No. 12/235,926 is a continuation-in-part of application No. 12/235,966, filed on Sep. 23, 2008, now Pat. No. 7,632,868.
Application No. 12/235,966 is a continuation-in-part of application No. 12/236,024, filed on Sep. 23, 2008.
Application No. 12/236,024 is a continuation-in-part of application No. PCT/US08/77357, filed on Sep. 23, 2008.
Application No. PCT/US08/77357 is a continuation-in-part of application No. 12/565,335, filed on Sep. 23, 2009.
Application No. 12/565,335 is a continuation-in-part of application No. PCT/US09/58040, filed on Sep. 23, 2009.
(60) Provisional application No. 61/242,320, filed on Sep. 14, 2009.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 60/984,198, filed on Oct. 31, 2007.
(60) Provisional application No. 61/099,226, filed on Sep. 23, 2008.
(60) Provisional application No. 61/099,226, filed on Sep. 23, 2008.

(51) Int. Cl. A61K 8/92; A61K 31/20; A61K 31/357; A61K 31/343; A61Q 1/10; A61P 17/14

(52) U.S. Cl. 424/70.7; 514/559; 514/464; 514/469

(57) ABSTRACT

The described invention relates to delivery of compositions comprising at least one prostaglandin analog to prevent or reduce hair loss (e.g. brittle hair growth, thin hair growth, short hair growth, sparse hair growth) or alopecia associated with chemotherapy.

### CROSS REFERENCE TO RELATED APPLICATIONS

This application claims the benefit of priority from U.S. 61/242,320, filed Sep. 14, 2009; is a continuation-in-part of U.S. Ser. No. 12/235,664, filed Sep. 23, 2008, now U.S. Pat. No. 7,550,508, which claims the benefit of priority from U.S. 60/984,198, filed Oct. 31, 2007; is a continuation-in-part of U.S. Ser. No. 12/235,683, filed Sep. 23, 2008, now U.S. Pat. No. 7,645,800, which claims the benefit of priority from U.S. 60/984,198, filed Oct. 31, 2007; is a continuation-in-part of U.S. Ser. No. 12/235,704, filed Sep. 23, 2008, now U.S. Pat. No. 7,514,474, which claims the benefit of priority from U.S. 60/984,198, filed Oct. 31, 2007; is a continuation-in-part of U.S. Ser. No. 12/235,736, filed Sep. 23, 2008, now U.S. Pat. No. 7,632,867, which claims the benefit of priority from U.S. 60/984,198, filed Oct. 31, 2007; is a continuation-in-part of U.S. Ser. No. 12/235,747, filed Sep. 23, 2008, now U.S. Pat. No. 7,553,874, which claims the benefit of priority from U.S. 60/984,198, filed Oct. 31, 2007; is a continuation-in-part of U.S. Ser. No. 12/235,762, filed Sep. 23, 2008, now U.S. Pat. No. 7,649,021, which claims the benefit of priority from U.S. 60/984,198, filed Oct. 31, 2007; is a continuation-in-part of U.S. Ser. No. 12/235,776, filed Sep. 23, 2008, now U.S. Pat. No. 7,517,912, which claims the benefit of priority from U.S. 60/984,198, filed Oct. 31, 2007; is a continuation-in-part of U.S. Ser. No. 12/235,791, filed Sep. 23, 2008, now U.S. Pat. No. 7,638,557, which claims the benefit of priority from U.S. 60/984,198, filed Oct. 31, 2007; is a continuation-in-part of U.S. Ser. No. 12/235,807, filed Sep. 23, 2008, now U.S. Pat. No. 7,553,875, which claims the benefit of priority from U.S. 60/984,198, filed Oct. 31, 2007; is a continuation-in-part of U.S. Ser. No. 12/235,887, filed Sep. 23, 2008, now U.S. Pat. No. 7,635,720, which claims the benefit of priority from U.S. 60/984,198, filed Oct. 31, 2007; is a continuation-in-part of U.S. Ser. No. 12/235,926, filed Sep. 23, 2008, now U.S. Pat. No. 7,541,382, which claims the benefit of priority from U.S. 60/984,198, filed Oct. 31, 2007; is a continuation-in-part of U.S. Ser. No. 12/235,966, filed Sep. 23, 2008, now U.S. Pat. No. 7,632,868, which claims the benefit of priority from U.S. 60/984,198, filed Oct. 31, 2007; is a continuation-in-part of U.S. Ser. No. 12/236,024, filed Sep. 23, 2008, which claims the benefit of priority from U.S. 60/984,198, filed Oct. 31, 2007; is a continuation-in-part of PCT/US08/77357, filed Sep. 23, 2008, which claims the benefit of priority from U.S. 60/984,198, filed Oct. 31, 2007; is a continuation-in-part of U.S. Ser. No. 12/565,335, filed Sep. 23, 2009, which claims the benefit of priority from U.S. 61/099,226, filed Sep. 23, 2008; and is a continuation-in-part of PCT/US09/58040, filed Sep. 23, 2009, which claims the benefit of priority from U.S. 61/099,226, filed Sep. 23, 2008; the contents of each of which are incorporated by reference in their entirety.

### FIELD OF THE INVENTION

The described invention relates to delivery of compositions comprising at least one prostaglandin analog to prevent or reduce hair loss (e.g. brittle hair growth, thin hair growth, short hair growth, sparse hair growth) or alopecia associated with chemotherapy.

### BACKGROUND OF THE INVENTION

### Cancer

Cancer is the general term frequently used to indicate any of various types of malignant neoplasms that (i) invade surrounding tissues, (ii) may metastasize to several sites, (iii) are likely to recur after attempted removal and (iv) cause death of the patient unless adequately treated. These properties of uncontrolled growth, invasion and metastasis differentiate malignant from benign tumors, which are self-limited, and do not invade or metastasis.

For example, breast cancer is a cancer that forms in tissues of the breast, usually the ducts (tubes that carry milk to the nipple) and lobules (glands that make milk). It occurs in both men and women, although male breast cancer is rare. The breasts sit on the chest muscles that cover the ribs. Each breast is made of 15-20 lobes, each of which contain many smaller lobules. Lobules contain groups of tiny glands that can produce milk, which flows from the lobules through thin tubes (ducts) to the nipple. The nipple is in the center of a dark area of skin called the areola. Fat fills the spaces between the lobules and ducts. The breasts also contain lymph vessels which lead to small, round organs called lymph nodes. Groups of lymph nodes are near the breast in the axilla (underarm), above the collarbone, in the chest behind the breastbone, and in many other parts of the body. Lymph nodes trap bacteria, cancer cells or other harmful substances. When breast cancer cells spread, the cancer cells often are found in lymph nodes near the breast and can spread to almost any other part of the body, most commonly bones, liver, lungs and brain. There are four stages of breast cancer. Stage 0 (carcinoma in situ) includes lobular carcinoma in situ (“LCIS”) and ductal carcinoma in situ (“DCIS”), where the cancerous cells are present in the lining of a lobule or duct, respectively. Stage 1 is an early stage of invasive breast cancer where the tumor is no more than 2 cm across and the cancer cells have not spread beyond the breast. In Stage II, the tumor is either (i) no more than 2 cm in diameter and the cancer has spread to the lymph nodes under the arm; (ii) between 2-4 cm in diameter and the cancer may have spread to the lymph nodes under the arm; or (iii) the cancer is larger than 5 cm in diameter and the cancer has not spread to the lymph nodes under the arm. Stage III may be a large tumor, but the cancer has not spread beyond the breast and nearby lymph nodes. It is locally advanced cancer. In Stage IIIA, the tumor may or may not be smaller than 5 cm in diameter and has spread to the lymph nodes under the arm. In Stage IIIB, the tumor has grown into the chest wall or the skin of the breast and the cancer has spread to the lymph nodes behind the breastbone. In Stage IIIC, the tumor is of any size and has spread to the lymph nodes under the arm, behind the breastbone, and under or above the collarbone. Stage IV is distant metastatic cancer where the cancer has spread to other parts of the body.

Another example is colorectal cancer, which refers to cancers of the colon and rectum. Colon cancer is a cancer that forms in the tissues of the colon (the longest part of the large intestine). Most colon cancers are adenocarcinomas (cancers that begin in the cells that make and release mucus and other fluids). Rectal cancer is a cancer that forms in the tissues of the rectum (the last several inches of the large intestine closest to the anus). When colorectal cancer spreads outside the colon or rectum, cancer cells often are found in nearby lymph nodes and elsewhere throughout the body, including the liver. There are 5 stages of colorectal cancer. In Stage 0, the cancer is found only in the innermost lining of the colon or rectum (carcinoma in situ). In Stage I, the tumor has grown into the inner wall of the colon or rectum, but not through it. Stage II tumors extend more deeply into or through the wall of the colon or rectum and may have invaded nearby tissue, but has not spread to the lymph nodes. In Stage III, the cancer has spread to nearby lymph nodes, but not to other parts of the body. In Stage IV, the cancer has spread to other parts of the body, such as the liver or lungs.

For some cancers, the best approach to treatment is a combination of surgery, radiation therapy, and chemotherapy. Generally, surgery or radiation therapy treats cancer that is confined locally, while chemotherapy also kills the cancer cells that have spread to distant sites. Sometimes radiation therapy or chemotherapy is given before surgery to shrink a tumor, thereby improving the opportunity for complete surgical removal. Radiation therapy and low-dose chemotherapy after surgery help to destroy any remaining cancer cells. The stage of the cancer often determines whether single therapy or a combination is needed.

### Cell-Cycle Kinetics

Although differences in the duration of the cell cycle occur between the cells of various types, all cells display a similar pattern during the division process. This cell cycle may be divided into four phases: (1) a presynthetic phase (“G₁”); (2) a phase in which the synthesis of DNA occurs (“S phase”); (3) an interval follows the termination of DNA synthesis, called the postsynthetic phase (“G₂”); and (4) the mitosis (“M”) phase, during which the G₂ cell, containing a double complement of DNA, divides into two daughter G₁ cells. Each daughter cell may immediately reenter the cell cycle or may pass into a nonproliferative stage (“G₀”). The G₀ cells of certain specialized tissues may differentiate into functional cells that no longer are capable of division.

Many of the most potent cytotoxic agents act by damaging DNA; their toxicity is greatest during the S, or DNA synthetic phase, of the cell cycle. Other agents, such as the vinca alkaloids and taxanes, block the formation of the mitotic spindle in M phase and have activity only against cells that are in the process of division. Accordingly, human neoplasms that are most susceptible to chemotherapeutic measures are those with a high percentage of cells undergoing division. Similarly, normal tissues that proliferate rapidly (bone marrow, hair follicles, and intestinal epithelium) are subject to damage by most antineoplastic drugs. Such toxicity often limits the usefulness of the drugs.

### Chemotherapy and Chemotherapeutic Agents

Chemotherapy, in its most general sense, refers to the treatment of disease by means of chemical substances or drugs. In popular usage, it refers to antineoplastic drugs used alone or in combination as a cytotoxic standardized regimen to treat cancer. In its non-oncological use, “chemotherapy” may refer, for example, to antibiotics.

Chemotherapy is employed as part of a multimodality approach to the initial treatment of many other tumors, including breast cancer, colon cancer and locally advanced stages of head and neck, lung, cervical, and esophageal cancer, soft tissue sarcomas, and pediatric solid tumors. The basic approaches to cancer treatment are constantly changing.

The majority of chemotherapeutic drugs can be divided into several categories including, but not limited to, (1) alkylating agents; (2) antimetabolites; (3) natural products; (4) hormones and related agents; (5) biologics; and (6) miscellaneous agents.

1. Alkylating Agents and their Side-Effects

Alkylating agents used in chemotherapy encompass a diverse group of chemicals that have in common the capacity to contribute, under physiological conditions, alkyl groups to biologically vital macromolecules, such as DNA. For several of the most valuable agents, such as cyclophosphamides and nitrosoureas, the active alkylating moieties are generated in vivo after complex metabolic reactions.

As shown in Table 1, there are five major types of alkylating agents used in chemotherapy of neoplastic diseases: (1) nitrogen mustards; (2) ethylenimimes; (3) alkyl sulfonates; (4) nitrosoureas; and (5) triazenes.

TABLE 1
Examples of Alkylating Agents Useful for Treating Neoplastic Diseases.

| | | | | Proposed Mechanism | Known Side- |
|---|---|---|---|---|---|
| Class | Type of Agent | Example | Neoplasms/Disease | of Action | Effects |
| Alkylating Agents | Triazene | Temozolomide | Glioma; malignant | temozolomide is | myelosuppression, |
| | | (Temodar ®) | melanoma | converted at | nausea, vomiting, |
| | | | | physiologic pH to | thrombocytopenia, |
| | | | | the short-lived | anorexia, insomnia, |
| | | | | active compound, | constipation, dry |
| | | | | monomethyl | skin, rash, and |
| | | | | triazeno imidazole | alopecia |
| | | | | carboxamide | |
| | | | | (MTIC). The | |
| | | | | cytotoxicity of | |
| | | | | MTIC is due | |
| | | | | primarily to | |
| | | | | methylation of | |
| | | | | DNA which results | |
| | | | | in inhibition of | |
| | | | | DNA replication | |
| Alkylating Agents | Alkyl Sulfonate | Busulfan | Chronic granulocytic | appears to act | myelosuppression |
| | | (Myleran ®) | leukemia | through the | and severe |
| | | | | alkylation of DNA | pancytopenia |
| | | | | | (defective capacity |
| | | | | | of the bone marrow |
| | | | | | to generate red |
| | | | | | blood cells), |
| | | | | | bronchopulmonary |
| | | | | | dysplasia, ovarian |
| | | | | | suppression, |
| | | | | | hyperpigmentation |
| | | | | | of the skin, and |
| | | | | | alopecia |
| Alkylating Agents | Nitrogen Mustard | Cyclophosamide | breast cancer; | In the liver, | nausea and |
| | | (Cytoxan ®) | different types of | cyclophosphamide | vomiting, anorexia, |
| | | | leukemia including | is converted to the | abdominal pain, |
| | | | acute lymphoblastic | active metabolites | hemorrhagic colitis, |
| | | | leukemia (“ALL”), | aldophosphamide | oral mucosal |
| | | | acute myeloid | and phosphoramide | ulceration, skin |
| | | | leukemia (“AML”), | mustard, which | rash, leukopenia, |
| | | | chronic lymphocytic | bind to DNA, | neutropenia, hair |
| | | | leukemia (“CLL”), | thereby inhibiting | depigmentation, |
| | | | and chronic | DNA replication | and alopecia |
| | | | myelogenous | and initiating cell | |
| | | | leukemia (“CML”); | death. | |
| | | | Hodgkin lymphoma; | | |
| | | | multiple myeloma; | | |
| | | | mycosis fungoides; | | |
| | | | neuroblastoma; non- | | |
| | | | Hodgkin lymphoma; | | |
| | | | ovarian cancer; and | | |
| | | | retinoblastoma. | | |
| Alkylating Agents | Nitrogen Mustard | Ifosamide | Acute and chronic | alkylates and forms | myelosuppression, |
| | | | lymphocytic | DNA crosslinks, | hematuria (blood in |
| | | | leukemias; Hodgkin 's | thereby preventing | the urine), central |
| | | | disease; non- | DNA strand | nervous system |
| | | | Hodgkin's | separation and | toxicity, renal |
| | | | lymphomas; multiple | DNA replication | impairment, and |
| | | | myeloma; | | alopecia |
| | | | neuroblastoma; | | |
| | | | breast, ovary, lung | | |
| | | | cancer; Wilm's | | |
| | | | tumor; cervix, testis | | |
| | | | cancer; soft-tissue | | |
| | | | sarcomas | | |
| Alkylating Agents | Nitrogen Mustard | Melphalan (L- | Multiple myeloma; | alkylates DNA at | bone marrow |
| | | sarcolysin; | breast, ovarian cancer | the N7 position of | suppression, |
| | | Alkeran ®) | | guanine and | hemolytic anemia, |
| | | | | induces DNA inter- | pulmonary fibrosis, |
| | | | | strand cross- | diarrhea, oral |
| | | | | linkages, resulting | ulceration, and |
| | | | | in the inhibition of | alopecia |
| | | | | DNA and RNA | |
| | | | | synthesis and | |
| | | | | cytotoxicity against | |
| | | | | both dividing and | |
| | | | | non-dividing tumor | |
| | | | | cells | |
| Alkylating Agents | Nitrosourea | Carmustine (BCNU; | Hodgkin's disease, | alkylates and cross- | headache, asthenia |
| | | Gliadel Wafer ®) | non-Hodgkin's | links DNA during | (weakness), fever, |
| | | | lymphomas, primary | all phases of the | abdominal pain, |
| | | | brain tumors, | cell cycle, resulting | nausea, vomiting, |
| | | | multiple myeloma, | in disruption of | constipation, |
| | | | malignant myeloma | DNA function, cell | hemiplegia |
| | | | | cycle arrest, and | (paralysis), |
| | | | | apoptosis. This | convulsion, |
| | | | | agent also | depression, anxiety, |
| | | | | carbamoylates | rash, and alopecia |
| | | | | proteins, including | |
| | | | | DNA repair | |
| | | | | enzymes, resulting | |
| | | | | in an enhanced | |
| | | | | cytotoxic effect | |
| Alkylating Agents | Nitrosurea | Lomustine | primary/metastatic | alkylates and | delayed |
| | | (CEENU) | brain tumors; | crosslinks DNA, | myelosuppression, |
| | | | Hodgkin's disease | thereby inhibiting | hepatotoxicity, |
| | | | | DNA and RNA | azotemia |
| | | | | synthesis. This | (accumulation of |
| | | | | agent also | nitrogenous waste |
| | | | | carbamoylates | in the blood), renal |
| | | | | DNA and proteins, | failure and alopecia |
| | | | | resulting in | |
| | | | | inhibition of DNA | |
| | | | | and RNA synthesis | |
| | | | | and disruption of | |
| | | | | RNA processing | |
| Alkylating Agents | Sulfhydryl | Mesna (Mesnex ®) | reduces the incidence | Mesna is converted | leukopenia, fatigue, |
| | | | of hemorrhagic | to a free thiol | fever, |
| | | | cystitis associated | compound in the | thrombcytopenia |
| | | | with certain | kidney, where it | (decrease in number |
| | | | chemotherapeutic | binds to and | of blood platelets), |
| | | | agents, such as | inactivates acrolein | granulocytopenia |
| | | | ifosfamide | and other urotoxic | (deficiency of blood |
| | | | | metabolites of | granulocytes), and |
| | | | | ifosfamide and | alopecia. |
| | | | | cyclophosphamide, | |
| | | | | thereby reducing | |
| | | | | their toxic effects | |
| | | | | on the urinary tract | |
| | | | | during urinary | |
| | | | | excretion. | |

Chemotherapeutic alkylating agents become strong electrophiles through the formation of carbonium ion intermediates or of transition complexes with the target molecules. This results in the formation of covalent linkages by alkylation of various nucleophilic moieties, such as phosphate, amino, sulfhydryl, hydroxyl, carboxyl, and imidazole groups. The chemotherapeutic and cytotoxic effects of alkylating agents are related directly to alkylation of DNA, which has several sites that are susceptible to the formation of a covalent bond.

The most important pharmacological actions of alkylating agents are those that disturb DNA synthesis and cell division. The capacity of these drugs to interfere with DNA integrity and function in rapidly proliferating tissues provides the basis for their therapeutic applications and for many of their toxic properties. Whereas certain alkylating agents may have damaging effects on tissues with normally low mitotic indices, such as the liver, kidney, and mature lymphocytes, they are most cytotoxic to rapidly proliferating tissues in which a large proportion of the cells are in division. These alkylating compounds may readily alkylate nondividing cells, but their cytotoxicity is enhanced markedly if DNA is damaged in cells programmed to divide. In contrast to many other antineoplastic agents, the effects of the alkylating drugs, although dependent on proliferation, are not cell-cycle-specific, and the drugs may act on cells at any stage of the cycle. However, the toxicity is usually expressed when the cell enters the S phase and the progression through the cycle is blocked. DNA alkylation itself may not be a lethal event if DNA repair enzymes can correct the lesions in DNA prior to the next cellular division.

Alkylating agents differ in their patterns of antitumor activity and in the sites and severity of their side effects. Most cause dose-limiting toxicity to bone marrow elements and to intestinal mucosa and alopecia. Most alkylating agents, including nitrogen mustard, melphalan, chloramucil, cyclophosphamide, and ifosfamide, produce an acute myelosuppression. Cyclophosphamide has lesser effects on peripheral blood platelet counts than do other alkylating agents. Busuflan suppresses all blood elements and may produce a prolonged and cumulative myelosuppression lasting months. BCNU and other chloroethylnitrosoureas cause delayed and prolonged suppression of both platelets and granulocytes.

Alkylating agents also suppress both cellular and humoral immunity, although immunosuppression is reversible at doses used in most anticancer protocols.

In addition to effects on the hematopoietic system, alkylating agents are highly toxic to dividing mucosal cells. The mucosal effects are particularly significant in high-dose chemotherapy protocols associated with bone marrow reconstitution; they may predispose a patient to bacterial sepsis arising from the gastrointestinal tract. Generally, mucosal and bone marrow toxicities occur predictably with conventional doses of these drugs; however other organ toxicities, although less common, can be irreversible and sometimes lethal. All alkylating agents have caused pulmonary fibrosis.

In high-dose protocols, a number of toxicities not seen at conventional doses becomes dose-limiting. For example, endothelial damage that may precipitate venoocclusive disease of the liver; the nitrosoureas, after multiple cycles of therapy, may lead to renal failure; ifosamide frequently causes a central neurotoxicity (manifest in the form of nausea and vomiting), with seizures, coma and sometimes death. Cyclophosamide and ifosfamide release a nephrotoxic and urotoxic metabolite, acrolein, which causes severe hemorrhagic cystitis, a side effect that in high-dose regimens can be prevented by coadministration of mesna (2-mercaptoethanesulfonate).

The more unstable alkylating agents (particularly nitrogen mustards and the nitrosoureas) have strong vesicant properties, damage veins with repeated use, and if extravasated, produce ulceration.

As a class of drugs, the alkylating agents are highly leukomogenic. Acute nonlymphocytic leukemia may affect up to 5% of patients treated on regimens containing alkylating drugs. Melphalan, the nitrosoureas, and procarbazine have the greatest propensity to cause leukemia. Additionally, all alkylating agents have toxic effects on the male and female reproductive systems.

Examples of alklyating agents include, but are not limited to, cyclophosamide (Cytotaxan®), a synthetic alkylating agent chemically related to the nitrogen mustards; temozolomide (Temodar®), a triazene analog of dacarbazine; busulfan (Myleran®), a synthetic derivative of dimethane sulfonate; ifosfamide (Ifex®), a synthetic analog of cyclophosaphamide; mesna (Mesnex®), a sulfhydryl compound; melphalan hydrochloride (Alkeran®), an orally available phenylalanine derivative of nitrogen mustard; and the nitrosoureas carmustine (BiCNU®) and lomustine (CEENU®).

### 2. Antimetabolites

Antimetabolites are a class of drugs that interfere with DNA and RNA growth by preventing purines (azathioprine, mercaptopurine) or pyrimidine from becoming incorporated into DNA during the S phase of the cell cycle, thus stopping normal development and division. Antimetabolites commonly are used to treat leukemias, tumors of the breast, ovary and the intestinal tract, as well as other cancers.

Antimetabolites include folic acid analogs, such as methotrexate and aminopterin; pyrimidine analogs, such as fluorouracil and fluorodeoxyuridine; cytarabine (cytosine arabinoside); and purine analogs, such as mercaptopurine, thioguanine, fludarabine phosphate, pentostatin (2′-deoxycoformycin), and cladribine. Table 2 presents examples of some antimetabolites useful for treating neoplastic diseases.

TABLE 2
Examples of Antimetabolites Useful for Treating Neoplastic Diseases

| | | | Proposed Mechanism | | Known Side- |
|---|---|---|---|---|---|
| Class | Type of Agent | Example | of Action | Neoplasms/Disease | Effects |
| Antimetabolites | Pyrimidine | 5-fluorouracil | Fluorouracil and its | palliative treatment | stomatitis, |
| | Analog | (fluorouracil; 5-FU) | metabolites possess a | of colorectal cancer, | diarrhea, nausea, |
| | | | number of different | breast cancer, | vomiting (emesis), |
| | | | mechanisms of | stomach cancer, and | leukopenia, and |
| | | | action. In vivo, | pancreatic cancer. In | alopecia. |
| | | | fluoruracil is | combination, with | |
| | | | converted to the | other drugs it is used | |
| | | | active metabolite 5- | to treat locally | |
| | | | fluoroxyuridine | advanced squamous | |
| | | | monophosphate (F- | cell carcinoma of the | |
| | | | UMP); replacing | head and neck, | |
| | | | uracil, F-UMP | gastric | |
| | | | incorporates into | adenocarcinoma, and | |
| | | | RNA and inhibits | Stage III colorectal | |
| | | | RNA processing, | cancer. | |
| | | | thereby inhibiting cell | | |
| | | | growth. Another | | |
| | | | active metabolite, 5- | | |
| | | | 5-fluoro-2′- | | |
| | | | deoxyuridine-5′-O- | | |
| | | | monophosphate (F- | | |
| | | | dUMP), inhibits | | |
| | | | thymidylate synthase, | | |
| | | | resulting in the | | |
| | | | depletion of | | |
| | | | thymidine | | |
| | | | triphosphate (TTP), | | |
| | | | one of the four | | |
| | | | nucleotide | | |
| | | | triphosphates used in | | |
| | | | the in vivo synthesis | | |
| | | | of DNA. Other | | |
| | | | fluorouracil | | |
| | | | metabolites | | |
| | | | incorporate into both | | |
| | | | RNA and DNA; | | |
| | | | incorporation into | | |
| | | | RNA results in major | | |
| | | | effects on both RNA | | |
| | | | processing and | | |
| | | | functions. | | |
| Antimetabolites | Pyrimidine | Capecitabine | As a prodrug, | metastatic (Stage III) | diarrhea, nausea, |
| | Analog | (Xeloda ®) | capecitabine is | colorectal cancer and | vomiting, |
| | | | selectively activated | metastatic breast | stomatitis, hand- |
| | | | by tumor cells to its | cancer. | and-foot- |
| | | | cytotoxic moiety, 5- | | syndrome, |
| | | | fluorouracil (5-FU); | | dermatitis, fatigue, |
| | | | subsequently, 5-FU is | | insomnia, |
| | | | metabolized to two | | anorexia, myalgia, |
| | | | active metabolites, 5- | | neutropenia, |
| | | | fluoro-2-deoxyuridine | | depression, and |
| | | | monophosphate | | alopecia. |
| | | | (FdUMP) and 5- | | |
| | | | fluorouridine | | |
| | | | triphosphate (FUTP) | | |
| | | | by both tumor cells | | |
| | | | and normal cells. | | |
| | | | FdUMP inhibits | | |
| | | | DNA synthesis and | | |
| | | | cell division by | | |
| | | | reducing normal | | |
| | | | thymidine | | |
| | | | production, while | | |
| | | | FUTP inhibits RNA | | |
| | | | and protein synthesis | | |
| | | | by competing with | | |
| | | | uridine triphosphate | | |
| | | | for incorporation into | | |
| | | | the RNA strand. | | |
| Antimetabolites | Pyrimidine | Gemcitabine | Gemcitabine is | pancreatic cancer, | myelosuppression, |
| | Analog | (gemcitabine | converted | ovarian cancer, breast | proteinuria, nausea |
| | | hydrochloride) | intracellularly to the | cancer, and non- | and vomiting, pain, |
| | | | active metabolites | small cell lung | fever, dyspnea, and |
| | | | difluorodeoxycytidine | cancer. | alopecia. |
| | | | di- and triphosphate | | |
| | | | (dFdCDP, dFdCTP). | | |
| | | | dFdCDP inhibits | | |
| | | | ribonucleotide | | |
| | | | reductase, thereby | | |
| | | | decreasing the | | |
| | | | deoxynucleotide pool | | |
| | | | available for DNA | | |
| | | | synthesis; dFdCTP is | | |
| | | | incorporated into | | |
| | | | DNA, resulting in | | |
| | | | DNA strand | | |
| | | | termination and | | |
| | | | apoptosis. | | |
| Antimetabolites | Pyrimidine | floxuridine (FUDR) | inhibits thymidylate | the palliative | diarrhea, enteritis, |
| | Analog | | synthetase, resulting | treatment of | stomatitis, anemia, |
| | | | in disruption of DNA | gastrointestinal | leukopenia, |
| | | | synthesis and | adenocarcinoma | thrombocytopenia |
| | | | cytotoxicity. This | metastatic to the | and alopecia |
| | | | agent is also | liver. | |
| | | | metabolized to | | |
| | | | fluorouracil and other | | |
| | | | metabolites that can | | |
| | | | be incorporated into | | |
| | | | RNA and inhibit the | | |
| | | | utilization of | | |
| | | | preformed uracil in | | |
| | | | RNA synthesis | | |
| Antimetabolites | Pyrimidine | Decitabine | incorporates into | Myelodysplastic | neutropenia, |
| | Analog | (Dacogen ®) | DNA and inhibits | syndromes including | thrombocytopenia, |
| | | | DNA | refractory anemia | nausea, |
| | | | methyltransferase, | and chronic | constipation, |
| | | | resulting in | myelomonocytic | hemorrhoids, |
| | | | hypomethylation of | leukemia | insomnia, anxiety, |
| | | | DNA and intra-S- | | and alopecia |
| | | | phase arrest of DNA | | |
| | | | replication | | |
| Antimetabolites | Purine Analog | Mercaptopurine (6- | a thiopurine- | Acute lymphocytic, | bone marrow |
| | | mercaptopurine; 6- | derivative | acute granulocytic, | toxicity, |
| | | MP; Purinethol ®) | antimetabolite with | and chronic | hepatotoxicity, |
| | | | antineoplastic and | granulocytic | hyperpigmentation, |
| | | | immunosuppressive | leukemias | and alopecia |
| | | | activities. | | |
| Antimetabolites | Folic Acid | Pemetrexed | binds to and inhibits | Mesothelioma, non- | bone marrow |
| | Analogs | disodium (Alimta ®) | the enzyme | small cell lung | suppression, |
| | | | thymidylate synthase | cancer | fatigue, vomiting, |
| | | | (TS) which catalyses | | nausea, |
| | | | the methylation of 2′- | | constipation, |
| | | | deoxyuridine-5′- | | dyspnea, |
| | | | monophosphate | | depression, rash, |
| | | | (dUMP) to 2′- | | myalgia, and |
| | | | deoxythymidine-5′- | | alopecia |
| | | | monophosphate | | |
| | | | (dTMP), an essential | | |
| | | | precursor in DNA | | |
| | | | synthesis | | |
| Antimetabolite | Folic Acid Analog | Methotrexate | binds to and inhibits | chorioadenoma | dizziness, |
| | | (methotrexate | the DHFR, resulting | destruens, | headache, seizures, |
| | | sodium, | in inhibition of purine | choriocarcinoma, | decreased appetite, |
| | | amethopterin, | nucleotide and | acute lymphoblastic | and alopecia |
| | | Folex ®, Mexate ®, | thymidylate synthesis | leukemia, breast | |
| | | Rheumatrex ®) | and, subsequently, | cancer, lung cancer, | |
| | | | inhibition of DNA | certain types of head | |
| | | | and RNA syntheses | and neck cancer, | |
| | | | | advanced non- | |
| | | | | Hodgkin lymphoma, | |
| | | | | and osteosarcoma; | |
| | | | | rheumatoid arthritis | |
| | | | | and psoriasis | |

2.1. Anti-Folates and their Side-Effects

Folic acid is an essential dietary factor from which is derived a series of tetrahydrofolate cofactors that provide single carbon groups for the synthesis of precursors of DNA (thymidylate and purines) and RNA (purines). The enzyme dihydrofolate reductase (“DHFR”) is the primary site of action of most anti-folates Inhibition of DHFR leads to toxic effects through partial depletion of tetrahydrofolate cofactors that are required for the synthesis of purines and thymidylate.

Examples of anti-folates include, but are not limited to, methotrexate and Pemetrexed disodium. The most commonly used anti-folate is methotrexate (methotrexate sodium, amethopterin, Folex®, Mexate®, Rheumatrex®, which is an antimetabolite and antifolate agent with antineoplastic and immunosuppressant activities. Pemetrexed disodium (Alimta®) is the disodium salt of a synthetic pyrimidine-based antifolate.

2.2. Pyrmidine Analogs and their Side-Effects

Pyrmidine analogs are a diverse group of drugs with the capacity to inhibit biosynthesis of pyrimidine nucleotides or to mimic these natural metabolites to such an extent that the analogs interfere with the synthesis or function of nucleic acids. Drugs in this group have been employed in the treatment of diverse afflictions, including neoplastic diseases, psoriasis and infections caused by fungi and DNA-containing viruses.

Examples of pyrimidine analogs include, but are not limited to, 5-Fluorouracil (fluorouracil, 5-FU, Adrucil®, Efudex®, Fluorplex®), an antimetabolite fluoropyrimidine analog of the nucleoside pyrimidine with antineoplastic activity; floxuridine, a fluorinated pyrimidine monophosphate analogue of 5-fluoro-2′-deoxyuridine-5′-phosphate (FUDR-MP) with antineoplastic activity; capecitabine (Xeloda®), an antineoplastic fluoropyrimidine carbamate; and gemcitabine hydrochloride (Gemzar®), the salt of an analog of the antimetabolite nucleoside deoxycytidine with antineoplastic activity.

2.3. Purine Analogs and their Side-Effects

Several analogs of natural purine bases, nucleosides and nucleotides useful in the treatment of malignant diseases (mercaptopurine, thioguanine) and for immunosuppressive (azatioprine) and antiviral (acyclovir, ganciclovir, vidarabine, zidovudine) therapies have been identified.

The purine analogs mercaptopurine and its derivative azatioprine are among the most clinically useful drugs of the antimetabolite class. Examples of purine analogs include, but are not limited to, mercaptopurine (Purinethol®), a thiopurine-derivative antimetabolite with antineoplastic and immunosuppressive activities; decitabine (Dacogen®), a cytidine antimetabolite analogue with potential antineoplastic activity; and dacarbazine (DTIC-DOME®), a triazene derivative with antineoplastic activity.

3. Natural Products and their Side-Effects

Many chemotherapeutic agents are found or derived from natural resources. Table 3 shows some chemotherapeutic drugs classified as natural products.

TABLE 3
Examples of Natural Products Useful to Treat Neoplastic Diseases

| | | | Proposed | | |
|---|---|---|---|---|---|
| | | | Mechanism of | | |
| Class | Type of Agent | Example | Action | Neoplasms/Disease | Known Side-Effects |
| Natural Products | Vinca Alkaloid | Vincristine | binds irreversibly | Acute lymphocytic | abdominal cramps, |
| | | (vincristine sulfate) | to microtubules | leukemia, | weight loss, nausea, |
| | | | and spindle | neuroblastoma, | vomiting, diarrhea, |
| | | | proteins in S | Wilm's tumor, | constipation, |
| | | | phase of the cell | rhabdomyosarcoma, | hypotension, dysuria |
| | | | cycle and | Hodgkin's disease, | (painful urination), rash, |
| | | | interferes with the | non-Hodgkin's | fever and alopecia |
| | | | formation of the | lymphomas, small- | |
| | | | mitotic spindle, | cell lung cancer | |
| | | | thereby arresting | | |
| | | | tumor cells in | | |
| | | | metaphase | | |
| Natural Products | Vinca Alkaloid | Vinblastine | binds to tubulin | Hodgkin's disease, | dyspnea, leukopenia, |
| | | (vinblastine sulfate, | and inhibits | non-Hodgkin's | anemia, |
| | | VLB) | microtubule | lymphomas, breast | thrombocyotpenia, |
| | | | formation, | and testis cancer | constipation, diarrhea |
| | | | resulting in | | and alopecia |
| | | | disruption of | | |
| | | | mitotic spindle | | |
| | | | assembly and | | |
| | | | arrest of tumor | | |
| | | | cells in the M | | |
| | | | phase of the cell | | |
| | | | cycle | | |
| Natural Products | Vinca Alkaloid | Vinorelbine tartrate | binds to tubulin, | Advanced non-small | granulocytopenia, |
| | | (Navelbine ®) | thereby inhibiting | cell lung cancer | allergic reactions, |
| | | | tubulin | | leukopenia, anemia, |
| | | | polymerization | | asthenia (weakness), |
| | | | into microtubules | | phlebitis, diarrhea, |
| | | | and spindle | | severe constipation, |
| | | | formation and | | paralytic ileus, |
| | | | resulting in | | intestinal obstruction, |
| | | | apoptosis of | | necrosis, and/or |
| | | | susceptible cancer | | perforation, dyspnea |
| | | | cells. | | (difficult breathing), |
| | | | | | chest pain, fatigue and |
| | | | | | alopecia |
| Natural Products | Taxane | Paclitaxel (Taxol ®) | inhibitor of | Ovarian, breast, lung, | neutropenia, mucositis, |
| | | | mitosis, differing | head and neck | leukopenia, |
| | | | from the vinca | cancer; used in | thrombocytopenia, |
| | | | alkaloids and | combination therapy | anemia, infections, |
| | | | colchicine | of cisplatin- | bleeding, hypotension, |
| | | | derivatives in that | refractory ovarian, | peripheral neuropathy, |
| | | | it promotes rather | breast, (non-small | nausea, vomiting, |
| | | | than inhibits | cell) lung, | diarrhea and alopecia |
| | | | microtubule | esophagus, bladder, | |
| | | | formation | and head and neck | |
| | | | | cancers | |
| Natural Products | Taxane | Docetaxel | binds to and | Ovarian, breast, lung, | arthralgia (joint pain), |
| | | (Taxotere ®) | stabilizes tubulin, | head and neck | myalgia, febrile |
| | | | thereby inhibiting | cancer; locally | neutropenia, stomatitis, |
| | | | microtubule | advanced or | nausea, vomiting, |
| | | | disassembly | metastatic breast | diarrhea, nail changes, |
| | | | which results in | cancer and non-small | and alopecia |
| | | | cell-cycle arrest | cell lung cancer, and | |
| | | | at the G2/M phase | used with other | |
| | | | and cell death | agents in the | |
| | | | | treatment of | |
| | | | | metastatic prostate | |
| | | | | cancer, advanced | |
| | | | | gastric | |
| | | | | adenocarcinoma, and | |
| | | | | locally advanced | |
| | | | | squamous cell | |
| | | | | carcinoma of the | |
| | | | | head and neck | |
| Natural Products | Epothilone | Ixabepilone | binds to tubulin | Non-Hodgkin's | headache, flaky or |
| | | (Ixempra ®, INN, | and promotes | lymphoma; breast | darkened skin, loss of |
| | | azaepothilone B) | tubulin | cancer | appetite, diarrhea, |
| | | | polymerization | | nausea, vomiting, |
| | | | and microtubule | | constipation, weakness, |
| | | | stabilization, | | hair loss and alopecia |
| | | | thereby arresting | | |
| | | | cells in the G2-M | | |
| | | | phase of the cell | | |
| | | | cycle and | | |
| | | | inducing tumor | | |
| | | | cell apoptosis | | |
| Natural Products | Anthracycline | Daunorubicin | daunorubicin | Acute granulocytic | cardiac toxicity, |
| | | (Cerubidine ®, | exhibits cytotoxic | and acute | including tachycardia, |
| | | daunomycin, | activity through | lymphocytic | arrhythmias, dyspnea, |
| | | rubidomycin) | topoisomerase- | leukemias | hypotension, pericardial |
| | | | mediated | | effusion; |
| | | | interaction with | | myelosuppression, |
| | | | DNA, thereby | | diarrhea, abdominal |
| | | | inhibiting DNA | | pain, hyperuricemia, |
| | | | replication and | | mucositis, stomatitis, |
| | | | repair and RNA | | gastrointestinal |
| | | | and protein | | disturbances, |
| | | | synthesis | | dermatological |
| | | | | | manifestations, and |
| | | | | | alopecia |
| Natural Products | Anthracycline | Epirubicin | intercalates into | Breast cancer | hematologic |
| | | (Ellence ®) | DNA and | | abnormalities, |
| | | | interacts with | | amenorrhea (absence of |
| | | | topoisomerase II, | | the menses), hot |
| | | | thereby inhibiting | | flashes, lethargy, fever, |
| | | | DNA replication | | gastrointestinal |
| | | | and repair and | | disturbances, infection, |
| | | | RNA and protein | | conjunctivitis/keratitis |
| | | | synthesis | | and alopecia |
| Natural Products | Anthracycline | Doxorubicin | intercalates | Soft-tissue, | myelosuppression, |
| | | (Doxil ®, | between base | osteogenic, and other | thrombocytopenia, |
| | | doxorubicin | pairs in the DNA | sarcomas; Hodgkin's | anemia, asthenia, |
| | | hydrochloride, | helix, thereby | disease, non- | nausea, vomiting, |
| | | Adriamycin ®, | preventing DNA | Hodgkin's | diarrhea, stomatitis, |
| | | Rubex ®) | replication and | lymphomas; acute | hand-foot syndrome |
| | | | ultimately | leukemias; breast, | and alopecia |
| | | | inhibiting protein | genitourinary, | |
| | | | synthesis; inhibits | thyroid, lung, | |
| | | | topoisomerase II | stomach cancer; | |
| | | | which results in | neuroblastoma | |
| | | | an increased and | | |
| | | | stabilized | | |
| | | | cleavable | | |
| | | | enzyme-DNA | | |
| | | | linked complex | | |
| | | | during DNA | | |
| | | | replication and | | |
| | | | subsequently | | |
| | | | prevents the | | |
| | | | ligation of the | | |
| | | | nucleotide strand | | |
| | | | after double- | | |
| | | | strand breakage | | |
| Natural Products | Anthracycline | Idarubicin | intercalates into | Acute myeloid | severe |
| | | (idarubicin | DNA and | leukemia | myelosuppression, |
| | | hydrochloride, | interferes with the | | nausea, vomiting, |
| | | Idamycin PFS ®) | activity of | | abdominal pain, |
| | | | topoisomerase II, | | diarrhea, rash and |
| | | | thereby inhibiting | | alopecia |
| | | | DNA replication, | | |
| | | | RNA | | |
| | | | transcription and | | |
| | | | protein synthesis | | |
| Natural Products | Anthracenedione | Mitoxantrone | stimulates the | Acute granulocytic | nausea, menstrual |
| | | (Novantrone ®) | formation of | leukemia, breast and | disorder, amenorrhea, |
| | | | strand breaks in | prostate cancer | upper respiratory |
| | | | DNA (mediated | | infection, urinary tract |
| | | | by topoisomerase | | infection, stomatitis, |
| | | | II) and also by | | acute |
| | | | intercalating with | | myelosuppression, |
| | | | DNA | | cardiac toxicity, |
| | | | | | mucositis, vomiting and |
| | | | | | alopecia |
| Natural Products | Antibiotic | Mitomycin | bioreduced | Stomach, cervix, | myelosuppression |
| | | (mitocyin C; | mitomycin C | colon, breast, | characterized by |
| | | Mutamycin ®) | generates oxygen | pancreas, bladder, | marked leukopenia and |
| | | | radicals, alkylates | head and neck cancer | thrombocytopenia, |
| | | | DNA, and | | cellulitis, stomatitis, |
| | | | produces | | skin necrosis, |
| | | | interstrand DNA | | renal/pulmonary/cardiac |
| | | | cross-links, | | toxicity, nausea, |
| | | | thereby inhibiting | | vomiting, diarrhea, |
| | | | DNA synthesis. | | dermatitis, fever, |
| | | | Preferentially | | malaise, hemolytic |
| | | | toxic to hypoxic | | uremic syndrome and |
| | | | cells, mitomycin | | alopecia |
| | | | C also inhibits | | |
| | | | RNA and protein | | |
| | | | synthesis at high | | |
| | | | concentrations | | |
| Natural Products | Antibiotic | Dactinomycin | intercalates | Choriocarcinoma, | malaise, fatigue, |
| | | (Cosmogen ®, | between adjacent | Wilm's tumor, | lethargy, fever, myalgia |
| | | actinomycin D) | guanine-cytosine | rhabdomyosarcoma, | (muscular rheumatism), |
| | | | base pairs, | testis, Kaposi's | proctitis (inflammation |
| | | | blocking the | sarcoma | of the rectum and anus), |
| | | | transcription of | | anorexia, nausea, |
| | | | DNA by RNA | | vomiting, diarrhea, |
| | | | polymerase; it | | glossitis, cheilitis, and |
| | | | also causes | | ulcerations of the oral |
| | | | single-strand | | mucosa, as well as |
| | | | DNA breaks, | | dermatological |
| | | | possibly via a | | symptoms including |
| | | | free-radical | | erythema, |
| | | | intermediate or an | | desquamation, |
| | | | interaction with | | inflammation, changes |
| | | | topoisomerase II | | in pigmentation, and |
| | | | | | alopecia |
| Natural Products | Camptothecin | Irinotecan | prodrug, is | Ovarian cancer, | severe neutropenia, |
| | | (Camptosar ®, | converted to a | small cell lung | febrile neutropenia, |
| | | irinotecan | biologically | cancer, colon cancer | acute diarrhea, |
| | | hydrochloride) | active metabolite | | diaphoresis, |
| | | | 7-ethyl-10- | | hypersalivation, |
| | | | hydroxy- | | abdominal cramps, |
| | | | camptothecin | | visual accommodation |
| | | | (SN-38) by a | | disturbances, |
| | | | carboxylesterase- | | lacrimation (tearing), |
| | | | converting | | rhinorrhea (runny nose), |
| | | | enzyme. One | | asymptomatic |
| | | | thousand-fold | | bradycardia, nausea and |
| | | | more potent than | | vomiting, fatigue, |
| | | | its parent | | vasodilation or skin |
| | | | compound | | flushing, mucositis, |
| | | | irinotecan, SN-38 | | liver transaminases |
| | | | inhibits | | elevation, blood |
| | | | topoisomerase I | | dyscrasias (an |
| | | | activity by | | imbalance of the blood |
| | | | stabilizing the | | constituents), asthenia, |
| | | | cleavable | | anorexia, fever, pain, |
| | | | complex between | | constipation, infection, |
| | | | topoisomerase I | | dyspnea, increased |
| | | | and DNA, | | bilirubin and alopecia |
| | | | resulting in DNA | | |
| | | | breaks that inhibit | | |
| | | | DNA replication | | |
| | | | and trigger | | |
| | | | apoptotic cell | | |
| | | | death | | |
| Natural Products | Camptothecin | Topotecan | during the S | Ovarian cancer, | neutropenia with or |
| | | (Hycamtin ®, | phase of the cell | small cell lung | without |
| | | topotecan | cycle, topotecan | cancer, colon cancer | thrombocytopenia, |
| | | hydrochloride) | selectively | | mucositis, diarrhea, |
| | | | stabilizes | | nausea and vomiting, |
| | | | topoisomerase I- | | elevated liver |
| | | | DNA covalent | | transaminases, fever, |
| | | | complexes, | | fatigue, rash, anorexia, |
| | | | inhibiting | | dyspnea (difficult |
| | | | religation of | | breathing), and alopecia |
| | | | topoisomerase I- | | |
| | | | mediated single- | | |
| | | | strand DNA | | |
| | | | breaks and | | |
| | | | producing | | |
| | | | potentially lethal | | |
| | | | double-strand | | |
| | | | DNA breaks | | |
| | | | when complexes | | |
| | | | are encountered | | |
| | | | by the DNA | | |
| | | | replication | | |
| | | | machinery | | |
| Natural Products | Epipodophyllotoxin | Etoposide | binds to and | Testis, small-cell | leukopenia, |
| | | (VePesid ®) | inhibits | lung and other lung, | myelosuppression, |
| | | | topoisomerase II | breast cancer; | anemia, stomatitis, |
| | | | and its function in | Hodgkin's disease, | hypotension, anorexia, |
| | | | ligating cleaved | non-Hodgkin's | thrombocytopenia, |
| | | | DNA molecules, | lymphomas, acute | fever, phlebitis, |
| | | | resulting in the | granulocytic | dermatitis, allergic |
| | | | accumulation of | leukemia, Kaposi's | reactions (such as |
| | | | single- or double- | sarcoma | anaphylaxis) and |
| | | | strand DNA | | alopecia |
| | | | breaks, the | | |
| | | | inhibition of | | |
| | | | DNA replication | | |
| | | | and transcription, | | |
| | | | and apoptotic cell | | |
| | | | death | | |
| Natural Products | Epipodophyllotoxin | Teniposide | forms a ternary | Testis, small-cell | myelosuppression, |
| | | (Vumon ®) | complex with the | lung and other lung, | nausea, vomiting, |
| | | | enzyme | breast cancer; | leukopenia, |
| | | | topoisomerase II | Hodgkin's disease, | neutropenia, |
| | | | and DNA, | non-Hodgkin's | thrombocytopenia, |
| | | | resulting in dose- | lymphomas, acute | anemia, mucositis, |
| | | | dependent single- | granulocytic | diarrhea, infection, |
| | | | and double- | leukemia, Kaposi's | bleeding, |
| | | | stranded breaks in | sarcoma | hypersensitivity |
| | | | DNA, DNA: | | reactions, rash, fever, |
| | | | protein cross- | | and alopecia |
| | | | links, inhibition | | |
| | | | of DNA strand | | |
| | | | religation, and | | |
| | | | cytotoxicity | | |
| Natural Products | Epipodophyllotoxin | Etoposide | binds to the | Testicular tumors, | nausea and vomiting, |
| | | phosphate | enzyme | small cell lung | loss of appetite, |
| | | (Etopophos ®) | topoisomerase II, | cancer | constipation, diarrhea, |
| | | | inducing double- | | abdominal pain, fatigue, |
| | | | strand DNA | | thinned or brittle hair, |
| | | | breaks, inhibiting | | and alopecia |
| | | | DNA repair, and | | |
| | | | resulting in | | |
| | | | decreased DNA | | |
| | | | synthesis and | | |
| | | | tumor cell | | |
| | | | proliferation. | | |
| | | | Cells in the S and | | |
| | | | G2 phases of the | | |
| | | | cell cycle are | | |
| | | | most sensitive to | | |
| | | | this agent. | | |

3.1. Antimitotic Drugs

3.1.1. Vinca Alkaloids and their Side-Effects

The vinca alkaloids, cell-cycle-specific agents that, in common with other drugs, such as colchicine, podophyllotoxin, and taxanes, block cells in mitosis, exerts their biological activities by specifically binding to tubulin, thereby blocking the ability of protein to polymerize into microtubules, and arresting cell division in metaphase through disruption of the microtubules of the mitotic apparatus. In the absence of an intact mitotic spindle, the chromosomes may disperse throughout the cytoplasm or may clump in unusual groupings. Both normal and malignant cells exposed to vinca alkaloids undergo changes characteristic of apoptosis.

Examples of vinca alkaloids include, but are not limited to, vincristine sulfate, a salt of a natural alkaloid isolated from the plant Vinca rosea Linn; vinblastine, a natural alkaloid isolated from the plant Vinca rosea Linn; and vinorelbine. Both vincristine and vinblastine, as well as the analog vinorelbine, have potent and selective antitumor effects, although their actions on normal tissue differ significantly.

3.1.2. Taxanes

The taxanes include, for example, but not limited to, paclitaxel, extracted from the Pacific yew tree Taxus brevifolia, and docetaxel (Taxotere®), a semi-synthetic, second-generation taxane derived from a compound found in the European yew tree Taxus baccata.

3.2. Epipodophyllotoxins and their Side-Effects

Podophyllotoxin is the active principle extracted from the mandrake plant Podophyllum peltatum from which two semisynthetic glycosides, etoposide and teniposide, have been developed.

3.3. Camptothecin Analogs and their Side-Effects

Camptothecins target the enzyme topoisomerase I. The parent compound, camptothecin, was first isolated from the Chinese tree Camptotheca acuminata. Although the parent camptothecin compound demonstrated antitumor activity, its severe and unpredictable toxicity, principally myelosuppression and hemorrhagic cystitis limited its use. The most widely used camptothecin analogs are irinotecan and toptecan, which are less toxic and more soluble.

3.4. Antibiotics and their Side-Effects

Antitumor antibiotics are compounds that have cytotoxic as well as antimicrobial properties. Most commonly used in neoplastic disease treatment are the actinomycins and anthracyclines. The actinomycins intercalate between adjacent guanine-cytosine base pairs of DNA. These interactions provide great stability to the actinomycin-DNA complex, and as a result block the transcription of DNA and RNA.

3.4.1. Actinomycin

An exemplary actinomycin includes Dactinomycin (Actinomycin D), produced by Streptomyces parvullus. This highly toxic agent inhibits rapidly proliferating cells of normal and neoplastic origin.

3.4.2. Anthracyclines

The anthracycline antibiotics and their derivatives are important antitumor agents. They are produced by the fungus Streptomyces peucetius var. caesius. Anthracyclines and anthracenediones can intercalate with DNA. Accordingly, many functions of DNA are affected, including DNA and RNA synthesis. Single-strand and double-strand breaks occur, as does sister chromatid exchange; thus these compounds are both mutagenic and carcinogenic. Scission of DNA is believed to be mediated by drug binding to DNA and topoisomerase II that prevents the resealing of DNA breaks created by the enzyme.

Examples of anthracyclines include, but are not limited to, idarubicin hydrochloride, a semisynthetic 4-demethoxy analog of daunorubicin (daunorubicin hydrochloride, daunomycin, rubidomycin; Cerubidine®); doxorubicin (doxorubicin hydrochloride, Adriamycin®, Rubex®); as well as several analogs of doxorubicin including valrubicin (Valstar®) (for intravescial therapy of BCG-refractory urinary bladder carcinoma) and epirubicin (4′-epidxorubicin, Ellence®) (as a component of adjuvant therapy following resection of early lymph-node-positive breast cancer).

Additional antibiotic antineoplastics include, but are not limited to, mitoxantrone (Novotrone®), an anthracenedione; and bleomycin antibiotics, fermentation products of Streptomyces verticillus that cleave DNA, and includes bleomycin sulfate (Blenoxane®); and mitomycin (mitomycin-C, Mutamycin®), an antibiotic isolated from Streptomyces caespitosus.

### 4. Biologics

Generally, the term “biologics” refers to compounds that are produced by biological processes, including those utilizing recombinant DNA technology. Biologic compounds include agents or approaches that beneficially affect a patient's biological response to a neoplasm. Included are agents that act indirectly to mediate their anti-tumor effects (e.g., by enhancing the immunological response to neoplastic cells) or directly on the tumor cells (e.g., differentiating agents).

Table 4 shows examples of chemotherapeutic agents that are classified as biologics.

TABLE 4
Examples of Biologics Useful for Treating Neoplastic Diseases

| | | | Proposed | | |
|---|---|---|---|---|---|
| | | | Mechanism of | | Known Side- |
| Class | Type of Agent | Example | Action | Neoplasms/Disease | Effects |
| Biologics | Granulocyte-Colony | Filgrastim | In vitro, G-CSF | Neutropenia | nausea and |
| | Stimulating Factor | (Neupogen ®) | expands the | | vomiting, skeletal |
| | | | population of | | pain, diarrhea, |
| | | | neutrophil | | neutropenic fever, |
| | | | granulocyte | | mucositis, fever, |
| | | | precursors, | | fatigue and |
| | | | augments | | alopecia |
| | | | granulocyte | | |
| | | | function by | | |
| | | | enhancing | | |
| | | | chemotaxis and | | |
| | | | antibody- | | |
| | | | dependent cellular | | |
| | | | cytotoxicity, and | | |
| | | | enhances the | | |
| | | | mobilization of | | |
| | | | stem cells in the | | |
| | | | peripheral blood | | |
| | | | following | | |
| | | | cytotoxic | | |
| | | | chemotherapy | | |
| Biologics | Granulocyte-Colony | Pegfilgrastim | binds to and | Neutropenia | bone pain, |
| | Stimulating Factor | (Neulasta ®) | activates specific | | diarrhea, pyrexia |
| | | | cell surface | | (fever), myaglia, |
| | | | receptors, | | headache, |
| | | | stimulating | | vomiting and |
| | | | neutrophil | | alopecia |
| | | | progenitor | | |
| | | | proliferation and | | |
| | | | differentiation and | | |
| | | | selected neutrophil | | |
| | | | functions. | | |
| | | | Conjugation of the | | |
| | | | cytokine with a | | |
| | | | branched | | |
| | | | polyethylene | | |
| | | | glycol molecule | | |
| | | | (pegylation) | | |
| | | | significantly | | |
| | | | increases its | | |
| | | | therapeutic half- | | |
| | | | life | | |
| Biologics | Monoclonal | Bevacizumab | binds to and | Colorectal cancer, | abdominal pain, |
| | Antibody | (Avastin ®) | inhibits the | non-small cell lung | headache, |
| | | | biologic activity of | cancer, breast cancer | hypertension, |
| | | | human vascular | | vomiting, |
| | | | endothelial growth | | anorexia, |
| | | | factor (“VEGF”) | | constipation, |
| | | | | | stomatitis, |
| | | | | | dizziness, upper |
| | | | | | respiratory |
| | | | | | infection, dyspnea, |
| | | | | | and alopecia |
| Biologics | Granulocyte- | Sargramostim | used following | Acute myelogenous | fever, abdominal |
| | Macrophage Colony | (Leukine ®) | induction | leukemia, | pain, headache, |
| | Stimulating Factor | | chemotherapy in | mobilization and | diarrhea, nausea, |
| | | | patients with acute | engraftment of | vomiting, |
| | | | myelogenous | peripheral blood | stomatitis, |
| | | | leukemia (AML) | progenitor cells | anorexia, rash, and |
| | | | to shorten the time | | alopecia |
| | | | to neutrophil | | |
| | | | recovery and to | | |
| | | | reduce the | | |
| | | | incidence of | | |
| | | | severe and life- | | |
| | | | threatening | | |
| | | | infections; rescue | | |
| | | | bone marrow graft | | |
| | | | failure or speed | | |
| | | | graft recovery in | | |
| | | | patients | | |
| | | | undergoing | | |
| | | | autologous bone | | |
| | | | marrow | | |
| | | | transplantation | | |
| Biologics | Epidermal growth | Panitumumab | attaches to the | Metastatic colorectal | fatigue, abdominal |
| | factor receptor | (Vectibix ®) | transmembrane | carcinoma | pain, nausea, |
| | antagonist | | epidermal growth | | diarrhea, |
| | | | factor (EGF) | | constipation, |
| | | | receptor and may | | vomiting, |
| | | | inhibit autocrine | | stomatitis, |
| | | | EGF stimulation | | hypomagnesemia, |
| | | | of tumor cells that | | severe skin |
| | | | express the EGF | | toxicity including |
| | | | receptor, thereby | | erythema, |
| | | | inhibiting tumor | | acneiform |
| | | | cell proliferation | | dermatitis, |
| | | | | | pruritus, skin |
| | | | | | exfoliation, rash, |
| | | | | | skin fissures, as |
| | | | | | well as severe |
| | | | | | toxicity to hair and |
| | | | | | the growth of |
| | | | | | eyelashes |

Examples of antineoplastic biologics include, but are not limited to, Filgrastim (Neupogen®), a recombinant granulocyte colony-stimulating factor (G-CSF); Pegfilgrastim (Neulasta®), the covalent conjugate of filgrastim and monomethoxypolyethylene glycol; Sargramostim (Leukine®), a recombinant granulocyte/macrophage colony-stimulating factor (GM-CSF);

In biotechnology applications, the term “monoclonal antibodies” (“mAb”) generally refers to identical monospecific immunoglobulin molecules derived from a laboratory procedure from a single cell clone that are capable of binding to an agonist. Fully human monoclonal antibodies have the amino acid sequence of an immunoglobulin of the human species. “Humanized” monoclonal antibodies are constructed from mouse monoclonal antibodies having the desired specificity, and often have complementarity determining regions of a mouse immunoglobulin while maintaining the framework and constant regions of a human antibody to prevent a human-antimouse neutralizing response.

Examples of antineoplastic monoclonal antibodies include, but are not limited to, Bevacizumab (Avastin®), a recombinant humanized monoclonal IgG₁ antibody that binds to and inhibits the biologic activity of human vascular endothelial growth factor (“VEGF”) in in vitro and in vivo assay systems, and Panitumumab (Vectibix®), a human monoclonal antibody produced in transgenic mice that attaches to the transmembrane epidermal growth factor (EGF) receptor.

### 5. Hormones and Related Agents

Several chemotherapeutic agents exert their therapeutic effect through interactions with hormones and related agents. Table 5 shows examples of several chemotherapeutic agents classified as hormone and related agents.

TABLE 5
Examples of Hormones and Antagonists Useful for Treating Neoplastic Diseases

| | | | Proposed | | |
|---|---|---|---|---|---|
| | | | Mechanism of | | Known Side- |
| Class | Type of Agent | Example | Action | Neoplasms/Disease | Effects |
| Hormones and | Progestin | Megestrol Acetate | Mimicking the | Endometrium, breast | diarrhea, |
| Antagonists | | (Megace ES ®) | action of | cancer; anorexia, | flatulence, rash, |
| | | | progesterone, | cachexia (wasting), or | impotence, |
| | | | megestrol binds to | other unexplained | abdominal pain, |
| | | | and activates | weight loss | moniliasis |
| | | | nuclear | | (itching), |
| | | | progesterone | | cardiomyopathy, |
| | | | receptors (PRs) in | | constipation, |
| | | | the reproductive | | leucopenia, |
| | | | system and | | depression, |
| | | | pituitary; ligand- | | neuropathy, |
| | | | receptor | | dyspnea, skin |
| | | | complexes are | | disorder, pruritus, |
| | | | translocated to the | | and alopecia |
| | | | nucleus where | | |
| | | | they bind to | | |
| | | | progesterone | | |
| | | | response elements | | |
| | | | (PREs) located on | | |
| | | | target genes. | | |
| | | | Megestrol's | | |
| | | | antineoplastic | | |
| | | | activity against | | |
| | | | estrogen- | | |
| | | | responsive tumors | | |
| | | | may be due, in | | |
| | | | part, to the | | |
| | | | suppression of | | |
| | | | pituitary | | |
| | | | gonadotropin | | |
| | | | production and the | | |
| | | | resultant decrease | | |
| | | | in ovarian | | |
| | | | estrogen | | |
| | | | secretion; | | |
| | | | interference with | | |
| | | | the estrogen | | |
| | | | receptor complex | | |
| | | | in its interaction | | |
| | | | with genes and; as | | |
| | | | part of the | | |
| | | | progesterone | | |
| | | | receptor complex, | | |
| | | | direct interaction | | |
| | | | with the genome | | |
| | | | and | | |
| | | | downregulation of | | |
| | | | specific estrogen- | | |
| | | | responsive genes. | | |
| | | | This agent may | | |
| | | | also directly kill | | |
| | | | tumor cells | | |
| Hormones and | Antiestrogen | Tamoxifen Citrate | When bound to | breast cancer, | hot flashes, |
| Antagonists | | (Nolvadex ®) | the ER, tamoxifen | especially | depression, nausea |
| | | | induces a change | postmenopausal | and vomiting, |
| | | | in the three- | women with | menstrual |
| | | | dimensional shape | estrogen-receptor | irregularities, |
| | | | of the receptor, | positive (ER+) | vaginal bleeding |
| | | | inhibiting its | metastatic breast | and discharge, |
| | | | binding to the | cancer or following | pruritus vulvae, |
| | | | estrogen- | primary tumor | and dermatitis; it |
| | | | responsive | therapy in the | may increase the |
| | | | element (“ERE”) | adjuvant setting; | risk of |
| | | | on DNA. Under | premenopausal | thromboembolic |
| | | | normal | women with ER+ | events. |
| | | | physiological | tumors. | Additional |
| | | | conditions, | | reported toxicities |
| | | | estrogen | | include diffuse |
| | | | stimulation | | thinning of hair, |
| | | | increases tumor | | changes in hair |
| | | | cell production of | | color and alopecia |
| | | | transforming | | |
| | | | growth factor β | | |
| | | | (“TGF-β”), an | | |
| | | | autocrine inhibitor | | |
| | | | of tumor cell | | |
| | | | growth. | | |
| | | | “Autocrine | | |
| | | | signaling” refers | | |
| | | | to a form of | | |
| | | | signaling in which | | |
| | | | a cell secretes a | | |
| | | | hormone or | | |
| | | | chemical | | |
| | | | messenger | | |
| | | | (autocrine agent) | | |
| | | | that binds to | | |
| | | | autocrine | | |
| | | | receptors on the | | |
| | | | same cell type, | | |
| | | | leading to changes | | |
| | | | in the cells. By | | |
| | | | blocking these | | |
| | | | pathways, the net | | |
| | | | effect of | | |
| | | | tamoxifen | | |
| | | | treatment is to | | |
| | | | decrease the | | |
| | | | autocrine | | |
| | | | stimulation of | | |
| | | | breast cancer | | |
| | | | growth. | | |
| Hormones and | Androgen | Fluoxymesterone | binds to and | Breast cancer; | amenorrhea, |
| Antagonists | | (Halotestin ®) | activates specific | testosterone | gynecomastia |
| | | | nuclear receptors, | replacement therapy | (abnormal |
| | | | resulting in an | in males with primary | enlargement of the |
| | | | increase in protein | hypogonadism or | breast in a male), |
| | | | anabolism, a | hypogonadotrophic | oligospermia |
| | | | decrease in amino | hypogonadism, as | (deficiency of |
| | | | acid catabolism, | well as palliation of | sperm), and male |
| | | | and retention of | androgen-responsive | pattern baldness |
| | | | nitrogen, | recurrent mammary | |
| | | | potassium, and | cancer in females | |
| | | | phosphorus. This | | |
| | | | agent also may | | |
| | | | competitively | | |
| | | | inhibit prolactin | | |
| | | | receptors and | | |
| | | | estrogen | | |
| | | | receptors, thereby | | |
| | | | inhibiting the | | |
| | | | growth of | | |
| | | | hormone- | | |
| | | | dependent tumor | | |
| | | | lines | | |
| Hormones and | Gonadotropin- | Leuprolide | binds to and | Prostate cancer; | lethargy, |
| Antagonists | releasing Hormone | (leuprolide acetate, | activates | endometriosis, | flatulence, |
| | Analog | Eligard ®) | gonadotropin- | anemia secondary to | constipation, |
| | | | releasing hormone | uterine leiomyomas | dyspepsia |
| | | | (GnRH) receptors. | and central | (indigestion), |
| | | | Continuous, | precocious puberty | decreased red |
| | | | prolonged | | blood cell count, |
| | | | administration of | | depression, |
| | | | leuprolide in | | vertigo, insomnia, |
| | | | males results in | | and alopecia |
| | | | pituitary GnRH | | |
| | | | receptor | | |
| | | | desensitization | | |
| | | | and inhibition of | | |
| | | | pituitary secretion | | |
| | | | of follicle | | |
| | | | stimulating | | |
| | | | hormone (FSH) | | |
| | | | and luteinizing | | |
| | | | hormone (LH), | | |
| | | | leading to a | | |
| | | | significant decline | | |
| | | | in testosterone | | |
| | | | production; in | | |
| | | | females, | | |
| | | | prolonged | | |
| | | | administration | | |
| | | | results in a | | |
| | | | decrease in | | |
| | | | estradiol | | |
| | | | production. This | | |
| | | | agent reduces | | |
| | | | testosterone | | |
| | | | production to | | |
| | | | castration levels | | |
| | | | and may inhibit | | |
| | | | androgen | | |
| | | | receptor-positive | | |
| | | | tumor progression | | |
| Hormones and | Somatostatin Analog | Octreotide acetate | suppresses the | Acromegaly, severe | diarrhea, |
| Antagonists | | (Sandostatine LAR | luteinizing | diarrhea/flushing | abdominal pain, |
| | | Depot ®) | hormone response | episodes associated | flatulence, |
| | | | to gonadotropin- | with metastatic | constipation, |
| | | | releasing | carcinoid tumors, | headache, anemia, |
| | | | hormone, | diarrhea associated | hypertension, and |
| | | | decreases | with VIP-secreting | alopecia |
| | | | splanchnic blood | tumors | |
| | | | flow, and inhibits | | |
| | | | the release of | | |
| | | | serotonin, gastrin, | | |
| | | | vasoactive | | |
| | | | intestinal peptide | | |
| | | | (VIP), secretin, | | |
| | | | motilin, pancreatic | | |
| | | | polypeptide, and | | |
| | | | thyroid | | |
| | | | stimulating | | |
| | | | hormone | | |

5.1. Antiestrogens

Antiestrogens are modulators of the estrogen receptor. Estrogens are the family of hormones that promote the development and maintenance of female sex characteristics.

Examples of antiestrogens include, but are not limited to, tamoxifen citrate (Nolvadex®), a competitive inhibitor of estradiol binding to the estrogen receptor (“ER”).

5.2. Gonadotropin-Releasing Hormone Analogs

Gonadotropin-releasing hormone (“GnRH”) analogs are synthetic peptide drugs modeled after human GnRH. They are designed to interact with GnRH receptor. The analogs of GnRH peptide include leuprolide (Lupron®, Eligard®), goserelin (Zoladex®), triptorelin (Trelstar Depot®) and buserelin (Suprefact®). These compounds have biphasic effects on the pituitary. Initially, they stimulate the secretion of both follicle-stimulating hormone (“FSH”) and luteinizing hormone (“LH”). However, with longer-term administration, cells become desensitized to the action of GnRH analogs. As a result, there is inhibition of the secretion of LH and FSH and the concentration of testosterone falls to castration levels in men and estrogen levels fall to postmenopausal values in women.

GnRH analogs have been used to treat prostatic carcinomas. They present several side-effects, including a transient “flare” of disease. Notwithstanding, leuprolide and goserelin have been used for the treatment of metastatic breast cancer. GnRH analogs also have been used in the treatment of endometriosis, anemia secondary to uterine leiomyomas and central precocious puberty. The primary side-effects of GnRH analogs are secondary to the reduction of sex control concentrations and include hot flashes, sweating, nausea, fatigue, and a decrease in bone and muscle mass.

Examples of gonadotropin-releasing hormone analogs include Leuprolide acetate, the salt of a synthetic nonapeptide analog of gonadotropin-releasing hormone.

5.3. Androgens and Antiandrogens

The term “androgen” as used herein refers to any natural or synthetic compound that promotes male characteristics. Examples of antineoplastic androgens include, but are not limited to, fluoxymesterone (Halotestin®), a halogenated derivative of 17-alpha-methyltestosterone.

Antiandrogens are competitive inhibitors that prevent the natural ligands of the androgen receptor from binding to the receptor. These compounds have activity of their own against prostate cancer. They also are effective in preventing the flare reaction induced by the testosterone surge that can occur with GnRH chemotherapy. The antiandrogens may be divided structurally and mechanistically into (1) steroidal and (2) nonsteroidal antiandrogens (“NSAAs”). The steroidal agents have some partial agonist activity at the androgen receptor. These include such compounds as cyproterone acetate (Androcur®) and megestrol acetate (“Megace®). Side-effects include loss of libido, decreased sexual potency, and low testosterone levels. The NSAAs inhibit the translocation of the androgen receptor to the nucleus from the cytoplasm of target cells, thus providing an antiproliferative effect. NSAAs include flutamide (Eulexin®), nilutamide (Nilandron®), and bicalutamide (Casodex®).

Additional antiandrogen agents, include, but are not limited to, megestrol acetate, the salt of megestrol, a synthetic derivative of the naturally occurring female sex hormone progesterone, with progestogenic, antiestrogenic, and antineoplastic activities.

5.4. Somatostatin Analog

Examples of somatostatin analogs include, but are not limited to, octreolide acetate (Sandostatin LAR® Depot), the salt of a synthetic long-acting cyclic octapeptide with pharmacologic properties mimicking those of the natural hormone somatostatin. Octreotide is a more potent inhibitor of growth hormone, glucagon, and insulin than somatostatin.

### 6. Miscellaneous Agents

Table 6 shows examples of other miscellaneous chemotherapeutic agents for treating neoplastic disease.

TABLE 6
Examples of Miscellaneous Agents Useful for Treating Neoplastic Diseases

| | | | Proposed | | |
|---|---|---|---|---|---|
| | | | Mechanism of | | Known Side- |
| Class | Type of Agent | Example | Action | Neoplasms/Disease | Effects |
| Miscellaneous | Kinase inhibitor | Sorafenib | blocks the enzyme | Hepatocellular | fatigue, weight |
| Agent | | (Nexavar ®) | RAF kinase, a | carcinoma, advanced | loss, |
| | | | critical component | renal cell carcinoma | rash/desquamation, |
| | | | of the | | hand-foot skin |
| | | | RAF/MEK/ERK- | | reaction, diarrhea, |
| | | | β signaling | | anorexia, nausea, |
| | | | cascade, thereby | | vomiting, |
| | | | blocking tumor | | abdominal pain, |
| | | | angiogenesis | | pruritus and |
| | | | | | alopecia |
| Miscellaneous | HER1/EGFR | Erlotinib (Tarceva ®) | competes with | Non-small cell lung | fatigue, rash, |
| Agent | tyrosine kinase | | ATP to reversibly | cancer, pancreatic | nausea, anorexia, |
| | inhibitor | | bind to the | cancer | diarrhea, |
| | | | intracellular | | abdominal pain, |
| | | | catalytic domain | | vomiting, pyrexia, |
| | | | of epidermal | | constipation, |
| | | | growth factor | | dyspnea, |
| | | | receptor (EGFR) | | stomatitis, |
| | | | tyrosine kinase, | | depression, |
| | | | thereby reversibly | | dyspepsia, anxiety, |
| | | | inhibiting EGFR | | neuropathy, |
| | | | phosphorylation | | flatulence and |
| | | | and blocking the | | alopecia |
| | | | signal | | |
| | | | transduction | | |
| | | | events and | | |
| | | | tumorigenic | | |
| | | | effects with EGFR | | |
| | | | activation | | |
| Miscellaneous | Platinum | Cisplatin | forms highly | ovarian cancer, non- | nephrotoxicity, |
| Agents | Coordination | | reactive, charged, | small cell lung | ototoxicity, nausea |
| | Complex | | platinum | cancer, and small cell | and vomiting, |
| | | | complexes which | lung cancer; cancer of | peripheral |
| | | | bind to | bladder, head and | neuropathy, |
| | | | nucleophilic | neck, and | myelosuppression, |
| | | | groups such as | endometrium | thrombocytopenia, |
| | | | GC-rich sites in | | anemia, |
| | | | DNA, inducing | | hyperuricemia, |
| | | | intrastrand and | | seizures, hemolytic |
| | | | interstrand DNA | | anemia, cardiac |
| | | | cross-links, as | | abnormalities, |
| | | | well as DNA- | | diarrhea, and |
| | | | protein cross- | | thinned or brittle |
| | | | links. These cross- | | hair |
| | | | links result in | | |
| | | | apoptosis and cell | | |
| | | | growth inhibition | | |
| Miscellaneous | Platinum | Carboplatin | when activated | ovarian cancer, non- | loss of appetite, |
| Agents | Coordination | | intracellularly | small cell lung | abdominal pain, |
| | Complex | | forms reactive | cancer, and small cell | diarrhea, |
| | | | platinum | lung cancer | constipation, |
| | | | complexes that | | nausea and |
| | | | bind to | | vomiting, and |
| | | | nucleophilic | | thinned or brittle |
| | | | groups such as | | hair |
| | | | GC-rich sites in | | |
| | | | DNA, thereby | | |
| | | | inducing | | |
| | | | intrastrand and | | |
| | | | interstrand DNA | | |
| | | | cross-links, as | | |
| | | | well as DNA- | | |
| | | | protein cross- | | |
| | | | links. These | | |
| | | | carboplatin- | | |
| | | | induced DNA and | | |
| | | | protein effects | | |
| | | | result in apoptosis | | |
| | | | and cell growth | | |
| | | | inhibition | | |
| Miscellaneous | Platinum | Oxaliplatin | alkylates | Advanced metastatic | rhinitis |
| Agents | Coordination | (Eloxatin ®) | macromolecules, | carcinoma of colon or | (inflammation of |
| | Complex | | forming both | rectum; colon cancer | the nose), epistaxis |
| | | | inter- and intra- | | (nose drips), |
| | | | strand platinum- | | weight increase, |
| | | | DNA crosslinks, | | conjunctivitis, |
| | | | which result in | | dyspnea, |
| | | | inhibition of DNA | | constipation, and |
| | | | replication and | | alopecia |
| | | | transcription and | | |
| | | | cell-cycle | | |
| | | | nonspecific | | |
| | | | cytotoxicity | | |
| Miscellaneous | EDTA derivative | Dexrazoxane | chelates iron, | Treatment of | nausea, vomiting, |
| Agents | | (TopoTarget ®, | limiting the | extravasation | diarrhea, |
| | | Zincard ®) | formation of free | resulting from IV | stomatitis, |
| | | | radical-generating | anthracycline | myelosuppression, |
| | | | anthracycline-iron | chemotherapy; | altered liver |
| | | | complexes, which | cardiomyopathy | function, fatigue |
| | | | may minimize | associated with | and alopecia |
| | | | anthracycline-iron | doxorubicin | |
| | | | complex-mediated | | |
| | | | oxidative damage | | |
| | | | to cardiac and soft | | |
| | | | tissues. This agent | | |
| | | | also inhibits the | | |
| | | | catalytic activity | | |
| | | | of topoisomerase | | |
| | | | II, which may | | |
| | | | result in tumor | | |
| | | | cell growth | | |
| | | | inhibition | | |
| Miscellaneous | Platelet-reducing | Anagrelide | Putatively | Thrombocythemia, | headache, diarrhea, |
| Agents | Agent | (Agrylin ®, | provides dose- | polycythemia, | asthenia |
| | | anagrelide | related reduction | chronic myelogenous | (weakness), |
| | | hydrochloride) | in platelet | leukemia, other | nausea, abdominal |
| | | | production | myeloproliferative | pain, dyspnea, |
| | | | resulting from a | disorders including | vomiting, pruritus, |
| | | | decrease in | myeloid metaplasia | dyspepsia, |
| | | | megakaryocyte | with myelofibrosis | depression, |
| | | | hypermaturation | | nervousness, and |
| | | | | | alopecia |
| Miscellaneous | Retinoids | Isotretinoin | binds to and | Severe recalcitrant | depression, |
| Agents | | (Accutane ®) | activates nuclear | nodular acne | vasculitis, |
| | | | retinoic acid | | inflammatory |
| | | | receptors (RARs); | | bowel disease, |
| | | | activated RARs | | anemia, decreases |
| | | | serve as | | in bone mineral |
| | | | transcription | | density, |
| | | | factors that | | pseudotumor |
| | | | promote cell | | cerebi, |
| | | | differentiation and | | hyperpigmentation, |
| | | | apoptosis | | hypopigmentation, |
| | | | | | hair abnormalities |
| | | | | | and alopecia |
| Miscellaneous | Retinoids | Bexarotene | selectively binds | Cutaneous | hypertension, |
| Agents | | (Targretin ®) | to and activates | manifestations of T- | constipation, |
| | | | retinoid X | cell lymphoma | flatulence, |
| | | | receptors (RXRs), | | dyspepsia, |
| | | | thereby inducing | | thrombocytopenia, |
| | | | changes in gene | | myalgia, |
| | | | expression that | | depression, |
| | | | lead to cell | | neuropathy, |
| | | | differentiation, | | rhinitis, dyspnea, |
| | | | decreased cell | | acne, urinary tract |
| | | | proliferation, | | infection and |
| | | | apoptosis of some | | alopecia |
| | | | cancer cell types, | | |
| | | | and tumor | | |
| | | | regression | | |
| Miscellaneous | Histone deacetylase | Vorinostat | binds to the | Cutaneous | fatigue, diarrhea, |
| Agents | inhibitor | (Zolinsa ®) | catalytic domain | manifestations of T- | nausea, dysgeusia |
| | | | of the histone | cell lymphoma | (impairment of the |
| | | | deacetylases | | sense of taste), |
| | | | (HDACs) | | thrombocytopenia, |
| | | | | | anorexia, vomiting, |
| | | | | | and alopecia |

6.1. Kinase Inhibitors

Antineoplastic kinase inhibitors include, but are not limited to, Sorafenib tosylate (Nexavar®), a synthetic compound that targets growth signaling and antiogenesis, and Erlotinib hydrochloride (Tarceva®), the salt of a quinazoline derivative with antineoplastic properties.

6.2. Platinum Coordination Complexes

Examples of antineoplastic agents that form platinum coordination complexes include, but are not limited to, Cisplatin (cis-diamminedichloroplatinum (II), Platinol-AQ®), a divalent inorganic water-soluble, platinum containing complex that appears to enter cells by diffusion and reacts with nucleic acids and proteins, is a component of several combination chemotherapy regimens. For example, it is used with bleomycin, etoposide and vinblastine for treating patients with advanced testicular cancer, and with paclitaxel, cyclophosphamide or doxorubicin for treating ovarian cancer.

Another antineoplastic agent that forms a platinum coordination complex is Carboplatin (CBDCA, JM-8), which has a mechanism and spectrum of clinical activity similar to cisplatin, but generally is less reactive than cisplatin.

An additional antineoplastic agent is Oxaliplatin (trans-1-diaminocyclohexane oxalatoplatinum), which, like cisplatin, has a wide range of antitumor activity and is active in ovarian cancer, germ-cell cancer and cervical cancer. Unlike cisplatin, oxaliplatin in combination with 5-fluorouracil is active in colorectal cancer.

6.3. EDTA Derivatives

Other antineoplastic agents include EDTA-derivatives. Such compounds include, but are not limited to, Dexrazoxane hydrochloride (Zincard®), the salt of a bisdioxopiperazine with iron-chelating, chemoprotective, cardioprotective, and antineoplastic activities.

6.4. Platelet-Reducing Agent Anagrelide hydrochloride (Agrlyin®) is a platelet-reducing agent used to treat thrombocythemia, secondary to myeloproliferative disorders, to reduce the elevated platelet count and the risk of thrombosis and to ameliorate associated symptoms including thrombo-hemorrhagic events.

6.5. Retinoids

Retinoids are a group of substances related to vitamin A and function like vitamin A in the body. Retinoids include, but are not limited to, bexarotene (Targretin®), a synthetic retinoic acid agent with potential antineoplastic, chemopreventive, teratogenic and embryotoxic properties; and isotretinoin (Accutane®), a naturally-occurring retinoic acid with potential antineoplastic activity.

6.6. Histone Deacetylase Inhibitors

The histone deacetylase inhibitor vorinostat (Zolinza®) is a synthetic hydroxamic acid derivative with antineoplastic activity, and a second generation polar-planar compound that binds to the catalytic domain of the histone deacetylases (HDACs). This allows the hydroxamic moiety to chelate zinc ion located in the catalytic pockets of HDAC, thereby inhibiting deacetylation and leading to an accumulation of both hyperacetylated histones and transcription factors. Hyperacetylation of histone proteins results in the upregulation of the cyclin-dependant kinase p21, followed by G₁ arrest. Hyperacetylation of non-histone proteins such as tumor suppressor p53, alpha tubulin, and heat-shock protein 90 produces additional anti-proliferative effects. Vorinostat also induces apoptosis and sensitizes tumor cells to cell death processes.

### 7. Chemotherapy Regimens

Chemotherapy regimens commonly are utilized to treat cancer and often are recommended as adjuvant therapy for high-risk cancer patients following surgery. Generally, it is believed that use of multiple drugs that work by different mechanisms of action decreases the likelihood that resistant cancer cells will develop. Further, when these drugs, with different effects, are combined, each drug can be used at its optimal dose without intolerable side-effects.

Standard chemotherapy regimens have been developed for treatment of several neoplasms. For example, a standard regimen for adjuvant treatment for breast cancer is a combination of Adriamycin®, Cytoxan® and Taxol®. This combination combines three different classes of anti-neoplastic agents: (1) Adriamycin® (Rubex®) is the trade name for doxorubicin; which is an anthracycline antibiotic; (2) Cytoxan® (Neosar) is the trade name for cyclophosphamide, which is an alkylating agent; and (3) Taxol® (Onxal™) is the trade name for paclitaxel, which is a plant alkaloid. Each chemotherapeutic agent has its own side-effects, which may vary in severity depending on the dose of the medications.

Other standard chemotherapy regimens have been developed, including those used in adjuvant therapy of colorectal cancer, such as, for example, the combination of infusional 5-fluoruracil, leucovorin and oxaliplatin (FOLFOX®), which combines three different antineoplastic agents: (1) 5-fluorouracil (5-FU, Adrucil®, Efudex®, Fluorplex®), which is a Pyrmidine analog; (2) leucovorin (folinic acid), which is a reduced folic acid; and (3) oxaliplatin (Eloxatin™), which is an alkylating agent. Another chemotherapeutic regimen for colorectal cancer utilizes FOLFOX® with bevacizumab (Avastin®, a monoclonal antibody and anti-angiogenesis drug). Another chemotherapeutic regimen for colorectal cancer utilizes infusional 5-fluoruracil, leucovorin, and irinotecan (FOLFIRI®) with bevacizumab. Irinotecan (Camptosar®, Camptothecin-11, CPT-11) is a plant alkaloid and topoisomerase I inhibitor.

### 8. Hair Loss or Alopecia as a Side-Effect of Chemotherapy

While alopecia is not the most serious of side-effects biologically, it has important emotional consequences to the patient. Hair is important to everyone's appearance and self-image. Both men and women report hair loss as one of the side effects they fear most after being diagnosed with cancer, however, it is an unfortunate reality that many cancer patients have to endure.

Not all chemotherapy drugs cause hair loss (e.g., brittle hair growth, thin hair growth, short hair growth, sparse hair growth) or alopecia, however it is a common side-effect. Chemotherapy hair loss may include hair on the scalp, face, axillary hair, pubic and body hair. Varying from brittle hair growth, thin hair growth, short hair growth, sparse hair growth to alopecia, chemotherapy hair loss occurs over a period of days or weeks. After completion of therapy, regrowth usually occurs in six to eight weeks, however it may be a different color, or texture.

Hair Biology

Hair, a filamentous outgrowth of protein found only on mammals, is integral to our body image and can have a profound influence on our self-esteem and self-confidence. The hair of non-human mammal species is commonly referred to as fur. In some species, hair is absent at certain stages of life.

Hair grows from hair follicles deep in the dermis and projects from the epidermis of the skin. Human skin has two types of hair: vellus hair and terminal hair. Much of human hair is short, underpigmented vellus hair rather than terminal hair. The most noticeable part of human hair is the hair on the head, which can grow longer than on most mammals and is more dense than most hair found elsewhere on the body. The term “scalp” refers to the integument of the upper part of the head, usually including the associated subcutaneous structures. The scalp is the anatomical area bordered by the face anteriorly and by the neck to the sides and posteriorly.

Vellus hair is short, fine, “peach fuzz” body hair. It is a very soft, generally pale, and short hair that grows in most places on the human body in both sexes. It usually is less than two cm long, and the follicles are not connected to sebaceous glands. It is observed most easily in women and children, as they have less terminal hair to obscure it. It also is found in pre-adolescents and in male pattern baldness.

Terminal hair is developed hair, which is generally longer, coarser, thicker and darker than the shorter and finer vellus hair. Phases of growth in terminal hair are more apparent than in vellus hair; it generally has a longer anagen phase. It has associated sebaceous glands, whereas a vellus hair may not. Under certain conditions, such as puberty, some vellus hair may become androgenic hair. Under other conditions, such as male pattern baldness, it may revert to a vellus-like state.

Each hair comprises two structures: the follicle in the skin and the shaft that is visible. The follicle contains several layers. At the base of the follicle is a projection called a papilla, which contains capillaries, or tiny blood vessels, that feed the cells. The living part of the hair, the area surrounding the papilla called the bulb, is the only part fed by the capillaries. The cells in the bulb divide every 23 to 72 hours, faster than any other cells in the body. The follicle is surrounded by two sheaths—an inner root sheath and an outer root sheath. These sheaths protect and mold the growing hair shaft. The inner root sheath follows the hair shaft and ends below the opening of a sebaceous (oil) gland, which produces sebum, a natural conditioner and sometimes an apocrine (scent) gland. The outer root sheath continues all the way up to the sebaceous gland. An erector pili muscle attaches below the sebaceous gland to a fibrous layer around the outer sheath. When this muscle contracts, it causes the hair to stand up.

The primary component of the hair fiber is keratin. Keratins are proteins, long chains (polymers) of amino acids. The hair shaft contains three layers of keratin. The inner layer, which is called the medulla, may not be present. The next layer is the cortex, which makes up the majority of the hair shaft. The outer layer is the cuticle, which is formed by tightly packed scales in an overlapping structure similar to roof shingles. Most hair conditioning products attempt to affect the cuticle. Pigment cells are distributed throughout the cortex and medulla giving the hair its characteristic color.

The term “eyebrow” refers to an area of coarse skin hairs above the eye that follows the shape of the brow ridges. The main function of the eyebrow is to prevent moisture, mostly salty sweat and rain, from flowing into the eye, an organ critical to sight. The typical curved shape of the eyebrow (with a slant on the side) and the direction in which eyebrow hairs are pointed, make sure that moisture has a tendency to flow sideways around the eyes, along the side of the head and along the nose. Eyebrows also prevent debris, such as dandruff and other small objects, from falling into the eyes, and provide a more sensitive sense for detecting objects being near the eye, like small insects. Eyebrows also have an important facilitative function in communication, strengthening facial expressions such as surprise, confusion, or anger.

The terms “eyelash” and “lash” are used interchangeably to refer to one of the hairs that grow at the edge of the eyelid. Eyelashes protect the eye from debris and provide a warning that an object (such as an insect or dust mite) is near the eye (which then is closed reflexively).

The inside of the nose contains small hairs called cilia. The cilia and nasal mucus clean the air drawn into the nose of the microscopic particles we inhale, including dust, pollen, and pollutants, for ultimate passage to the lungs.

There are three stages of hair growth: catagen, telogen, and anagen.

Anagen is the active growth phase of the hair during which the cells in the root of the hair are dividing rapidly. Anagen hairs are anchored deeply into the subcutaneous fat and cannot be pulled out easily. When a new hair is formed, it pushes the club hair up the follicle and eventually out. During this phase the hair grows about 1 cm every 28 days. Scalp hair stays in this active phase of growth for 2-6 years. Human subjects that have difficulty growing their hair beyond a certain length have a short active phase of growth. Human subjects that have very long hair have a long active phase of growth. The hair on the arms, legs, eyelashes, and eyebrows have a very short active growth phase of about 30-45 days, which is why they are so much shorter than scalp hair.

The anagen phase is followed by a catagen phase. The catagen phase is a transitional stage that lasts for about 2-3 weeks. About 3% of all hairs are in this phase at any time. During this time, growth stops and the outer root sheath shrinks and attaches to the root of the hair. This is the formation of what is known as a club hair.

After catagen, the hair goes into a telogen phase. Telogen is the resting phase, which accounts for 10-15% of all hairs. It lasts for about 100 days for hairs on the scalp and much longer for hairs on the eyebrow, eyelash, arm and leg. During this phase, the hair follicle is completely at rest and the club hair is completely formed. As compared with anagen hair, telogen hair is located higher in the skin and can be pulled out relatively easily. Pulling out a hair in this phase will reveal a solid, hard, dry, white material at the root. Normally, about 25-100 telogen hairs are shed each day.

In the normal scalp, approximately 80 to 90 percent of follicles are growing (anagen), about 5 to 10 percent are resting (telogen), and 1 to 3 percent are undergoing involution (catagen). Each day, up to 75 hairs in telogen are shed from the scalp and about the same number of follicles enter anagen.

### 9. Depression

Depression is a condition of general emotional dejection and withdrawal. The symptoms vary from person to person. Some people say that depression feels like a black curtain of despair coming down over their lives. Many feel as if they have no energy and cannot concentrate, while others feel irritable all the time for no apparent reason.

Serious medical conditions, such as cancer, can contribute to depression, partly because of the physical weakness and stress they bring on. Depression can make medical conditions worse, since it weakens the immune system and can make pain harder to bear. In some cases, depression can be caused by medications used to treat medical conditions.

Generally, symptoms of depression include, but are not limited to, constant feelings of sadness, irritability, or tension; decreased interest or pleasure in usual activities or hobbies; loss of energy, feeling tired despite lack of activity; a change in appetite, with significant weight loss or weight gain; a change in sleeping patterns, such as difficulty sleeping, early morning awakening or sleeping too much; restlessness or feeling slowed down; decreased ability to make decisions or concentrate; feelings of worthlessness, hopelessness, or guilt; and thoughts of suicide or death.

The term “depression” also may refer to a wide variety of abnormal variations in an individual's mood. If changes in an individual's mood are persistent and cause distress or impairment in functioning, then a mood disorder may be present. Individuals with mood disorders experience extremes of emotions, for example sadness, that are higher in intensity and longer in duration than normal.

The most widely used criteria for diagnosing depressive conditions are found in the American Psychiatric Association's revised fourth edition of the Diagnostic and Statistical Manual of Mental Disorders (DSM-IV-TR), and the World Health Organization's International Statistical Classification of Diseases and Related Health Problems (ICD-10) which uses the name recurrent depressive disorder, the contents of which are incorporated herein by reference.

For many patients hair loss is and inevitable consequence associated with chemotherapy, and little recourse is available to obviate the anxieties of chemotherapy-induced hair loss before hair loss becomes a reality. Many cancer patients who experience chemotherapy hair loss choose to use wigs, hair pieces, scarves, hats and turbans. Some cancer patients leave their head uncovered while others use something to cover their head in public and leave it uncovered at home. Those suffering from hair loss often experience embarrassment and fear being ridiculed by others because they look different. Some may take to wearing oversized eyeglasses in an attempt to hide the absence of eyelashes and/or eyebrows.

While several therapies to grow hair are available, including topical minoxidil (Rogain®), antiandrogen agents, including the androgen-receptor blockers spironolactone, cyproterone acetate, and flutamide, and the 5α-reductase inhibitor finasteride (Propecia®, Merck & Co.), preparations of progesterone and/or estrogen, and hair transplantation, none prevent hair loss during treatment with a chemotherapeutic agent.

### 10. Prostaglandin Analogs

Prostaglandin analogs have been shown to be effective in treating epithelial-related conditions (see U.S. Pat. Nos. 7,553,875; 7,553,874; 7,550,508; 7,541,382; 7,517,912; and 7,514,474, incorporated in their entirety herein by reference).

Prostaglandins are a family of a group of lipid compounds that are derived enzymatically in the body from essential fatty acids. Every prostaglandin contains 20 carbon atoms, including a 5-carbon ring. Prostaglandins have a wide variety of effects, including, but not limited to, muscular constriction mediating inflammation, calcium movement, hormone regulation and cell growth control. Prostaglandins act on a variety of cells, including vascular smooth muscle cells (causing constriction or dilation), platelets (causing aggregation or disaggregation), and spinal neurons (causing pain).

The basic chemical structure of naturally occurring prostaglandins, as shown below, reveals that prostaglandins generally consist of a cyclopentane ring and two side chains:

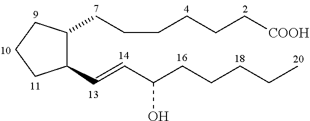

The upper side chain (or “alpha chain”) generally contains 7 carbon atoms. The lower side chain (or “omega chain”) generally contains 8 carbon atoms. The end of the alpha chain normally is a carboxylic acid moiety. The side chains may contain 1 to 3 double bonds, most frequently 2, the double bonds being situated between carbon atoms 5 and 6 on the alpha chain and between bonds 13 and 14 on the omega chain. The double bond on the alpha chain generally exhibits cis-configuration, whereas the double bond on the omega chain generally exhibits trans-configuration. A substituent group on carbon 15 in the omega chain is preferred for maximal biological activity. In naturally occurring prostaglandins this substituent is hydroxyl.

Different classes of prostaglandins are identified by suffixes A, B, C, D, E, F or J depending on the functionalities of the five-membered ring, that is the configuration and substituents of the cyclopentane ring. Prostaglandins A, B and C probably are not naturally occurring but rather are artificial prostaglandins; nevertheless, they exert considerable biologic activity.

The configuration of and functionalities attached to the cyclopentane ring are important for selectivity to different prostaglandin receptors. The various configurations include:

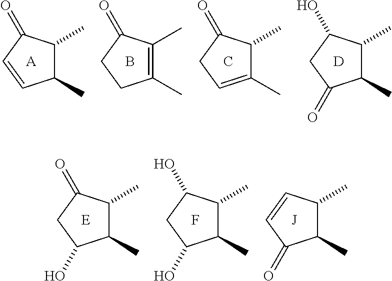

Structures of some of the known prostaglandins are presented below.

10.1. Prostaglandin A

The chemical structure of prostaglandin A2 is shown below wherein a hashed line represents a substituent below this paper's plane; and wherein a bold wedge represents a substituent above this paper's plane:

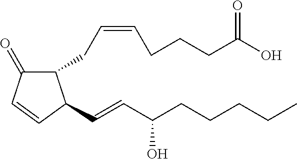

10.2. Prostaglandin B

The chemical structure of known prostaglandin B2 is shown below:

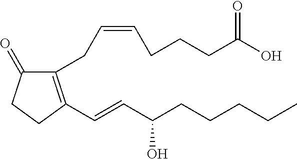

10.3. Prostaglandin D

The chemical structure of known prostaglandin D2 is shown below wherein a hashed line represents a substituent below this paper's plane; wherein a bold wedge represents a substituent above this paper's plane:

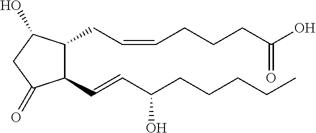

10.4. Prostaglandin E

The chemical structure of known prostaglandin E₁ (11α,13E,15S)-11,15-dihydroxy-9-oxoprosta-13-en-1-oic acid) (Alprostadil) is shown below wherein a hashed line represents a substituent below this paper's plane; wherein a bold wedge represents a substituent above this paper's plane:

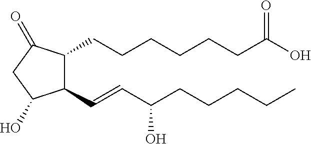

The chemical structure of known prostaglandin E2 (9-oxo-11α,15S-dihydroxy-prosta-5Z,13E-dien-1-oic acid) (Dinoprostone) is shown below wherein a hashed line represents a substituent below this paper's plane; wherein a bold wedge represents a substituent above this paper's plane:

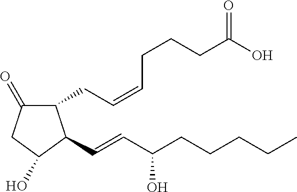

10.5. Prostaglandin F

The general chemical structure of known prostaglandin Fₐₜ is shown below wherein a hashed line represents a substituent below this paper's plane; wherein a bold wedge represents a substituent above this paper's plane; and wherein the dashed lines represent a single or double bond which can be in the cis or trans configuration:

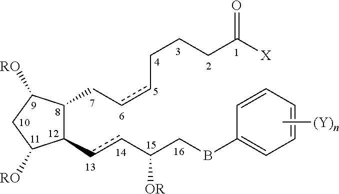

There are several commercially available prostaglandin F analogs. For example, latanoprost [(1R,2R,3R,5S)3,5-dihydroxy-2-[(3R)-3-hydroxy-5-phenylpentyl]cyclopentyl]-5-heptenoate], marketed by Pfizer as Xalatan® is a prostaglandin analog in which R is H, B is —CH₂—, n is 0, X is OCH(CH₃)₂, and the dashed lines represent a double bond. See U.S. Pat. No. 6,262,105, issued to Johnstone. Although Johnstone reported the stimulating effect of this drug on eyebrow and eyelash hair growth and pigmentation, Latanoprost works poorly on eyelashes.

Another example, is bimatoprost (cyclopentane N-ethyl heptenamide-5-cis-2-(3α-hydroxy-5-phenyl-1-trans-pentenyl)-3,5-dihydroxy, [1α, 2β, 3α, 5α], sold by Allergan, Inc. of Irvine, Calif. as Lumigan®, a 0.03% ophthalmic solution for treating glaucoma. Bimatoprost is a prostaglandin analog in which R is H, B is —CH₂—, n is 0, X is NHC₂H₅ and the dashed lines represent a double bond. U.S. Published Application No. 2003/0147823. Bimatoprost, which also has been found effective to increase the growth of eyelashes when applied in the FDA approved manner, dissolves best for use on eyelashes but has negative side effects—e.g., redness and discoloration along the periocular skin; eye irritation; and foreign body sensation. In addition, bimtoprost has the highest incidence of hyperemia.

Another synthetic prostaglandin analog used for treatment of glaucoma is isopropyl (Z)-7-[(1-R,2-R,3-R,5-S)-3,5-dihydroxy-2-[(1E,3R)-3-hydroxy-4-[(α,α,α-trifluoro-m-tolyl)oxy]-1-butenyl]cyclopentyl]-5-heptenoate, or Travaprost (TRAVATAN® Alcon), which is available as a 0.004% ophthalmic solution. Travoprost is a prostaglandin analog in which R is H, B is O, Y is CF₃, X is OCH(CH₃)₂, and the dashed lines represent a double bond. Travaprost does not work well for eyelash growth, taking longer than other like products.

10.6. Prostaglandin H

The chemical structure of known prostaglandin H2 is shown below wherein a hashed line represents a substituent below this paper's plane; wherein a bold wedge represents a substituent above this paper's plane:

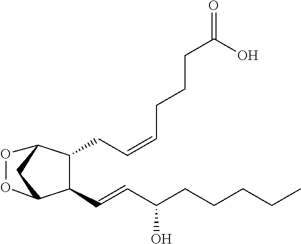

10.7. Prostaglandin J

The chemical structure of known prostaglandin J2 is shown below wherein a hashed bond represents a substituent below this paper's plane; wherein a bold wedge represents a substituent above this paper's plane:

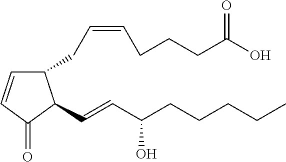

10.8. Prostacyclin

The chemical structure of prostacyclin ((Z)-5-((3aR,4R,5R,6aS)-5-hydroxy-4-((S,E)-3-hydroxyoct-1-enyl)hexahydro-2H-cyclopenta[b]furan-2-ylidene)pentanoic acid) (PGI₂) is shown below wherein a hashed line represents a substituent below this paper's plane; wherein a bold wedge represents a substituent above this paper's plane:

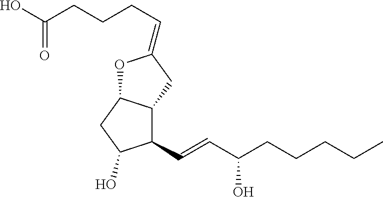

The described invention provides compositions and methods comprising at least one prostaglandin analog that are protective, in that their used prevents hair loss and other hair-related side-effects associated with chemotherapy.

### SUMMARY OF THE INVENTION

The described invention relates to delivery of compositions comprising at least one prostaglandin analog to prevent or reduce hair loss (e.g. brittle hair growth, thin hair growth, short hair growth, sparse hair growth) or alopecia associated with chemotherapy.

According to one aspect, the described invention provides a method for preventing or reducing a hair-related side effect caused by treatment of a subject in need thereof with at least one chemotherapeutic agent, wherein the side effect is selected from the group consisting of sparse hair growth, brittle hair growth, short hair growth, thin hair growth, alopecia and hair depigmentation, the method comprising the steps: (a) providing a topical composition, the topical composition comprising a first component and an optional second component, the first component comprising: (i) at least one compound of Formula I or a pharmaceutically acceptable salt, hydrate, solvate, prodrug or metabolite thereof,

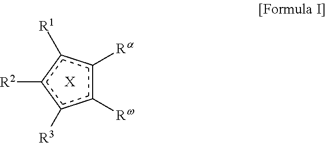

wherein ring X is selected from

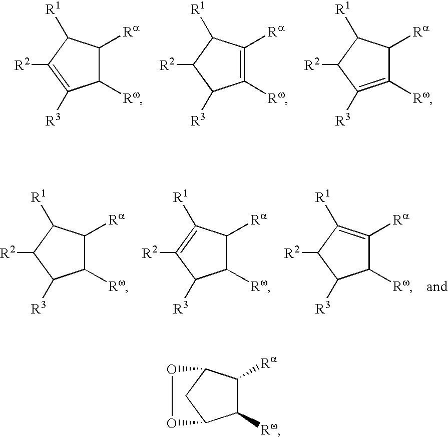

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each, wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1˜25 or a pharmaceutically acceptable salt thereof; —P(O)(OH)₂ or a pharmaceutically acceptable salt thereof, wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups, wherein R^{α} is

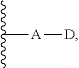

wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one

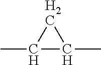

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof, wherein each R⁷ is independently H, F, or straight chain or branched C₁˜C₅ alkyl, wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups, wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂NR⁹₂; —NR⁹S(O)₂R¹³; —OS(O)₂NR⁹₂; —C(O)NHS(O)₂R¹³; —S(O)₂R¹³; —SO₃H; —PO₃H₂;

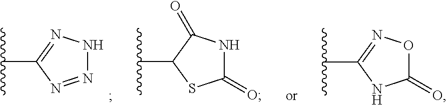

wherein R^{ω} is

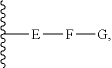

wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof, wherein F is —CH₂—, —O—; —S—; —S(O)—; —S(O₂)—; —C(O)—; —C(O)O—; —C(O)S—; —C(O)NR⁹—; —NR⁹—; or a covalent bond, wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups, wherein each R⁸ is independently selected from the group consisting of: H; a pharmaceutically acceptable cation optionally selected from the group consisting of sodium, potassium, magnesium, calcium or an organic cation optionally selected from the group consisting of an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester optionally selected from the group consisting of a lower alkyl ester, a lower acyloxy-alkyl ester optionally selected from the group consisting of acetoxymethyl, acetoxyethyl, aminocarbonyloxymethyl, pivaloyloxymethyl or pivaloyloxyethyl ester, a lactonyl ester optionally selected from the group consisting of a phthalidyl or thiophthalidyl ester, a lower alkoxyacyloxyalkyl ester optionally selected from the group consisting of a methoxycarbonyloxymethyl, ethoxycarbonyloxyethyl or isopropoxycarbonyloxyethyl ester, an alkoxyalkyl ester, choline ester or acylamino alkyl ester optionally selected from the group consisting of an acetamidomethyl ester; or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups, wherein each R⁹ is independently selected from the group consisting of: H; a C₁˜C₂₀ straight chain or branched acyclic hydrocarbon group containing zero to four C═C or C≡C bonds wherein each C═C bond independently can be of E or Z configuration; a C₁˜C₂₀ straight chain or branched acyl group containing zero to four C═C or C≡C bonds wherein each C═C bond independently can be of E or Z configuration; —(CH₂)_{q}OH, —(CH₂)_{q}OR¹⁴, —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl optionally selected from the group consisting of acetoxymethyl, acetoxyethyl, aminocarbonyloxymethyl, pivaloyloxymethyl or pivaloyloxyethyl, lactonyl optionally selected from the group consisting of a phthalidyl or thiophthalidyl, lower alkoxyacyloxyalkyl optionally selected from the group consisting of a methoxycarbonyloxymethyl, ethoxycarbonyloxyethyl or isopropoxycarbonyloxyethyl, or acylamino alkyl optionally selected from the group consisting of acetamidomethyl; or -J-K; or —NR⁹₂ can be a cycloamido radical optionally selected from the group consisting of 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like, wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl, wherein each R¹¹ is independently H or —C(O)R¹⁶, wherein each R¹² is independently R¹⁶ or —C(O)R¹⁶, wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen), wherein each R¹⁴ is independently straight chain or branched C₁˜C₆ alkyl or —CH₂OCH₃, wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂;

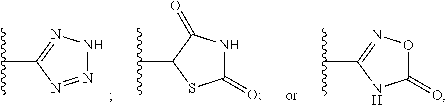

wherein each R¹⁶ is independently H or straight chain or branched C₁˜C₆ alkyl, phenyl or —CH₂OCH₃, wherein each R¹⁷ is independently straight chain or branched C₁˜C₆ alkyl; —C(O)OR¹⁶; phenyl; F; Cl; Br; I; CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂N(R¹⁶)₂; —NR¹⁶S(O)₂R¹³, with the proviso that, if R¹⁷ is —NR¹⁶S(O)₂R¹³, R¹, R², R³, R^{α} and R^{ω} are selected such that the molecular weight of the compound of Formula I does not exceed about 2000 atomic mass units, and wherein not more than four of R⁷ are other than H or F and not more than four of R⁷ are F; and wherein each chiral center or allenic moiety independently possesses any relative or absolute stereoconfiguration or comprises any mixture thereof; and (ii) a carrier; (b) topically administering a hair-protective amount of the topical composition onto an epithelial-related surface of the subject concurrent with administration of at least one chemotherapeutic agent; and (c) stimulating hair growth on the epithelial-related surface to which the topical composition has been applied. According to one embodiment, the at least one compound of Formula I is diastereomerically pure. According to another embodiment, the at least one compound of Formula I is a mixture of diastereomers in any ratio. According to another embodiment, the at least one compound of Formula I is enantiomerically pure. According to another embodiment, the at least one compound of Formula I is a mixture of enantiomers in any ratio, including a racemate. According to another embodiment, the at least one compound of Formula I is diastereomerically and enantiomerically pure. According to another embodiment, the at least one compound of Formula I is a mixture of diastereomers and enantiomers in any ratio. According to another embodiment, the at least one compound of Formula I has one or more hydrogen atoms replaced by deuterium. According to another embodiment, the optional second component is an imidazole analog according to Formula III, or a hydrate, solvate, salt, zwitterion, N-oxide, tautomer, prodrug, or metabolite thereof:

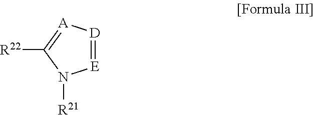

wherein A is N; NR²⁰; NR⁶³ or CH; wherein D is N or CR²³; wherein E is N; NR⁶³ or CR²⁴; with the proviso that, if A is CH, then at least one among D and E is a nitrogen-containing moiety; with the further proviso that A and E are not simultaneously both NR⁶³; wherein R²³ is H; F, Cl, Br; I; —NO₂; —N(R⁴²)₂; straight chain or branched C₁˜C₆ alkyl, alkenyl or alkoxy; or phenyl; wherein R²⁴ is H; F, Cl, Br; I; —NO₂; —N(R⁴₂)₂; straight chain or branched C₁˜C₆ alkyl, alkenyl or alkoxy; or phenyl; or wherein R²³ and R²⁴ together are —CR⁶²═CR⁶²—CR⁶²═CR⁶²—; wherein R²⁰ is selected from the group consisting of: H; straight chain or branched C₁˜C₁₂ alkyl;

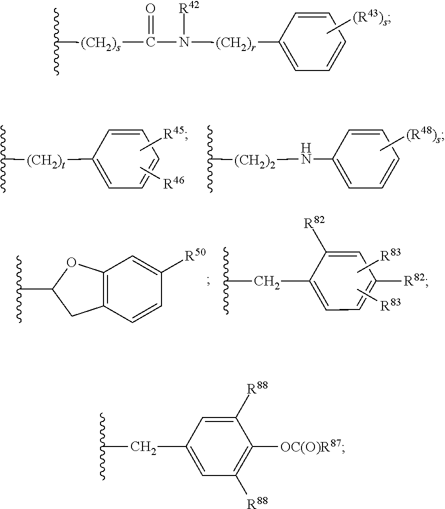

—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula III is an imidazolium or triazolium salt with a pharmaceutically acceptable counter anion or an internal salt (zwitterion); or wherein R⁶³ is a moiety that is readily cleaved in vivo, wherein R⁶³ optionally is selected from the group consisting of —CHR⁴²OC(═O)(O)ₙR⁵⁵ or —C(R⁴²)₂OP(═O)(OR¹⁰⁶)₂ and the compound of Formula III is a prodrug and an imidazolium or triazolium salt with a pharmaceutically acceptable counter ion or an internal salt (zwitterion); wherein s is 1 or 2; wherein t is an integer from 1 to 4 inclusive; wherein u is 2 or 3; wherein R²² is H; F, Cl, Br; I; —NO₂, —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical containing one S and one or two N atoms, said heterocyclic radical optionally selected from the group consisting of thiazolyl or thiazol-4-yl; wherein R²¹ is H; or —(CR²⁵R²⁶)ₘ—CR²⁷R²⁸-Q-R²⁹; wherein each m is independently an integer from 0 to 4 inclusive; wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups; wherein R²⁶ is H; or straight chain or branched C₁˜C₆ alkyl; wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(═NH)N(R⁶⁸)OR⁶⁸; —CF(R⁶⁷)C(═NH)N(R⁶⁸)OH; or —CF(R⁶⁷)C(═N—OR⁶⁹)NH₂; wherein each r is independently an integer from 0 to 2 inclusive; wherein each L is independently α-tetralyl; or phenyl, said phenyl being optionally substituted with up to three groups selected from the group consisting of: straight chain or branched C₁˜C₆ alkyl, straight chain or branched C₁˜C₆ alkoxy, —C≡N, —NO₂, or —NH₂; wherein R²⁸ is H; F; Cl; Br; —OR⁴²; —OC(═O)R¹⁰⁰;

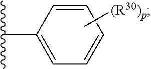

straight chain or branched C₁˜C₆ alkyl; R61; or —OP(═O)(OH)₂ or a pharmaceutically acceptable salt or zwitterion form thereof; wherein each Q is independently a covalent bond; —O—; —S—; —S(O)—; or —S(O)₂—; wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; straight chain or branched C₁˜C₆ alkyl optionally substituted with —CO₂H; or

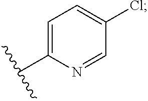

wherein each p is independently an integer from 0 to 3 inclusive; wherein each T is independently a covalent bond; —CH₂CH₂—; —CH═CH—; —(O)ₙC(R⁴²)₂—; —CH₂O—; —SCH₂—; —CH₂S—; —OCH₂CH₂O—; —CH(CH₃)—; —CH₂—; —CF₂—; —C(R⁹³)₂—; or

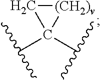

wherein each v is independently an integer from 1 to 3 inclusive; wherein each U is independently a covalent bond;

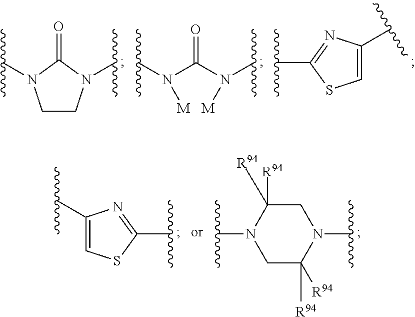

wherein each V is independently H; —S(O)₂CH₃; —C(O)NH₂; —CH₂C≡CH; —CH═CH₂; —S(O)ₙ—R⁵⁵;

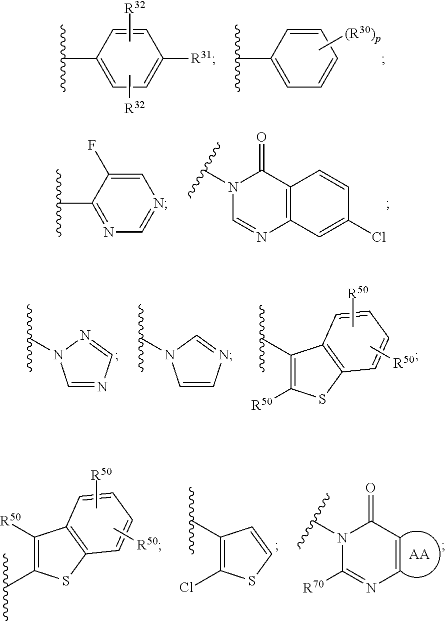

—R⁵⁹; —R⁶¹; or -LL-R⁹⁵; wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings; wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴; —OC(O)N(R⁵⁴)₂; —SH; —SR⁴¹; —S(O)ᵣR⁵⁸; —CH═CH—CO₂H;

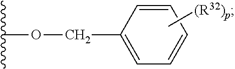

straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to —CH₂C≡N, —CH₂CH₂C≡N, —CH₂CH₂CH₂C≡N, and —CH₂CH(CH₃)C≡N; straight chain or branched C₁˜C₆ halogenoalkyl optionally selected from the group consisting of —CF₃, —CF₂CF₃, —CH₂CF₃, —CHF, —CHF, —CH₂Cl, and —CH₂Br; straight chain or branched C₁˜C₅ halogenoalkoxy optionally selected from the group consisting of —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₈ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy; wherein each R³¹ is independently H; F; Cl; Br; I; —C≡N; —NO₂; —CF₃; —N═C═S; —N(R⁴²)₂; —NH—C(═O)-(M)ₙ-R⁵⁴; —NH—C(═S)—NH—R⁵⁴; straight chain or branched C₁˜C₆ alkoxy or alkylthio; straight chain or branched C₁˜C₁₂ alkyl; —(CH₂)ᵣ—R⁹⁹;

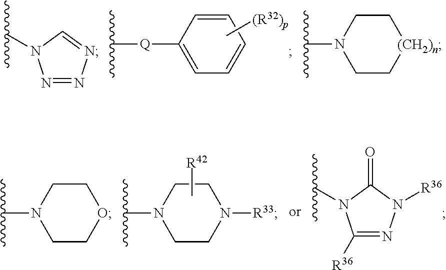

wherein each M is independently O or NH, with the proviso that when M is O and n is 1, R⁵⁴ is other than H; wherein each R³² is independently H; F; Cl; Br; I; —C≡N; —NO₂; straight chain or branched C₁˜C₁₂ alkyl; straight chain or branched C₁˜C₄ alkoxy; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; straight chain or branched C₁˜C₃ halogenoalkoxy optionally selected from the group consisting of —OCF₃, —OCF₂CF₃, —OCH₂CF₃, —OCHF₂, —OCH₂F; straight chain or branched C₁˜C₄ alkylthio or haloalkylthio; straight chain or branched C₁˜C₄ alkylthionyl or haloalkylthionyl; or straight chain or branched C₁˜C₄ alkylsulfonyl or haloalkylsulfonyl; wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)ₙC(O)OR³⁴; —C(O)O-phenyl; —(CH₂)ₙC(O)N(R⁴²)₂; —CH₂C(S)N(R⁴²)₂; —CH₂C(S)SR³⁴; —(CH₂)n-phenyl; benzoyl, wherein said benzoyl is optionally substituted with one or two R⁴⁶ groups; or

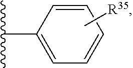

or, preferably,

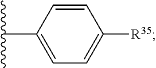

wherein each R³⁴ is independently straight chain or branched C₁˜C₆ alkyl; wherein each R³⁵ is independently H;

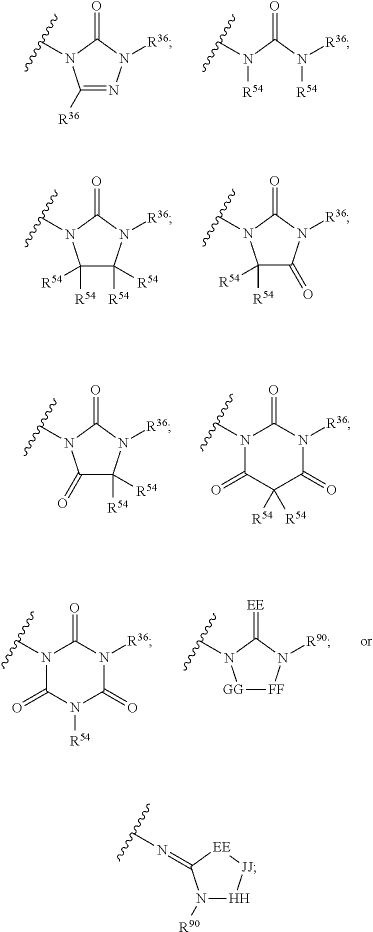

wherein each R³⁶ is independently H; straight chain or branched C₃˜C₇ alkenyl, with the proviso that the olefinic double bond is not located a to N; straight chain or branched C₃˜C₇ alkynyl, with the proviso that the alkynyl C≡C bond is not located α to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or

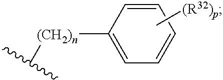

wherein each n is independently 0 or 1; wherein R²⁶ and R²⁸ together with the two carbon atoms to which they attach can be C═C; wherein R²⁷ and R²⁸ together can be

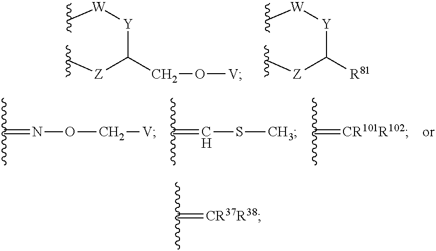

wherein W is —CH₂— and Y is —CH₂—; W is —O— and Y is —CH₂—; or W is —CH₂— and Y is —O—; wherein Z is —CH₂— or —O—; wherein R²⁷ together with -Q-R²⁹ can be

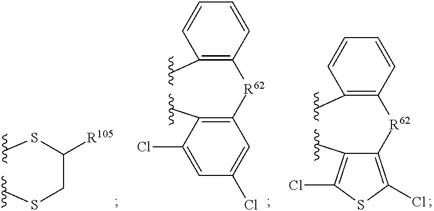

or, when R²⁷ and R²⁹ each independently represents a cycloalkyl, aryl or heterocyclic ring and Q is a covalent bond, R²⁷ together with -Q-R²⁹ can be

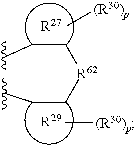

wherein each R³⁷ is independently H; C₁˜C₂ alkyl or phenyl; wherein each R³⁸ is independently H or C₁˜C₂ alkyl;
wherein each R³⁹ is independently H; straight chain or branched C₁˜C₅ alkyl; straight chain or branched C₁˜C₅ alkanoyl; or straight chain or branched C₁˜C₅ haloalkanoyl optionally selected from the group consisting of —C(O)CF₃, —C(O)CF₂CF₃, —C(O)CH₂CF₃, —C(O)CHF₂, and —C(O)CH₂F; wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J; wherein each R⁴¹ is independently straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J; wherein each q is independently an integer from 0 to 4 inclusive; wherein each G¹ is independently a covalent bond; —O—; or —S—; wherein each J is independently phenyl, 2-thienyl or 3-thienyl wherein said phenyl, 2-thienyl or 3-thienyl is optionally substituted with one to three groups selected from the group consisting of: F; Cl; Br; I; —NO₂; straight chain or branched C₁˜C₅ alkyl; straight chain or branched C₁˜C₅ alkoxy; wherein each R⁴² is independently H; or straight chain or branched C₁˜C₆ alkyl; wherein each R⁴³ is independently H; F; Cl; Br; I; —NO₂; straight chain or branched C₁˜C₆ alkyl; straight chain or branched C₁˜C₆ alkoxy; —CF₃; or —O— phenyl; wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the group consisting of: F, Cl, Br, I, straight chain or branched C₁˜C₆ alkyl, or straight chain or branched C₁˜C₆ alkoxy; wherein each R⁴⁵ is independently H; F; Cl; Br; I; —NO₂; straight chain or branched C₁˜C₆ alkyl; straight chain or branched C₁˜C₆ alkoxy; or —S(O)₂NH₂; wherein each R⁴⁶ is independently H; F; Cl; Br; I; straight chain or branched C₁˜C₆ alkyl; straight chain or branched C₁˜C₆ alkoxy; or —C≡N; wherein each R⁴⁷ is independently a phenyl group optionally substituted with one to three F, Cl, Br or I; wherein each R⁴⁸ is independently H; F; Cl; Br; I; or straight chain or branched C₁˜C₆ alkyl; wherein each R⁴⁹ is independently 1-naphthyl; 2-naphthyl; or phenyl, wherein said phenyl is optionally substituted with one or two groups, each independently selected from the group consisting of: F, Cl, Br, I, straight chain or branched C₁˜C₆ alkyl, straight chain or branched C₁˜C₆ alkoxy, or —CH₂CH═CH₂; wherein each R⁵⁰ is independently H; F; Cl; Br; or I; wherein each R⁵¹ is independently phenyl, wherein said phenyl is optionally substituted with one or two groups, each independently selected from the group consisting of: F, Cl, Br, I, or straight chain or branched C₁˜C₆ alkyl; wherein each R⁵² is independently phenyl, wherein said phenyl is optionally substituted with one or two groups, each independently selected from the group consisting of: F, Cl, Br, or I; wherein each R⁵³ is independently straight chain or branched C₁˜C₈ alkyl optionally substituted with C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; straight chain or branched C₃˜C₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl C≡C bond is located β, γ or δ with respect to the ether oxygen; wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl; wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle can be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups; wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle can be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle can be aromatic or non-aromatic; methylenedioxy; —C≡N; —NO₂; —CF₃; —N(R⁵⁴)₂; —OR⁵⁴; —SH; ═O; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; —C(═NH)N(R⁵⁴)OH; —C(═N—OR⁵⁴)NH₂; —S(O)₂N(R⁵⁴)₂; —NR⁵⁴S(O)₂R⁵⁴; —C(O)NHS(O)₂R⁵⁴; —S(O)₂R⁵⁴; —SO₃H; or —PO₃H₂; wherein each R⁵⁷ is independently F; Cl; Br; I; straight chain or branched C₁˜C₆ alkyl; straight chain or branched C₁˜C₆ alkoxy; —C≡N; —CF₃; —NO₂, —NH₂; or —NHC(O)R⁶⁶, wherein said R⁶⁶ is straight chain or branched C₂-C₁₂ alkyl; wherein each R⁵⁸ is independently straight chain or branched C₁˜C₂₀ alkyl; C₃˜C₈ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups; wherein each R⁵⁹ is independently straight chain or branched C₁˜C₇ alkyl, alkenyl or alkynyl, said alkyl, alkenyl or alkynyl being substituted with one or more —R⁶⁰—R⁶¹; each R⁶⁰ is independently a covalent bond or —O—; wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; wherein each R⁶² is independently a covalent bond; —CH₂—CH₂—; —CH═CH—; —O—; or —S—; wherein each R⁶⁵ is independently straight chain or branched C₁˜C₁₂ acyclic hydrocarbon group, which can be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or C≡C bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups; wherein each R⁶⁷ is independently H; F; or straight chain or branched C₁˜C₆ alkyl, said alkyl group being optionally substituted by one or more R³⁰; wherein each R⁶⁸ is independently R³⁶, R⁵⁴ or R⁶¹; wherein each R⁶⁹ is independently H; or straight chain or branched C₁˜C₆ alkyl, said alkyl group being optionally substituted by one or more R³⁰; wherein each R⁷⁰ is independently H; straight chain or branched C₁˜C₄ alkyl; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; or cyclopropyl; wherein each R⁷¹ is independently R⁵⁶; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)rR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶;

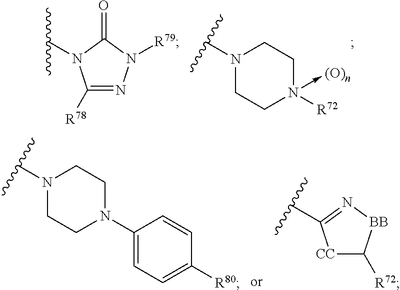

wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy; wherein each R⁷³ is independently H; straight chain or branched C₁˜C₄ alkyl; C₃˜C₆ cycloalkyl; —C(O)R⁷²; or —C(O)CF₃; wherein each R⁷⁴ is independently straight chain or branched C₁˜C₄ alkyl; wherein each R⁷⁵ is independently H; —C(O)NH₂; —C(O)CH₃; —C≡N; —SO₂NHR⁷²; —SO₂R⁷²; —OR⁷²; —OC(O)R⁷²; or straight chain or branched —(C₁˜C₄ alkyl)-NH₂; wherein each R⁷⁶ is independently phenyl optionally substituted with one or more R⁷⁷ groups; wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²;

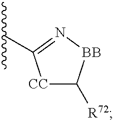

or phenyl, said phenyl being optionally substituted with one or more F, Cl, Br, I, —C≡N, —NO₂, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy; wherein each R⁷⁸ is independently H or —CH₃; wherein each R⁷⁹ is independently H; isopropyl; cyclopentyl; cyclopropyl; 2-butyl; 3-pentyl; 3-hydroxy-2-butyl; or 2-hydroxy-3-pentyl; wherein each R⁸⁰ is independently F; Cl; Br; I; —C≡N; —NO₂; —NH₂; straight chain or branched C₁˜C₄ halogenoalkyl; straight chain or branched C₁˜C₄ halogenoalkoxy; or

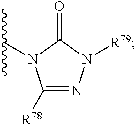

wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —C≡N, —NO₂, phenyl, phenylmethyl, benzoyl, halobenzoyl, —C(O)R⁴², —C(O)OR⁴², and —CF₃, with the proviso that when multiple substituents are present only 1 thereof can be selected from the group consisting of phenyl, phenylmethyl, benzoyl and halobenzoyl; wherein each R⁸² is independently H or —OR⁸⁴; wherein each R⁸³ is independently H; F; Cl; Br; R⁷⁴; —OR⁷⁴; —SR⁷⁴; —CH₂SC(═O)R⁷⁴; —COOH; or —C(═O)OCH₃; wherein each R⁸⁴ is independently a group that is easily hydrolyzable under physiological conditions, optionally at the acyl residue of an amino acid or a group represented by the formula —C(═O)R⁸⁵ or —P(═O)(OR⁸⁶)₂; wherein R⁸⁴ is optionally selected from the group consisting of formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like; wherein each R⁸⁵ is independently H; —OR⁷⁴; or straight chain or branched C₁˜C₆ alkyl or alkenyl, wherein said alkyl or alkenyl can be optionally interrupted by a hydrolyzable functional group such as carboxamide or ester, wherein said alkyl or alkenyl can be optionally substituted with —COOH, —NH₂, —NHR⁷⁴, —N(R⁷⁴)₂, or R⁶¹, wherein R⁶¹ is optionally selected from the group consisting of phenyl, methoxyphenyl, pyridyl, pyrazinyl or furyl; wherein each R⁸⁶ is independently H or R⁷⁴; wherein each R⁸⁷ is independently straight chain or branched C₁˜C₅ alkyl; R⁶¹; pyridyl; pyrrolidinyl; or -DD—NH—R⁸⁹; wherein each R⁸⁸ is independently H; F; Cl; Br; or —OR⁷⁴; wherein each DD is independently straight chain or branched C₁˜C₄ alkylene; —CH₂NHC(═O)CH₂—; —CH(NH₂)CH₂CH₂CH₂—; wherein each R⁸⁹ is independently H or straight chain or branched C₁˜C₅ alkyl; wherein each BB is independently —CH₂— or —C(═O)—; wherein each CC is independently NH or O; wherein each EE is independently O, S or N—R⁹¹; wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃˜C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched; wherein each KK is independently O or S; wherein each R⁹¹ is independently H or straight chain or branched C₁˜C₆ alkyl; wherein each R⁹² is independently H; straight chain or branched C₁˜C₆ alkyl; C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—; wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; wherein each GG is independently —C(═O)—; or —CH₂—, optionally substituted with up to two radicals selected from the group consisting of straight chain or branched C₁˜C₆ alkyl and straight chain or branched C₁˜C₆ alkoxy; or FF and GG, taken together, form a bivalent radical of formula —N═CH—(FFGG′); wherein HH has the same meaning as FF; wherein JJ has the same meaning as GG; or HH and JJ, taken together, form a bivalent radical of formula —N═CH—(FFGG′) or —CH═CH—(HHJJ′); wherein the nitrogen atom in the bivalent radical FFGG′ is connected to NR⁹⁰; wherein one hydrogen in said radical FFGG′ and up to two hydrogens in radical HHJJ′ can be replaced by a straight chain or branched C₁˜C₆ alkyl radical; wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group optionally selected from the group consisting of —CH₂CH₂—, —CH₂CH₂CH₂—, —CH₂CH₂CH₂CH₂— and the like; wherein each R⁹⁴ is independently H or C₁˜C₄ alkyl which is unsubstituted or substituted by 1-3 substituents each independently selected from the group consisting of halogen, hydroxyl, C₁˜C₄ alkoxy, and amino; or two R⁹⁴ groups attached to the same carbon atom together form ═S; wherein each LL is independently a covalent bond; —C(═O)—; —C(═S)—; —SO₂—; or —N═N—; wherein each R⁹⁵ is independently selected from the group consisting of i) hydrogen, ii) CN iii) CHO iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl, v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido, vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl, vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and viii) a group of the formula

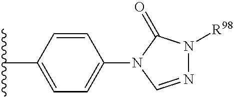

wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which can be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl; wherein each MM is independently an ethylene group optionally substituted with R91; or MM is —NN═OO—, wherein NN and OO are the same or different and are each independently N or CR⁹¹; wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶; wherein each R¹⁰⁰ is independently —R⁵⁴ or —OR⁵⁴; wherein each R¹⁰¹ is independently H; F; Cl; Br; or I; wherein each R¹⁰² is independently F; Cl; Br; I; —SR¹⁰³; or —OR¹⁰⁴; wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which can be the same or different; wherein each R¹⁰⁴ is independently straight chain or branched C₁˜C₁₂ alkyl; or phenyl; wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰; wherein each R¹⁰⁶ is independently H, a pharmaceutically acceptable cation, or null (affording an azolium phosphate zwitterion); wherein each chiral center independently may possess any relative or absolute stereoconfiguration or be any mixture thereof, unless specified otherwise herein; and wherein each olefinic C═C bond that is capable of E/Z isomerism independently may possess either the E or Z stereoconfiguration or be any mixture thereof, unless specified otherwise; wherein the imidazole analog improves efficacy of the at least one prostaglandin analog of Formula I when the composition is delivered to a subject refractory to treatment by a composition containing the prostaglandin analog of Formula I alone. According to another embodiment, the imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof. According to another embodiment, the imidazole analog is miconazole or ketoconazole or a pharmaceutically acceptable salt, hydrate, solvate, prodrug or metabolite thereof. According to another embodiment, the at least one compound of formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog. According to another embodiment, the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-(S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-(R,E)-3-hydroxy-4-(3-(trifluoromethyl)phenoxy)but-1-enyl)-5-oxocyclopent-3-enyl)-N-methylhept-5-enamide (Z)-7-((1R,2S)-2-(R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-(S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-(R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof. According to another embodiment, the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the optional second component of the composition comprises an additional active agent selected from the group consisting of a protective agent, an emollient, an astringent, an irritant, a keratolytic, a sun screening agent, a sun tanning agent, an antibiotic agent, a non-imidazole analog antifungal agent, an imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant, a chemotherapeutic agent, an anti-histamine, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent, a hypo-pigmenting agent, or a combination thereof. According to another embodiment, the at least one chemotherapeutic agent is at least one agent selected from the group consisting of an alkylating agent, an antimetabolite, a natural product, a biologic agent, a hormone or hormone-related agent, and a miscellaneous agent. According to another embodiment, the at least one chemotherapeutic agent is an alkylating agent, and wherein the side-effect of treatment with the alkylating agent is alopecia. According to another embodiment, the alkylating agent is selected from the group consisting of temozolomide, busulfan, ifosamide, melphalan hydrochloride, carmustine, lomustine and cyclophosphamide. According to another embodiment, the at least one chemotherapeutic agent is an antimetabolite, and wherein the side-effect of treatment with the antimetabolite is alopecia. According to another embodiment, the antimetabolite is selected from the group consisting of 5-fluorouracil, capecitabine, gemcitabine, floxuridine, decitabine, mercaptopurine, pemetrexed disodium, methotrexate and dacarbazine. According to another embodiment, the at least one chemotherapeutic agent is a natural product, and wherein the side-effect of treatment with the natural product is alopecia. According to another embodiment, the natural product is selected from the group consisting of vincristine, vinblastine, vinorelbine tartrate, paclitaxel, docetaxel, ixabepilone, daunorubicin, epirubicin, doxorubicin, idarubicin, mitoxantrone, mitomycin, dactinomycin, irinotecan, topotecan, etoposide, teniposide, etoposide phosphate, and bleomycin sulfate. According to another embodiment, the at least one chemotherapeutic is a biologic agent, and wherein the side-effect of treatment with the biologic agent is alopecia. According to another embodiment, the biologic agent is selected from the group consisting of filgrastim, pegfilgrastim, bevacizumab, sargramostim and panitumumab. According to another embodiment, the at least one chemotherapeutic agent is a hormone or a hormone-related agent, and wherein the side-effect of treatment with the hormone or the hormone-related agent is alopecia. According to another embodiment, the hormone or the hormone-related agent is selected from the group consisting of megestrol acetate, fluoxymesterone, leuprolide, octreotide acetate, tamoxifen citrate and fluxymesterone. According to another embodiment, the at least one chemotherapeutic agent is a miscellaneous agent, and wherein the side-effect of treatment with the miscellaneous agent is alopecia. According to another embodiment, the miscellaneous agent is selected from the group consisting of sorafenib, erlotinib, oxaliplatin, dexrazoxane, anagrelide, isotretinoin, bexarotene and vorinostat. According to another embodiment, the topical composition is administered concurrently with a chemotherapy regimen for treating breast cancer. According to another embodiment, the chemotherapy regimen is an Adriamycin-Cytoxan-Taxol regimen. According to another embodiment, the topical composition is administered concurrently with a chemotherapy regimen for treating colorectal cancer. According to another embodiment, the chemotherapy regimen comprises a combination of infusional 5-fluoruracil, leucovorin and oxaliplatin (FOLFOX®). According to another embodiment, the chemotherapeutic regimen for colorectal cancer comprises FOLFOX® with bevacizumab. According to another embodiment, the chemotherapy regimen comprises infusional 5-fluoruracil, leucovorin, and irinotecan (FOLFIRI®) with bevacizumab. According to another embodiment, the composition is formulated as a mascara.

According to another aspect, the described invention provides a method for preventing or reducing hair loss due to a chemotherapy insult in a subject in need thereof, the method comprising the steps: (a) providing a topical composition, the topical composition comprising a first component and an optional second component, the first component comprising: (i) at least one compound of Formula I or a pharmaceutically acceptable salt, hydrate, solvate, prodrug or metabolite thereof,

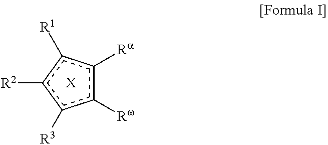

wherein ring X is selected from

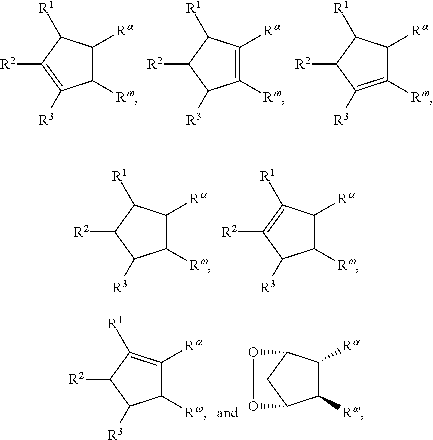

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each, wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1˜25 or a pharmaceutically acceptable salt thereof; —P(O)(OH)₂ or a pharmaceutically acceptable salt thereof, wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups, wherein R^{α} is

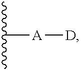

wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one

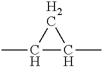

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof, wherein each R⁷ is independently H, F, or straight chain or branched C₁˜C₅ alkyl, wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups, wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; —NO₂; —SR¹⁰; —CH═NOR¹⁰; —C(═O)NR¹¹OR¹²; —S(O)₂NR⁹₂; —NR⁹S(O)₂R¹³; —OS(O)₂NR⁹₂; —C(O)NHS(O)₂R¹³; —S(O)₂R¹³; —SO₃H; —PO₃H₂;

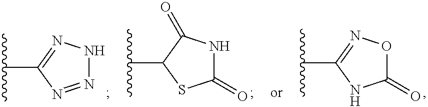

wherein R^{ω} is

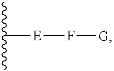

wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof, wherein F is —CH₂—, —O—; —S—; —S(O)—; —S(O₂)—; —C(O)—; —C(O)O—; —C(O)S—; —C(O)NR⁹—; —NR⁹—; or a covalent bond, wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups, wherein each R⁸ is independently selected from the group consisting of: H; a pharmaceutically acceptable cation optionally selected from the group consisting of sodium, potassium, magnesium, calcium or an organic cation optionally selected from the group consisting of an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester optionally selected from the group consisting of a lower alkyl ester, a lower acyloxy-alkyl ester optionally selected from the group consisting of acetoxymethyl, acetoxyethyl, aminocarbonyloxymethyl, pivaloyloxymethyl or pivaloyloxyethyl ester, a lactonyl ester optionally selected from the group consisting of a phthalidyl or thiophthalidyl ester, a lower alkoxyacyloxyalkyl ester optionally selected from the group consisting of a methoxycarbonyloxymethyl, ethoxycarbonyloxyethyl or isopropoxycarbonyloxyethyl ester, an alkoxyalkyl ester, choline ester or acylamino alkyl ester optionally selected from the group consisting of an acetamidomethyl ester; or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups, wherein each R⁹ is independently selected from the group consisting of: H; a C₁˜C₂₀ straight chain or branched acyclic hydrocarbon group containing zero to four C═C or C≡C bonds wherein each C═C bond independently can be of E or Z configuration; a C₁˜C₂₀ straight chain or branched acyl group containing zero to four C═C or C≡C bonds wherein each C═C bond independently can be of E or Z configuration; —(CH₂)_{q}OH, —(CH₂)_{q}OR¹⁴, —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl optionally selected from the group consisting of acetoxymethyl, acetoxyethyl, aminocarbonyloxymethyl, pivaloyloxymethyl or pivaloyloxyethyl, lactonyl optionally selected from the group consisting of a phthalidyl or thiophthalidyl, lower alkoxyacyloxyalkyl optionally selected from the group consisting of a methoxycarbonyloxymethyl, ethoxycarbonyloxyethyl or isopropoxycarbonyloxyethyl, or acylamino alkyl optionally selected from the group consisting of acetamidomethyl; or -J-K; or —NR⁹₂ can be a cycloamido radical optionally selected from the group consisting of 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like, wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl, wherein each R¹¹ is independently H or —C(O)R¹⁶, wherein each R¹² is independently R¹⁶ or —C(O)R¹⁶, wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen), wherein each R¹⁴ is independently straight chain or branched C₁˜C₆ alkyl or —CH₂OCH₃, wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂;

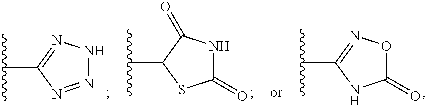

wherein each R¹⁶ is independently H or straight chain or branched C₁˜C₆ alkyl, phenyl or —CH₂OCH₃, wherein each R¹⁷ is independently straight chain or branched C₁˜C₆ alkyl; —C(O)OR¹⁶; phenyl; F; Cl; Br; I; CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂N(R¹⁶)₂; —NR¹⁶S(O)₂R¹³, with the proviso that, if R¹⁷ is —NR¹⁶S(O)₂R¹³, R¹, R², R³, R^{α} and R^{ω} are selected such that the molecular weight of the compound of Formula I does not exceed about 2000 atomic mass units, and wherein not more than four of R⁷ are other than H or F and not more than four of R⁷ are F; and wherein each chiral center or allenic moiety independently possesses any relative or absolute stereoconfiguration or comprises any mixture thereof; and (ii) a carrier; (b) topically administering a hair-protective amount of the topical composition onto an epithelial-related surface of the subject concurrent with administration of at least one chemotherapeutic agent to the subject; (c) stimulating hair growth on the epithelial-related surface to which the topical composition has been applied; and wherein the epithelial-related disorder is selected from the group consisting of sparse hair growth, short hair growth, thin hair growth, brittle hair growth, alopecia, and hair depigmentation. According to one embodiment, the at least one compound of Formula I is diastereomerically pure. According to another embodiment, the at least one compound of Formula I is a mixture of diastereomers in any ratio. According to another embodiment, the at least one compound of Formula I is enantiomerically pure. According to another embodiment, the at least one compound of Formula I is a mixture of enantiomers in any ratio, including a racemate. According to another embodiment, the at least one compound of Formula I is diastereomerically and enantiomerically pure. According to another embodiment, the at least one compound of Formula I is a mixture of diastereomers and enantiomers in any ratio. According to another embodiment, the at least one compound of Formula I has one or more hydrogen atoms replaced by deuterium. According to another embodiment, the optional second component is an imidazole analog according to Formula III, or a hydrate, solvate, salt, zwitterion, N-oxide, tautomer, prodrug, or metabolite thereof:

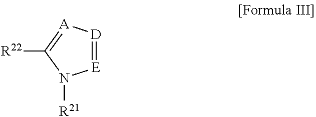

wherein A is N; NR²⁰; NR⁶³ or CH; wherein D is N or CR²³; wherein E is N; NR⁶³ or CR²⁴; with the proviso that, if A is CH, then at least one among D and E is a nitrogen-containing moiety; with the further proviso that A and E are not simultaneously both NR⁶³; wherein R²³ is H; F, Cl, Br; I; —NO₂; —N(R⁴²)₂; straight chain or branched C₁˜C₆ alkyl, alkenyl or alkoxy; or phenyl; wherein R²⁴ is H; F, Cl, Br; I; —NO₂; —N(R⁴₂)₂; straight chain or branched C₁˜C₆ alkyl, alkenyl or alkoxy; or phenyl; or wherein R²³ and R²⁴ together are —CR⁶²═CR⁶²—CR⁶²═CR⁶²—; wherein R²⁰ is selected from the group consisting of: H; straight chain or branched C₁˜C₁₂ alkyl;

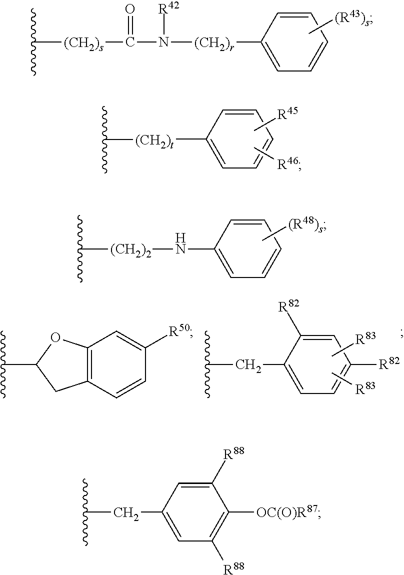

—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula III is an imidazolium or triazolium salt with a pharmaceutically acceptable counter anion or an internal salt (zwitterion); or wherein R⁶³ is a moiety that is readily cleaved in vivo, wherein R⁶³ optionally is selected from the group consisting of —CHR⁴²OC(═O)(O)ₙR⁵⁵ or —C(R⁴²)₂OP(═O)(OR¹⁰⁶)₂ and the compound of Formula III is a prodrug and an imidazolium or triazolium salt with a pharmaceutically acceptable counter ion or an internal salt (zwitterion); wherein s is 1 or 2; wherein t is an integer from 1 to 4 inclusive; wherein u is 2 or 3; wherein R²² is H; F, Cl, Br; I; —NO₂, —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical containing one S and one or two N atoms, said heterocyclic radical optionally selected from the group consisting of thiazolyl or thiazol-4-yl; wherein R²¹ is H; or —(CR²⁵R²⁶)ₘ—CR²⁷R²⁸-Q-R²⁹; wherein each m is independently an integer from 0 to 4 inclusive; wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups; wherein R²⁶ is H; or straight chain or branched C₁˜C₆ alkyl; wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(═NH)N(R⁶⁸)OR⁶⁸; —CF(R⁶⁷)C(═NH)N(R⁶⁸)OH; or —CF(R⁶⁷)C(═N—OR⁶⁹)NH₂; wherein each r is independently an integer from 0 to 2 inclusive; wherein each L is independently α-tetralyl; or phenyl, said phenyl being optionally substituted with up to three groups selected from the group consisting of: straight chain or branched C₁˜C₆ alkyl, straight chain or branched C₁˜C₆ alkoxy, —C≡N, —NO₂, or —NH₂; wherein R²⁸ is H; F; Cl; Br; —OR⁴²; —OC(═O)R¹⁰⁰;

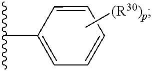

straight chain or branched C₁˜C₆ alkyl; R⁶¹; or —OP(═O)(OH)₂ or a pharmaceutically acceptable salt or zwitterion form thereof; wherein each Q is independently a covalent bond; —O—; —S—; —S(O)—; or —S(O)₂—; wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; straight chain or branched C₁˜C₆ alkyl optionally substituted with —CO₂H; or

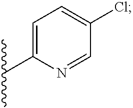

wherein each p is independently an integer from 0 to 3 inclusive; wherein each T is independently a covalent bond; —CH₂CH₂—; —CH═CH—; —(O)—C(R⁴²)₂—; —CH₂O—; —SCH₂—; —CH₂S—; —OCH₂CH₂O—; —CH(CH₃)—; —CH₂—; —CF₂—; —C(R⁹³)₂—; or

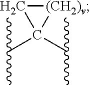

wherein each v is independently an integer from 1 to 3 inclusive; wherein each U is independently a covalent bond;

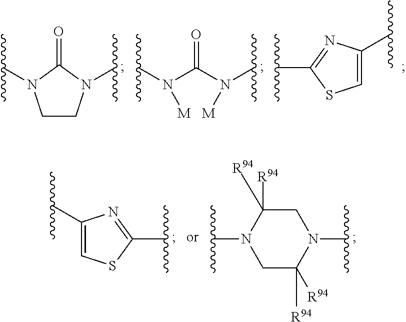

wherein each V is independently H; —S(O)₂CH₃; —C(O)NH₂; —CH₂C≡CH; —CH═CH₂; —S(O)ᵣ—R⁵⁵;

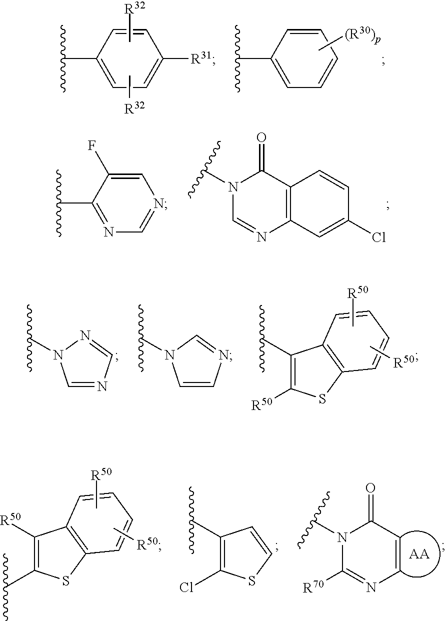

—R⁵⁹; —R⁶¹; or -LL-R⁹⁵; wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings; wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴; —OC(O)N(R⁵⁴)₂: —SH; —SR⁴¹: —S(O)ᵣR⁵⁸; —CH═CH—CO₂H;

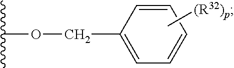

straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to —CH₂C≡N, —CH₂CH₂C≡N, —CH₂CH₂CH₂C≡N, and —CH₂CH(CH₃)C≡N; straight chain or branched C₁˜C₆ halogenoalkyl optionally selected from the group consisting of —CF₃, —CF₂CF₃, —CH₂CF₃, —CH₂Cl, and —CH₂Br; straight chain or branched C₁˜C₅ halogenoalkoxy optionally selected from the group consisting of —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₈ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy; wherein each R³¹ is independently H; F; Cl; Br; I; —C≡N; —NO₂; —CF₃; —N═C═S; —N(R⁴²)₂; —NH—C(═O)-(M)ₙ-R⁵⁴; —NH—C(═S)—NH—R⁵⁴; straight chain or branched C₁˜C₆ alkoxy or alkylthio; straight chain or branched C₁˜C₁₂ alkyl; —(CH₂)ᵣ—R⁹⁹;

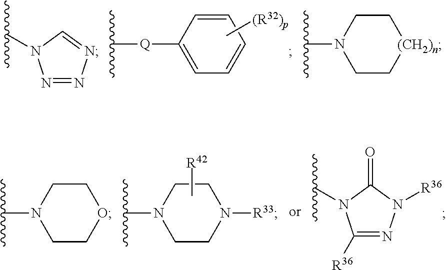

wherein each M is independently O or NH, with the proviso that when M is O and n is 1, R⁵⁴ is other than H; wherein each R³² is independently H; F; Cl; Br; I; —C≡N; —NO₂; straight chain or branched C₁˜C₁₂ alkyl; straight chain or branched C₁˜C₄ alkoxy; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; straight chain or branched C₁˜C₃ halogenoalkoxy optionally selected from the group consisting of —OCF₃, —OCF₂CF₃, —OCH₂CF₃, —OCHF₂, —OCH₂F; straight chain or branched C₁˜C₄ alkylthio or haloalkylthio; straight chain or branched C₁˜C₄ alkylthionyl or haloalkylthionyl; or straight chain or branched C₁˜C₄ alkylsulfonyl or haloalkylsulfonyl; wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)—C(O)OR³⁴; —C(O)O-phenyl; —(CH₂)—C(O)N(R⁴²)₂; —CH₂C(S)N(R⁴²)₂; —CH₂C(S)SR³⁴; —(CH₂)ₙ-phenyl; benzoyl, wherein said benzoyl is optionally substituted with one or two R⁴⁶ groups; or

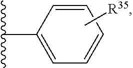

or, preferably,

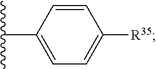

wherein each R³⁴ is independently straight chain or branched C₁˜C₆ alkyl; wherein each R³⁵ is independently H;

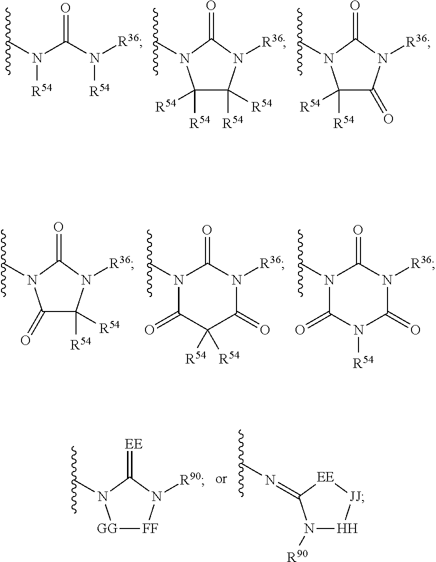

wherein each R³⁶ is independently H; straight chain or branched C₃˜C₇ alkenyl, with the proviso that the olefinic double bond is not located a to N; straight chain or branched C₃˜C₇ alkynyl, with the proviso that the alkynyl CC bond is not located α to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or

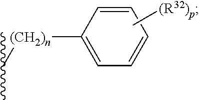

wherein each n is independently 0 or 1; wherein R²⁶ and R²⁸ together with the two carbon atoms to which they attach can be C═C; wherein R²⁷ and R²⁸ together can be

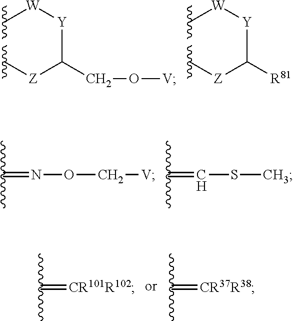

wherein W is —CH₂— and Y is —CH₂—; W is —O— and Y is —CH₂—; or W is —CH₂— and Y is —O—; wherein Z is —CH₂— or —O—; wherein R²⁷ together with -Q-R²⁹ can be

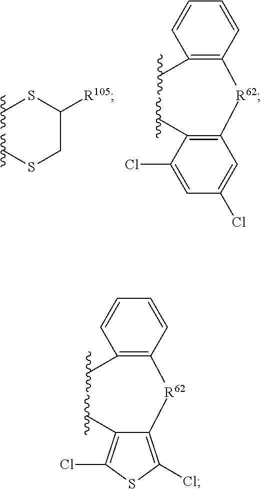

or, when R²⁷ and R²⁹ each independently represents a cycloalkyl, aryl or heterocyclic ring and Q is a covalent bond, R²⁷ together with -Q-R²⁹ can be

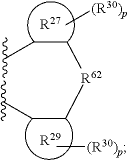

wherein each R³⁷ is independently H; C₁˜C₂ alkyl or phenyl; wherein each R³⁸ is independently H or C₁˜C₂ alkyl;
wherein each R³⁹ is independently H; straight chain or branched C₁˜C₅ alkyl; straight chain or branched C₁˜C₅ alkanoyl; or straight chain or branched C₁˜C₅ haloalkanoyl optionally selected from the group consisting of —C(O)CF₃, —C(O)CF₂CF₃, —C(O)CH₂CF₃, —C(O)CHF₂, and —C(O)CH₂F; wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J; wherein each R⁴¹ is independently straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J; wherein each q is independently an integer from 0 to 4 inclusive; wherein each G¹ is independently a covalent bond; —O—; or —S—; wherein each J is independently phenyl, 2-thienyl or 3-thienyl wherein said phenyl, 2-thienyl or 3-thienyl is optionally substituted with one to three groups selected from the group consisting of: F; Cl; Br; I; —NO₂; straight chain or branched C₁˜C₅ alkyl; straight chain or branched C₁˜C₅ alkoxy; wherein each R⁴² is independently H; or straight chain or branched C₁˜C₆ alkyl; wherein each R⁴³ is independently H; F; Cl; Br; I; —NO₂; straight chain or branched C₁˜C₆ alkyl; straight chain or branched C₁˜C₆ alkoxy; —CF₃; or —O— phenyl; wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the group consisting of: F, Cl, Br, I, straight chain or branched C₁˜C₆ alkyl, or straight chain or branched C₁˜C₆ alkoxy; wherein each R⁴⁵ is independently H; F; Cl; Br; I; —NO₂; straight chain or branched C₁˜C₆ alkyl; straight chain or branched C₁˜C₆ alkoxy; or —S(O)₂NH₂; wherein each R⁴⁶ is independently H; F; Cl; Br; I; straight chain or branched C₁˜C₆ alkyl; straight chain or branched C₁˜C₆ alkoxy; or —C≡N; wherein each R⁴⁷ is independently a phenyl group optionally substituted with one to three F, Cl, Br or I; wherein each R⁴⁸ is independently H; F; Cl; Br; I; or straight chain or branched C₁˜C₆ alkyl; wherein each R⁴⁹ is independently 1-naphthyl; 2-naphthyl; or phenyl, wherein said phenyl is optionally substituted with one or two groups, each independently selected from the group consisting of: F, Cl, Br, I, straight chain or branched C₁˜C₆ alkyl, straight chain or branched C₁˜C₆ alkoxy, or —CH₂CH═CH₂; wherein each R⁵⁰ is independently H; F; Cl; Br; or I; wherein each R⁵¹ is independently phenyl, wherein said phenyl is optionally substituted with one or two groups, each independently selected from the group consisting of: F, Cl, Br, I, or straight chain or branched C₁˜C₆ alkyl; wherein each R⁵² is independently phenyl, wherein said phenyl is optionally substituted with one or two groups, each independently selected from the group consisting of: F, Cl, Br, or I; wherein each R⁵³ is independently straight chain or branched C₁˜C₈ alkyl optionally substituted with C₃˜C₇ cycloalkyl, C₆-C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; straight chain or branched C₃˜C₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl C≡C bond is located β, γ or δ with respect to the ether oxygen; wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl; wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle can be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups; wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle can be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle can be aromatic or non-aromatic; methylenedioxy; —C≡N; —NO₂; —CF₃; —N(R⁵⁴)₂; —OR⁵⁴; —SH; ═O; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; —C(═NH)N(R⁵⁴)OH; —C(═N—OR⁵⁴)NH₂; —S(O)₂N(R⁵⁴)₂; —NR⁵⁴S(O)₂R⁵⁴; —C(O)NHS(O)₂R⁵⁴; —S(O)₂R⁵⁴; —SO₃H; or —PO₃H₂; wherein each R⁵⁷ is independently F; Cl; Br; I; straight chain or branched C₁˜C₆ alkyl; straight chain or branched C₁˜C₆ alkoxy; —C≡N; —CF₃; —NO₂, —NH₂; or —NHC(O)R⁶⁶, wherein said R⁶⁶ is straight chain or branched C₂˜C₁₂ alkyl; wherein each R⁵⁸ is independently straight chain or branched C₁˜C₂₀ alkyl; C₃˜C₈ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups; wherein each R⁵⁹ is independently straight chain or branched C₁˜C₇ alkyl, alkenyl or alkynyl, said alkyl, alkenyl or alkynyl being substituted with one or more —R⁶⁰—R⁶¹; each R⁶⁰ is independently a covalent bond or —O—; wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; wherein each R⁶² is independently a covalent bond; —CH₂—CH₂—; —CH═CH—; —O—; or —S—; wherein each R⁶⁵ is independently straight chain or branched C₁˜C₁₂ acyclic hydrocarbon group, which can be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or C≡C bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups; wherein each R⁶⁷ is independently H; F; or straight chain or branched C₁˜C₆ alkyl, said alkyl group being optionally substituted by one or more R³⁰; wherein each R⁶⁸ is independently R³⁶, R⁵⁴ or R⁶¹; wherein each R⁶⁹ is independently H; or straight chain or branched C₁˜C₆ alkyl, said alkyl group being optionally substituted by one or more R³⁰; wherein each R⁷⁰ is independently H; straight chain or branched C₁˜C₄ alkyl; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; or cyclopropyl; wherein each R⁷¹ is independently R⁵⁶; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)rR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶;

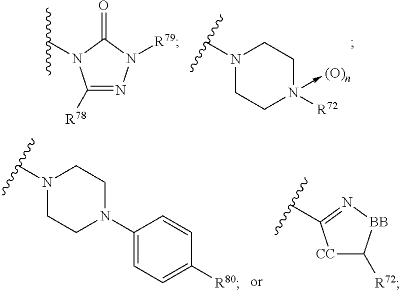

wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy; wherein each R⁷³ is independently H; straight chain or branched C₁˜C₄ alkyl; C₃˜C₆ cycloalkyl; —C(O)R⁷²; or —C(O)CF₃; wherein each R⁷⁴ is independently straight chain or branched C₁˜C₄ alkyl; wherein each R⁷⁵ is independently H; —C(O)NH₂; —C(O)CH₃; —C≡N; —SO₂NHR⁷²; —SO₂R⁷²; —OR⁷²; —OC(O)R⁷²; or straight chain or branched —(C₁˜C₄ alkyl)-NH₂; wherein each R⁷⁶ is independently phenyl optionally substituted with one or more R⁷⁷ groups; wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²;

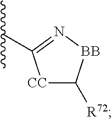

or phenyl, said phenyl being optionally substituted with one or more F, Cl, Br, I, —C≡N, —NO₂, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy; wherein each R⁷⁸ is independently H or —CH₃; wherein each R⁷⁹ is independently H; isopropyl; cyclopentyl; cyclopropyl; 2-butyl; 3-pentyl; 3-hydroxy-2-butyl; or 2-hydroxy-3-pentyl; wherein each R⁸⁰ is independently F; Cl; Br; I; —C≡N; —NO₂; —NH₂; straight chain or branched C₁˜C₄ halogenoalkyl; straight chain or branched C₁˜C₄ halogenoalkoxy; or

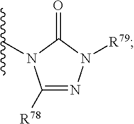

wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —C≡N, —NO₂, phenyl, phenylmethyl, benzoyl, halobenzoyl, —C(O)R⁴², —C(O)OR⁴², and —CF₃, with the proviso that when multiple substituents are present only 1 thereof can be selected from the group consisting of phenyl, phenylmethyl, benzoyl and halobenzoyl; wherein each R⁸² is independently H or —OR⁸⁴; wherein each R⁸³ is independently H; F; Cl; Br; R⁷⁴; —OR⁷⁴; —SR⁷⁴; —CH₂SC(═O)R⁷⁴; —COOH; or —C(═O)OCH₃; wherein each R⁸⁴ is independently a group that is easily hydrolyzable under physiological conditions, optionally at the acyl residue of an amino acid or a group represented by the formula —C(═O)R⁸⁵ or —P(═O)(OR⁸⁶)₂; wherein R⁸⁴ is optionally selected from the group consisting of formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like; wherein each R⁸⁵ is independently H; —OR⁷⁴; or straight chain or branched C₁˜C₆ alkyl or alkenyl, wherein said alkyl or alkenyl can be optionally interrupted by a hydrolyzable functional group such as carboxamide or ester, wherein said alkyl or alkenyl can be optionally substituted with —COOH, —NH₂, —NHR⁷⁴, —N(R⁷⁴)₂, or R⁶¹, wherein R⁶¹ is optionally selected from the group consisting of phenyl, methoxyphenyl, pyridyl, pyrazinyl or furyl; wherein each R⁸⁶ is independently H or R⁷⁴; wherein each R⁸⁷ is independently straight chain or branched C₁˜C₅ alkyl; R⁶¹; pyridyl; pyrrolidinyl; or -DD—NH—R⁸⁹; wherein each R⁸⁸ is independently H; F; Cl; Br; or —OR⁷⁴; wherein each DD is independently straight chain or branched C₁˜C₄ alkylene; —CH₂NHC(═O)CH₂—; —CH(NH₂)CH₂CH₂CH₂—; wherein each R⁸⁹ is independently H or straight chain or branched C₁˜C₅ alkyl; wherein each BB is independently —CH₂— or —C(═O)—; wherein each CC is independently NH or O; wherein each EE is independently O, S or N—R⁹¹; wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃˜C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched; wherein each KK is independently O or S; wherein each R⁹¹ is independently H or straight chain or branched C₁˜C₆ alkyl; wherein each R⁹² is independently H; straight chain or branched C₁˜C₆ alkyl; C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—; wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; wherein each GG is independently —C(═O)—; or —CH₂—, optionally substituted with up to two radicals selected from the group consisting of straight chain or branched C₁˜C₆ alkyl and straight chain or branched C₁˜C₆ alkoxy; or FF and GG, taken together, form a bivalent radical of formula —N═CH—(FFGG′); wherein HH has the same meaning as FF; wherein JJ has the same meaning as GG; or HH and JJ, taken together, form a bivalent radical of formula —N═CH—(FFGG′) or —CH═CH—(HHJJ′); wherein the nitrogen atom in the bivalent radical FFGG′ is connected to NR⁹⁰; wherein one hydrogen in said radical FFGG′ and up to two hydrogens in radical HHJJ′ can be replaced by a straight chain or branched C₁˜C₆ alkyl radical; wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group optionally selected from the group consisting of —CH₂CH₂—, —CH₂CH₂CH₂—, —CH₂CH₂CH₂CH₂— and the like; wherein each R⁹⁴ is independently H or C₁˜C₄ alkyl which is unsubstituted or substituted by 1-3 substituents each independently selected from the group consisting of halogen, hydroxyl, C₁˜C₄ alkoxy, and amino; or two R⁹⁴ groups attached to the same carbon atom together form ═S; wherein each LL is independently a covalent bond; —C(═O)—; —C(═S)—; —SO₂—; or —N═N—; wherein each R⁹⁵ is independently selected from the group consisting of i) hydrogen, ii) CN iii) CHO iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl, v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido, vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl, vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and viii) a group of the formula

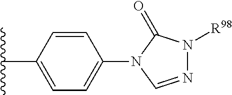

wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which can be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl; wherein each MM is independently an ethylene group optionally substituted with R⁹¹; or MM is —NN═OO—, wherein NN and OO are the same or different and are each independently N or CR⁹¹; wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶; wherein each R¹⁰⁰ is independently —R⁵⁴ or —OR⁵⁴; wherein each R¹⁰¹ is independently H; F; Cl; Br; or I; wherein each R¹⁰² is independently F; Cl; Br; I; —SR¹⁰³; or —OR¹⁰⁴; wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which can be the same or different; wherein each R¹⁰⁴ is independently straight chain or branched C₁˜C₁₂ alkyl; or phenyl; wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰; wherein each R¹⁰⁶ is independently H, a pharmaceutically acceptable cation, or null (affording an azolium phosphate zwitterion); wherein each chiral center independently may possess any relative or absolute stereoconfiguration or be any mixture thereof, unless specified otherwise herein; and wherein each olefinic C═C bond that is capable of E/Z isomerism independently may possess either the E or Z stereoconfiguration or be any mixture thereof, unless specified otherwise; wherein the imidazole analog improves efficacy of the at least one prostaglandin analog of Formula I when the composition is delivered to a subject refractory to treatment by a composition containing the prostaglandin analog of Formula I alone. According to another embodiment, the imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof. According to another embodiment, the imidazole analog is miconazole or ketoconazole or a pharmaceutically acceptable salt, hydrate, solvate, prodrug or metabolite thereof. According to another embodiment, the at least one compound of formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog. According to another embodiment, the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-(S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-(R,E)-3-hydroxy-4-(3-(trifluoromethyl)phenoxy)but-1-enyl)-5-oxocyclopent-3-enyl)-N-methylhept-5-enamide (Z)-7-((1R,2S)-2-(R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-(S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-(R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof. According to another embodiment, the prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z) —N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂ N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z) —N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z) —N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof. According to another embodiment, the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof. According to another embodiment, the optional second component of the composition comprises an additional active agent selected from the group consisting of a protective agent, an emollient, an astringent, an irritant, a keratolytic, a sun screening agent, a sun tanning agent, an antibiotic agent, a non-imidazole analog antifungal agent, an imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant, a chemotherapeutic agent, an anti-histamine, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent, a hypo-pigmenting agent, or a combination thereof. According to another embodiment, the at least one chemotherapeutic agent is at least one agent selected from the group consisting of an alkylating agent, an antimetabolite, a natural product, a biologic agent, a hormone or hormone-related agent, and a miscellaneous agent. According to another embodiment, the at least one chemotherapeutic agent is an alkylating agent, and wherein the side-effect of treatment with the alkylating agent is alopecia. According to another embodiment, the alkylating agent is selected from the group consisting of temozolomide, busulfan, ifosamide, melphalan hydrochloride, carmustine, lomustine and cyclophosphamide. According to another embodiment, the at least one chemotherapeutic agent is an antimetabolite, and wherein the side-effect of treatment with the antimetabolite is alopecia. According to another embodiment, the antimetabolite is selected from the group consisting of 5-fluorouracil, capecitabine, gemcitabine, floxuridine, decitabine, mercaptopurine, pemetrexed disodium, methotrexate and dacarbazine. According to another embodiment, the at least one chemotherapeutic agent is a natural product, and wherein the side-effect of treatment with the natural product is alopecia. According to another embodiment, the natural product is selected from the group consisting of vincristine, vinblastine, vinorelbine tartrate, paclitaxel, docetaxel, ixabepilone, daunorubicin, epirubicin, doxorubicin, idarubicin, mitoxantrone, mitomycin, dactinomycin, irinotecan, topotecan, etoposide, teniposide, etoposide phosphate, and bleomycin sulfate. According to another embodiment, the at least one chemotherapeutic is a biologic agent, and wherein the side-effect of treatment with the biologic agent is alopecia. According to another embodiment, the biologic agent is selected from the group consisting of filgrastim, pegfilgrastim, bevacizumab, sargramostim and panitumumab. According to another embodiment, the at least one chemotherapeutic agent is a hormone or a hormone-related agent, and wherein the side-effect of treatment with the hormone or the hormone-related agent is alopecia. According to another embodiment, the hormone or the hormone-related agent is selected from the group consisting of megestrol acetate, fluoxymesterone, leuprolide, octreotide acetate, tamoxifen citrate and fluxymesterone. According to another embodiment, the at least one chemotherapeutic agent is a miscellaneous agent, and wherein the side-effect of treatment with the miscellaneous agent is alopecia. According to another embodiment, the miscellaneous agent is selected from the group consisting of sorafenib, erlotinib, oxaliplatin, dexrazoxane, anagrelide, isotretinoin, bexarotene and vorinostat. According to another embodiment, the topical composition is administered concurrently with a chemotherapy regimen for treating breast cancer. According to another embodiment, the chemotherapy regimen is an Adriamycin-Cytoxan-Taxol regimen. According to another embodiment, the topical composition is administered concurrently with a chemotherapy regimen for treating colorectal cancer. According to another embodiment, the chemotherapy regimen comprises a combination of infusional 5-fluoruracil, leucovorin and oxaliplatin (FOLFOX®). According to another embodiment, the chemotherapeutic regimen for colorectal cancer comprises FOLFOX® with bevacizumab. According to another embodiment, the chemotherapy regimen comprises infusional 5-fluoruracil, leucovorin, and irinotecan (FOLFIRI®) with bevacizumab. According to another embodiment, the composition is formulated as a mascara.

### DETAILED DESCRIPTION

### Glossary

The term “acne” as used herein refers to an inflammatory disease of the sebaceous glands, characterized by comedones and pimples. The term “anti-acne” as used herein refers to agents that alleviate the symptoms of acne. Examples of anti-acne agents include, without limitation, keratolytics, such as salicylic acid, sulfur, glycolic, pyruvic acid, resorcinol, and N-acetylcysteine; and retinoids such as retinoic acid and its derivatives (e.g., cis and trans, esters).

The term “active” refers to the ingredient, component or constituent of the compositions of the described invention responsible for the intended therapeutic effect.

The phrase “additional active ingredient” as used herein refers to an agent, other than a compound of the described composition, that exerts a pharmacological, dermatological or any other beneficial activity. It is to be understood that “other beneficial activity” can be one that is only perceived as such by the subject using the inventive compositions. Such additional active agents include, but are not limited to, an antifungal agent, an antibiotic, an antiviral agent, an antiprotozoal agent, an anesthetic agent, a chemotherapeutic agent, a vitamin, a hormone and a steroid.

The terms “administering” or “administration” as used herein are used interchangeably to mean the giving or applying of a substance and include in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing the conventional nontoxic pharmaceutically acceptable carriers, adjuvants, and vehicles as desired, or may be locally administered by means such as, but not limited to, injection, implantation, grafting, topical application, or parenterally. The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents. Additional administration may be performed, for example, intravenously, pericardially, orally, via implant, transmucosally, transdermally, intramuscularly, subcutaneously, intraperitoneally, intrathecally, intralymphatically, intralesionally, or epidurally. Administering can be performed, for example, once, a plurality of times, and/or over one or more extended periods either as individual unit doses or in the form of a treatment regimen comprising multiple unit doses of multiple drugs and/or substances.

The term “adjuvant therapy” refers to a treatment added to a curative treatment to prevent recurrence of clinical cancer from microscopic residual disease; the additional therapy given to enhance or extend primary therapy's effect, as in chemotherapy's addition to a surgical regimen.

The term “alopecia” is a medical term for the absence or loss of hair including eyelashes, eyebrows, and scalp hair, as a result of illness, functional disorder, or hereditary disposition. For example, the term “Alopecia adnata” refers to underdevelopment of the eyelashes. Alopecia frequently occurs in patients undergoing treatment for cancer or suffering from other diseases, such as AIDS, where cell-killing, or cytotoxic, drugs are used. As used herein, hair loss, culminating in alopecia, includes sparse hair growth, short hair growth, thin hair growth, brittle hair growth, and hair depigmentation.

The term “alopecia greata” refers to a common condition of undetermined etiology characterized by circumscribed, nonscarring, usually asymmetrical areas of baldness on the scalp, eyebrows, and bearded portion of the face.

The term “anagen effluvium” refers to the hair loss associated with chemotherapeutic agents that cause immediate destruction and release of anagen hair.

The term “androgenic alopecia” refers to a gradual decrease of scalp hair density in adults with transformation of terminal to vellus hairs, which become lost as a result of familial increased susceptibility of hair follicles to androgen secretion following puberty

The term “anesthetic agents” as used herein refers to agents that resulting in a reduction or loss of sensation. Non-limiting examples of anesthetic drugs that are suitable for use in the context of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-dandruff agents” as used herein refers to agents that reduce, eliminate or prevent a scurf from forming on skin, especially of the scalp, that comes off in small white or grayish scales. Exemplary anti-dandruff ingredients usable in context of the described invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, and ciclopiroxolamine, and mixtures thereof.

The term “anti-fungal agent” as used herein means any of a group of chemical substances having the capacity to inhibit the growth of or to destroy fungi. Anti-fungal agents include, but are not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconawle, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “antihistamine agent” as used herein refers to any of various compounds that counteract histamine in the body and that are used for treating allergic reactions (such as hay fever) and cold symptoms. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine.

The term “anti-irritant” as used herein refers to an agent that prevents or reduces soreness, roughness, or inflammation of a bodily part.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation, include pyrimethamine (Daraprim®) sulfadiazine, and Leucovorin.

The term “antimetabolite” as used herein refers to any substance that interferes with growth of an organism by competing with or substituting for an essential nutrient in an enzymatic process. “Antimetabolite” also may refer to certain substances used to prevent or reduce proliferation of cells by interfering with normal metabolic activity.

The term “antipruritic agents” as used herein refers to those substances that reduce, eliminate or prevent itching. Antipruritic agents include, without limitation, pharmaceutically acceptable salts of methdilazine and trimeprazine.

The term “anti-oxidant agent” as used herein refers to a substance that inhibits oxidation or reactions promoted by oxygen or peroxides. Non-limiting examples of anti-oxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename TroloxR), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

The term “anti-skin atrophy actives” refers to substances effective in replenishing or rejuvenating the epidermal layer by promoting or maintaining the natural process of desquamation. Non-limiting examples of antiwrinkle and antiskin atrophy actives which can be used in context of the described invention include retinoic acid its prodrugs and its derivatives (e.g., cis and trans) and analogues; salicylic acid and derivatives thereof, sulfur-containing D and L amino acids and their derivatives and salts, particularly the N-acetyl derivatives, a preferred example of which is N-acetyl L-cysteine; thiols, e.g. ethane thiol; alpha-hydroxy acids, e.g. glycolic acid, and lactic acid; phytic acid, lipoic acid; lysophosphatidic acid, and skin peel agents (e.g., phenol and the like).

The term “astringents” generally refer to protein precipitants, that have such a low cell penetrability that the action essentially is limited to the cell surface and interstitial spaces. Astringents are locally applied. The astringent action is accompanied by contraction and wrinkling of the tissue and by blanching. Astringents are used therapeutically to arrest hemorrhage by coagulating the blood, to promote healing, to toughen the skin or to decrease sweating. The principal components of astringents are salts of aluminum, zinc, manganese, iron or bismuth.

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

The term “appearance” as used herein refers to an outward aspect or presentation of oneself.

The term “associate” or any of its grammatical forms as used herein refers to joining, connecting, or combining to, either directly, indirectly, actively, inactively, inertly, non-inertly, completely or incompletely.

The term “brittle” as used herein refers to being easily cracked, fractured, snapped or otherwise damaged when subject to stress.

The term “carbohydrate” as used herein refers to aldehyde or ketone compounds with multiple hydroxyl groups. The term “monosaccharide” or “simple sugar” refers to a carbohydrate that does not hydrolyze. Non-limiting examples of monosaccharides include glucose (dextrose), fructose, galactose, xylose and ribose. Monosaccharides are the building blocks of disaccharides like sucrose (common sugar) and polysaccharides (such as cellulose and starch). Further, each carbon atom that supports a hydroxyl group (except for the first and last) is chiral, giving rise to a number of isomeric forms all with the same chemical formula.

The terms “cancer” or “malignancy” as used herein refer to diseases in which abnormal cells divide without control and can invade nearby tissues. Cancer cells also can spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in cells of the immune system. Central nervous system cancers are cancers that begin in the tissues of the brain and spinal cord.

The term “carrier” as used herein describes a material that does not cause significant irritation to an organism and does not abrogate the biological activity and properties of the compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both.

The term “caustic agents” as used herein refers to substances capable of destroying or eating away epithelial tissue by chemical action. Caustic agents can be used to remove dead skin cells. For example, beta-hydroxy acids, naturally derived acids with a strong keratolytic effect, are useful for problem skin, acne or peeling.

The terms “chelating agent”, “chelant” or “chelator” refer to a chemical that forms soluble complex molecules with certain metal ions thereby inactivating the ions.

The term “chemotherapeutic agent” as used herein refers to chemicals useful in the treatment or control of a disease. Non-limiting examples of chemotherapeutic agents usable in context of the described invention, including those in Tables 1-6, include temozolomide, busulfan, ifosamide, melphalan, carmustine, lomustine, mesna, 5-fluorouracil, capecitabine, gemcitabine, floxuridine, decitabine, mercaptopurine, pemetrexed disodium, methotrexate, vincristine, vinblastine, vinorelbine tartrate, paclitaxel, docetaxel, ixabepilone, daunorubicin, epirubicin, doxorubicin, idarubicin, amrubicin, pirarubicin, mitoxantrone, etoposide, etoposide phosphate, teniposide, mitomycin C, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

The phrases “chemotherapy hair loss” or “chemotherapy-related hair loss” as used herein refers to an adverse effect towards hair that includes at least one of the following: short hair growth, sparse hair growth, brittle hair growth, thin hair growth, hair loss, alopecia, and hair depigmentation that results from exposure to at least one chemotherapeutic agent.

The term “chemotherapy insult” as used herein refers to an injury or trauma resulting, directly or indirectly, from exposure to a chemotherapeutic agent.

The term “chemotherapy regimen” (“combination chemotherapy”) means chemotherapy with more than one drug in order to benefit from the dissimilar toxicities of the more than one drug. A principle of combination cancer therapy is that different drugs work through different cytotoxic mechanisms; since they have different dose-limiting adverse effects, they can be given together at full doses.

The term “cicatricial alopecia”, (or “scarring alopecia”) refers to a collection of hair loss disorders While there are many forms of scarring alopecia, the common theme is a potentially permanent and irreversible destruction of hair follicles and their replacement with scar tissue. Examples include bullous diseases, chemical alopecia, discoid lupus erythematosus, folliculitis (severe), lichen planopilaris, dissecting cellulitis, and tumors.

The term “color” as used herein refers to the quality of an object or substance with respect to light reflected or absorbed by the object or substance. The three characteristics of color are hue, intensity, and value. “Hue” refers to a gradation, tint, or variety of a color. “Intensity”, “chroma”, and “saturation” are used interchangeably to refer to the strength or sharpness of a color. A color is full in intensity only when pure and unmixed. “Value” refers to a degree of lightness or darkness in a color.

The term “compatible” as used herein means that the components of a composition are capable of being combined with each other in a manner such that there is no interaction that would substantially reduce the efficacy of the composition under ordinary use conditions.

The term “concurrent” as used herein refers to occurring, or to operating, before, during or after an event, episode or time period.

The term “condition”, as used herein, refers to a variety of health states and is meant to include disorders or diseases caused by any underlying mechanism or disorder, injury, and the promotion of healthy tissues and organs.

The term “configuration” refers to the three-dimensional shape of a molecule. In order to represent three-dimensional configurations on a two-dimensional surface, perspective drawings in which the direction of a bond is specified by the line connecting the bonded atoms are used. Formula A shows an illustrative perspective drawing:

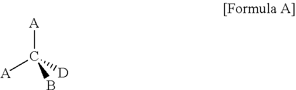

In formula A, the focus of configuration is a carbon (C) atom so the lines specifying bond directions will originate there. A simple straight line represents a bond lying approximately in the surface plane, as shown by the two bonds to substituent “A.” A wedge shaped bond is directed in front of this plane (thick end toward the viewer), as shown by the bond to substituent “B.” A hatched bond is directed in back of the plane (away from the viewer), as shown by the bond to substituent “D.” In some formulas, a dashed line represents a single or double bond which can be in the cis or trans configuration (not shown here).

The term “contact” as used herein refers to a state or condition of touching or of being in immediate or local proximity. The term “contacting” as used herein refers to bringing or putting in contact, or to being in or coming into contact. Contacting a composition to a target destination, such as, but not limited to, an organ, tissue, cell, or tumor, may occur by any means of administration known to the skilled artisan.

The term “controlled release” is intended to refer to any drug-containing formulation in which the manner and profile of drug release from the formulation are regulated.

The term “cosmetic composition’ as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The phrase “cosmetically acceptable carrier” as used herein refers to a substantially non-toxic carrier, conventionally useable for the topical administration of cosmetics, with which compounds will remain stable and bioavailable.

The term “colorant” as used herein refers to substance, dye, pigment, ink or paint that colors or modifies the hue of something. Colorants include pigments or dyes or a combination thereof as the cosmetic benefit requires.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.” For example, delayed absorption of a parenterally administered drug form may be accomplished by dissolving or suspending the drug in an oil vehicle.

The term “demulcents” as used herein refers to protective agents employed primarily to alleviate irritation, particularly mucous membranes or abraded tissues. They often are applied to the surface in a viscid, sticky preparation that covers the area readily and may be medicated. A number of chemical substances possess demulcent properties. These substances include, but are not limited to, the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

The term “depression” as used herein means a condition of general emotional dejection and withdrawal; a sadness greater and more prolonged than that warranted by any objective reason. The term “clinical depression” means a depression so severe as to be considered abnormal, either because of no obvious environmental causes, or because the reaction to unfortunate life circumstances is more intense or prolonged than generally would be expected.

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.”

The term “disease” or “disorder”, as used herein, refers to an impairment of health or a condition of abnormal functioning.

The term “distressed” as used herein means affected with or suffering at least one symptom of depression or a mood disorder, anxiety, sorrow, mental sorrow, worry, stress, strain, shame, uncomfortable self-conscious, embarrassment, humiliation, trouble, and/or combination thereof.

The term “drug” as used herein refers to a therapeutic agent or any substance, other than food, used in the prevention, diagnosis, alleviation, treatment, or cure of disease.

The terms “drying agent” or “desiccant” as used herein refers to a substance that has an affinity for water such that it will extract the water from other materials.

The term “emollients” as used herein generally refers to bland, fatty or oleaginous materials, which can be applied locally, particularly to the skin. Emollients increase the tissue moisture content, thereby rendering the skin softer and more pliable. Increased moisture content in the skin can be achieved by preventing water loss with an occlusive water-immiscible barrier, by increasing the water-holding capacity in the skin with humectants, or by altering the desquamation of the outermost skin layer, the stratum corneum. Useful emollients include, but are not limited to, lanolin, spermaceti, mineral oil, paraffin, petrolatum, white ointment, white petroleum, yellow ointment. Also included are vegetable oils, waxes, cetyl alcohol, glycerin, hydrophilic petrolatum, isopropyl myristate, myristyl alcohol, and oleyl alcohol.

The term “emulsifiers” as used herein are agents that promote the formation and stabilization of an emulsion.

The term “emulsion” as used herein refers to a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

The term “enhance” as used herein in its various grammatical forms refers to an increase or intensify in quality or quantity, or to make better or augment.

The terms “epithelia” or “epithelial” or “epithelial tissues” as used herein are meant to include skin and mucosal membranes. Thus, the described invention offers compositions useful for treating a condition of the skin or a mucosal membrane, such as, but not limited to, that of a nose, an eye, a face, and a scalp

The term “eyebrow” refers to an area of coarse skin hairs above the eye that follows the shape of the brow ridges.

The terms “eyelash” and “lash” are used interchangeably to refer to one of the hairs that grow at the edge of the eyelid.

The phrase “new excipient” as used herein means any inactive ingredient that is intentionally added to the composition of the described invention and is not intended to exert therapeutic effects at the intended dosage, although it may act to improve product delivery. A new excipient is not fully qualified by existing safety data with respect to the currently proposed level of exposure, duration of exposure or route of administration. Additional characteristics of new excipients can be found in the Guidance for Industry Nonclinical Studies for the Safety Evaluation of Pharmaceutical Excipients issued by the US Food and Drug Administration Center for Drug Evaluation and Research, in May, 2005, herein incorporated by reference.

The term “growth” as used herein refers to a process of becoming larger, longer or more numerous, or an increase in size, number, or volume.

The term “hair loss’ as used herein refers to a decline in the amount of hair as a result of sparse hair growth, short hair growth, thin hair growth brittle hair growth, or alopecia.

The term “hormone” as used herein refers to natural substances produced by organs of the body that travel by blood to trigger activity in other locations or their synthetic analogs. Examples include, but are not limited to, calciferol (Vitamin D3) and its products, androgens, estrogens and progesterones.

The term “hydrate” as used herein refers to a compound formed by the addition of water or its elements to another molecule. The water usually is split off by heat, yielding the anhydrous compound.

The term “hypopigmenting agents” as used herein refers to substances capable of depigmenting the skin. Examples of hypopigmenting agents include, but are not limited to, hydroquinones, mequinol, and various protease inhibitors including serine protease inhibitors, active soy and retinoic acid.

The term “imidazole” (1,3-diazacyclopenta-2,4-diene) refers to a five-membered aromatic heterocycle having the following structure:

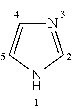

It exists in two equivalent tautomeric forms due to a hydrogen atom that may be located on either of the two nitrogen atoms.

The N-3 nitrogen atom of imidazole, which possesses a non-bonding pair of electrons, is unusually basic for an sp²-hybridized nitrogen atom. Its conjugate acid, which is called an imidazolium ion and is stabilized by resonance, has a pKₐ of approximately 7.0, as depicted below. Consequently, imidazole readily interconverts between its conjugate base and conjugate acid forms under physiological conditions, i.e. aqueous conditions near neutral pH. Furthermore, imidazole's Lewis basicity, which can be enhanced by complete or partial deprotonation of N-1, makes it an excellent ligand for many metal ions, including those that occur in biological systems.

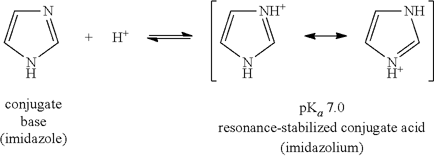

Histidine, one of the 20 endogenous amino acids that are most commonly found in proteins, contains an imidazole ring in its sidechain, which exhibits the moderate basicity and affinity for metals ions described above for imidazole itself. Due to these properties, histidine residues are essential for the normal function of many enzymes, receptors and other proteins. For example, histidine residues serve as facilitators of proton transfer in the active sites of many enzymes. Histidine residues also play several key roles in the cooperative binding and release of oxygen by hemoglobin. Decarboxylation of histidine affords histamine, an important neurotransmitter in which the imidazole moiety is essential for binding to histamine receptors.

Synthetic imidazoles are present in many fungicides, antiprotozoal and antihypertensive agents. Imidazole also is part of the theophylline molecule, found in tea leaves and coffee beans, and stimulates the central nervous system. A preservative system for ophthalmic solutions comprising imidazole and a hydrogen peroxide source has been shown to be effective against fungi and bacteria (U.S. Pat. No. 6,565,894).

Examples of known imidazoles include, but are not limited to, histidines, the antimicrobial agents bifonazole, butoconazole, chlorimidazole, hlordantoin, croconazole, clotrimazole, democonazole, eberconazole, econazole, elubiol, enilconazole, fenticonazole, flutrimazole, isocanazole, ketoconazole, lanoconazole, lombazole, miconazole, neticonazole, NND-502, omoconazole, oxiconazole, parconazole, sertaconazole, sulconazole, tiabendazole, and tioconazole, and the thromboxane synthase inhibitors 7-(1-imidazolyl)hepatanoic acid, ozagrel, and 1-benzyl imidazole.

Other nitrogen-containing 5-membered aromatic heterocycles can be considered analogs of imidazole. The term “imidazole analogs” is used herein to describe imidazoles and related 5-membered aromatic heterocycles that contain at least two nitrogen atoms in the ring. Such heterocycles are exemplified, but not limited to, 1,2,4-triazole, 1,3,4-triazole, 1,2,3-triazole, tetrazole and pyrazole, as well as thiadiazoles and oxadiazoles. Several triazoles are useful, particularly as fungicides, including albaconazole, CAS RN 214543-30-3, fluconazole, genaconzole, hydroxyitraconazole, isavuconazole, itraconazole, pramiconazole, ravuconazole, saperconazole, SYN 2869, T 8581, TAK 456, terconazole, vibunazole, voriconazole, pramiconazole, and posaconazole.

Miconazole, for example, which commonly is applied topically to the skin or to mucus membranes to treat fungal infections, such as athlete's foot and jock itch, and for vaginal yeast infections, is commercially available as a cream, lotion, powder, spray liquid, and spray powder for skin applications. Miconazole is an imidazole of the structure:

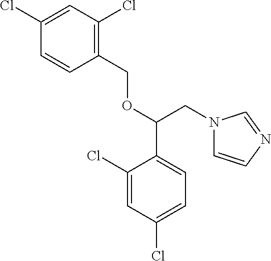

Miconazole's antifungal activity (and that of the other azole antifungals) is believed to be due to inhibition of ergosterol synthesis, specifically by inhibiting the cytochrome P450-dependent lanosterol 14α-demethylase enzyme.

Ketoconazole, an imidazole anti-fungal agent having the structure:

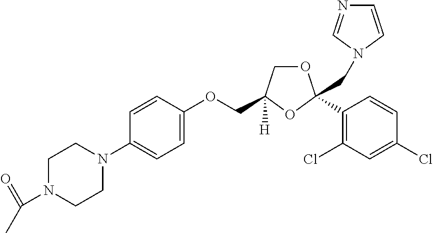

has been found to be effective in the treatment of seborrheic dermatitis. One open-label study of minoxodil 2% with ketoconazole 2% shampoo for androgenetic alopecia in men reportedly showed comparable growth in both groups, with both achieving better growth than unmedicated shampoo alone. Similar results were seen in a mouse model comparing topical ketoconazole 2% to a placebo. Ketoconazole also has been used to treat hirsutism in women, with some success. The mechanism of action is not understood.

The term “implant” refers to any device or material inserted or placed, permanently or temporarily, into or onto a subject and used for the administration or delivery of a therapeutic agent(s) or substance.

The term “improve” (or improving) as used herein refers to bring into a more desirable or excellent condition.

The term “induction regimen” (“induction chemotherapy”) as used herein means use of chemotherapy as initial treatment before surgery or radiotherapy of a malignancy.

The term “injury,” as used herein, refers to damage or harm to a structure or function of the body caused by an outside agent or force, which may be physical or chemical.

The term “intense” as used herein in its various grammatical forms refers to being of an extreme kind, very great, as in strength, keenness or severity; strong, acute, profound, or vehement, as sensations, feelings or emotions.

The term “intensity” as used herein refers to the quality or conditions of being intense.

The term “irritant” as used herein refers to a material that acts locally on the skin to induce, based on irritant concentration, hyperemia (meaning an excess of blood in an area or body part, usually indicated by red, flushed color or heat in the area), inflammation, and desiccation. Irritant agents include, but are not limited to, alcohol, aromatic ammonia spirits, benzoin tincture, camphor capsicum, and coal tar extracts.

The term “keratolytics” (desquamating agents) as used herein refers to an agent that acts to remove outer layers of the stratum corneum. They are particularly useful in hyperkeratotic areas. The keratolytics include, but are not limited to, benzoyl peroxide, fluorouracil, resorcinol, salicylic acid, tretinoin, and the like.

The term “long-term” release, as used herein, means delivery of therapeutic levels of an active ingredient for at least 7 days, or for about 30 to about 60 days.

The term “mascara” refers to a cosmetic used to darken, lighten, color, thicken, lengthen and define eyelashes.

The term “mammalian cell” as used herein refers to a cell derived from an animal of the class Mammalia. As used herein, mammalian cells may include normal, abnormal and transformed cells. Examples of mammalian cells utilized within the present invention, include, but are not limited to, neurons, epithelial cells, muscle cells, blood cells, immune cells, stem cells, osteocytes, endothelial cells and blast cells. Cells may be utilized in vivo or in vitro.

The term “metastasis” as used herein refers to the transference of organisms or of malignant or cancerous cells, producing disease manifestations, from one part of the body to other parts.

The term “modulate” as used herein means to regulate, alter, adapt, or adjust to a certain measure or proportion.

The term “moisturizing agent” as used herein refers to a substance that adds or restores moisture to the skin.

The term “mood” as used herein refers to the pervasive feeling, tone and internal emotional state of an individual which, when impaired, can markedly influence virtually all aspects of a person's behavior or his or her perception of external events.

The term “myelosuppression” as used herein refers to arrest or slowing of the bone marrow's production of blood cells and platelets.

The term “neoplasm” as used herein refers to an abnormal proliferation of genetically altered cells. A malignant neoplasm (or malignant tumor) is synonymous with cancer. A benign neoplasm (or benign tumor) is a tumor (solid neoplasm) that stops growing by itself, does not invade other tissues and does not form metastases.

The term “nonscarring alopecia” refers to hair loss without permanent destruction of the hair follicle.

The term “non-steroidal anti-inflammatory agents” refers to a large group of agents that are aspirin-like in their action, including, but not limited to, ibuprofen, naproxen sodium, and acetaminophen. Additional examples of non-steroidal anti-inflammatory agents that are usable in the context of the present invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents.

The term “penetration enhancer” as used herein refers to an agent known to accelerate the delivery of a substance through the skin.

The term “pharmaceutical composition” as used herein refers to a composition that is employed to prevent, reduce in intensity, cure or otherwise treat a target condition, syndrome, disorder or disease.

The term “pharmaceutically acceptable carrier” as used herein refers to any substantially non-toxic carrier conventionally useable for administration of pharmaceuticals in which the isolated polypeptide of the present invention will remain stable and bioavailable.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio.

The term “peptide” as used herein refers to a molecule of two or more amino acid chemically linked together. A peptide may refer to a polypeptide, protein or peptidomimetic.

The terms “polypeptide” and “protein” are used herein in their broadest sense to refer to a sequence of subunit amino acids, amino acid analogs, or peptidomimetics. The subunits are linked by peptide bonds, except where noted. The polypeptides described herein may be chemically synthesized or recombinantly expressed. Polypeptides of the described invention are chemically synthesized. Synthetic polypeptides, prepared using the well known techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl)amino acid resin with the standard deprotecting, neutralization, coupling and wash protocols of the original solid phase procedure of Merrifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptides can be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., (3-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well. In some embodiments, the peptide is of any length or size.

Use herein of the terms “peptide”, “peptides”, “polypeptide”, “peptidomimetic” or “protein” should be taken to include reference to “derivatives” of such compounds, unless the context requires otherwise, and to include “prodrugs.”

The term “peptidomimetic” as used herein refers to a small protein-like chain designed to mimic a peptide. A peptidomimetic typically arises from modification of an existing peptide in order to alter the molecule's properties.

The term “prevent” as used herein refers to the keeping, hindering or averting of an event, act, or action from happening, occurring or arising.

The term “prodrug” as used herein means a peptide or derivative, which is in an inactive form, and, which is converted to an active form by biological conversion following administration to a subject.

The term “protect” as used herein refers to defend, preserve, or guard from attack, invasion, loss, insult, injury or harm. The term “protection” as used herein refers to the act of protecting or the state of being protected.

“A protective” as used herein is an agent that isolates the exposed surface of the skin or other membrane from harmful or annoying stimuli.

The term “reduce” or “reducing” as used herein refers to limit occurrence of a disorder in an individual(s) at risk of developing the disorder.

The term “refractory” as used herein refers to the state of being unaffected, unresponsive, resistant or not fully responsive.

The term “rubefacient” as used herein refers to an agent that induces hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

The term “sclerosant” as used herein refers to an agent used as a chemical irritant injected into a vein in sclerotherapy. Examples of sclerosants include, but are not limited to, morrhuate sodium, sodium tetradecyl sulfate, laureth 9 and ethanolamine oleate.

The term “side-effect” as used herein refers to a result of a drug or other therapy in addition to, or in extension of, the desired therapeutic effect.

The term “steroidal anti-inflammatory agent”, as used herein, refers to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The term “solubilizing agents” as used herein refers to those substances that enable solutes to dissolve.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “subject” or “individual” or “patient” are used interchangeably to refer to a member of an animal species of mammalian origin, including but not limited to, a mouse, a rat, a cat, a goat, sheep, horse, hamster, ferret, pig, a dog, a guinea pig, a platypus, a rabbit and a primate, such as, for example, a monkey, ape, or human.

The phrase “subject in need thereof” as used herein refers to a patient that (i) will be administered at least one chemotherapeutic agent, (ii) is receiving at least one chemotherapeutic agent; or (iii) has received at least one chemotherapeutic agent, unless the context and usage of the phrase indicates otherwise.

The term “surfactants” as used herein refers to surface-active substances, such as a detergent.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The term “syndrome,” as used herein, refers to a pattern of symptoms indicative of some disease or condition.

The term “telogen effluvium” refers to a condition resulting from an abrupt shift of large numbers of anagen hairs to telogen hairs on the scalp, with a corresponding change in the ratio of anagen hair to telogen hair from the normal ratio of 90:10 to 70:30.

The term “therapeutic agent” as used herein refers to a drug, molecule, nucleic acid, protein, composition or other substance that provides a therapeutic effect. The terms “therapeutic agent” and “active agent” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation.

The term “therapeutically effective amount” or an “amount effective” or “hair protective amount” are used interchangeably and means that one or more of the active agents of the present invention is an amount that is sufficient to provide a therapeutic effect. Generally, an effective amount of the active agents that can be employed ranges from about 0.000001 mg/kg body weight to about 100 mg/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods.

The term “thickeners” as used herein refer to agents that make the composition of the described invention dense or viscous in consistency.

The term “topical” as used herein refers to administration of an inventive composition at, or immediately beneath, the point of application.

The phrase “topically applying” as used herein describes application onto one or more surface(s) including epithelial surfaces. “Topically applying” refers to direct application to the area of the surface to be affected. The composition may be applied by pouring, dropping, or spraying, if a liquid; rubbing on, if an ointment, lotion, cream, gel, or the like; dusting, if a powder; spraying, if a liquid or aerosol composition; or by any other appropriate means.

The terms “treat” or “treating” as used herein refer to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “tumor” means any abnormal swelling, lump or mass. “Tumor” is used synonymously with neoplasm, specifically solid neoplasm.

The term “vitamin” as used herein, refers to any of various organic substances essential in minute quantities to the nutrition of most animals act especially as coenzymes and precursors of coenzymes in the regulation of metabolic processes. Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

The term “vitaminize” (or “vitaminized”) as used herein refers to provide or supplement with vitamins. For example, the phrases “vitaminized peptides” or “vitaminized proteins” as used herein refer to peptides or proteins supplemented with or complexed with vitamins.

### Substituents

The term “aliphatic” as used herein, denotes a straight- or branched-chain arrangement of constituent carbon atoms, including, but not limited to paraffins (alkanes), which are saturated, olefins (alkenes or alkadienes), which are unsaturated, and acetylenes (alkynes), which contain a triple bond. In complex structures, the chains may be branched or cross-linked.

The term “lower” as used herein refers to a group having between one and six carbons.

The term “alkyl” as used herein refers to a straight or branched chain hydrocarbon having from 1 to 25 carbon atoms, optionally substituted with substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may containing one or more O, S, S(O), or S(O)₂ moieties. Examples of “alkyl” as used herein include, but are not limited to, methyl, ethyl, propyl, decyl, undecyl, octadecyl, nonadecyl, eicosyl, heneicosyl, decosyl, tricosyl, tetracosyl, and pentacosyl, n-butyl, t-butyl, n-pentyl, isobutyl, and isopropyl, and the like. In some embodiments of the described invention, the prostaglandin analog has an alkyl of 1, 2, 3, 4, 5, 6, 7, 8, 9, 10, 11, or 12 carbon atoms.

The term “alkylene” as used herein refers to a straight or branched chain divalent hydrocarbon radical having from one to 25 carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may containing one or more O, S, S(O), or S(O)₂ moieties. Examples of “alkylene” as used herein include, but are not limited to, methylene, ethylene, and the like.

The term “alkenyl,” as used herein, denotes a monovalent, straight (unbranched) or branched hydrocarbon chain having one or more double bonds therein where the double bond can be unconjugated or conjugated to another unsaturated group (e.g., a polyunsaturated alkenyl) and can be unsubstituted or substituted, with multiple degrees of substitution being allowed. It may be optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may containing one or more O, S, S(O), or S(O)₂ moeities. For example, and without limitation, the alkenyl can be vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, decenyl, undecenyl, dodecenyl, heptadecenyl, octadecenyl, nonadecenyl, eicosenyl, heneicosenyl, docosenyl, tricosenyl, tetracisenyl, pentacosenyl, phytyl, the branched chain isomers thereof, and polyunsaturated alkenes including octadec-9,12,-dienyl, octadec-9,12,15-trienyl, and eicos-5,8,11,14-tetraenyl.

The term “alkenylene” as used herein refers to a straight or branched chain divalent hydrocarbon radical having from 2 to 25 carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may containing one or more O, S, S(O), or S(O)₂ moeities. Examples of “alkenylene” as used herein include, but are not limited to, ethene-1,2-diyl, propene-1,3-diyl, methylene-1,1-diyl, and the like. Except where specifically indicated otherwise, any olefinic double bond of an alkenyl group that is capable of cis-trans isomerism may possess independently either the E or Z configuration.

The term “alkynyl” as used herein refers to a hydrocarbon radical having from 2 to 25 carbons and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may containing one or more O, S, S(O), or S(O)₂ moeities.

The term “alkynylene” as used herein refers to a straight or branched chain divalent hydrocarbon radical having from 2 to 25 carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may containing one or more O, S, S(O), or S(O)2 moieties. Examples of “alkynylene” as used herein include, but are not limited to, ethyne-1,2-diyl, propyne-1,3-diyl, and the like.

The term “aryl” as used herein refers to a benzene ring or to an optionally substituted benzene ring system fused to one or more optionally substituted benzene rings, with multiple degrees of substitution being allowed. Substituents include, but are not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-napthyl, 1-naphthyl, 1-anthracenyl, and the like.

It should be understood that wherever the terms “alkyl” or “aryl” or either of their prefix roots appear in a name of a substituent, they are to be interpreted as including those limitations given above for alkyl and aryl. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

The term “arylene” as used herein refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

The terms “carbamates” or “urethanes” as used herein refer to a group of organic compounds sharing a common functional group having the general structure —NH(CO)O—.

The term “cycloalkyl” (used interchangeably with “aliphatic cyclic” herein) as used herein refers to a alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

The term “cycloalkylene” as used herein refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

The terms “heterocycle” and “heterocyclic” as used herein are used interchangeably to refer to a three to twelve-membered heterocyclic ring optionally possessing one or more degrees of unsaturation, containing one or more heteroatomic substitutions selected from —S—, —SO—, —SO₂—, —O—, or —N—, optionally substituted with substitutents, including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring optionally may be fused to one or more of another “heterocyclic” ring(s). Examples of “heterocyclic” include, but are not limited to, pyrrole, furan, thiophene, imidazole, oxazole, thiazole, pyrazole, 3-pyrroline, pyrrolidine, pyridine, pyrimidine, purine, quinoline, isoquinoline, carbazole and the like.

The term “C-linked heterocycle” means a heterocycle that is bonded through a carbon atom, e.g.—(CH2)ₙ-heterocycle where n is 1, 2 or 3 or —C<heterocycle where C< represents a carbon atom in a heterocycle ring. Similarly, R moieties that are N-linked heterocycles mean a heterocycle that is bonded through a heterocycle ring nitrogen atom, e.g. —N<heterocycle where N< represents a nitrogen atom in a heterocycle ring. A variable group such as an R moiety that is bonded to a Formula I compound, a Formula II compound, or a Formula III compound, can be a C-linked heterocycle or a N-linked heterocycle, These heterocycles include those listed below or described elsewhere herein.

Examples of heterocycles include by way of example and not limitation pyridyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, .beta.-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at the nitrogen atom or position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Typically, nitrogen bonded heterocycles include 1-aziridyl, 1-azetedyl, 1-pyrrolyl, 1-imidazolyl, 1-pyrazolyl, and 1-piperidinyl and structures such as and tautomers of any of these.

The term “heterocyclylene” as used herein refers to a three to twelve-membered heterocyclic ring diradical optionally having one or more degrees of unsaturation containing one or more heteroatoms selected from S, SO, SO₂, O, or N, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-dyil, and the like.

The term “heteroaryl” as used herein refers to a five- to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

The term “heteroarylene” as used herein refers to a five- to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

The term “hydrate” as used herein refers to a compound formed by the addition of water or its elements to another molecule. The water usually can be split off by heating, yielding the anhydrous compound.

The term “isomer” as used herein refers to one of two or more molecules having the same number and kind of atoms and hence the same molecular weight, but differing in chemical structure. Isomers may differ in the connectivities of the atoms (structural isomers), or they may have the same atomic connectivities but differ only in the arrangement or configuration of the atoms in space (stereoisomers). Stereoisomers may include, but are not limited to, E/Z double bond isomers, enantiomers, and diastereomers. Structural moieties that, when appropriately substituted, can impart stereoisomerism include, but are not limited to, olefinic, imine or oxime double bonds; tetrahedral carbon, sulfur, nitrogen or phosphorus atoms; and allenic groups. Enantiomers are non-superimposable mirror images. A mixture of equal parts of the optical forms of a compound is known as a racemic mixture or racemate. Diastereomers are stereoisomers that are not mirror images. The invention provides for each pure stereoisomer of any of the compounds described herein. Such stereoisomers may include enantiomers, diastereomers, or E or Z alkene, imine or oxime isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3-5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J). In addition the compounds of the described invention may be present as enantiomers, diasteriomers, isomers or two or more of the compounds may be present to form a racemic or diastereomeric mixture.

The term “fused cycloalkylaryl” as used herein refers to a cycloalkyl group fused to an aryl group, the two having two atoms in common, and wherein the aryl group is the point of substitution. Examples of “fused cycloalkylaryl” used herein include, but are not limited to, 5-indanyl, 5,6,7,8-tetrahydro-2-naphthyl,

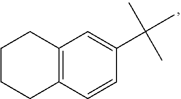

and the like.

The term “fused cycloalkylarylene” as used herein refers to a fused cycloalkylaryl, wherein the aryl group is divalent. Examples include, but are not limited to,

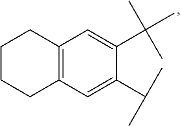

and the like.

The term “fused arylcycloalkyl” as used herein refers to an aryl group fused to a cycloalkyl group, the two having two atoms in common, and wherein the cycloalkyl group is the point of substitution. Examples of “fused arylcycloalkyl” used herein include, but are not limited to, 1-indanyl, 2-indanyl, 1-(1,2,3,4-tetrahydronaphthyl),

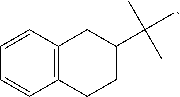

and the like.

The term “fused arylcycloalkylene” as used herein refers to a fused arylcycloalkyl, wherein the cycloalkyl group is divalent. Examples include, but are not limited to,

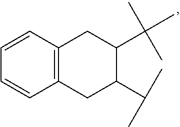

and the like.

The term “fused heterocyclylaryl” as used herein refers to a heterocyclyl group fused to an aryl group, the two having two atoms in common, and wherein the aryl group is the point of substitution. Examples of “fused heterocyclylaryl” used herein include, but are not limited to, 3,4-methylenedioxy-1-phenyl,

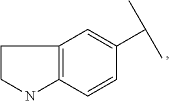

and the like

The term “fused heterocyclylarylene” as used herein refers to a fused heterocyclylaryl, wherein the aryl group is divalent. Examples include, but are not limited to,

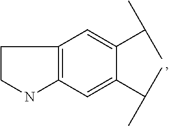

and the like.

The term “fused arylheterocyclyl” as used herein refers to an aryl group fused to a heterocyclyl group, the two having two atoms in common, and wherein the heterocyclyl group is the point of substitution. Examples of “fused arylheterocyclyl” used herein include, but are not limited to, 2-(1,3-benzodioxolyl),

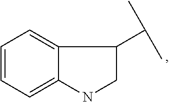

and the like.

The term “fused arylheterocyclylene” as used herein refers to a fused arylheterocyclyl, wherein the heterocyclyl group is divalent. Examples include, but are not limited to,

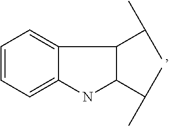

and the like.

The term “fused cycloalkylheteroaryl” as used herein refers to a cycloalkyl group fused to a heteroaryl group, the two having two atoms in common, and wherein the heteroaryl group is the point of substitution. Examples of “fused cycloalkylheteroaryl” used herein include, but are not limited to, 5-aza-6-indanyl,

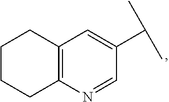

and the like.

The term “fused cycloalkylheteroarylene” as used herein refers to a fused cycloalkylheteroaryl, wherein the heteroaryl group is divalent. Examples include, but are not limited to,

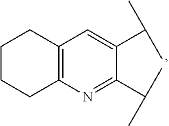

and the like.

The term “fused heteroarylcycloalkyl” as used herein refers to a heteroaryl group fused to a cycloalkyl group, the two having two atoms in common, and wherein the cycloalkyl group is the point of substitution. Examples of “fused heteroarylcycloalkyl” used herein include, but are not limited to, 5-aza-1-indanyl,

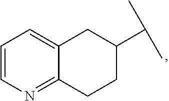

and the like.

The term “fused heteroarylcycloalkylene” as used herein refers to a fused heteroarylcycloalkyl, wherein the cycloalkyl group is divalent. Examples include, but are not limited to,

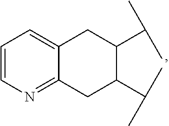

and the like.

The term “fused heterocyclylheteroaryl” as used herein refers to a heterocyclyl group fused to a heteroaryl group, the two having two atoms in common, and wherein the heteroaryl group is the point of substitution. Examples of “fused heterocyclylheteroaryl” used herein include, but are not limited to, 1,2,3,4-tetrahydro-beta-carbolin-8-yl,

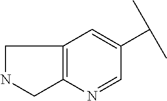

and the like.

The term “fused heterocyclylheteroarylene” as used herein refers to a fused heterocyclylheteroaryl, wherein the heteroaryl group is divalent. Examples include, but are not limited to,

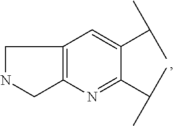

and the like.

The term “fused heteroarylheterocyclyl” as used herein refers to a heteroaryl group fused to a heterocyclyl group, the two having two atoms in common, and wherein the heterocyclyl group is the point of substitution. Examples of “fused heteroarylheterocyclyl” used herein include, but are not limited to, -5-aza-2,3-dihydrobenzofuran-2-yl,

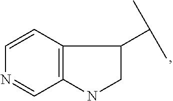

and the like.

The term “fused heteroarylheterocyclylene” as used herein refers to a fused heteroarylheterocyclyl, wherein the heterocyclyl group is divalent. Examples include, but are not limited to,

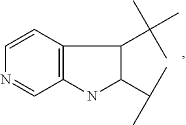

and the like.

The term “acid isostere” as used herein refers to a substituent group which will ionize at physiological pH to bear a net negative charge. Examples of such “acid isosteres” include but are not limited to heteroaryl groups such as but not limited to isoxazol-3-ol-5-yl, 1H-tetrazole-5-yl, or 2H-tetrazole-5-yl. Such acid isosteres include but are not limited to heterocyclyl groups such as but not limited to imidazolidine-2,4-dione-5-yl, imidazolidine-2,4-dione-1-yl, 1,3-thiazolidine-2,4-dione-5-yl, or 5-hydroxy-4H-pyran-4-on-2-yl.

As used herein, the term “direct bond” or “covalent bond”, where part of a structural variable specification, refers to the direct joining of the substituents flanking (preceding and succeeding) the variable taken as a “direct bond.”

The term “O-linked moiety” means a moiety that is bonded through an oxygen atom. Thus, when an R group is an O-linked moiety, that R is bonded through oxygen and it can thus be an ether, an ester (e.g., —O—C(O)-optionally substituted alkyl), a carbonate or a carbamate (e.g., —O—C(O)—NH₂ or —O—C(O)—NH-optionally substituted alkyl). Similarly, the term “S-linked moiety” means a moiety that is bonded through a sulfur atom. Thus, when an R group is an S-linked moiety, that R is bonded through sulfur and it can thus be a thioether (e.g., —S-optionally substituted alkyl), a thioester (—S—C(O)-optionally substituted alkyl) or a disulfide (e.g., —S—S-optionally substituted alkyl). The term “N-linked moiety” means a moiety that is bonded through a nitrogen atom. Thus, when an R group is an N-linked moiety, the R group is bonded through nitrogen and one or more of these can thus be an N-linked amino acid such as —NH—CH₂—COOH, a carbamate such as —NH—C(O)—O-optionally substituted alkyl, an amine such as —NH-optionally substituted alkyl, an amide such as —NH—C(O)-optionally substituted alkyl or —N3. The term “C-linked moiety” means a moiety that is bonded through a carbon atom. When one or more R group is bonded through carbon, one or more of these can thus be -optionally substituted alkyl such as —CH₂—CH₂—O—CH₃, —C(O)-optionally substituted alkyl hydroxyalkyl, mercaptoalkyl, aminoalkyl or ═CH-optionally substituted alkyl.

The term “alkoxy” as used herein refers to the group RₐO—, where Rₐ is alkyl.

The term “alkenyloxy” as used herein refers to the group RₐO—, where Rₐ is alkenyl.

The term “alkynyloxy” as used herein refers to the group RₐO—, where Rₐ is alkynyl.

The term “alkylsulfanyl” as used herein refers to the group RₐS—, where Rₐ is alkyl.

The term “alkenylsulfanyl” as used herein refers to the group RₐS—, where Rₐ is alkenyl.

The term “alkynylsulfanyl” as used herein refers to the group RₐS—, where Rₐ is alkynyl.

The term “alkylsulfenyl” as used herein refers to the group RₐS(O)—, where Rₐ is alkyl.

The term “alkenylsulfenyl” as used herein refers to the group RₐS(O)—, where Rₐ is alkenyl.

The term “alkynylsulfenyl” as used herein refers to the group RₐS(O)—, where Rₐ is alkynyl.

The term “alkylsulfonyl” as used herein refers to the group RₐSO₂—, where Rₐ is alkyl.

The term “alkenylsulfonyl” as used herein refers to the group RₐSO₂—, where Rₐ is alkenyl.

The term “alkynylsulfonyl” as used herein refers to the group RₐSO₂—, where Rₐ is alkynyl.

The term “acyl” as used herein refers to the group RₐC(O)—, where Rₐ is alkyl, alkenyl, alkynyl, cycloalkyl, cycloalkenyl, or heterocyclyl.

The term “aroyl” as used herein refers to the group RₐC(O)—, where Rₐ is aryl.

The term “heteroaroyl” as used herein refers to the group RₐC(O)—, where Rₐ is heteroaryl.

The term “alkoxycarbonyl” as used herein refers to the group RₐOC(O)—, where Rₐ is alkyl.

The term “acyloxy” as used herein refers to the group RₐC(O)O—, where Rₐ is alkyl, alkenyl, alkynyl, cycloalkyl, cycloalkenyl, or heterocyclyl.

The term “aroyloxy” as used herein refers to the group RₐC(O)O—, where Rₐ is aryl.

The term “heteroaroyloxy” as used herein refers to the group RₐC(O)O—, where Rₐ is heteroaryl.

The term “substituted” as used herein refers to substitution with the named substituent or substituents, multiple degrees of substitution being allowed unless otherwise stated.

The terms “contain” or “containing” as used herein refer to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, —CH₂—SO₂—CH₂—, —CH₂—NH—CH₃ and so forth.

The term “oxo” as used herein refers to the substituent ═O.

The term “halogen” or “halo” as used herein includes iodine, bromine, chlorine and fluorine.

The term “mercapto” as used herein refers to the substituent —SH.

The term “carboxy” as used herein refers to the substituent —COOH.

The term “cyano” as used herein refers to the substituent —CN.

The term “aminosulfonyl” as used herein refers to the substituent —SO₂NH₂.

The term “carbamoyl” as used herein refers to the substituent —C(O)NH2.

The term “sulfanyl” as used herein refers to the substituent —S—.

The term “sulfenyl” as used herein refers to the substituent —S(O)—.

The term “sulfonyl” as used herein refers to the substituent —S(O)2-.

The term “ethoxy” as used herein refers to the substituent —O—CH₂CH₃.

The term “methoxy” as used herein refers to the substituent —O—CH₃.

As used herein, the term “optionally” means that the subsequently described event(s) may or may not occur, and includes both event(s) which occur and events that do not occur.

### Method for Preventing or Reducing a Side Effect of a Chemotherapeutic Agent

According to one aspect, the described invention provides a method for preventing a hair-related side-effect associated with treatment of a subject with a chemotherapeutic agent, wherein the hair-related side effect is selected from the group consisting of sparse hair growth, short hair growth, brittle hair growth, thin hair growth, hair loss, alopecia and hair depigmentation, the method comprising steps

(a) providing a topical composition, the topical composition comprising a first component and an optional second component, the first component comprising:

(i) at least one compound of Formula I or a pharmaceutically acceptable salt, hydrate, solvate, prodrug or metabolite thereof,

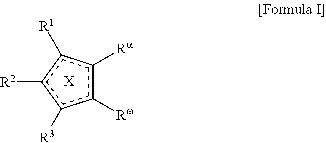

wherein ring X is selected from

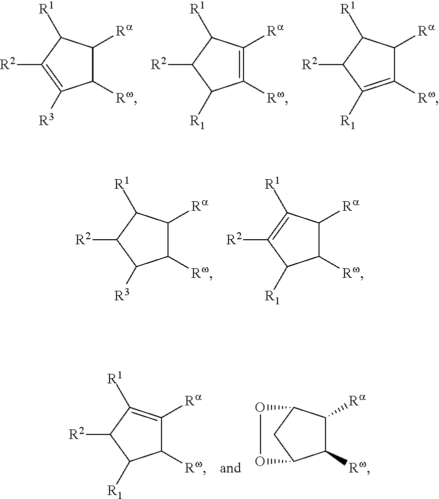

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each,

wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1˜25 or a pharmaceutically acceptable salt thereof; —P(O)(OH)₂ or a pharmaceutically acceptable salt thereof,

wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle may be aromatic or non-aromatic, said cycloalkyl, aryl or heterocycle being optionally substituted with one to three R¹⁷ groups,

wherein R^{α} is

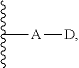

wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one

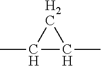

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof,

wherein each R⁷ is independently H, F, or straight chain or branched C₁˜C₅ alkyl,

wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,

wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; NO₂; —SR¹⁰; —CH═NOR¹⁰; —C(═O)NR¹¹OR¹²; —S(O)₂NR⁹₂; —NR⁹S(O)₂R¹³; —OS(O)₂NR⁹₂; —C(O)NHS(O)₂R¹³; —S(O)₂R¹³; —SO₃H; —PO₃H₂;

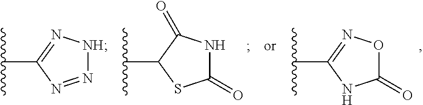

wherein R^{ω} is

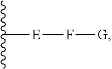

wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or CC bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof,

wherein F is —CH₂—, —O—; —S—; —S(O)—; —S(O₂)—; —C(O)—; —C(O)O—; —C(O)S—; —C(O)NR⁹—; —NR⁹—; or a covalent bond,

wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,

wherein each R⁸ is independently selected from the following: H; a pharmaceutically acceptable cation including, but not limited to, sodium, potassium, magnesium, calcium or an organic cation including but not limited to an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including, but not limited to, acetoxymethyl, acetoxyethyl, aminocarbonyloxymethyl, pivaloyloxymethyl or pivaloyloxyethyl ester), a lactonyl ester (including, but not limited to, a phthalidyl or thiophthalidyl ester), a lower alkoxyacyloxyalkyl ester (including, but not limited to, a methoxycarbonyloxymethyl, ethoxycarbonyloxyethyl or isopropoxycarbonyloxyethyl ester), an alkoxyalkyl ester, choline ester or acylamino alkyl ester (including, but not limited to, an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,

wherein each R⁹ is independently selected from the following: H; a C₁˜C₂₀ straight chain or branched acyclic hydrocarbon group containing zero to four C═C or C≡C bonds wherein each C═C bond independently can be of E or Z configuration; a C₁˜C₂₀ straight chain or branched acyl group containing zero to four C═C or C≡C bonds wherein each C═C bond independently can be of E or Z configuration; —(CH₂)_{q}OH, —(CH₂)_{q}OR¹⁴, —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl (including, but not limited to, acetoxymethyl, acetoxyethyl, aminocarbonyloxymethyl, pivaloyloxymethyl or pivaloyloxyethyl), lactonyl (including, but not limited, to a phthalidyl or thiophthalidyl), lower alkoxyacyloxyalkyl (including, but not limited to, a methoxycarbonyloxymethyl, ethoxycarbonyloxyethyl or isopropoxycarbonyloxyethyl), or acylamino alkyl (including, but not limited to, acetamidomethyl); or -J-K; or —NR⁹₂ can be a cycloamido radical (including, but not limited to, 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like),

wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl

wherein each R¹¹ is independently H or —C(O)R¹⁶,

wherein each R¹² is independently R¹⁶ or —C(O)R¹⁶,

wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L—O-M (“O” being oxygen),

wherein each R¹⁴ is independently straight chain or branched C₁˜C₆ alkyl or —CH₂OCH₃,

wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂;

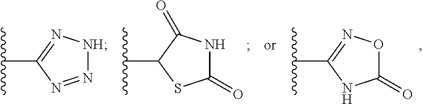

wherein each R¹⁶ is independently H or straight chain or branched C₁˜C₆ alkyl, phenyl or —CH₂OCH₃,

wherein each R¹⁷ is independently straight chain or branched C₁˜C₆ alkyl; —C(O)OR¹⁶; phenyl; F; Cl; Br; I; CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; —NO₂; —S(O)₂N(R¹⁶)₂; —NR¹⁶S(O)₂R¹³, with the proviso that, if R¹⁷ is —NR¹⁶S(O)₂R¹³, R¹, R², R³, R^{α} and R^{ω} are selected such that the molecular weight of the compound of Formula I does not exceed about 2000 atomic mass units, and

wherein not more than four of R⁷ are other than H or F and not more than four of R⁷ are F; and

wherein each chiral center or allenic moiety independently possesses any relative or absolute stereoconfiguration or comprises any mixture thereof; and

(ii) a carrier;

(b) topically administering the topical composition onto an epithelial surface of a subject in need thereof concurrent with administration of at least one chemotherapeutic agent;

(c) stimulating hair growth on an epithelial surface to which the topical composition has been applied.

In some embodiments, the at least one compound of Formula I is diastereomerically pure.

In some embodiments, the at least one compound of Formula I is a mixture of diastereomers in any ratio.

In some embodiments, the at least one compound of Formula I is enantiomerically pure.

In some embodiments, the at least one compound of Formula I is a mixture of enantiomers in any ratio, including a racemate.

In some embodiments, the at least one compound of Formula I is diastereomerically and enantiomerically pure.

In some embodiments, the at least one compound of Formula I is a mixture of diastereomers and enantiomers in any ratio.

In some embodiments, the at least one compound of Formula I has one or more hydrogen atoms replaced by deuterium.

In some embodiments, the compound of the described invention is a compound of Formula (II) or a solvate, hydrate, salt, prodrug or metabolite of Formula (II):

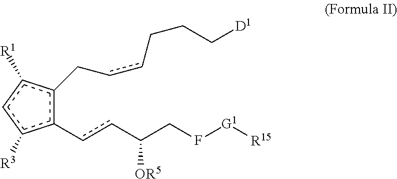

wherein G¹ is phenyl or a 6-membered heterocycle; said heterocycle being aromatic or non-aromatic; said heterocycle containing at least two heteroatoms selected from the group consisting of N, S and O; said phenyl or heterocycle being optionally substituted with one to three R¹⁵ groups; and

D¹ is —C(O)OR⁸ or —C(O)NR⁹₂.

The following are nonlimiting embodiments depicting cyclopentane ring X of the compound of Formula II, wherein R¹ is ═O and R³ is H, which has the structure:

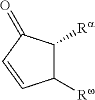

According to one embodiment, R⁵ is H, D¹ is —C(O)NR⁹₂, R⁹₂ is H and a nitrogen-linked cyclopropyl group, F is —O—, G¹ is phenyl and there is no R¹⁵. This compound of Formula II has the structure:

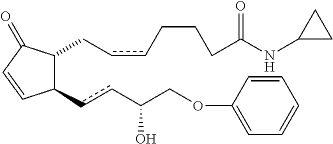

wherein a hashed line represents a substituent behind the plane of this paper, a bold wedge represents a substituent above the plane of this paper, and a dashed line represents a single or double bond which can be in the cis or trans configuration. In one embodiment, the compound in which the dashed lines are double bonds with the stereoconfigurations as drawn is 16-phenoxy 17, 18,19,20-tetranor PGA₂ N-cyclopropylamide. A compound where the C₅-C₆ dashed line is a single bond, and the C₁₃-C₁₄ dashed line is a double bond with the stereoconfigurations as drawn is 16-phenoxy 17, 18,19,20-tetranor PGA₁ N-cyclopropylamide.

According to another embodiment, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

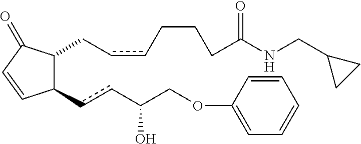

wherein a hashed line represents a substituent behind the plane of this paper, a bold wedge represents a substituent above the plane of this paper, wherein the dashed lines represent a single bond or a double bond which can be in the cis or trans configuration; wherein when the dashed lines represent a double bond and the 5,6 bond is in a cis configuration and the 13,14 bond is in a trans configuration, a compound with the stereoconfigurations as drawn is 16-phenoxy-17,18,19,20-tetranor PGA₂ N-cyclopropylmethylamide. A compound where the C₅-C₆ dashed line is a single bond, and the C₁₃-C₁₄ dashed line is a double bond with the stereoconfigurations as drawn is 16-phenoxy 17,18,19,20-tetranor PGA₁ N-cyclopropyl methylamide.

According to another embodiment, R⁵ is H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

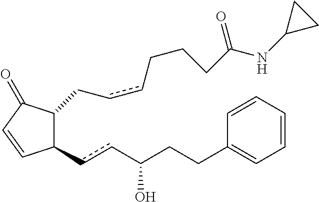

wherein a hashed line represents a substituent below the plane of this paper, a bold wedge represents a substituent above the plane of this paper, the dashed lines represent a single or a double bond; wherein when the dashed lines represent a double bond which can be in the cis or trans configuration. A compound with the stereoconfigurations as drawn is 17-phenyl-18,19,20 trinor PGA₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGA₁ N-cyclopropylamide.

According to another embodiment, R⁵ is H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

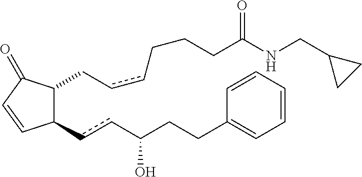

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn is 17-phenyl-18,19,20-trinor PGA₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGA₁ N-cyclopropylmethylamide.

According to another embodiment, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

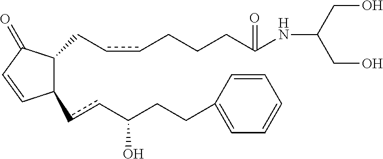

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfiguration as drawn is 17-phenyl-18,19,20-trinor PGA₂ (N-(1,3-dihydroxypropan-2-yl))amide. A compound wherein the C₅-C₆ dashed line is a single bond and the C₁₃-C₁₄ dashed line is a double bond with the stereoconfigurations as drawn is 17-phenyl-18,19,20-trinor PGA₁(N-(1,3-dihydroxypropan-2-yl))amide.

The following are nonlimiting embodiments depicting cyclopentane ring X of the compound of Formula II, wherein R¹ is ═O, and R³ is H, which has the structure:

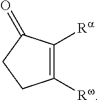

In some embodiments, R⁵ is H, D¹ is —C(O)NR⁹₂, R⁹₂ is H and a nitrogen-linked-cyclopropyl group, F is —O—, G¹ is phenyl and there is no R¹⁵. This compound of Formula II has the structure:

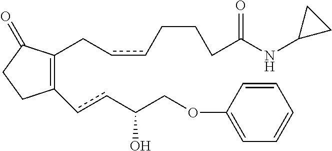

wherein a hashed line represents a substituent below the plane of this paper, and a dashed line represents a single or a double bond which can be in the cis or trans configuration. A compound where the dashed lines are double bonds with the stereoconfigurations as drawn is 16-phenoxy-17, 18,19,20-tetranor PGB₂ N-cyclopropylamide. A compound where the C₅-C₆ dashed line is a single bond, and the C₁₃-C₁₄ dashed line is a double bond with the stereoconfigurations as drawn is 16-phenoxy-17, 18,19,20-tetranor PGB₁ N-cyclopropylamide.

In some embodiments, R⁵ is H, D¹ is —C(O)NR⁹₂, R⁹² is H and a nitrogen-linked-cyclopropylmethyl group, F is —O—, G¹ is phenyl and there is no R¹⁵. This compound of Formula II has the structure:

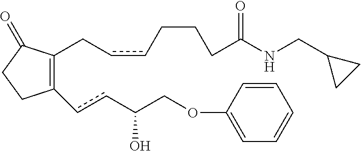

wherein a hashed line represents a substituent below the plane of this paper, and a dashed line represents a single or a double bond which can be in the cis or trans configuration. A compound, where the dashed lines are double bonds with the stereoconfigurations as drawn is 16-phenoxy-17,18,19,20-tetranor PGB₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGB₁ N-cyclopropylmethylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This compound of Formula II has the structure:

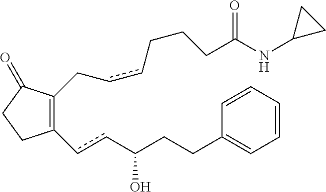

wherein a hashed line represents a substituent below the plane of this paper, and a dashed line represents a single bond or a double bond which can be in the cis or trans configuration. A compound where the dashed lines are double bonds with the stereoconfigurations as drawn is 17-phenyl-18,19,20-trinor PGB₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGB₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula II has the structure:

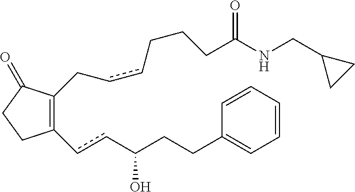

wherein a hashed line represents a substituent below the plane of this paper, and a dashed line represents a single bond or a double bond which can be in the cis or trans configuration. A compound, where the dashed lines are double bonds with the stereoconfigurations as drawn is 17-phenyl-18,19,20-trinor PGB₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGB₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

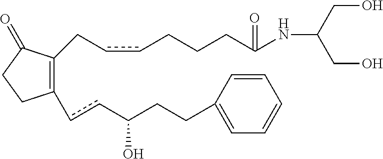

wherein a hashed line represents a substituent below the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGB₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGB₁ (N-(1,3-dihydroxypropan-2-yl))amide.

The following are nonlimiting embodiments depicting cyclopentane ring X of the compound of Formula II, wherein R¹ is ═O, R² is H, and R³ is H, which has the structure:

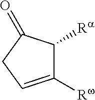

In some embodiments, R⁵ is H, D¹ is —C(O)NR⁹₂, R⁹₂ is H and a nitrogen-linked —N-linked cyclopropyl group, F is —O—, G¹ is phenyl and there is no R¹⁵. This compound of Formula II has the structure:

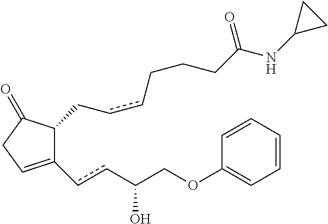

wherein a hashed line represents a substituent below the plane of this paper, and a dashed line represents a single bond or a double bond which can be in the cis or trans configuration. This compound, where the dashed lines are double bonds with the stereoconfigurations as drawn is 16-phenoxy-17,18,19,20-tetranor PGC₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGC₁ N-cyclopropylamide.

In some embodiments, R⁵ is H, D¹ is —C(O)NR⁹₂, R⁹₂ is H and a nitrogen-linked-cyclopropylmethyl group, F is —O—, G¹ is phenyl and there is no R¹⁵. This compound of Formula II has the structure:

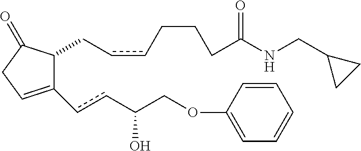

wherein a hashed line represents a substituent below the plane of this paper, and a dashed line represents a single bond or a double bond which can be in the cis or trans configuration. The compound where the dashed lines are double bonds with the stereoconfigurations as drawn is 16-phenoxy-17,18,19,20-tetranor PGC₂N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGC₁ N-cyclopropylmethylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This the compound of Formula II has the structure:

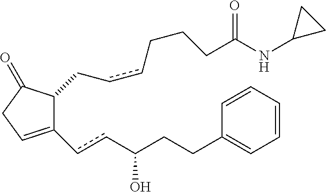

wherein a hashed line represents a substituent below the plane of this paper, and a dashed line represents a single bond or a double bond which can be in the cis or trans configuration. The compound where the dashed lines are double bonds with the stereoconfigurations as drawn is 17-phenyl-18,19,20-trinor PGC₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGC₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula II has the structure:

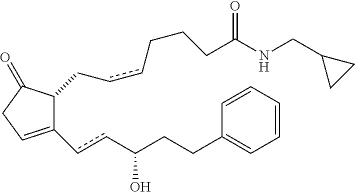

wherein a hashed line represents a substituent below the plane of this paper, and a dashed line represents a single bond or a double bond which can be in the cis or trans configuration. The compound, where the dashed lines are double bonds with the stereoconfigurations as drawn is 17-phenyl-18,19,20-trinor PGC₂ N-cyclopropylmethyl amide. A compound where the C₅-C₆ dashed line is a single bond, and the C₁₃-C₁₄ dashed line is a double bond with the stereoconfigurations as drawn is 17-phenyl-18,19,20-trinor PGC₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

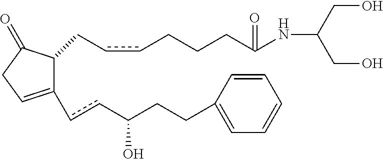

wherein a hashed line represents a substituent below the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGC₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGC₁ (N-(1,3-dihydroxypropan-2-yl))amide.

The following are nonlimiting embodiments depicting cyclopentane ring X of the compound of Formula II, wherein R¹ is OH, and R³ is ═O, which has the structure:

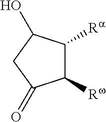

In some embodiments, R⁵ is H, D¹ is —C(O)NR⁹₂, R⁹₂ is H and a nitrogen-linked-cyclopropyl group, F is —O—, G¹ is phenyl and there is no R¹⁵. This compound of Formula II has the structure:

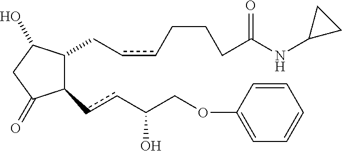

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 16-phenoxy-17,18,19,20-tetranor PGD₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGD₁ N-cyclopropylamide.

In some embodiments, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

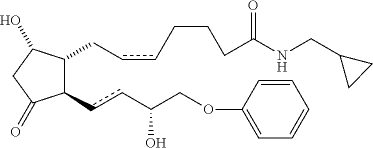

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 16-phenoxy-17,18-,19,20-tetranor PGD₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGD₁ N-cyclopropylmethylamide.

In some embodiments, R⁵ is H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

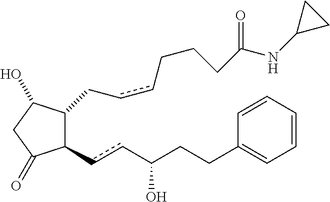

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGD₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGD₁ N-cyclopropylamide.

In some embodiments, R⁵ is H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

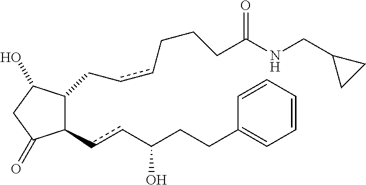

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn is 17-phenyl-18,19,20-trinor PGD₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGD₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

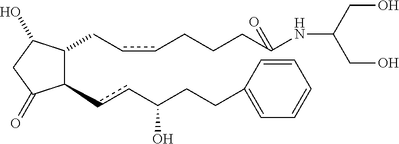

wherein a hashed line represents a substituent below the plane of this paper, a bold wedge represents a substituent above the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGD₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGD₁ (N-(1,3-dihydroxypropan-2-yl))amide.

The following are nonlimiting embodiments depicting cyclopentane ring X of the compound of Formula II, wherein R¹ is ═O, and R³ is OH or OR⁴, which has the structure:

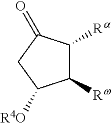

In one embodiment, R⁴ and R⁵ are both H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

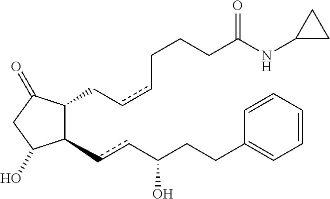

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds, with the stereoconfigurations as drawn is 17-phenyl-18,19,20-trinor PGE₂ N-cyclopropylamide. A compound wherein the C₅-C₆ dashed line is a single bond, and the C₁₃-C₁₄ dashed line is a double bond with the stereoconfigurations as drawn is 17-phenyl-18,19,20-trinor PGE₁ N-cyclopropylamide.

In another embodiment, R⁴ and R⁵ are both H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

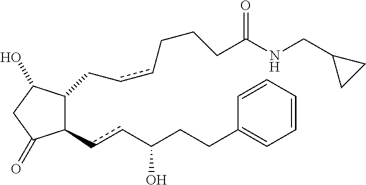

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn, is 17-phenyl-18,19,20-trinor PGE₂ N-cyclopropylmethylamide. A compound wherein the C₅-C₆ dashed line is a single bond, and the C₁₃-C₁₄ dashed line is a double bond with the stereoconfigurations as drawn is 17-phenyl-18,19,20-trinor PGE₁ N-cyclopropylmethylamide.

In yet another embodiment, R⁴ is —PO₃⁻² with pharmaceutically acceptable counter ion(s) such as 2 Na⁺, R⁵ is H, F is O, G¹ is phenyl, there is no R¹⁵, D¹ is —C(O)OR⁸, and R⁸ is a cyclopropyl group. This compound of Formula II has the following structure (counterions not shown):

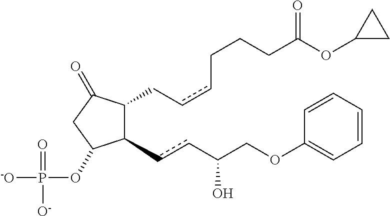

wherein a hashed line represents a substituent below the plane of this paper, a bold wedge represents a substituent above the plane of this paper, and a dashed line represents a single bond or a double bond which can be in the cis or trans configuration.

The following are nonlimiting embodiments depicting cyclopentane ring X of the compound of Formula II, wherein R¹ is ═O, and R³ is OH or OR⁴, which has the structure:

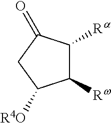

In another embodiment, R⁴ and R⁵ are H, D¹ is —C(O)NR⁹₂, F is ═O, R⁹₂ is H and a nitrogen-linked cyclopropyl group, F is —O—, G¹ is phenyl, and there is no R¹⁵. This compound of Formula II has the structure:

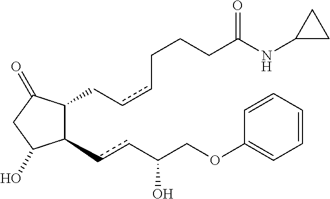

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds with the stereoconfigurations as drawn is 16-phenoxy-17,18,19,20-tetranor PGE₂ N-cyclopropylamide. A compound wherein the C₅-C₆ dashed line is a single bond, and the C₁₃-C₁₄ dashed line is a double bond with the stereoconfigurations as drawn is 16-phenoxy-17,18,19,20-tetranor PGE₁ N-cyclopropylamide.

In another embodiment, R⁵ is H, D¹ is —C(O)NR⁹₂, R⁹₂ is H and a nitrogen-linked cyclopropylmethyl group, F is —O—, G¹ is phenyl and there is no R¹⁵.

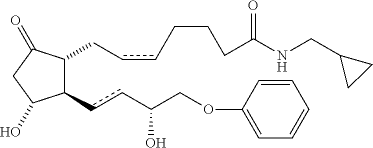

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds, with the stereoconfigurations as drawn is 16-phenoxy-17,18,19,20-tetranor PGE₂ N-cyclopropylmethylamide. A compound wherein the C₅-C₆ dashed line is a single bond, and the C₁₃-C₁₄ dashed line is a double bond with the stereoconfigurations as drawn is 16-phenoxy-17,18,19,20-tetranor PGE₁ N-cyclopropylmethylamide.

In another embodiment, R⁵ is H, D¹ is —C(O)NR⁹₂, R⁹₂ is H and a nitrogen-linked cyclopropyl group, F is CH₂, G¹ is pyrimidin-2-yl and there is no R¹⁵. This compound of Formula II has the structure:

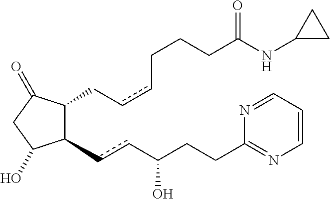

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn, is 17-pyrimidin-2-yll-18,19,20-trinor PGE₂ N-cyclopropylamide. A compound wherein the C₅-C₆ dashed line is a single bond, and the C₁₃-C₁₄ dashed line is a double bond with the stereoconfigurations as drawn is 17-pyrimidin-2-yl-18,19,20-trinor PGE₁ N-cyclopropylamide.

In another embodiment, R³ is —OR⁴ and R⁴ is —PO₃H₂, R⁵ is H, F is —S(O)—, G¹ is phenyl, there is no R¹⁵, D¹ is —C(O)OR⁸, and R⁸ is a cyclopropyl group. This compound of Formula II has the structure:

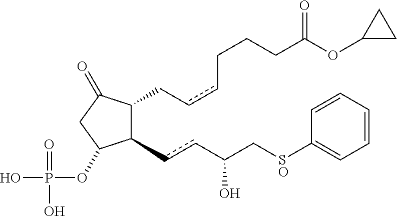

wherein a hashed line represents a substituent below the plane of this paper, a bold wedge represents a substituent above the plane of this paper, and a dashed line represents a single bond or a double bond which can be in the cis or trans configuration.

In yet another embodiment, R³ is OR⁴ where R⁴ is a monosaccharide (e.g., glucose), R⁵ is H, D¹ is —C(O)NR⁹₂, R⁹₂ is H and a nitrogen-linked ethyl group; F is —CH₂—, G¹ is phenyl, and there is no R¹⁵. This compound of Formula II has the structure:

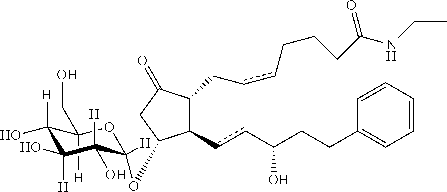

wherein a hashed line represents a substituent below the plane of this paper, a bold wedge represents a substituent above the plane of this paper, and a dashed line represents a single bond or a double bond which can be in the cis or trans configuration.

In yet another embodiment, R³ is OR⁴ where R⁴ is a monosaccharide (e.g., glucose), R⁵ is H, D¹ is —C(O)NR⁹₂, R⁹₂ is H and a nitrogen-linked ethyl group; F is —CH₂—, G¹ is 1,3-oxazinan-2-yl, and R¹⁵ is a cyclopropyl group linked to a nitrogen heteroatom as shown. This compound of Formula II has the structure:

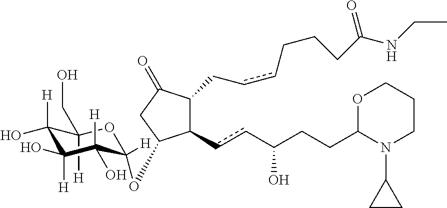

wherein a hashed line represents a substituent below the plane of this paper, a bold wedge represents a substituent above the plane of this paper, and a dashed line represents a single bond or a double bond which can be in the cis or trans configuration.

In some embodiments, R⁴ and R⁵ are H, D¹ is —C(O)NR⁹₂, R⁹₂ is H and a nitrogen-linked —CH(CH₂OH)₂ group, F is CH₂, G¹ is pyrimidin-2-yl and there is no R¹⁵ the compound of Formula II has the structure:

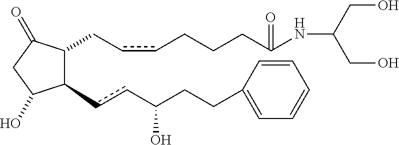

wherein a hashed line represents a substituent below the plane of this paper, a bold wedge represents a substituent above the plane of this paper, and a dashed line represents a double bond in the cis or trans configuration. A compound wherein the dashed lines are double bonds with the stereoconfigurations as drawn is 17-phenyl-18,19,20-trinor PGE₂ (N-(1,3-dihydroxypropan-2-yl)) amide. A compound wherein the C₅-C₆ dashed line is a single bond and the C₁₃-C₁₄ dashed line is a double bond with the stereoconfigurations as drawn is 17-phenyl-18,19,20-trinor PGE₁ (N-(1,3-dihydroxypropan-2-yl)) amide.

The following are nonlimiting embodiments depicting cyclopentane ring X of the compound of Formula II, wherein R¹ is OH or OR⁴, and R³ is OH or OR⁴, which has the structure:

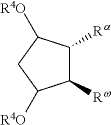

In one embodiment, R⁵ is H, D¹ is —C(O)NR⁹₂, R⁹₂ is H and a nitrogen-linked cyclopropyl group, F is —CH₂—, G¹ is phenyl, and there is no R¹⁵ group. This compound of Formula II has the structure:

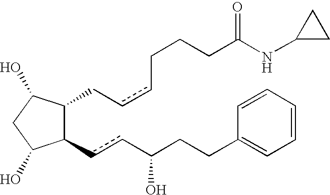

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 17-phenyl-18,19,20-trinor PGF_{2α} N-cyclopropylamide. A compound wherein the C₅-C₆ dashed line is a single bond and the C₁₃-C₁₄ dashed line is a double bond with the stereoconfigurations as drawn is 17-phenyl-18,19,20-trinor PGF_{2α} N-cyclopropylamide.

In another embodiment, R⁵ is H, D¹ is —C(O)NR⁹₂, R⁹₂ is H and a nitrogen-linked cyclopropylmethyl group, F is —CH₂—, G¹ is phenyl, and there is no R¹⁵. This compound of formula II has the structure:

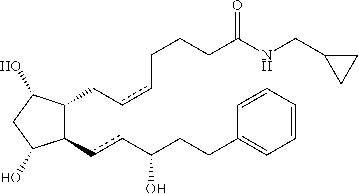

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A A compound where the dashed lines are double bonds with the stereoconfigurations as drawn is 17-phenyl-18,19,20-trinor PGF_{2α} N-cyclopropylmethylamide. A compound wherein the C₅-C₆ dashed line is a single bond and the C₁₃-C₁₄ dashed line is a double bond with the stereoconfigurations as drawn is 17-phenyl-18,19,20-trinor PGF_{2α} N-cyclopropylmethylamide.

In yet another embodiment, R¹ is OR⁴, where R⁴ is PO₃²⁻ with pharmaceutically acceptable counterion(s) such as 2 Na⁺, D¹ is —C(O)OR⁸, R⁵ is H, F is —O—, G¹ is phenyl, there is no R¹⁵, and R⁸₂ is a cyclopropyl group. This compound of Formula II has the structure:

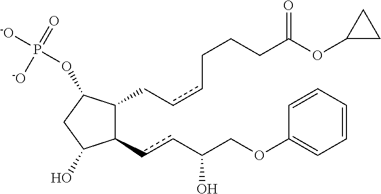

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In another embodiment, R⁵ is H, D¹ is —C(O)NR⁹₂, R⁹₂ is H and a nitrogen-linked cyclopropyl group, F is —O—, G¹ is phenyl, there is no R¹⁵. This compound of Formula II has the structure:

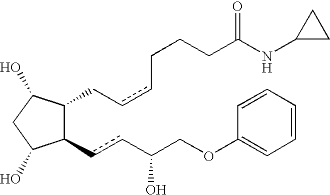

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. This compound, where the dashed lines are double bonds with the stereoconfigurations as drawn, is 16-phenoxy-17,18,19,20-tetranor PGF_{2α} N-cyclopropylamide. A compound wherein the C₅-C₆ dashed line is a single bond and the C₁₃-C₁₄ dashed line is a double bond with the stereoconfigurations as drawn is 16-phenoxy-17,18,19,20-tetranor PGF_{2α} N-cyclopropylamide.

In another embodiment, R⁵ is H, D¹ is —C(O)NR⁹₂, R⁹₂ is H and a nitrogen-linked cyclopropylmethyl group, F is —O—, G¹ is phenyl, and there is no R¹⁵. This compound of formula II, has the structure:

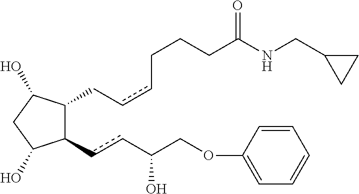

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn is 16-phenoxy-17,18,19,20-tetranor PGF_{2α} N-cyclopropylmethylamide. A compound wherein the C₅-C₆ dashed line is a single bond and the C₁₃-C₁₄ dashed line is a double bond with the stereoconfigurations as drawn is 16-phenoxy-17,18,19,20-tetranor PGF_{2α} N-cyclopropylmethylamide.

In another embodiment, R⁵ is H, D¹ is —C(O)NR⁹₂, R⁹₂ is H and a nitrogen-linked cyclopropyl group, F is CH₂, G¹ is pyrimidin-2-yl, and there is no R¹⁵. This compound of Formula II has the structure:

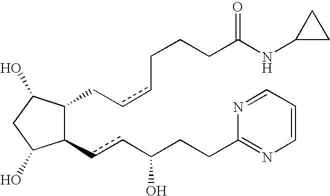

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 17-pyrimidin-2-yl-18,19,20-trinor PGF₂ N-cyclopropylamide. A compound wherein the C₅-C₆ dashed line is a single bond and the C₁₃-C₁₄ dashed line is a double bond with the stereoconfigurations as drawn is 17-pyrimidin-2-yl-18,19,20-trinor PGF_{2α} N-cyclopropylamide.

In another embodiment, R¹ is OR⁴ where R⁴ is —(PO₂(OH))ₛH, where s is 1˜25 or a pharmaceutically acceptable salt thereof, R⁵ is H, F is —S(O)—, G¹ is phenyl, D¹ is —C(O)OR⁸, and R⁸ is a cyclopropyl group. This compound of Formula II has the structure:

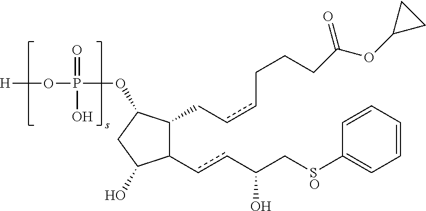

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound, when the dashed lines are double bonds with the stereoconfigurations as drawn, is a poly-hydroxyphosphoryloxy derivative of cyclopropyl 7-((1R,3R,5S)-3-hydroxy-2-((3R,E)-3-hydroxy-4-(phenylsulfinyl)but-1-enyl)-5-(phosphonooxy)cyclopentyl)heptanoate. A compound wherein the C₅-C₆ dashed line is a single bond and the C₁₃-C₁₄ dashed line is a double bond with the stereoconfigurations as drawn is cyclopropyl 7-((1R,3R,5S)-3-hydroxy-5-phosphonooxy-2-((3R,E)-3-hydroxy-4-(phenylsulfinyl)but-1-enyl)cyclopentyl)heptanoate.

In yet another embodiment, R¹ is OR⁴ and R⁴ is a monosaccharide (e.g., glucose), R⁵ is H, D¹ is —C(O)NR⁹₂, R⁹₂ is H and a nitrogen-linked ethyl group; F is —CH₂—, G¹ is phenyl, and there is no R¹⁵. This compound of Formula II has the structure:

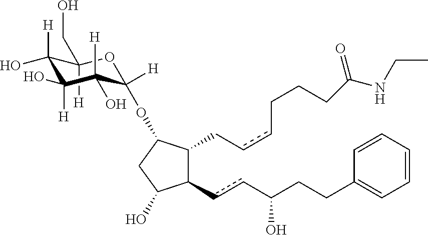

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In yet another embodiment, R¹ is OR⁴ and R⁴ is a monosaccharide (e.g., glucose), R⁵ is H, D¹ is —C(O)NR⁹₂, R⁹₂ is H and a nitrogen-linked ethyl group; F is —CH₂—, G¹ is 1,3-oxazinan-2-yl and R¹⁵ is a cyclopropyl group. This compound of Formula II has the structure:

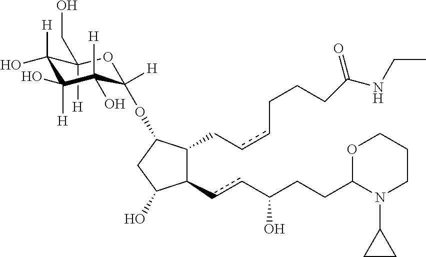

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In another embodiment, R⁵ is H, D¹ is —C(O)NR⁹₂, R⁹₂ is H and cyclopropyl; F is —O—; G¹ is phenyl and R¹⁵ is Cl. This compound of Formula II has the structure:

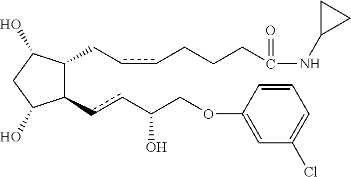

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound, where the dashed lines are double bonds, is 16-(3-chlorophenoxy)-17,18,19,20-tetranor PGF2α N-cyclopropylamide. A compound wherein the C₅-C₆ dashed line is a single bond and the C₁₃-C₁₄ dashed line is a double bond with the stereoconfigurations as drawn is 16-(3-chlorophenoxy)-17,18,19,20-tetranor PGF_{2α} N-cyclopropylamide.

In another embodiment, R⁵ is H, D¹ is —C(O)NR⁹₂, R⁹₂ is H and N-cyclopropylmethylamide; F is —O—; G¹ is phenyl; and R¹⁵ is Cl. This compound of Formula II has the structure:

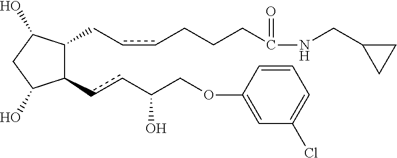

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound, where the dashed lines are double bonds with the stereoconfigurations as drawn, is 16-(3-chlorophenoxy)-17,18,19,20-tetranor PGF2α-N-cyclopropylmethylamide. A compound wherein the C₅-C₆ dashed line is a single bond and the C₁₃-C₁₄ dashed line is a double bond with the stereoconfigurations as drawn is 16-(3-chlorophenoxy)-17,18,19,20-tetranor PGF_{2α} N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

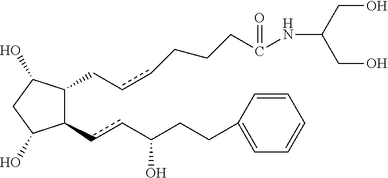

wherein a hashed line represents a substituent below the plane of this paper, a bold wedge represents a substituent above the plane of this paper, and a dashed line represents a single bond or a double bond having the configuration indicated. A compound wherein the dashed lines are double bonds with the stereoconfigurations as drawn is 17-phenyl-18,19,20-trinor PGF2α (N-(1,3-dihydroxypropan-2-yl))amide. A compound wherein the C₅-C₆ dashed line is a single bond and the C₁₃-C₁₄ dashed line is a double bond with the stereoconfigurations as drawn is 17-phenyl-18,19,20-trinor PGF_{2α} (N-(1,3-dihydroxypropan-2-yl))amide.

In some embodiments, the compound of Formula II has the structure:

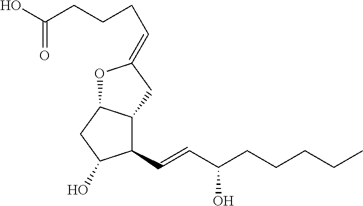

wherein a hashed line represents a substituent below this paper's plane; and wherein a bold wedge represents a substituent above this paper's plane. This compound with the stereoconfigurations as drawn is ((Z)-5-((3aR,4R,5R,6aS)-5-hydroxy-4-((S,E)-3-hydroxyoct-1-enyl)hexahydro-2H-cyclopenta[b]furan-2-ylidene)pentanoic acid) (PGI₂).

The following are nonlimiting embodiments depicting cyclopentane ring X of the compound of Formula II, wherein R¹ is H, R² is H, and R³ is ═O, which has the structure:

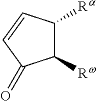

In some embodiments, R⁵ is H, D¹ is —C(O)NR⁹₂, R⁹² is H and a nitrogen-linked-cyclopropyl group, F is —O—, G¹ is phenyl and there is no R¹⁵. This compound of Formula II has the structure:

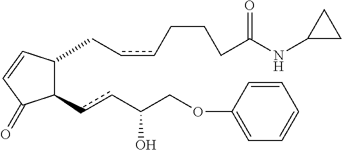

wherein a hashed line represents a substituent below the plane of this paper, a bold wedge represents a substituent above the plane of this paper, and a dashed line represents a single or a double bond. A compound where the dashed lines are double bonds with the stereoconfigurations as drawn is 16-phenoxy-17,18,19,20-tetranor PGJ₂ N-cyclopropylamide. A compound wherein the C₅-C₆ dashed line is a single bond and the C₁₃-C₁₄ dashed line is a double bond with the stereoconfigurations as drawn is 16-phenoxy-17,18,19,20-tetranor PGJ₁ N-cyclopropylamide.

In some embodiments, R⁵ is H, D¹ is —C(O)NR⁹₂, R⁹₂ is H and a nitrogen-linked-cyclopropylmethyl group, F is —O—, G¹ is phenyl and there is no R¹⁵. This compound of Formula II has the structure:

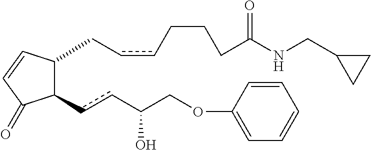

wherein a hashed line represents a substituent below the plane of this paper, a bold wedge represents a substituent above the plane of this paper, and a dashed line represents a single or a double bond having the configuration indicated. A compound where the dashed lines are double bonds with the stereoconfigurations as drawn is 16-phenoxy-17,18,19,20-tetranor PGJ₂ N-cyclopropylmethylamide. A compound wherein the C₅-C₆ dashed line is a single bond and the C₁₃-C₁₄ dashed line is a double bond with the stereoconfigurations as drawn is 16-phenoxy 17,18,19,20-tetranor PGJ₁ N-cyclopropylmethylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This compound of Formula II has the structure:

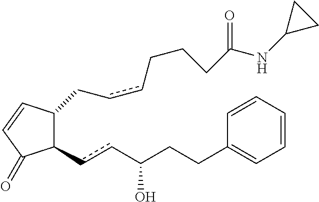

wherein a hashed line represents a substituent below the plane of this paper, a bold wedge represents a substituent above the plane of this paper, and a dashed line represents a single or a double bond. A compound, where the dashed lines are double bonds with the stereoconfigurations as drawn is 17-phenyl-18,19,20-trinor PGJ₂ N-cyclopropylamide. A compound wherein the C₅-C₆ dashed line is a single bond and the C₁₃-C₁₄ dashed line is a double bond with the stereoconfigurations as drawn is 17-phenyl-18,19,20-trinor PGJ₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula II has the structure:

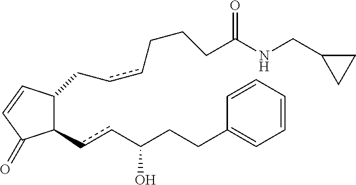

wherein a hashed line represents a substituent below the plane of this paper, a bold wedge represents a substituent above the plane of this paper, and a dashed line represents a single bond or a double bond. A compound where the dashed lines are double bonds with the stereoconfigurations as drawn is 17-phenyl-18,19,20-trinor PGJ₂ N-cyclopropylmethylamide. A compound wherein the C₅-C₆ dashed line is a single bond and the C₁₃-C₁₄ dashed line is a double bond with the stereoconfigurations as drawn is 17-phenyl,18,19,20-trinor PGJ₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

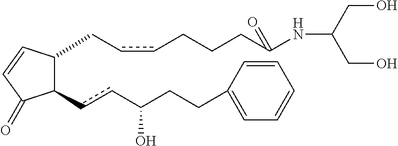

wherein a hashed line represents a substituent below the plane of this paper, a bold wedge represents a substituent above the plane of this paper, and a dashed line represents a single bond or a double bond. A compound wherein the dashed lines are double bonds with the stereoconfigurations as drawn is 17-phenyl-18,19,20-trinor PGJ₂ (N-(1,3-dihydroxypropan-2-yl)) amide. A compound wherein the C₅-C₆ dashed line is a single bond and the C₁₃-C₁₄ dashed line is a double bond with the stereoconfigurations as drawn is 17-phenyl-18,19,20-trinor PGJ₁ (N-(1,3-dihydroxypropan-2-yl)) amide.

According to one embodiment, the compound of formula I is latanoprost. According to another embodiment, the compound of formula I is travoprost. According to another embodiment, the compound of formula I is travoprost ethylamide. According to another embodiment, the compound of formula I is bimatoprost. According to another embodiment, the compound of formula I is fluprostenol. According to another embodiment, the compound of formula I is fluprostenol isopropyl ester. According to another embodiment, the compound of formula I is fluprostenol methyl amide. According to another embodiment, the compound of formula I is 9-keto fluprostenol isopropyl ester. According to another embodiment, the compound of formula I is cloprostenol. According to another embodiment, the compound of formula I is cloprostenol isopropyl ester. According to another embodiment, the compound of formula I is chloprostenol methyl amide. According to another embodiment, the compound of formula I is 17 phenyl-18,19,20-trinor prostaglandin E2 (N-(1,3-dihydroxypropan-2-yl))amide. According to another embodiment, the compound of formula I is 17 phenyl-18,19,20-trinor prostaglandin F2α (N-(1,3-dihydroxypropan-2-yl))amide.

According to another embodiment, the optional second component of the topical composition is an additional active agent. Additional active agents optionally included in the topical compositions according to the described invention include, without limitation, one or more, in any combination, a protective, an emollient, an astringent, an irritant, a keratolytic, a sun screening agent, a sun tanning agent, an antibiotic agent, a non-imidazole analog antifungal agent, an imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant, a chemotherapeutic agent, an anti-histamine, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent.

According to some embodiments, the optional second component of the topical composition is an imidazole analog according to Formula III, or a hydrate, solvate, salt, zwitterion, N-oxide, tautomer, prodrug or metabolite thereof:

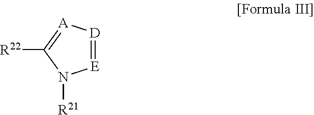

wherein A is N; NR²⁰; NR⁶³ or CH;

wherein D is N or CR²³;

wherein E is N; NR⁶³ or CR²⁴;

with the proviso that, if A is CH, then at least one among D and E is a nitrogen-containing moiety;

with the further proviso that A and E are not simultaneously both NR⁶³;

wherein R²³ is H; F, Cl, Br; I; —NO₂; —N(R⁴²)₂; straight chain or branched C₁˜C₆ alkyl, alkenyl or alkoxy; or phenyl;

wherein R²⁴ is H; F, Cl, Br; I; —NO₂; —N(R⁴₂)₂; straight chain or branched C₁˜C₆ alkyl, alkenyl or alkoxy; or phenyl;

or wherein R²³ and R²⁴ together are CR⁶²═CR⁶²—CR⁶²═CR⁶²—;

wherein R²⁰ is selected from the following: H; straight chain or branched C₁˜C₁₂ alkyl;

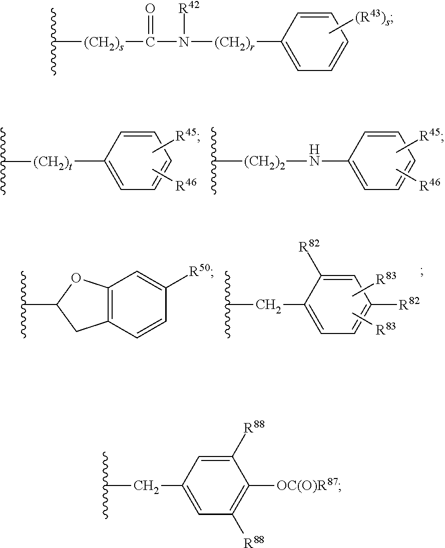

—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula III is an imidazolium or triazolium salt with a pharmaceutically acceptable counter anion or an internal salt (zwitterion); or

wherein R⁶³ is a moiety that is readily cleaved in vivo, exemplified by, but not limited to, —CHR⁴²OC(═O)(O)ₙR⁵⁵ or —C(R⁴²)₂OP(═O)(OR¹⁰⁶)₂ and the compound of Formula III is a prodrug and an imidazolium or triazolium salt with a pharmaceutically acceptable counter ion or an internal salt (zwitterion);

wherein s is 1 or 2;

wherein t is an integer from 1 to 4 inclusive;

wherein u is 2 or 3;

wherein R²² is H; F, Cl, Br; I; —NO₂; —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical preferably containing one S and one or two N atoms, said heterocyclic radical being preferably thiazolyl and more preferably thiazol-4-yl;

wherein R²¹ is H; or —(CR²⁵R²⁶)ₘ—CR²⁷R²⁸-Q-R²⁹;

wherein each m is independently an integer from 0 to 4 inclusive;

wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;

wherein R²⁶ is H; or straight chain or branched C₁˜C₆ alkyl;

wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(═NH)N(R⁶⁸)OR⁶⁸; —CF(R⁶⁷)C(═NH)N(R⁶⁸)OH; or —CF(R⁶⁷)C(═N—OR⁶⁹)NH₂;

wherein each r is independently an integer from 0 to 2 inclusive;

wherein each L is independently α-tetralyl; or phenyl, said phenyl being optionally substituted with up to three groups selected from the following: straight chain or branched C₁˜C₆ alkyl, straight chain or branched C₁˜C₆ alkoxy, —C≡N, —NO₂, or —NH₂;

wherein R²⁸ is H; F; Cl; Br; —OR⁴²; —OC(═O)R¹⁰⁰;

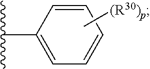

straight chain or branched C₁˜C₆ alkyl; R⁶¹; or —OP(═O)(OH)₂ or a pharmaceutically acceptable salt or zwitterion form thereof;

wherein each Q is independently a covalent bond; —O—; —S—; —S(O)—; or —S(O)₂—;

wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; straight chain or branched C₁˜C₆ alkyl optionally substituted with —CO₂H; or

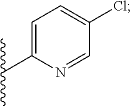

wherein each p is independently an integer from 0 to 3 inclusive;

wherein each T is independently a covalent bond; —CH₂CH₂—; —CH═CH—; —(O)ₙC(R⁴²)₂—; —CH₂O—; —SCH₂—; —CH₂S—; —OCH₂CH₂O—; —CH(CH₃)—; —CH₂—; —CF₂—; —C(R⁹³)₂—; or

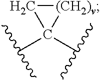

wherein each v is independently an integer from 1 to 3 inclusive;

wherein each U is independently a covalent bond;

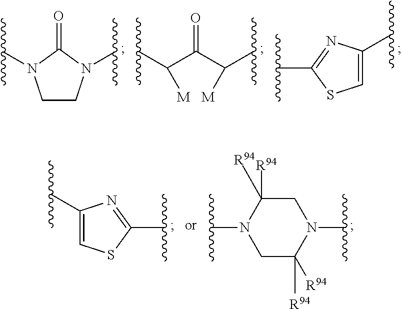

wherein each V is independently H; —S(O)₂CH₃; —C(O)NH₂; —CH₂C≡CH; —CH═CH₂; —S(O)ᵣ—R⁵⁵;

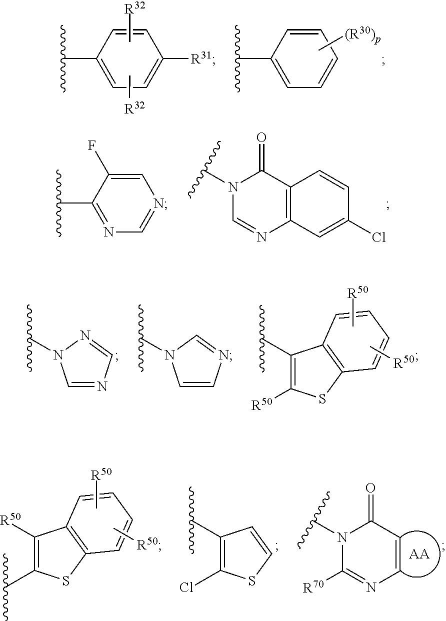

### —R⁵⁹; —R⁶¹; or -LL-R⁹⁵;

wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings;

wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴; —OC(O)N(R⁵⁴)₂; —SH; —SR⁴¹; —S(O)ᵣR⁵⁸; —CH═CH—CO₂H;

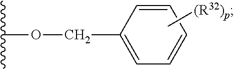

straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to —CH₂C≡N, —CH₂CH₂C≡N, —CH₂CH₂CH₂C≡N, and —CH₂CH(CH₃)C≡N; straight chain or branched C₁˜C₆ halogenoalkyl exemplified by, but not limited to —CF₃, —CF₂CF₃, —CH₂CF₃, —CHF₂, —CH₂F, —CH₂Cl, and —CH₂Br; straight chain or branched C₁˜C₅ halogenoalkoxy exemplified by, but not limited to —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₈ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy;

wherein each R³¹ is independently H; F; Cl; Br; I; —C≡N; NO₂; —CF₃; —N═C═S; —N(R⁴²)₂; —NH—C(═O)-(M)ₙ-R⁵⁴; —NH—C(═S)—NH—R⁵⁴; straight chain or branched C₁˜C₆ alkoxy or alkylthio; straight chain or branched C₁˜C₁₂ alkyl; —(CH₂)ᵣ—R⁹⁹;

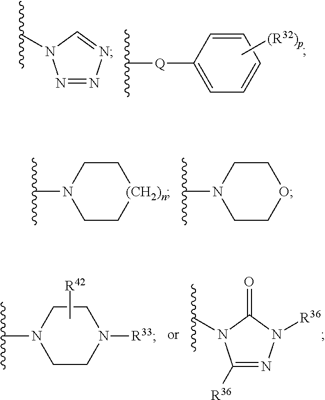

wherein each M is independently O or NH, with the proviso that when M is O and n is 1, R⁵⁴ is other than H;

wherein each R³² is independently H; F; Cl; Br; I; —C≡N; NO₂; straight chain or branched C₁˜C₁₂ alkyl; straight chain or branched C₁˜C₄ alkoxy; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; straight chain or branched C₁˜C₃ halogenoalkoxy exemplified by, but not limited to —OCF₃, —OCF₂CF₃, —OCH₂CF₃, —OCHF₂, —OCH₂F; straight chain or branched C₁˜C₄ alkylthio or haloalkylthio; straight chain or branched C₁˜C₄ alkylthionyl or haloalkylthionyl; or straight chain or branched C₁˜C₄ alkylsulfonyl or haloalkylsulfonyl;

wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)—C(O)OR³⁴; —C(O)O-phenyl; —(CH₂)—C(O)N(R⁴²)₂; —CH₂C(S)N(R⁴²)₂; —CH₂C(S)SR³⁴; —(CH₂)n-phenyl; benzoyl, wherein said benzoyl is optionally substituted with one or two R⁴⁶ groups; or

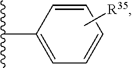

or, preferably,

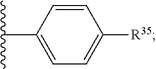

wherein each R³⁴ is independently straight chain or branched C₁˜C₆ alkyl;

wherein each R³⁵ is independently H;

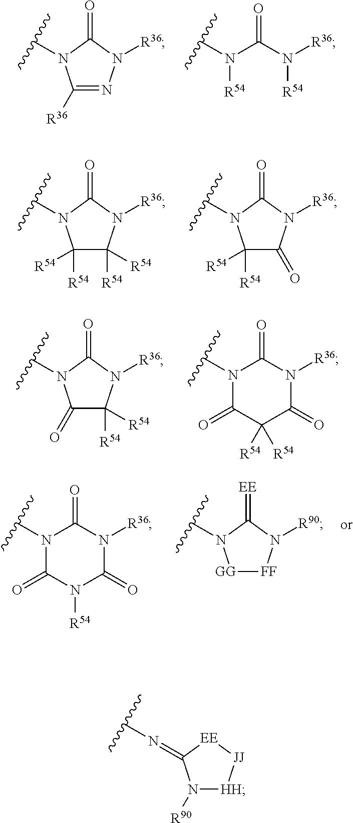

wherein each R³⁶ is independently H; straight chain or branched C₃˜C₇ alkenyl, with the proviso that the olefinic double bond is not located a to N; straight chain or branched C₃˜C₇ alkynyl, with the proviso that the alkynyl C≡C bond is not located a to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or

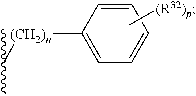

wherein each n is independently 0 or 1;

wherein R²⁶ and R²⁸ together with the two carbon atoms to which they attach can be C═C;

wherein R²⁷ and R²⁸ together can be

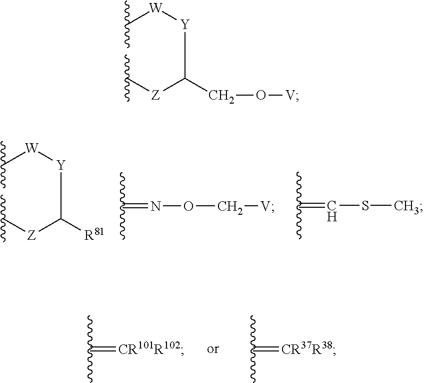

wherein W is —CH₂— and Y is —CH₂—; W is —O— and Y is —CH₂—; or W is —CH₂— and Y is —O—;

wherein Z is —CH₂— or —O—;

wherein R²⁷ together with -Q-R²⁹ can be

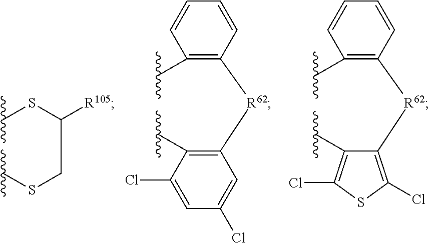

or, when R²⁷ and R²⁹ each independently represents a cycloalkyl, aryl or heterocyclic ring and Q is a covalent bond, R²⁷ together with -Q-R²⁹ can be

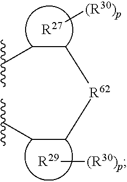

wherein each R³⁷ is independently H; C₁˜C₂ alkyl or phenyl;

wherein each R³⁸ is independently H or C₁˜C₂ alkyl;

wherein each R³⁹ is independently H; straight chain or branched C₁˜C₅ alkyl; straight chain or branched C₁˜C₅ alkanoyl; or straight chain or branched C₁˜C₅ haloalkanoyl exemplified by, but not limited to —C(O)CF₃, —C(O)CF₂CF₃, —C(O)CH₂CF₃, —C(O)CHF₂, and —C(O)CH₂F;

wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;

wherein each R⁴¹ is independently straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;

wherein each q is independently an integer from 0 to 4 inclusive;

wherein each G is independently a covalent bond; —O—; or —S—;

wherein each J is independently phenyl, 2-thienyl or 3-thienyl wherein said phenyl, 2-thienyl or 3-thienyl is optionally substituted with one to three groups selected from the following: F; Cl; Br; I; —NO₂; straight chain or branched C₁˜C₅ alkyl; straight chain or branched C₁˜C₅ alkoxy;

wherein each R⁴² is independently H; or straight chain or branched C₁˜C₆ alkyl;

wherein each R⁴³ is independently H; F; Cl; Br; I; —NO₂; straight chain or branched C₁˜C₆ alkyl; straight chain or branched C₁˜C₆ alkoxy; —CF₃; or —O-phenyl;

wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the following: F, Cl, Br, I, straight chain or branched C₁˜C₆ alkyl, or straight chain or branched C₁˜C₆ alkoxy;

wherein each R⁴⁵ is independently H; F; Cl; Br; I; —NO₂; straight chain or branched C₁˜C₆ alkyl; straight chain or branched C₁˜C₆ alkoxy; or —S(O)₂NH₂;

wherein each R⁴⁶ is independently H; F; Cl; Br; I; straight chain or branched C₁˜C₆ alkyl; straight chain or branched C₁˜C₆ alkoxy; or —C≡N;

wherein each R⁴⁷ is independently a phenyl group optionally substituted with one to three F, Cl, Br or I;

wherein each R⁴⁸ is independently H; F; Cl; Br; I; or straight chain or branched C₁˜C₆ alkyl;

wherein each R⁴⁹ is independently 1-naphthyl; 2-naphthyl; or phenyl, wherein said phenyl is optionally substituted with one or two groups, each independently selected from the following: F, Cl, Br, I, straight chain or branched C₁˜C₆ alkyl, straight chain or branched C₁˜C₆ alkoxy, or —CH₂CH═CH₂;

wherein each R⁵⁰ is independently H; F; Cl; Br; or I;

wherein each R⁵¹ is independently phenyl, wherein said phenyl is optionally substituted with one or two groups, each independently selected from the following: F, Cl, Br, I, or straight chain or branched C₁˜C₆ alkyl;

wherein each R⁵² is independently phenyl, wherein said phenyl is optionally substituted with one or two groups, each independently selected from the following: F, Cl, Br, or I;

wherein each R⁵³ is independently straight chain or branched C₁˜C₈ alkyl optionally substituted with C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; straight chain or branched C₃—O₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl C≡C bond is located β, γ or δ with respect to the ether oxygen;

wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl;

wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle can be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups;

wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle can be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle can be aromatic or non-aromatic; methylenedioxy; —C≡N; —NO₂; —CF₃; —N(R⁵⁴)₂; —OR⁵⁴; —SH; ═O; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; —C(═NH)N(R⁵⁴)OH; —C(═N—OR⁵⁴)NH₂; —S(O)₂N(R⁵⁴)₂; —NR⁵⁴S(O)₂R⁵⁴; —C(O)NHS(O)₂R⁵⁴; —S(O)₂R⁵⁴; —SO₃H; or —PO₃H₂;

wherein each R⁵⁷ is independently F; Cl; Br; I; straight chain or branched C₁˜C₆ alkyl; straight chain or branched C₁˜C₆ alkoxy; —C≡N; —CF₃; NO₂; —NH₂; or —NHC(O)R⁶⁶, wherein said R⁶⁶ is straight chain or branched C₂˜C₁₂ alkyl;

wherein each R⁵⁸ is independently straight chain or branched C₁˜C₂₀ alkyl; C₃˜C₈ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups;

wherein each R⁵⁹ is independently straight chain or branched C₁˜C₇ alkyl, alkenyl or alkynyl, said alkyl, alkenyl or alkynyl being substituted with one or more —R⁶⁰—R⁶¹;

wherein each R⁶⁰ is independently a covalent bond or —O—;

wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups;

wherein each R⁶² is independently a covalent bond; —CH₂—CH₂—; —CH═CH—; —O—; or —S—;

wherein each R⁶⁵ is independently straight chain or branched C₁˜C₁₂ acyclic hydrocarbon group, which can be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or C≡C bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;

wherein each R⁶⁷ is independently H; F; or straight chain or branched C₁˜C₆ alkyl, said alkyl group being optionally substituted by one or more R³⁰;

wherein each R⁶⁸ is independently R³⁶, R⁵⁴ or R⁶¹;

wherein each R⁶⁹ is independently H; or straight chain or branched C₁˜C₆ alkyl, said alkyl group being optionally substituted by one or more R³⁰;

wherein each R⁷⁰ is independently H; straight chain or branched C₁˜C₄ alkyl; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; or cyclopropyl;

wherein each R⁷¹ is independently R⁵⁶; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)rR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶;

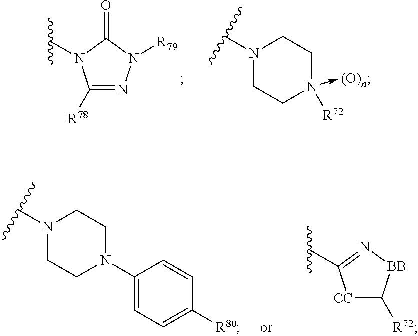

wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;

wherein each R⁷³ is independently H; straight chain or branched C₁˜C₄ alkyl;

C₃˜C₆ cycloalkyl; —C(O)R⁷²; or —C(O)CF₃;

wherein each R⁷⁴ is independently straight chain or branched C₁˜C₄ alkyl;

wherein each R⁷⁵ is independently H; —C(O)NH₂; —C(O)CH₃; —C≡N; —SO₂NHR⁷²; —SO₂R⁷²; —OR⁷²; —OC(O)R⁷²; or straight chain or branched —(C₁˜C₄ alkyl)-NH₂;

wherein each R⁷⁶ is independently phenyl optionally substituted with one or more R⁷⁷ groups;

wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²;

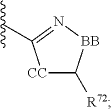

or phenyl, said phenyl being optionally substituted with one or more F, Cl, Br, I, —C≡N, —NO₂, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;

wherein each R⁷⁸ is independently H or —CH₃;

wherein each R⁷⁹ is independently H; isopropyl; cyclopentyl; cyclopropyl; 2-butyl; 3-pentyl; 3-hydroxy-2-butyl; or 2-hydroxy-3-pentyl;

wherein each R⁸⁰ is independently F; Cl; Br; I; —C≡N; —NO₂; —NH₂; straight chain or branched C₁˜C₄ halogenoalkyl; straight chain or branched C₁˜C₄ halogenoalkoxy; or

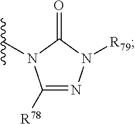

wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —C≡N, —NO₂, phenyl, phenylmethyl, benzoyl, halobenzoyl, —C(O)R⁴², —C(O)OR⁴², and —CF₃, with the proviso that when multiple substituents are present only 1 thereof can be selected from the group consisting of phenyl, phenylmethyl, benzoyl and halobenzoyl;

wherein each R⁸² is independently H or —OR⁸⁴;

wherein each R⁸³ is independently H; F; Cl; Br; R⁷⁴; —OR⁷⁴; —SR⁷⁴; —CH₂SC(═O)R⁷⁴; —COOH; or —C(═O)OCH₃;

wherein each R⁸⁴ is independently a group that is easily hydrolyzable under physiological conditions, preferably the acyl residue of an amino acid or a group represented by the formula —C(═O)R⁸⁵ or —P(═O)(OR⁸⁶)₂; wherein R⁸⁴ is exemplified by, but not limited to, formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like;

wherein each R⁸⁵ is independently H; —OR⁷⁴; or straight chain or branched C₁˜C₆ alkyl or alkenyl, wherein said alkyl or alkenyl can be optionally interrupted by a hydrolyzable functional group such as carboxamide or ester, wherein said alkyl or alkenyl can be optionally substituted with —COOH, —NH₂, —NHR⁷⁴, —N(R⁷⁴)₂, or R⁶¹, wherein R⁶¹ is exemplified by, but not limited to, phenyl, methoxyphenyl, pyridyl, pyrazinyl or furyl;

wherein each R⁸⁶ is independently H or R⁷⁴;

wherein each R⁸⁷ is independently straight chain or branched C₁˜C₅ alkyl; R⁶¹; pyridyl; pyrrolidinyl; or -DD-NH—R⁸⁹;

wherein each R⁸⁸ is independently H; F; Cl; Br; or —OR⁷⁴;

wherein each DD is independently straight chain or branched C₁˜C₄ alkylene; —CH₂NHC(═O)CH₂—; —CH(NH₂)CH₂CH₂CH₂—;

wherein each R⁸⁹ is independently H or straight chain or branched C₁˜C₅ alkyl;

wherein each BB is independently —CH₂— or —C(═O)—;

wherein each CC is independently NH or O;

wherein each EE is independently O, S or N—R⁹¹;

wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃˜C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched;

wherein each KK is independently O or S;

wherein each R⁹¹ is independently H or straight chain or branched C₁˜C₆ alkyl;

wherein each R⁹² is independently H; straight chain or branched C₁˜C₆ alkyl;

C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—;

wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃;

wherein each GG is independently —C(═O)—; or —CH₂—, optionally substituted with up to two radicals selected from the group consisting of straight chain or branched C₁˜C₆ alkyl and straight chain or branched C₁˜C₆ alkoxy;

or FF and GG, taken together, form a bivalent radical of formula —N═CH—(FFGG′);

wherein HH has the same meaning as FF;

wherein JJ has the same meaning as GG;

or HH and JJ, taken together, form a bivalent radical of formula —N═CH—(FFGG′) or —CH═CH—(HHJJ′);

wherein the nitrogen atom in the bivalent radical FFGG′ is connected to NR⁹⁰;

wherein one hydrogen in said radical FFGG′ and up to two hydrogens in radical HHJJ′ can be replaced by a straight chain or branched C₁˜C₆ alkyl radical;

wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group exemplified by, but not limited to —CH₂CH₂—, —CH₂CH₂CH₂—, —CH₂CH₂CH₂CH₂— and the like;

wherein each R⁹⁴ is independently H or C₁˜C₄ alkyl which is unsubstituted or substituted by 1-3 substituents each independently selected from the group consisting of halogen, hydroxyl, C₁˜C₄ alkoxy, and amino; or two R⁹⁴ groups attached to the same carbon atom together form ═S;

wherein each LL is independently a covalent bond; —C(═O)—; —C(═S)—; —SO₂—; or —N═N—;

wherein each R⁹⁵ is independently selected from the group consisting of

i) hydrogen,

ii) CN

iii) CHO

iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl,

v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido,

vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl,

vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and

viii) a group of the formula

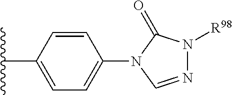

wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which can be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl;

wherein each MM is independently an ethylene group optionally substituted with R⁹¹; or MM is —NN═OO—, wherein NN and OO are the same or different and are each independently N or CR⁹¹;

wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶;

wherein each R¹⁰⁰ is independently —R⁵⁴ or —OR⁵⁴;

wherein each R¹⁰¹ is independently H; F; Cl; Br; or I;

wherein each R¹⁰² is independently F; Cl; Br; I; —SR¹⁰³; or —OR¹⁰⁴;

wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which can be the same or different;

wherein each R¹⁰⁴ is independently straight chain or branched C₁˜C₁₂ alkyl; or phenyl;

wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰;

wherein each R¹⁰⁶ is independently H, a pharmaceutically acceptable cation, or null (affording an azolium phosphate zwitterion);

wherein each chiral center independently may possess any relative or absolute stereoconfiguration or be any mixture thereof, unless specified otherwise herein; and

wherein each olefinic C═C bond that is capable of E/Z isomerism independently may possess either the E or Z stereoconfiguration or be any mixture thereof, unless specified otherwise herein.

In some embodiments, the at least one compound of Formula III is diastereomerically pure.

In some embodiments, the at least one compound of Formula III is a mixture of diastereomers in any ratio.

In some embodiments, the at least one compound of Formula III is enantiomerically pure.

In some embodiments, the at least one compound of Formula III is a mixture of enantiomers in any ratio, including a racemate.

In some embodiments, the at least one compound of Formula III is diastereomerically and enantiomerically pure.

In some embodiments, the at least one compound of Formula III is a mixture of diastereomers and enantiomers in any ratio.

In some embodiments, the at least one compound of Formula III has one or more hydrogen atoms replaced by deuterium.

In some embodiments, the at least one compound of Formula III is a pharmaceutically acceptable salt derived from the combination of a compound of Formula III with an acid or a base.

In some embodiments, the at least one compound of Formula III is an N-oxide form of an amine.

In some embodiments, the at least one compound of Formula III is a zwitterion.

In some embodiments, the at least one compound of Formula III is a tautomer of a structure described herein.

Examples of compounds of Formula III include, but are not limited to, the antifungal agents bifonazole, butoconazole, croconazole, econazole, enilconazole, clotrimazole, flutrimazole, isoconazole, fenticonazole, ketoconazole, sulconazole, tioconazole, oxiconazole, sertaconazole, miconazole, chlormidazole, omoconazole, posaconazole, elubiol, voriconazole, eberconazole, CAS RN 214543-30-3, democonazole, genaconazole, vibunazole, lombazole, pramiconazole, T 8581, fluconazole, albaconazole, tiabendazole, parconazole, ravuconazole, saperconazole, itraconazole, hydroxyitraconazole, TAK 456, terconazole, SYN 2869, neticonazole, NND-502, lanoconazole, fosfluconazole; and the thromboxane synthase inhibitors 7-(1-imidazolyl)heptanoic acid, ozagrel, and 1-benzyl imidazole.

Other compounds of interest not contained within Formula III include the antifungal chlordantoin; non-azole thromboxane synthase inhibitors, such as terbinafine, furegrelate, BM 567, and analogs of terbinafine, such as amorolfine, butenafine, naftifine; and Epoxygenase inhibitors, such as MS-PPOH, and PPOH.

According to some embodiments, an imidazole analog of the described invention is a compound of Formula IV, or a hydrate, solvate, salt, zwitterion, N-oxide, tautomer, prodrug or metabolite of Formula IV, having the structure:

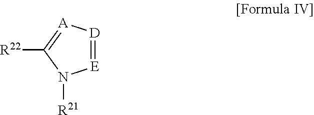

wherein each A is independently N or NR²⁰;

wherein each D is independently CR²³;

wherein each E is independently N or CR²⁴;

wherein R²³ is H;

wherein R²⁴ is H;

or wherein R²³ and R²⁴ together are —CH═CH—CH═CH—;

wherein R²⁰ is H and the compound of Formula IV is an imidazolium or triazolium salt with a pharmaceutically acceptable counter anion; or

wherein R²⁰ is a moiety that is readily cleaved in vivo, exemplified by, but not limited to, —CH₂OC(O)CH₃, and the compound of Formula IV is a prodrug and an imidazolium or triazolium salt with a pharmaceutically acceptable counter anion;

wherein R²² is H; —CH₃; or

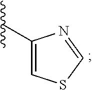

wherein R²¹ is H; —(CR²⁵R²⁶)m-CR²⁷R²⁸-Q-R²⁹;

wherein each m is independently 0 or 1;

wherein R²⁵ is H; —CH₃; or —C≡N;

wherein R²⁶ is H;

wherein R²⁷ is -T-U—V;

wherein R²⁸ is H; —OH; or

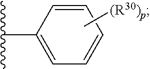

wherein each Q is independently a covalent bond or —S—;

wherein R²⁹ is

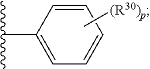

or straight chain or branched C₃˜C₁₀ alkyl optionally substituted with —CO₂H;

wherein each p is independently an integer from 0 to 3 inclusive;

wherein each T is independently a covalent bond; —CH₂CH₂—; —OCH₂—; —CH₂O—; —SCH₂—; —CH₂S—; —OCH₂CH₂O—; —CH(CH₃)—; —CH₂—; or —CF₂—;

wherein each U is independently a covalent bond;

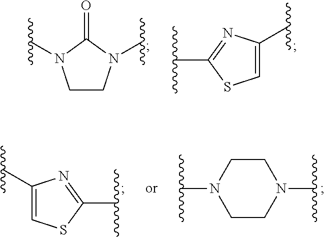

wherein each V is independently H; —S(O)₂CH₃; —C(O)NH₂; —CH₂C≡CH; —CH═CH₂;

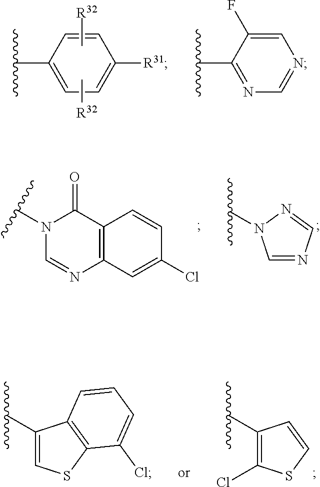

wherein each R³⁰ is independently F, Cl, —CH═CH—CO₂H;

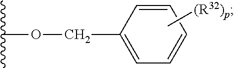

or straight chain or branched C₁˜C₁₀ alkoxy;

wherein each R³¹ is independently H; F; Cl; Br; I; —C≡N;

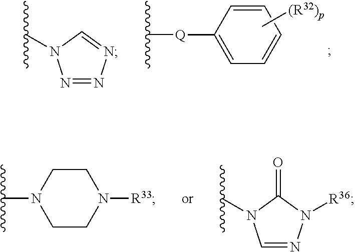

wherein each R³² is independently H; F; Cl; or —OCF₃;

wherein each R³³ is independently straight chain or branched C₁˜C₆ alkyl; —C(O)R³⁴; —C(O)OR³⁴;

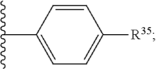

wherein each R³⁴ is independently straight chain or branched C₁˜C₆ alkyl;

wherein each R³⁵ is independently H;

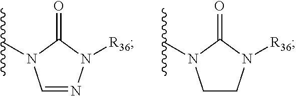

wherein each R³⁶ is independently straight chain or branched C₁˜C₆ alkyl optionally substituted with —OH; or

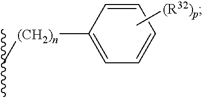

wherein each n is independently 0 or 1;

wherein R²⁶ and R²⁸ together with the two carbon atoms to which they attach can be C═C;

wherein R²⁷ and R²⁸ together can be

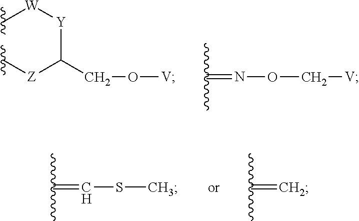

wherein W is —CH₂— and Y is —CH₂—; W is —O— and Y is —CH₂—; or W is —CH₂— and Y is —O—;

wherein Z is —CH₂— or —O—;

wherein R²⁷ together with -Q-R²⁹ can be

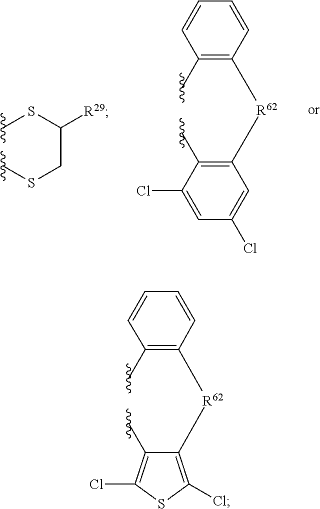

wherein each R⁶² is independently —CH₂—CH₂— or —CH═CH—.

In some embodiments, each chiral center independently possesses any relative or absolute stereoconfiguration or is any mixture thereof, unless specified otherwise herein.

In some embodiments, each olefinic C═C bond that is capable of E/Z isomerism independently can possess either the E or Z stereoconfiguration or be any mixture thereof, unless specified otherwise herein.

In some embodiments, the at least one compound of Formula IV is diastereomerically pure.

In some embodiments, the at least one compound of Formula IV is a mixture of diastereomers in any ratio.

In some embodiments, the at least one compound of Formula IV is enantiomerically pure.

In some embodiments, the at least one compound of Formula IV is a mixture of enantiomers in any ratio, including a racemate.

In some embodiments, the at least one compound of Formula IV is diastereomerically and enantiomerically pure.

In some embodiments, the at least one compound of Formula IV is a mixture of diastereomers and enantiomers in any ratio.

In some embodiments, the at least one compound of Formula IV has one or more hydrogen atoms replaced by deuterium.

In some embodiments, the at least one compound of Formula IV is a pharmaceutically acceptable salt derived from the combination of a compound of formula III with an acid or a base.

In some embodiments, the at least one compound of Formula IV is an N-oxide form of an amine described herein.

In some embodiments, the at least one compound of Formula IV is a zwitterion.

In some embodiments, the at least one compound of Formula IV is a tautomer of a structure described herein.

Examples of imidazole analogs according to Formula IV include, but are not limited to, histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole.

According to one embodiment, the compound of Formula IV, is miconazole or a solvate, a hydrate, a salt, a zwitterion, an N-oxide, a tautomer, a prodrug or a metabolite of miconazole. According to another embodiment, the compound of Formula IV, is ketoconazole or a solvate, a hydrate, a salt, a zwitterion, an N-oxide, a tautomer, a prodrug or a metabolite of miconazole.

According to another embodiment, the at least one chemotherapeutic agent is an alkylating agent for which alopecia is a side-effect. According to another embodiment, the at least one chemotherapeutic agent is temozolomide. According to another embodiment, the at least one chemotherapeutic agent is busulfan. According to another embodiment, the at least one chemotherapeutic agent is ifosamide. According to another embodiment, the at least one chemotherapeutic agent is melphalan hydrochloride. According to another embodiment, the at least one chemotherapeutic agent is carmustine. According to another embodiment, the at least one chemotherapeutic agent is lomustine. According to another embodiment, the at least one chemotherapeutic agent is cyclophosphamide.

According to another embodiment, the at least one chemotherapeutic agent is an antimetabolite for which alopecia is a side-effect. According to another embodiment, the at least one chemotherapeutic agent is 5-fluorouracil. According to another embodiment, the at least one chemotherapeutic agent is capecitabine. According to another embodiment, the at least one chemotherapeutic agent is gemcitabine. According to another embodiment, the at least one chemotherapeutic agent is floxuridine. According to another embodiment, the at least one chemotherapeutic agent is decitabine. According to another embodiment, the at least one chemotherapeutic agent is mercaptopurine. According to another embodiment, the at least one chemotherapeutic agent is pemetrexed disodium. According to another embodiment, the at least one chemotherapeutic agent is methotrexate. According to another embodiment, the at least one chemotherapeutic agent is dacarbazine.

According to another embodiment, the at least one chemotherapeutic agent is a natural product for which alopecia is a side-effect, wherein the natural product is a vinca alkaloid, a taxane, an epothilone, an anthracycline, an antibiotic, a camptothecin, or an epididophylotoxin. According to another embodiment, the at least one chemotherapeutic agent is vincristine. According to another embodiment, the at least one chemotherapeutic agent is vinblastine. According to another embodiment, the at least one chemotherapeutic agent is vinorelbine tartrate. According to another embodiment, the at least one chemotherapeutic agent is paclitaxel. According to another embodiment, the at least one chemotherapeutic agent is docetaxel. According to another embodiment, the at least one chemotherapeutic agent is ixabepilone. According to another embodiment, the at least one chemotherapeutic agent is daunorubicin. According to another embodiment, the at least one chemotherapeutic agent is epirubicin. According to another embodiment, the at least one chemotherapeutic agent is doxorubicin. According to another embodiment, the at least one chemotherapeutic agent is idarubicin. According to another embodiment, the at least one chemotherapeutic agent is mitoxantrone. According to another embodiment, the at least one chemotherapeutic agent is mitomycin. According to another embodiment, the at least one chemotherapeutic agent is dactinomycin. According to another embodiment, the at least one chemotherapeutic agent is irinotecan. According to another embodiment, the at least one chemotherapeutic agent is topotecan. According to another embodiment, the at least one chemotherapeutic agent is etoposide. According to another embodiment, the at least one chemotherapeutic agent is teniposide. According to another embodiment, the at least one chemotherapeutic agent is etoposide phosphate. According to another embodiment, the at least one chemotherapeutic agent is bleomycin sulfate.

According to another embodiment, the at least one chemotherapeutic agent is a biologic for which alopecia is a side-effect. According to another embodiment, the at least one chemotherapeutic agent is filgrastim. According to another embodiment, the at least one chemotherapeutic agent is pegfilgrastim. According to another embodiment, the at least one chemotherapeutic agent is bevacizumab. According to another embodiment, the at least one chemotherapeutic agent is sargramostim. According to another embodiment, the at least one chemotherapeutic agent is panitumumab.

According to another embodiment, the at least one chemotherapeutic agent is a hormone or related agent for which alopecia is a side-effect. According to another embodiment, the at least one chemotherapeutic agent is megestrol acetate. According to another embodiment, the at least one chemotherapeutic agent is fluoxymesterone. According to another embodiment, the at least one chemotherapeutic agent is leuprolide. According to another embodiment, the at least one chemotherapeutic agent is octreotide acetate. According to another embodiment, the at least one chemotherapeutic agent is tamoxifen citrate. According to another embodiment, the at least one chemotherapeutic agent is fluxymesterone.

According to another embodiment, the at least one chemotherapeutic agent is a miscellaneous agent for which alopecia is a side-effect. According to another embodiment, the at least one chemotherapeutic agent is sorafenib. According to another embodiment, the at least one chemotherapeutic agent is erlotinib. According to another embodiment, the at least one chemotherapeutic agent is oxaliplatin. According to another embodiment, the at least one chemotherapeutic agent is dexrazoxane. According to another embodiment, the at least one chemotherapeutic agent is anagrelide. According to another embodiment, the at least one chemotherapeutic agent is isotretinoin. According to another embodiment, the at least one chemotherapeutic agent is bexarotene. According to another embodiment, the at least one chemotherapeutic agent is vorinostat.

According to another embodiment, the topical composition is administered concurrently with a chemotherapy regimen for treating colorectal cancer. According to some such embodiments, the chemotherapy regimen is a combination of infusional 5-fluoruracil, leucovorin and oxaliplatin (FOLFOX®). This regimen combines three different antineoplastic agents: (1) 5-fluorouracil; (2) leucovorin; and oxaliplatin. According to some such embodiments, the chemotherapeutic regimen for colorectal cancer utilizes FOLFOX® with bevacizumab (Avastin®, a monoclonal antibody and anti-angiogenesis drug). According to some such embodiments, the chemotherapy regimen utilizes infusional 5-fluoruracil, leucovorin, and irinotecan (FOLFIRI®) with bevacizumab.

According to some embodiments, the optional second component is an additional active agent. In some such embodiments, the additional active agent is a therapeutic agent. According to some such embodiments, the therapeutic agent is an anti-fungal agent. According to some such embodiments, the therapeutic agent is an antihistamine. According to some such embodiments, the therapeutic agent is an anti-protozoal agent. According to some such embodiments, the therapeutic agent is an antimetabolite. According to some such embodiments, the therapeutic agent is an antipruritic agent. According to some such embodiments, the therapeutic agent is an anti-oxidant. According to some such embodiments, the therapeutic agent is an anti-viral agent. According to some such embodiments, the therapeutic agent is a chelating agent. According to some such embodiments, the therapeutic agent is a chemotherapeutic agent. According to some such embodiments, the therapeutic agent is a new excipient. According to some such embodiments, the therapeutic agent is a hormone. According to some such embodiments, the therapeutic agent is a non-steroidal anti-inflammatory agent. According to some such embodiments, the therapeutic agent is a peptide. According to some such embodiments, the therapeutic agent is a polypeptide. According to some such embodiments, the therapeutic agent is a prodrug. According to some such embodiments, the therapeutic agent is a peptidomimetic. According to some such embodiments, the therapeutic agent is a steroidal anti-inflammatory agent. According to some such embodiments, the therapeutic agent is a vitamin.

According to another embodiment, the at least one chemotherapeutic agent is of a therapeutically effective amount.

According to another embodiment, the at least one chemotherapeutic agent is a component of a chemotherapy regimen for treating a cancer.

According to some such embodiments, the cancer is breast cancer.

According to some such embodiments, the chemotherapy regimen comprises doxorubicin and cyclophosphamide. This regimen may be referred to as the “AC” chemotherapy regimen.

According to some such embodiments, the chemotherapy regimen comprises doxorubicin, cyclophosphamide, and paclitaxel. This regimen may be referred to as the “AC+Taxol” chemotherapy regimen.

According to some such embodiments, the chemotherapy regimen comprises doxorubicin, cyclophosphamide, and docetaxel. This regimen may be referred to as the “AC+Taxotere” chemotherapy regimen.

According to some such embodiments, the chemotherapy regimen comprises doxorubicin and paclitaxel (or docetaxel). This regimen may be referred to as the “AT” chemotherapy regimen.

According to some such embodiments, the chemotherapy regimen comprises cyclophosphamide, doxorubicin, and fluorouracil. This regimen may be referred to as the “CAF” chemotherapy regimen.

According to some such embodiments, the chemotherapy regimen comprises cyclophosphamide, methotrexate and fluorouracil. This regimen may be referred to as the “CMF” chemotherapy regimen.

According to some such embodiments, the chemotherapy regimen comprises epirubicin and cyclophosphamide. This regimen may be referred to as the “EC” chemotherapy regimen.

According to some such embodiments, the chemotherapy regimen comprises fluorouracil, epirubicin and cyclophosphamide. This regimen may be referred to as the “FEC” chemotherapy regimen.

According to another embodiment, the cancer is colon cancer.

According to some such embodiments, the chemotherapy regimen is a combination of infusional 5-fluoruracil, leucovorin and oxaliplatin (FOLFOX®). This regimen combines three different antineoplastic agents: (1) 5-fluorouracil; (2) leucovorin; and oxaliplatin. According to some such embodiments, the chemotherapeutic regimen for colorectal cancer utilizes FOLFOX® with bevacizumab (Avastin®, a monoclonal antibody and anti-angiogenesis drug). According to some such embodiments, the chemotherapy regimen utilizes infusional 5-fluoruracil, leucovorin, and irinotecan (FOLFIRI®) with bevacizumab.

According to another embodiment, the epithelial-related surface is an eyebrow. According to another embodiment, the epithelial surface onto which the composition is applied is a scalp. According to another embodiment, the epithelial-related surface onto which the composition is applied is an eyelid. According to another embodiment, the epithelial surface onto which the composition is applied is a face. According to another embodiment, the epithelial surface onto which the composition is applied is above a lip. According to another embodiment, the epithelial surface onto which the composition is applied is a chin.

According to another embodiment, the composition is formulated as a mascara. According to another embodiment, the composition is formulated as an ophthalmic.

Method for Protecting Hair from Chemotherapy Insults

According to another aspect, the described invention provides a method to protect hair of a subject in need thereof from chemotherapy insult, the method comprising steps:

(a) providing a topical composition comprising a first component and an optional second component,

the first component comprising

(i) at least one compound of Formula I or a pharmaceutically acceptable salt, hydrate, solvate, prodrug or metabolite thereof,

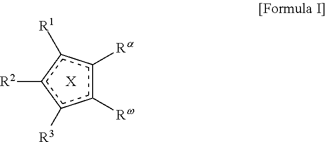

wherein ring X is selected from

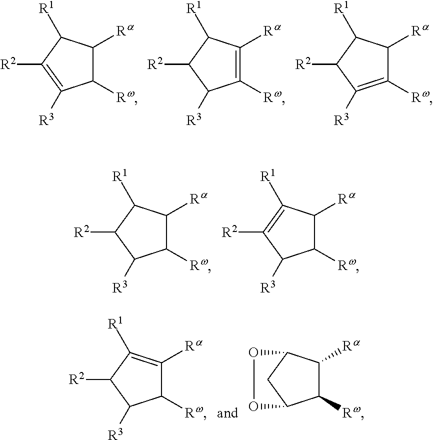

wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each,

wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1˜25 or a pharmaceutically acceptable salt thereof; —P(O)(OH)₂ or a pharmaceutically acceptable salt thereof,

wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,

wherein R^{α} is

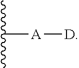

wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one

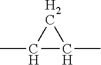

in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof,

wherein each R⁷ is independently H, F, or straight chain or branched C₁˜C₅ alkyl,

wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,

wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; NO₂; —SR¹⁰; —CH═NOR¹⁰; —C(═O)NR¹¹OR¹²; —S(O)₂NR⁹₂; —NR⁹S(O)₂R¹³; —OS(O)₂NR⁹₂; —C(O)NHS(O)₂R¹³; —S(O)₂R¹³; —SO₃H; —PO₃H₂;

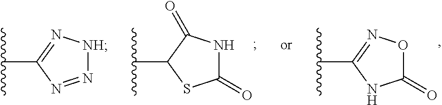

wherein R^{ω} is

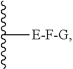

wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or CC bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof,

wherein F is —CH₂—, —O—; —S—; —S(O)—; —S(O₂)—; —C(O)—; —C(O)O—; —C(O)S—; —C(O)NR⁹—; —NR⁹—; or a covalent bond,

wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,

wherein each R⁸ is independently selected from the following: H; a pharmaceutically acceptable cation including, but not limited to, sodium, potassium, magnesium, calcium or an organic cation including but not limited to an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester including but not limited to a lower alkyl ester, a lower acyloxy-alkyl ester (including, but not limited to, acetoxymethyl, acetoxyethyl, aminocarbonyloxymethyl, pivaloyloxymethyl or pivaloyloxyethyl ester), a lactonyl ester (including, but not limited to, a phthalidyl or thiophthalidyl ester), a lower alkoxyacyloxyalkyl ester (including, but not limited to, a methoxycarbonyloxymethyl, ethoxycarbonyloxyethyl or isopropoxycarbonyloxyethyl ester), an alkoxyalkyl ester, choline ester or acylamino alkyl ester (including, but not limited to, an acetamidomethyl ester); or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,

wherein each R⁹ is independently selected from the following: H; a C₁˜C₂₀ straight chain or branched acyclic hydrocarbon group containing zero to four C═C or C≡C bonds wherein each C═C bond independently can be of E or Z configuration; a C₁˜C₂₀ straight chain or branched acyl group containing zero to four C═C or C≡C bonds wherein each C═C bond independently can be of E or Z configuration; —(CH₂)_{q}OH, —(CH₂)_{q}OR¹⁴, —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl (including, but not limited to, acetoxymethyl, acetoxyethyl, aminocarbonyloxymethyl, pivaloyloxymethyl or pivaloyloxyethyl), lactonyl (including, but not limited, to a phthalidyl or thiophthalidyl), lower alkoxyacyloxyalkyl (including, but not limited to, a methoxycarbonyloxymethyl, ethoxycarbonyloxyethyl or isopropoxycarbonyloxyethyl), or acylamino alkyl (including, but not limited to, acetamidomethyl); or -J-K; or —NR⁹₂ can be a cycloamido radical (including, but not limited to, 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like),

wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl

wherein each R¹¹ is independently H or —C(O)R¹⁶,

wherein each R¹² is independently R¹⁶ or —C(O)R¹⁶,

wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen),

wherein each R¹⁴ is independently straight chain or branched C₁˜C₆ alkyl or —CH₂OCH₃,

wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂;

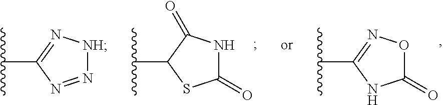

wherein each R¹⁶ is independently H or straight chain or branched C₁˜C₆ alkyl, phenyl or —CH₂OCH₃,

wherein each R¹⁷ is independently straight chain or branched C₁˜C₆ alkyl; —C(O)OR¹⁶; phenyl; F; Cl; Br; I; CF3; —C(O)N(R16)2; —OR16; —N(R16)C(O)R16; —N(R16)2; —C(O)R16; —OC(O)R16; —C≡N; —NO2; —S(O)2N(R16)2; —NR16S(O)2R13, with the proviso that, if R17 is —NR16S(O)2R13, R¹, R², R³, R^{α} and R^{ω} are selected such that the molecular weight of the compound of Formula I does not exceed about 2000 atomic mass units, and

wherein not more than four of R⁷ are other than H or F and not more than four of R⁷ are F; and

wherein each chiral center or allenic moiety independently possesses any relative or absolute stereoconfiguration or comprises any mixture thereof;

the optional second component comprising an additional active agent; and

a carrier;

(b) topically administering the topical composition onto an epithelial surface of a subject in need prior to administration of at least one chemotherapeutic agent;

(c) stimulating hair growth on an epithelial surface to which the topical composition has been applied; and

(d) preventing an epithelial-related disorder from occurring on the epithelial surface of step (b), wherein the epithelial-related disorder is selected from the group consisting of sparse hair growth, short hair growth, brittle hair growth, thin hair growth, alopecia and hair depigmentation.

In some embodiments, the at least one compound of Formula I is a mixture of diastereomers in any ratio.

In some embodiments, the at least one compound of Formula I is enantiomerically pure.

In some embodiments, the at least one compound of Formula I is a mixture of enantiomers in any ratio, including a racemate.

In some embodiments, the at least one compound of Formula I is diastereomerically and enantiomerically pure.

In some embodiments, the at least one compound of Formula I is a mixture of diastereomers and enantiomers in any ratio.

In some embodiments, the at least one compound of Formula I has one or more hydrogen atoms replaced by deuterium.

According to some embodiments, the compound of formula I is latanoprost. According to another embodiment, the compound of formula I is travoprost. According to another embodiment, the compound of formula I is travoprost ethylamide. According to another embodiment, the compound of formula I is bimatoprost. According to another embodiment, the compound of formula I is fluprostenol. According to another embodiment, the compound of formula I is fluprostenol isopropyl ester. According to another embodiment, the compound of formula I is fluprostenol methyl amide. According to another embodiment, the compound of formula I is 9-keto fluprostenol isopropyl ester. According to another embodiment, the compound of formula I is cloprostenol. According to another embodiment, the compound of formula I is cloprostenol isopropyl ester. According to another embodiment, the compound of formula I is chloprostenol methyl amide. According to another embodiment, the compound of formula I is 17 phenyl-18,19,20-trinor prostaglandin E2 (N-(1,3-dihydroxypropan-2-yl))amide. According to another embodiment, the compound of formula I is 17 phenyl-18,19,20-trinor prostaglandin F2α (N-(1,3-dihydroxypropan-2-yl))amide.

In some embodiments, when R² is H, G is phenyl or heterocycle and D is —C(O)NR⁹₂ or —C(O)OR⁸, the compound of the described invention is a compound of Formula (II) or a solvate, hydrate, salt, prodrug or metabolite of Formula (II):

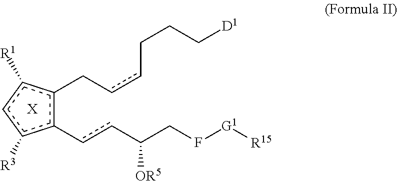

wherein G¹ is phenyl or a 6-membered heterocycle; said heterocycle being aromatic or non-aromatic; said heterocycle containing at least two heteroatoms selected from the group consisting of N, S and O; said phenyl or heterocycle being optionally substituted with one to three R¹⁵ groups;

wherein D¹ is —C(O)OR⁸ or —C(O)NR⁹₂.

The following are nonlimiting embodiments depicting cyclopentane ring X of the compound of Formula II, wherein R¹ is ═O and R³ is H, which has the structure:

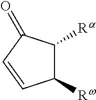

In one embodiment, R⁵ is H, D is —C(O)NR⁹₂, R⁹₂ is H and a nitrogen-linked cyclopropyl group, F is —O—, G is phenyl and there is no R¹⁵. This compound of Formula II has the structure:

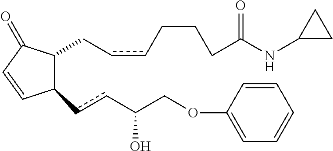

wherein a hashed line represents a substituent behind the plane of this paper, a bold wedge represents a substituent above the plane of this paper, and a dashed line represents a single or double bond which can be in the cis or trans configuration. In one embodiment, a compound in which the dashed lines are double bonds with the stereoconfigurations as drawn is 16-phenoxy-17, 18,19,20-tetranor PGA₂ N-cyclopropylamide. A compound where the C₅-C₆ dashed line is a single bond, and the C₁₃-C₁₄ dashed line is a double bond with the stereoconfigurations as drawn is 16-phenoxy-17, 18,19,20-tetranor PGA₁ N-cyclopropylamide.

In one embodiment, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

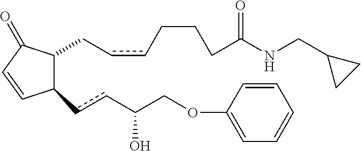

wherein a hashed line represents a substituent behind the plane of this paper, a bold wedge represents a substituent above the plane of this paper, wherein the dashed lines represents a single bond or a double bond in cis or trans configuration. A compound where the 5,6 bond is in a cis configuration and the 13,14 bond is in a trans configuration with the stereoconfigurations as drawn is 16-phenoxy-17,18,19,20-tetranor PGA₂ N-cyclopropylmethylamide. A compound where the C₅-C₆ dashed line is a single bond, and the C₁₃-C₁₄ dashed line is a double bond with the stereoconfigurations as drawn is 16-phenoxy 17,18,19,20-tetranor PGA₁ N-cyclopropyl methylamide.

In some embodiments, R⁵ is H, D¹ is —C(O)NR⁹₂, R⁹₂ is H and a nitrogen-linked-cyclopropyl group, F is —CH₂—, G¹ is phenyl and there is no R¹⁵. This compound of Formula II has the structure:

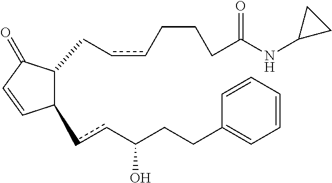

wherein a hashed line represents a substituent below the plane of this paper, a bold wedge represents a substituent above the plane of this paper, and the dashed lines represent a single bond or a double bond which can be in the cis or trans configuration. A compound with the stereoconfigurations as drawn is 17-phenyl-18,19,20 trinor PGA₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGA₁ N-cyclopropylamide.

In some embodiments, R⁵ is H, D¹ is —C(O)NR⁹₂, R⁹₂ is H and a nitrogen-linked-cyclopropylmethyl group, F is —CH₂—, G¹ is phenyl and there is no R¹⁵. This compound of Formula II has the structure:

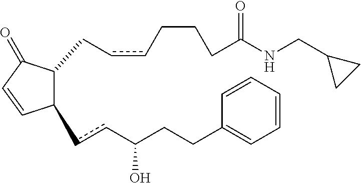

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn is 17-phenyl-18,19, 20-trinor PGA₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGA₁ N-cyclopropylmethylamide.

In some embodiments, R⁵ is H, D′ is —C(O)NR⁹₂, R⁹₂ is H and a nitrogen-linked —CH(CH₂OH)₂ group, F is —CH₂—, G′ is phenyl and there is no R¹⁵. This compound of Formula II has the structure:

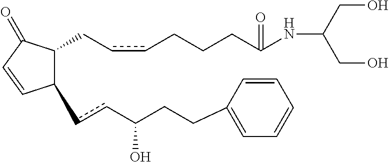

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGA₂ (N-(1,3-dihydroxypropan-2-yl))amide. A compound wherein the C₅-C₆ dashed line is a single bond and the C₁₃-C₁₄ dashed line is a double bond with the stereoconfigurations as drawn is 17-phenyl-18,19,20-trinor PGA₁ (N-(1,3-dihydroxypropan-2-yl))amide.

The following are nonlimiting embodiments depicting cyclopentane ring X of the compound of Formula II, wherein R¹ is ═O, and R³ is H, which has the structure:

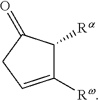

In some embodiments R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This vb v, the compound of Formula II has the structure:

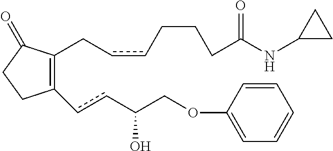

wherein a hashed line represents a substituent below the plane of this paper, and a dashed line represents a double bond having the configuration indicated. This compound, where the dashed lines are double bonds with the stereoconfigurations as drawn is 16-phenoxy-17, 18, 19,20-tetranor PGB₂ N-cyclopropylamide. A compound where the C₅-C₆ dashed line is a single bond, and the C₁₃-C₁₄ dashed line is a double bond with the stereoconfigurations as drawn is 16-phenoxy-17, 18,19,20-tetranor PGB₁ N-cyclopropylamide.

In some embodiments, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

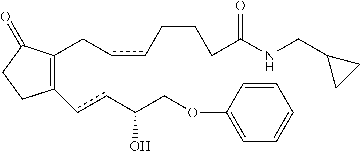

wherein a hashed line represents a substituent below the plane of this paper, and a dashed line represents a single bond or a double bond with a cis or trans configuration. A compound, where the dashed lines are double bonds with the stereoconfigurations as drawn is 16-phenoxy-17,18,19,20-tetranor PGB₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGB₁ N-cyclopropylmethylamide.

In some embodiments, R⁵ is H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

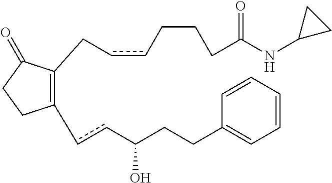

wherein a hashed line represents a substituent below the plane of this paper, and a dashed line represents a single bond or a double bond which can be in the cis or trans configuration. A compound where the dashed lines are double bonds with the stereoconfigurations as drawn is 17-phenyl-18,19,20-trinor PGB₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGB₁ N-cyclopropylamide.

In some embodiments, R⁵ is H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

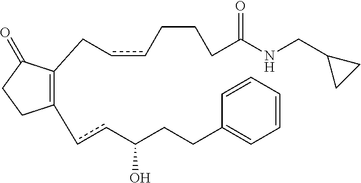

wherein a hashed line represents a substituent below the plane of this paper, and a dashed line represents a single bond or a double bond which can be in the cis and trans configuration. A compound where the dashed lines are double bonds with the stereoconfigurations as drawn is 17-phenyl-18,19,20-trinor PGB₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGB₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

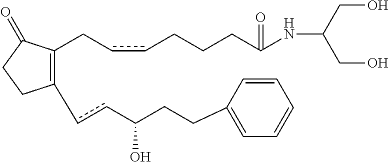

wherein a hashed line represents a substituent below the plane of this paper, and a dashed line represents a single bond or a double bond having the configuration indicated. A compound wherein the dashed lines are double bonds with the stereoconfigurations as drawn is 17-phenyl-18,19,20-trinor PGB₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGB₁ (N-(1,3-dihydroxypropan-2-yl))amide.

The following are nonlimiting embodiments depicting cyclopentane ring X of the compound of Formula II, wherein R¹ is ═O, R² is H, and R³ is H, which has the structure:

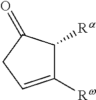

In some embodiments, R⁵ is H, D¹ is —C(O)NR⁹₂, R⁹₂ is H and a nitrogen-linked cyclopropyl group, F is —O—, G¹ is phenyl and there is no R¹⁵. This compound of Formula II has the structure:

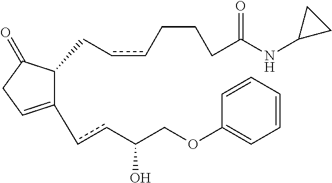

wherein a hashed line represents a substituent below the plane of this paper, and a dashed line represents a single bond or a double bond which can be in the cis or trans configuration. A compound where the dashed lines are double bonds with the stereoconfigurations as drawn is 16-phenoxy-17,18,19,20-tetranor PGC₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGC₁ N-cyclopropylamide.

In some embodiments, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

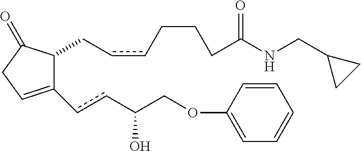

wherein a hashed line represents a substituent below the plane of this paper, and a dashed line represents a single bond or a double bond which can be in the cis or trans configuration. A compound, where the dashed lines are double bonds with the stereoconfigurations as drawn is 16-phenoxy-17,18,19,20-tetranor PGC₂N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGC₁ N-cyclopropylmethylamide.

In some embodiments, R⁵ is H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

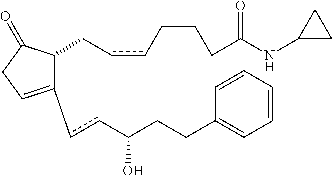

wherein a hashed line represents a substituent below the plane of this paper, and a dashed line represents a single bond or a double bond which can be in the cis and trans configuration. A compound where the dashed lines are double bonds with the stereoconfigurations as drawn is 17-phenyl-18,19,20-trinor PGC₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGC₁ N-cyclopropylamide.

In some embodiments, R⁵ is H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

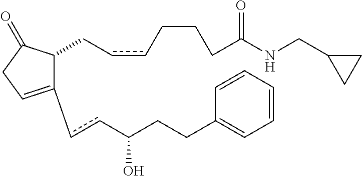

wherein a hashed line represents a substituent below the plane of this paper, and a dashed line represents a single bond or a double bond which can be in the cis or trans configuration. A compound, where the dashed lines are double bonds with the stereoconfigurations as drawn is 17-phenyl-18,19,20-trinor PGC₂ N-cyclopropylmethyl amide. A compound where the C₅-C₆ dashed line is a single bond, and the C₁₃-C₁₄ dashed line is a double bond with the stereoconfigurations as drawn is 17-phenyl-18,19,20-trinor PGC₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

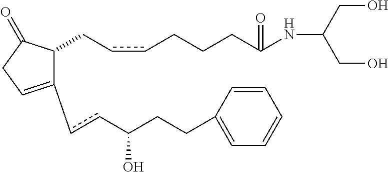

wherein a hashed line represents a substituent below the plane of this paper, a bold wedge represents a substituent above the plane of this paper, and a dashed line represents a single or a double bond which can be in the cis or trans configuration. A compound wherein the dashed lines are double bonds with the stereoconfigurations as drawn is 17-phenyl-18,19,20-trinor PGC₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGC₁ (N-(1,3-dihydroxypropan-2-yl))amide.

The following are nonlimiting embodiments depicting cyclopentane ring X of the compound of Formula II, wherein R¹ is OH, and R³ is ═O, which has the structure:

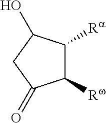

In some embodiments, R⁵ is H, D¹ is —C(O)NR⁹₂, R⁹₂ is H and a nitrogen-linked cyclopropyl group, F is —O—, G¹ is phenyl and there is no R¹⁵. This compound of Formula II has the structure:

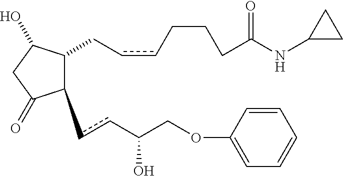

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 16-phenoxy-17,18,19,20-tetranor PGD₂ N-cyclopropylamide. A compound where the C₅-C₆ dashed line is a single bond, and the C₁₃-C₁₄ dashed line is a double bond with the stereoconfigurations as drawn is 16-phenoxy-17,18,19,20-tetranor PGD₁ N-cyclopropylamide.

In some embodiments, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

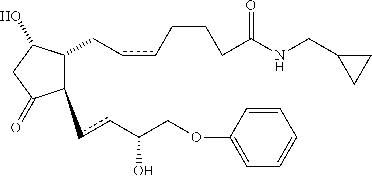

wherein a hashed line represents a substituent below the plane of this paper, a bold wedge represents a substituent above the plane of this paper, and a dashed line represents a single or a double bond which can be in the cis or trans configuration. A compound where the dashed lines are double bonds with the stereoconfigurations as drawn is 16-phenoxy-17,18-,19,20-tetranor PGD₂ N-cyclopropylmethylamide. A compound where the C₅-C₆ dashed line is a single bond, and the C₁₃-C₁₄ dashed line is a double bond with the stereoconfigurations as drawn is 16-phenoxy-17,18,19,20-tetranor PGD₁ N-cyclopropylmethylamide.

In some embodiments, R⁵ is H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

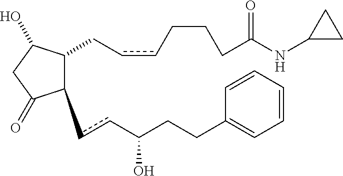

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGD₂ N-cyclopropylamide. A compound where the C₅-C₆ dashed line is a single bond, and the C₁₃-C₁₄ dashed line is a double bond with the stereoconfigurations as drawn is 17-phenyl-18,19,20-trinor PGD₁ N-cyclopropylamide.

In some embodiments, R⁵ is H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

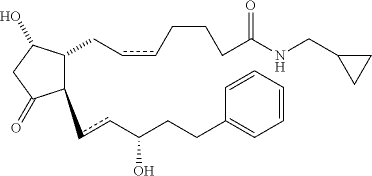

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGD₂ N-cyclopropylmethylamide. A compound where the C₅-C₆ dashed line is a single bond, and the C₁₃-C₁₄ dashed line is a double bond with the stereoconfigurations as drawn is 17-phenyl-18,19,20-trinor PGD₁ N-cyclopropylmethylamide.

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

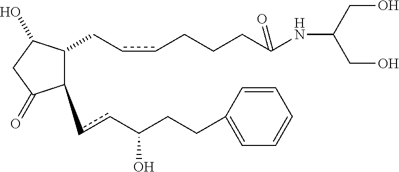

wherein a hashed line represents a substituent below the plane of this paper, a bold wedge represents a substituent above the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGD₂ (N-(1,3-dihydroxypropan-2-yl))amide. A compound wherein the C₅-C₆ dashed line is a single bond, and the C₁₃-C₁₄ dashed line is a double bond with the stereoconfigurations as drawn is 17-phenyl-18,19,20-trinor PGD₁ (N-(1,3-dihydroxypropan-2-yl))amide.

The following are nonlimiting embodiments depicting cyclopentane ring X of the compound of Formula II, wherein R¹ is ═O, and R³ is OH or OR⁴, which has the structure:

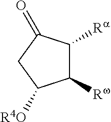

In one embodiment, R⁴ and R⁵ are both H, D¹ is —C(O)NR⁹₂, R⁹₂ is H and a nitrogen-linked cyclopropyl group, F is —CH₂—, G¹ is phenyl and there is no R¹⁵. This compound of Formula II has the structure:

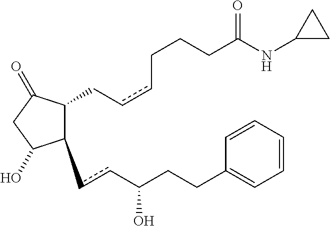

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds with the stereoconfigurations as drawn is 17-phenyl-18,19,20-trinor PGE₂ N-cyclopropylamide. A compound wherein the C₅-C₆ dashed line is a single bond, and the C₁₃-C₁₄ dashed line is a double bond with the stereoconfigurations as drawn is 17-phenyl-18,19,20-trinor PGE₁ N-cyclopropylamide.

In another embodiment, R⁴ and R⁵ are both H, D¹ is —C(O)NR⁹₂, R⁹₂ is H and a nitrogen-linked cyclopropylmethyl group, F is —CH₂—, G¹ is phenyl and there is no R¹⁵. This compound of Formula II has the structure:

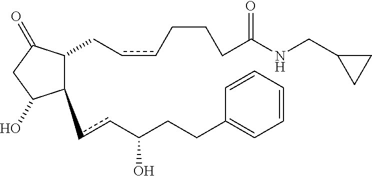

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds with the stereoconfigurations as drawn, is 17-phenyl-18,19,20-trinor PGE₂ N-cyclopropylmethylamide. A compound wherein the C₅-C₆ dashed line is a single bond, and the C₁₃-C₁₄ dashed line is a double bond with the stereoconfigurations as drawn is 17-phenyl-18,19,20-trinor PGE₁ N-cyclopropylmethylamide.

In yet another embodiment, R⁴ is —PO₃⁻² with pharmaceutically acceptable counter ion(s) such as 2 Na⁺, R⁵ is H, F is O, G¹ is phenyl, there is no R¹⁵, D¹ is —C(O)OR⁸, and R⁸ is a cyclopropyl group. This compound of Formula II has the following structure (counterions not shown):

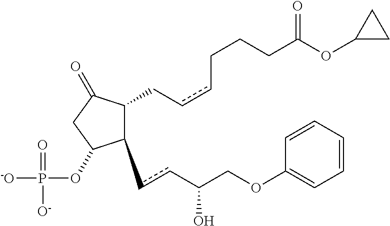

wherein a hashed line represents a substituent below the plane of this paper, a bold wedge represents a substituent above the plane of this paper, and a dashed line represents a single bond or a double bond which can be in the cis or trans configuration.

In another embodiment, R⁵ is H, D¹ is —C(O)NR⁹₂, F is ═O, R⁹₂ is H and a nitrogen-linked cyclopropyl group, F is —O—, G¹ is phenyl, and there is no R¹⁵. This compound of Formula II has the structure:

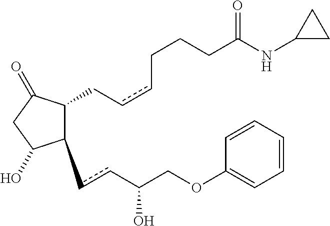

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds with the stereoconfigurations as drawn is 16-phenoxy-17,18,19,20-tetranor PGE₂ N-cyclopropylamide. A compound wherein the C₅-C₆ dashed line is a single bond, and the C₁₃-C₁₄ dashed line is a double bond with the stereoconfigurations as drawn is 16-phenoxy-17,18,19,20-tetranor PGE₁ N-cyclopropylamide.

In another embodiment, R⁵ is H, D¹ is —C(O)NR⁹₂, R⁹₂ is H and a nitrogen-linked cyclopropylmethyl group, F is —O—, G¹ is phenyl and there is no R¹⁵. This compound of Formula II has the structure:

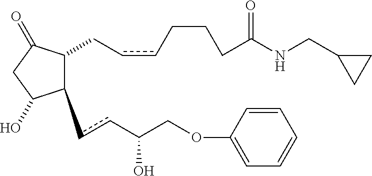

wherein a hashed line represents a substituent below the plane of this paper, a bold wedge represents a substituent above the plane of this paper, and a dashed line represents a single bond or a double bond which can be in the cis or trans configuration. A compound where the dashes represent double bonds with the stereoconfigurations as drawn is 16-phenoxy-17,18,19,20-tetranor PGE₂ N-cyclopropylmethylamide. A compound wherein the C₅-C₆ dashed line is a single bond, and the C₁₃-C₁₄ dashed line is a double bond with the stereoconfigurations as drawn is 16-phenoxy-17,18,19,20-tetranor PGE₁ N-cyclopropylmethylamide.

In another embodiment, R⁵ is H, D¹ is —C(O)NR⁹₂, R⁹₂ is H and a nitrogen-linked cyclopropyl group, F is CH₂, G¹ is pyrimidin-2-yl and there is no R¹⁵. This compound of Formula II has the structure:

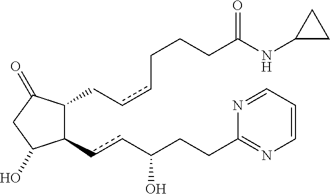

wherein a hashed line represents a substituent below the plane of this paper, a bold wedge represents a substituent above the plane of this paper, and a dashed line represents a single bond or a double bond which can be in the cis or trans configuration. A compound, where the dashed lines are double bonds, is 17-pyrimidin-2-yl-18,19,20-trinor PGE₂ N-cyclopropylamide. A compound wherein the C₅-C₆ dashed line is a single bond, and the C₁₃-C₁₄ dashed line is a double bond with the stereoconfigurations as drawn is 17-pyrimidin-2-yl-18,19,20-trinor PGE₁ N-cyclopropylamide.

In another embodiment, R³ is —OR⁴ and R⁴ is —PO₃H₂, R⁵ is H, F is —S(O)—, G¹ is phenyl, there is no R¹⁵, D¹ is —C(O)OR⁸, and R⁸ is a cyclopropyl group. This compound of Formula II has the structure:

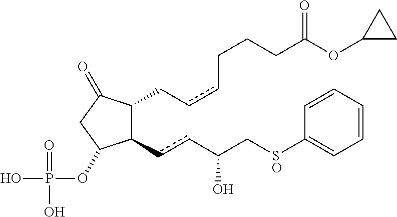

wherein a hashed line represents a substituent below the plane of this paper, a bold wedge represents a substituent above the plane of this paper, and a dashed line represents a single bond or a double bond which can be in the cis or trans configuration.

In yet another embodiment, R³ is OR⁴ where R⁴ is a monosaccharide (e.g., glucose), R⁵ is H, D¹ is —C(O)NR⁹₂, R⁹₂ is H and a nitrogen-linked ethyl group; F is —CH₂—, G¹ is phenyl, and there is no R¹⁵. This compound of Formula II has the structure:

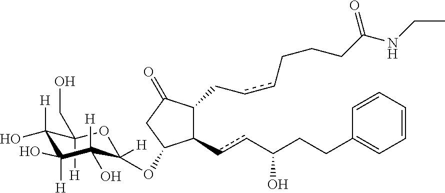

wherein a hashed line represents a substituent below the plane of this paper, a bold wedge represents a substituent above the plane of this paper, and a dashed line represents a single bond or a double bond which can be in the cis or trans configuration.

In yet another embodiment, R³ is OR⁴ where R⁴ is a monosaccharide (e.g., glucose), R⁵ is H, D¹ is —C(O)NR⁹₂, R⁹₂ is H and a nitrogen-linked ethyl group; F is —CH₂—, G¹ is 1,3-oxazinan-2-yl, and R¹⁵ is a cyclopropyl group linked to a nitrogen heteroatom as shown. This compound of Formula II has the structure:

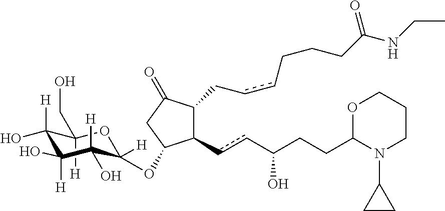

wherein a hashed line represents a substituent below the plane of this paper, a bold wedge represents a substituent above the plane of this paper, and a dashed line represents a single bond or a double bond which can be in the cis or trans configuration. A

In some embodiments, R⁵ is H, D¹ is —C(O)NR⁹₂, R⁹₂ is H and a nitrogen-linked —CH(CH₂OH)₂ group, F is —CH₂—, G¹ is phenyl and there is no R¹⁵. This compound of Formula II has the structure:

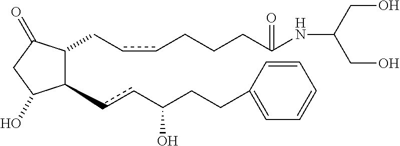

wherein a hashed line represents a substituent below the plane of this paper, a bold wedge represents a substituent above the plane of this paper, and a dashed line represents a double bond having the configuration indicated. A compound wherein the dashed lines are double bonds with the stereoconfigurations as drawn is 17-phenyl-18,19,20-trinor PGE₂ (N-(1,3-dihydroxypropan-2-yl)) amide. A compound wherein the C₅-C₆ dashed line is a single bond and the C₁₃-C₁₄ dashed line is a double bond with the stereoconfigurations as drawn is 17-phenyl-18,19,20-trinor PGE₁ (N-(1,3-dihydroxypropan-2-yl)) amide.

The following are nonlimiting embodiments depicting cyclopentane ring X of the compound of Formula II, wherein R¹ is OH or OR⁴, and R³ is OH or OR⁴, which has the structure:

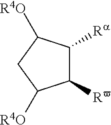

In one embodiment, R⁵ is H, D¹ is —C(O)NR⁹₂, R⁹₂ is H and a nitrogen-linked cyclopropyl group, F is —CH₂—, G¹ is phenyl, and there is no R¹⁵ group. This compound of Formula II, the structure:

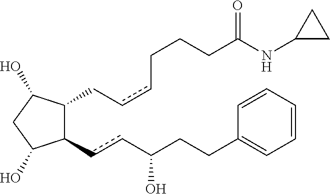

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn is 17-phenyl-18,19,20-trinor PGF_{2α} N-cyclopropylamide. A compound wherein the C₅-C₆ dashed line is a single bond and the C₁₃-C₁₄ dashed line is a double bond with the stereoconfigurations as drawn is 17-phenyl-18,19,20-trinor PGF_{2α} N-cyclopropylamide.

In another embodiment, R⁵ is H, D¹ is —C(O)NR⁹₂, R⁹₂ is H and a nitrogen-linked cyclopropylmethyl group, F is —CH₂—, G¹ is phenyl, and there is no R¹⁵. This compound has the structure:

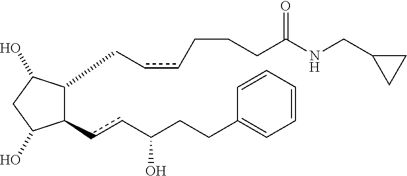

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 17-phenyl-18,19,20-trinor PGF_{2α} N-cyclopropylmethylamide. A compound wherein the C₅-C₆ dashed line is a single bond and the C₁₃-C₁₄ dashed line is a double bond with the stereoconfigurations as drawn is 17-phenyl-18,19,20-trinor PGF_{2α} N-cyclopropylmethylamide.

In yet another embodiment, R¹ is OR⁴, where R⁴ is PO₃²⁻ with pharmaceutically acceptable counterion(s) such as 2 Na⁺, D¹ is —C(O)OR⁸, R⁵ is H, F is —O—, G¹ is phenyl, there is no R¹⁵, and R⁸₂ is a cyclopropyl group. This compound of Formula II has the structure:

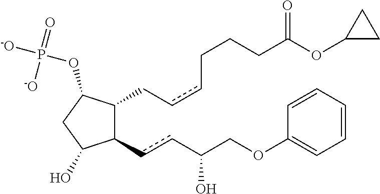

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In another embodiment, R⁵ is H, D¹ is —C(O)NR⁹₂, R⁹₂ is H and a nitrogen-linked cyclopropyl group, F is —O—, G¹ is phenyl, there is no R¹⁵. This compound of Formula II has the structure:

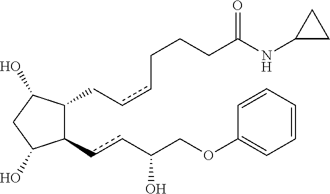

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn is 16-phenoxy-17,18,19,20-tetranor PGF_{2α} N-cyclopropylamide. A compound wherein the C₅-C₆ dashed line is a single bond and the C₁₃-C₁₄ dashed line is a double bond with the stereoconfigurations as drawn is 16-phenoxy-17,18,19,20-tetranor PGF_{2α} N-cyclopropylamide.

In another embodiment, R⁵ is H, D¹ is —C(O)NR⁹₂, R⁹₂ is H and a nitrogen-linked cyclopropylmethyl group, F is —O—, G¹ is phenyl, and there is no R¹⁵. This compound, has the structure:

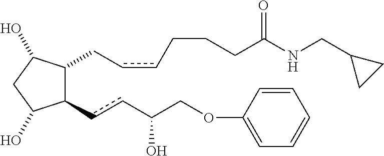

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 16-phenoxy-17,18,19,20-tetranor PGF_{2α} N-cyclopropylmethylamide A compound wherein the C₅-C₆ dashed line is a single bond and the C₁₃-C₁₄ dashed line is a double bond with the stereoconfigurations as drawn is 16-phenoxy-17,18,19,20-tetranor PGF_{2α} N-cyclopropylmethylamide.

In another embodiment, R⁵ is H, D¹ is —C(O)NR⁹₂, R⁹₂ is H and a nitrogen-linked cyclopropyl group, F is CH₂, G¹ is pyrimidin-2-yl, and there is no R¹⁵. This compound of Formula II has the structure:

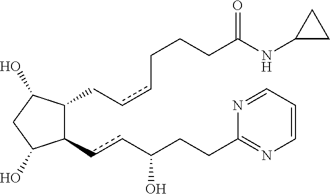

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 17-pyrimidin-2-yl-18,19,20-trinor PGF₂ N-cyclopropylamide. A compound wherein the C₅-C₆ dashed line is a single bond and the C₁₃-C₁₄ dashed line is a double bond with the stereoconfigurations as drawn is 17-pyrimidin-2-yl-18,19,20-trinor PGF_{2α} N-cyclopropylamide.

In another embodiment, R¹ is OR⁴ where R⁴ is —(PO₂(OH))ₛH, where s is 1-25 or a pharmaceutically acceptable salt thereof, R⁵ is H, F is —S(O)—, G¹ is phenyl, D¹ is —C(O)OR⁸, and R⁸ is a cyclopropyl group. This compound of Formula II has the structure:

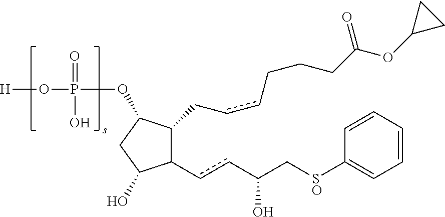

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn is a poly-hydroxyphosphoryloxy derivative of cyclopropyl 7-((1R,3R,5S)-3-hydroxy-2-((3R,E)-3-hydroxy-4-(phenylsulfinyl)but-1-enyl)-5-(phosphonooxy)cyclopentyl)heptanoate. A compound wherein the C₅-C₆ dashed line is a single bond and the C₁₃-C₁₄ dashed line is a double bond with the stereoconfigurations as drawn is cyclopropyl 7-((1R,3R,5S)-3-hydroxy-5-phosphonooxy-2-((3R,E)-3-hydroxy-4-(phenylsulfinyl)but-1-enyl)cyclopentyl)heptanoate.

In yet another embodiment, R¹ is OR⁴ and R⁴ is a monosaccharide (e.g., glucose), R⁵ is H, D¹ is —C(O)NR⁹₂, R⁹₂ is H and a nitrogen-linked ethyl group; F is —CH₂—, G¹ is phenyl, and there is no R¹⁵. This compound of Formula II has the structure:

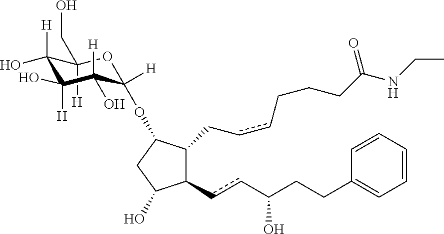

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In yet another embodiment, R¹ is OR⁴ and R⁴ is a monosaccharide (e.g., glucose), R⁵ is H, D¹ is —C(O)NR⁹₂, R⁹₂ is H and a nitrogen-linked ethyl group; F is —CH₂—, G¹ is 1,3-oxazinan-2-yl and R¹⁵ is a cyclopropyl group. This compound of Formula II has the structure:

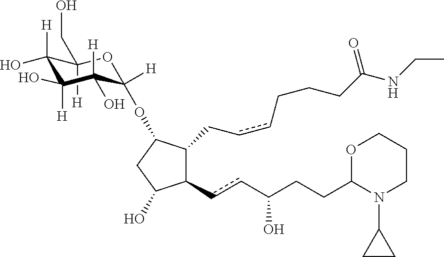

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane.

In another embodiment, R⁵ is H, D¹ is —C(O)NR⁹₂, R⁹₂ is H and cyclopropyl; F is —O—; G¹ is phenyl and R¹⁵ is Cl. This compound of Formula II has the structure:

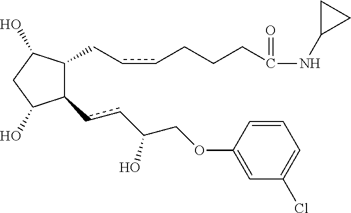

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 16-(3-chlorophenoxy)-17,18,19,20-tetranor PGF2α N-cyclopropylamide. A compound wherein the C₅-C₆ dashed line is a single bond and the C₁₃-C₁₄ dashed line is a double bond with the stereoconfigurations as drawn is 16-(3-chlorophenoxy)-17,18,19,20-tetranor PGF_{2α} N-cyclopropylamide.

In another embodiment, R⁵ is H, D¹ is —C(O)NR⁹₂, R⁹₂ is H and N-cyclopropylmethylamide;

F is —O—; G¹ is phenyl; and R¹⁵ is Cl. This compound of Formula II has the structure:

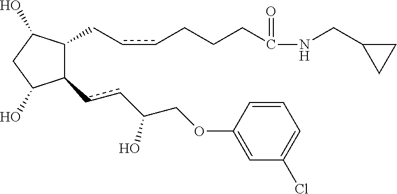

wherein the simple straight line represents a bond lying approximately in the surface plane; the dashed lines represent a single or double bond which can be in the cis or trans configuration, wherein there is at least one double bond; the bold wedge shaped bond is directed to the front of surface plane; and the hatched bond is directed in the back of the surface plane. A compound where the dashed lines are double bonds with the stereoconfigurations as drawn, is 16-(3-chlorophenoxy)-17,18,19,20-tetranor PGF2α-N-cyclopropylmethylamide. A compound wherein the C₅-C₆ dashed line is a single bond and the C₁₃-C₁₄ dashed line is a double bond with the stereoconfigurations as drawn is 16-(3-chlorophenoxy)-17,18,19,20-tetranor PGF_{2α} N-cyclopropylmethylamide.

In some embodiments, R⁵ is H, D¹ is —C(O)NR⁹₂, R⁹₂ is H and a nitrogen-linked —CH(CH₂OH)₂ group; F is —CH₂—; G¹ is phenyl; and R¹⁵ is Cl. This compound of Formula II has the structure:

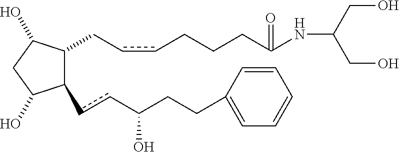

wherein a hashed line represents a substituent below the plane of this paper, a bold wedge represents a substituent above the plane of this paper, and a dashed line represents a double bond having the configuration indicated. A compound wherein the dashed lines are double bonds with the stereoconfigurations as drawn is 17-phenyl-18,19,20-trinor PGF2α (N-(1,3-dihydroxypropan-2-yl))amide. A compound wherein the C₅-C₆ dashed line is a single bond and the C₁₃-C₁₄ dashed line is a double bond with the stereoconfigurations as drawn is 17-phenyl-18,19,20-trinor PGF_{2α} (N-(1,3-dihydroxypropan-2-yl))amide.

In some embodiments, the compound of Formula II has the structure:

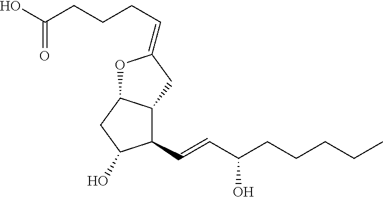

wherein a hashed line represents a substituent below this paper's plane; wherein a bold wedge represents a substituent above this paper's plane, with the stereoconfigurations as drawn is ((Z)-5-((3aR,4R,5R,6aS)-5-hydroxy-4-((S,E)-3-hydroxyoct-1-enyl)hexahydro-2H-cyclopenta[b]furan-2-ylidene)pentanoic acid) (PGI₂).

The following are nonlimiting embodiments depicting cyclopentane ring X of the compound of Formula II, wherein R¹ is H, R² is H, and R³ is ═O, which has the structure:

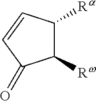

In some embodiments, R⁵ is H, D¹ is —C(O)NR⁹₂, R⁹₂ is H and a nitrogen-linked cyclopropyl group; F is —O—; G¹ is phenyl; and there is no R¹⁵. This compound of Formula II has the structure:

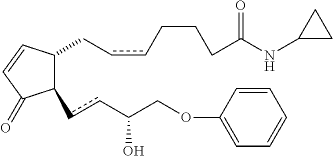

wherein a hashed line represents a substituent below the plane of this paper, a bold wedge represents a substituent above the plane of this paper, and a dashed line represents a single bond or a double bond which can be in cis or trans configuration. A compound where the dashed lines are double bonds with the stereoconfigurations as drawn is 16-phenoxy-17,18,19,20-tetranor PGJ₂ N-cyclopropylamide. A compound wherein the C₅-C₆ dashed line is a single bond and the C₁₃-C₁₄ dashed line is a double bond with the stereoconfigurations as drawn is 16-phenoxy-17,18,19,20-tetranor PGJ₁ N-cyclopropylamide.

In some embodiments, R⁵ is H, D¹ is —C(O)NR⁹₂, R⁹₂ is H and a nitrogen-linked cyclopropylmethyl group; F is —O—; G¹ is phenyl; and there is no R¹⁵. This compound of Formula II has the structure:

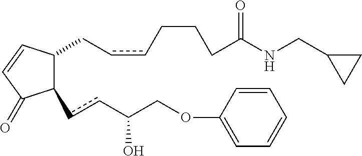

wherein a hashed line represents a substituent below the plane of this paper, a bold wedge represents a substituent above the plane of this paper, and a dashed line represents a double bond having the configuration indicated. A compound, where the dashed lines are double bonds with the stereoconfigurations as drawn is 16-phenoxy-17,18,19,20-tetranor PGJ₂ N-cyclopropylmethylamide. A compound wherein the C₅-C₆ dashed line is a single bond and the C₁₃-C₁₄ dashed line is a double bond with the stereoconfigurations as drawn is 16-phenoxy 17,18,19,20-tetranor PGJ₁ N-cyclopropylmethylamide.

In some embodiments, R⁵ is H, D¹ is —C(O)NR⁹₂, R⁹₂ is H and a nitrogen-linked cyclopropyl group; F is —CH₂—; G¹ is phenyl; and there is no R¹⁵. This compound of Formula II has the structure:

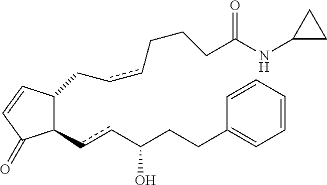

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGJ₂ N-cyclopropylamide. A compound wherein the C₅-C₆ dashed line is a single bond and the C₁₃-C₁₄ dashed line is a double bond with the stereoconfigurations as drawn is 17-phenyl-18,19,20-trinor PGJ₁ N-cyclopropylamide.

In some embodiments, R⁵ is H, D¹ is —C(O)NR⁹₂, R⁹₂ is H and a nitrogen-linked cyclopropylmethyl group; F is —CH₂—; G¹ is phenyl; and there is no R¹⁵. This compound of Formula II has the structure:

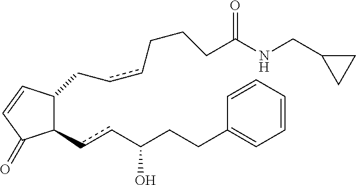

wherein a hashed line represents a substituent below the plane of this paper, a bold wedge represents a substituent above the plane of this paper, and a dashed line represents a single bond or a double bond which can be in the cis or trans configuration. A compound where the dashed lines are double bonds with the stereoconfigurations as drawn is 17-phenyl-18,19,20-trinor PGJ₂ N-cyclopropylmethylamide. A compound wherein the C₅-C₆ dashed line is a single bond and the C₁₃-C₁₄ dashed line is a double bond with the stereoconfigurations as drawn is 17-phenyl-18,19,20-trinor PGJ₁ N-cyclopropylmethylamide.

In some embodiments, R⁵ is H, D¹ is —C(O)NR⁹₂, R⁹₂ is H and a nitrogen-linked —CH(CH₂OH)₂ group; F is —CH₂—; G¹ is phenyl; and there is no R¹⁵. This compound of Formula II has the structure:

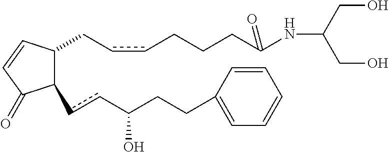

wherein a hashed line represents a substituent below the plane of this paper, a bold wedge represents a substituent above the plane of this paper, and a dashed line represents a single bond or a double bond which can be in the cis or trans configuration. A compound wherein the dashed lines are double bonds with the stereoconfigurations as drawn is 17-phenyl-18,19,20-trinor PGJ₂ (N-(1,3-dihydroxypropan-2-yl)) amide. A compound wherein the C₅-C₆ dashed line is a single bond and the C₁₃-C₁₄ dashed line is a double bond with the stereoconfigurations as drawn is 17-phenyl-18,19,20-trinor PGJ₁ (N-(1,3-dihydroxypropan-2-yl)) amide.

According to another embodiment, the at least one chemotherapeutic agent is an alkylating agent for which alopecia is a side-effect. According to another embodiment, the at least one chemotherapeutic agent is temozolomide. According to another embodiment, the at least one chemotherapeutic agent is busuflan. According to another embodiment, the at least one chemotherapeutic agent is melphalan hydrochloride. According to another embodiment, the at least one chemotherapeutic agent is carmustine. According to another embodiment, the at least one chemotherapeutic agent is lomustine. According to another embodiment, the at least one chemotherapeutic agent is mesna. According to another embodiment, the at least one chemotherapeutic agent is ifosfamide. According to another embodiment, the at least one chemotherapeutic agent is mesnex. According to another embodiment, the at least one chemotherapeutic agent is dacarbazine.

According to another embodiment, the at least one chemotherapeutic agent is an antimetabolite for which alopecia is a side-effect. According to another embodiment, the at least one chemotherapeutic agent is 5-fluorouracil. According to another embodiment, the at least one chemotherapeutic agent is capecitabine. According to another embodiment, the at least one chemotherapeutic agent is mercaptopurine. According to another embodiment, the at least one chemotherapeutic agent is gemcitabine hydrochloride. According to another embodiment, the at least one chemotherapeutic agent is pemetrexed disodium. According to another embodiment, the at least one chemotherapeutic agent is floxuridine. According to another embodiment, the at least one chemotherapeutic agent is decitabine. According to another embodiment, the at least one chemotherapeutic agent is anagrelide hydrochloride. According to another embodiment, the at least one chemotherapeutic agent is vorinostat. According to another embodiment, the at least one chemotherapeutic agent is octreotide acetate.

According to another embodiment, the at least one chemotherapeutic agent is a natural product for which alopecia is a side-effect, wherein the natural product is a vinca alkaloid, a taxane, an epothilone, an anthracycline, an antibiotic, a camptothecin, or an epididophylotoxin. According to another embodiment, the at least one chemotherapeutic agent is bexarotene. According to another embodiment, the at least one chemotherapeutic agent is isotretinoin. According to another embodiment, the at least one chemotherapeutic agent is irinotecan hydrochloride. According to another embodiment, the at least one chemotherapeutic agent is topotecan hydrochloride. According to another embodiment, the at least one chemotherapeutic agent is teniposide. According to another embodiment, the at least one chemotherapeutic agent is etoposide. According to another embodiment, the at least one chemotherapeutic agent is etoposide phosphate. According to another embodiment, the at least one chemotherapeutic agent is mitoxanthrone. According to another embodiment, the at least one chemotherapeutic agent is mitomycin. According to another embodiment, the at least one chemotherapeutic agent is dactinomycin. According to another embodiment, the at least one chemotherapeutic agent is daunorubicin hydrochloride. According to another embodiment, the at least one chemotherapeutic agent is epirubicin hydrochloride. According to another embodiment, the at least one chemotherapeutic agent is doxorubicin hydrochloride. According to another embodiment, the at least one chemotherapeutic agent is idarubicin hydrochloride. According to another embodiment, the at least one chemotherapeutic agent is dexrazoxane. According to another embodiment, the at least one chemotherapeutic agent is paclitaxel. According to another embodiment, the at least one chemotherapeutic agent is docetaxel. According to another embodiment, the at least one chemotherapeutic agent is ixabepilone. According to another embodiment, the at least one chemotherapeutic agent is vincristine sulfate. According to another embodiment, the at least one chemotherapeutic agent is vinblastine sulfate. According to another embodiment, the at least one chemotherapeutic agent is vinorelbine tartrate.

According to another embodiment, the at least one chemotherapeutic agent is a platinum coordination complex for which alopecia is a side-effect. According to another embodiment, the at least one chemotherapeutic agent is oxaliplatin.

According to another embodiment, the at least one chemotherapeutic agent is a hormone or related agent for which alopecia is a side-effect. According to another embodiment, the at least one chemotherapeutic agent is erlotinib. According to another embodiment, the at least one chemotherapeutic agent is sorafenib. According to another embodiment, the at least one chemotherapeutic agent is filgrastim. According to another embodiment, the at least one chemotherapeutic agent is pegfilgrastim. According to another embodiment, the at least one chemotherapeutic agent is bevacizumab. According to another embodiment, the at least one chemotherapeutic agent is sargramostim. According to another embodiment, the at least one chemotherapeutic agent is panitumumab. According to another embodiment, the at least one chemotherapeutic agent is megestrol acetate. According to another embodiment, the at least one chemotherapeutic agent is fluoxymesterone. According to another embodiment, the at least one chemotherapeutic agent is leuprolide acetate.

According to another embodiment, the at least one chemotherapeutic agent is a component of a chemotherapy regimen for treating a cancer.

According to some such embodiments, the cancer is breast cancer.

According to some such embodiments, the chemotherapy regimen comprises doxorubicin and cyclophosphamide. This regimen may be referred to as the “AC” chemotherapy regimen.

According to some such embodiments, the chemotherapy regimen comprises doxorubicin, cyclophosphamide, and paclitaxel. This regimen may be referred to as the “AC+Taxol” chemotherapy regimen.

According to some such embodiments, the chemotherapy regimen comprises doxorubicin, cyclophosphamide, and docetaxel. This regimen may be referred to as the “AC+Taxotere” chemotherapy regimen.

According to some such embodiments, the chemotherapy regimen comprises doxorubicin and paclitaxel (or docetaxel). This regimen may be referred to as the “AT” chemotherapy regimen.

According to some such embodiments, the chemotherapy regimen comprises cyclophosphamide, doxorubicin, and fluorouracil. This regimen may be referred to as the “CAF” chemotherapy regimen.

According to some such embodiments, the chemotherapy regimen comprises cyclophosphamide, methotrexate and fluorouracil. This regimen may be referred to as the “CMF” chemotherapy regimen.

According to some such embodiments, the chemotherapy regimen comprises epirubicin and cyclophosphamide. This regimen may be referred to as the “EC” chemotherapy regimen.

According to some such embodiments, the chemotherapy regimen comprises fluorouracil, epirubicin and cyclophosphamide. This regimen may be referred to as the “FEC” chemotherapy regimen.

According to another embodiment, the cancer is colon cancer.

According to some such embodiments, the chemotherapy regimen is a combination of infusional 5-fluoruracil, leucovorin and oxaliplatin (FOLFOX®). This regimen combines three different antineoplastic agents: (1) 5-fluorouracil; (2) leucovorin; and oxaliplatin. According to some such embodiments, the chemotherapeutic regimen for colorectal cancer utilizes FOLFOX® with bevacizumab (Avastin®, a monoclonal antibody and anti-angiogenesis drug). According to some such embodiments, the chemotherapy regimen utilizes infusional 5-fluoruracil, leucovorin, and irinotecan (FOLFIRI®) with bevacizumab.

According to another embodiment, the epithelial-related surface is an eyebrow. According to another embodiment, the epithelial surface onto which the composition is applied is a scalp. According to another embodiment, the epithelial surface onto which the composition is applied is an eyelid. According to another embodiment, the epithelial surface onto which the composition is applied is of a face. According to another embodiment, the epithelial surface onto which the composition is applied is above a lip. According to another embodiment, the epithelial-related surface onto which the composition is applied is a chin.

According to another embodiment, the composition is formulated as a mascara. According to some such embodiments, the composition is formulated as an ophthalmic composition to restore pigmentation to depigmented hair.

According to some embodiments, the optional second component is an additional active agent. According to some such embodiments, the additional active agent is a therapeutic agent. According to some such embodiments, the therapeutic agent is an anti-fungal agent. According to some such embodiments, the therapeutic agent is an antihistamine. According to some such embodiments, the therapeutic agent is an anti-protozoal agent. According to some such embodiments, the therapeutic agent is an antimetabolite. According to some such embodiments, the therapeutic agent is an antipruritic agent. According to some such embodiments, the therapeutic agent is an anti-oxidant. According to some such embodiments, the therapeutic agent is an anti-viral agent. According to some such embodiments, the therapeutic agent is a chelating agent. According to some such embodiments, the therapeutic agent is a chemotherapeutic agent. According to some such embodiments, the therapeutic agent is a new excipient. According to some such embodiments, the therapeutic agent is a hormone. According to some such embodiments, the therapeutic agent is a non-steroidal anti-inflammatory agent. According to some such embodiments, the therapeutic agent is a peptide. According to some such embodiments, the therapeutic agent is a polypeptide. According to some such embodiments, the therapeutic agent is a prodrug. According to some such embodiments, the therapeutic agent is a peptidomimetic. According to some such embodiments, the therapeutic agent is a steroidal anti-inflammatory agent. According to some such embodiments, the therapeutic agent is a vitamin.

According to some embodiments, the optional second component of the topical composition is an imidazole analog according to Formula III, or a hydrate, solvate, salt, zwitterion, N-oxide, tautomer, prodrug or metabolite thereof:

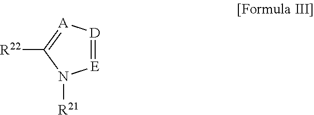

wherein A is N; NR²⁰; NR⁶³ or CH;

wherein D is N or CR²³;

wherein E is N; NR⁶³ or CR²⁴;

with the proviso that, if A is CH, then at least one among D and E is a nitrogen-containing moiety;

with the further proviso that A and E are not simultaneously both NR⁶³;

wherein R²³ is H; F, Cl, Br; I; —NO₂; —N(R⁴²)₂; straight chain or branched C₁˜C₆ alkyl, alkenyl or alkoxy; or phenyl;

wherein R²⁴ is H; F, Cl, Br; I; —NO₂; —N(R⁴₂)₂; straight chain or branched C₁˜C₆ alkyl, alkenyl or alkoxy; or phenyl;

or wherein R²³ and R²⁴ together are CR⁶²═CR⁶²—CR⁶²═CR⁶²—;

wherein R²⁰ is selected from the following: H; straight chain or branched C₁˜C₁₂ alkyl;

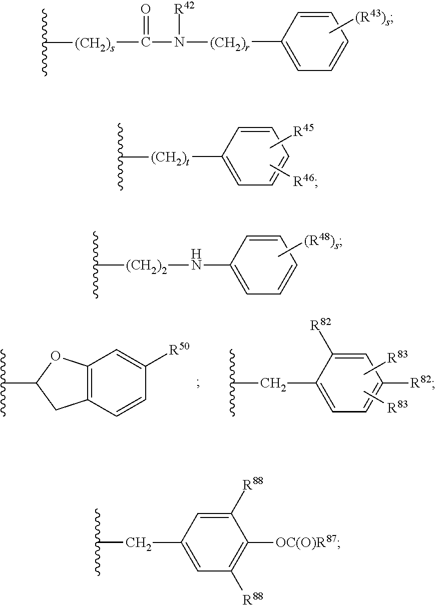

—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula III is an imidazolium or triazolium salt with a pharmaceutically acceptable counter anion or an internal salt (zwitterion); or

wherein R⁶³ is a moiety that is readily cleaved in vivo, exemplified by, but not limited to, —CHR⁴²OC(═O)(O)—R⁵⁵ or —C(R⁴²)₂OP(═O)(OR¹⁰⁶)₂ and the compound of Formula III is a prodrug and an imidazolium or triazolium salt with a pharmaceutically acceptable counter ion or an internal salt (zwitterion);

wherein s is 1 or 2;

wherein t is an integer from 1 to 4 inclusive;

wherein u is 2 or 3;

wherein R²² is H; F, Cl, Br; I; —NO₂; —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical preferably containing one S and one or two N atoms, said heterocyclic radical being preferably thiazolyl and more preferably thiazol-4-yl;

wherein R²¹ is H; or —(CR²⁵R²⁶)ₘ—CR²⁷R²⁸-Q-R²⁹;

wherein each m is independently an integer from 0 to 4 inclusive;

wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;

wherein R²⁶ is H; or straight chain or branched C₁˜C₆ alkyl;

wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(═NH)N(R⁶⁸)OR⁶⁸; —CF(R⁶⁷)C(═NH)N(R⁶⁸)OH; or —CF(R⁶⁷)C(═N—OR⁶⁹)NH₂;

wherein each r is independently an integer from 0 to 2 inclusive;

wherein each L is independently α-tetralyl; or phenyl, said phenyl being optionally substituted with up to three groups selected from the following: straight chain or branched C₁˜C₆ alkyl, straight chain or branched C₁˜C₆ alkoxy, —C≡N, —NO₂, or —NH₂;

wherein R²⁸ is H; F; Cl; Br; —OR⁴²; —OC(═O)R¹⁰⁰;

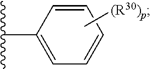

straight chain or branched C₁˜C₆ alkyl; R⁶¹; or —OP(═O)(OH)₂ or a pharmaceutically acceptable salt or zwitterion form thereof;

wherein each Q is independently a covalent bond; —O—; —S—; —S(O)—; or —S(O)₂—;

wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; straight chain or branched C₁˜C₆ alkyl optionally substituted with —CO₂H; or

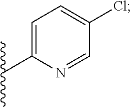

wherein each p is independently an integer from 0 to 3 inclusive;

wherein each T is independently a covalent bond; —CH₂CH₂—; —CH═CH—; —(O)—C(R⁴²)₂—; —CH₂O—; —SCH₂—; —CH₂S—; —OCH₂CH₂O—; —CH(CH₃)—; —CH₂—; —CF₂—; —C(R⁹³)₂—; or

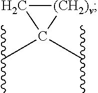

wherein each v is independently an integer from 1 to 3 inclusive;

wherein each U is independently a covalent bond;

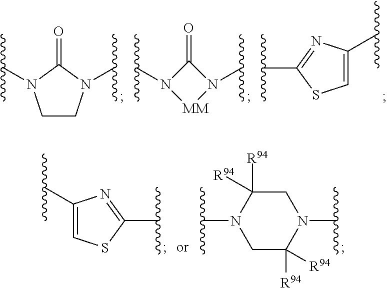

wherein each V is independently H; —S(O)₂CH₃; —C(O)NH₂; —CH₂C≡CH; —CH═CH₂; —S(O)ᵣ—R⁵⁵;

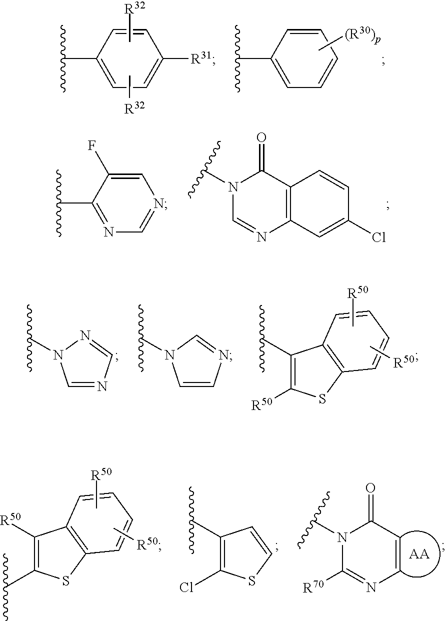

### —R⁵⁹; —R⁶¹; or -LL-R⁹⁵;

wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings;

wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴: —OC(O)N(R⁵⁴)₂; —SH; —SR⁴¹; —S(O)ᵣR⁵⁸; —CH═CH—CO₂H;

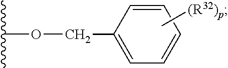

straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to —CH₂C≡N, —CH₂CH₂C≡N, —CH₂CH₂CH₂C≡N, and —CH₂CH(CH₃)C≡N; straight chain or branched C₁˜C₆ halogenoalkyl exemplified by, but not limited to —CF₃, —CF₂CF₃, —CH₂CF₃, —CHF₂, —CH₂F, —CH₂Cl, and —CH₂Br; straight chain or branched C₁˜C₅ halogenoalkoxy exemplified by, but not limited to —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₈ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy;

wherein each R³¹ is independently H; F; Cl; Br; I; —C≡N; NO₂; —CF₃; —N═C═S; —N(R⁴²)₂; —NH—C(═O)-(M)ₙ-R⁵⁴; —NH—C(═S)—NH—R⁵⁴; straight chain or branched C₁˜C₆ alkoxy or alkylthio: straight chain or branched C₁˜C₁₂ alkyl: —(CH₂)ᵣ—R⁹⁹;

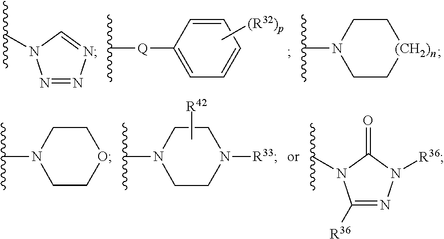

wherein each M is independently O or NH, with the proviso that when M is O and n is 1, R⁵⁴ is other than H;

wherein each R³² is independently H; F; Cl; Br; I; —C≡N; —NO₂; straight chain or branched C₁˜C₁₂ alkyl; straight chain or branched C₁˜C₄ alkoxy; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; straight chain or branched C₁˜C₃ halogenoalkoxy exemplified by, but not limited to —OCF₃, —OCF₂CF₃, —OCH₂CF₃, —OCHF₂, —OCH₂F; straight chain or branched C₁˜C₄ alkylthio or haloalkylthio; straight chain or branched C₁˜C₄ alkylthionyl or haloalkylthionyl; or straight chain or branched C₁˜C₄ alkylsulfonyl or haloalkylsulfonyl;

wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)—C(O)OR³⁴; —C(O)O-phenyl; —(CH₂)—C(O)N(R⁴²)₂; —CH₂C(S)N(R⁴²)₂; —CH₂C(S)SR³⁴; —(CH₂)ₙ-phenyl; benzoyl, wherein said benzoyl is optionally substituted with one or two R⁴⁶ groups; or

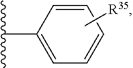

or, preferably,

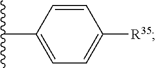

wherein each R³⁴ is independently straight chain or branched C₁˜C₆ alkyl; —

wherein each R³⁵ is independently H;

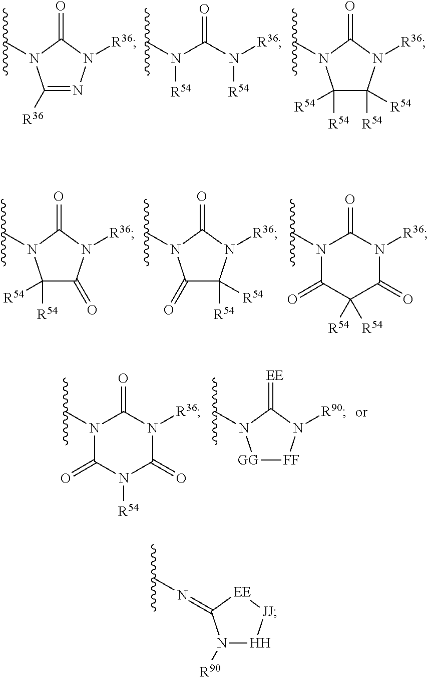

wherein each R³⁶ is independently H; straight chain or branched C₃˜C₇ alkenyl, with the proviso that the olefinic double bond is not located α to N; straight chain or branched C₃˜C₇ alkynyl, with the proviso that the alkynyl CC bond is not located α to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or

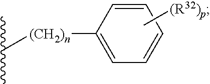

wherein each n is independently 0 or 1;

wherein R²⁶ and R²⁸ together with the two carbon atoms to which they attach can be C═C;

wherein R²⁷ and R²⁸ together can be

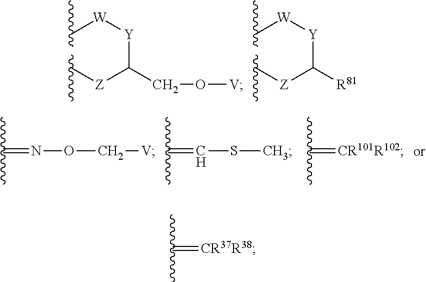

wherein W is —CH₂— and Y is —CH₂—; W is —O— and Y is —CH₂—; or W is —CH₂— and Y is —O—;

wherein Z is —CH₂— or —O—;

wherein R²⁷ together with -Q-R²⁹ can be

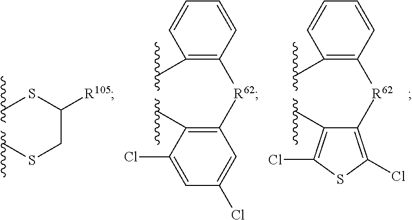

or, when R²⁷ and R²⁹ each independently represents a cycloalkyl, aryl or heterocyclic ring and Q is a covalent bond, R²⁷ together with -Q-R²⁹ can be

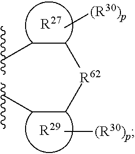

wherein each R³⁷ is independently H; C₁˜C₂ alkyl or phenyl;

wherein each R³⁸ is independently H or C₁˜C₂ alkyl;

wherein each R³⁹ is independently H; straight chain or branched C₁˜C₅ alkyl; straight chain or branched C₁˜C₅ alkanoyl; or straight chain or branched C₁˜C₅ haloalkanoyl exemplified by, but not limited to —C(O)CF₃, —C(O)CF₂CF₃, —C(O)CH₂CF₃, —C(O)CHF₂, and —C(O)CH₂F;

wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;

wherein each R⁴¹ is independently straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;

wherein each q is independently an integer from 0 to 4 inclusive;

wherein each G is independently a covalent bond; —O—; or —S—;

wherein each J is independently phenyl, 2-thienyl or 3-thienyl wherein said phenyl, 2-thienyl or 3-thienyl is optionally substituted with one to three groups selected from the following: F; Cl; Br; I; —NO₂; straight chain or branched C₁˜C₅ alkyl; straight chain or branched C₁˜C₅ alkoxy;

wherein each R⁴² is independently H; or straight chain or branched C₁˜C₆ alkyl;

wherein each R⁴³ is independently H; F; Cl; Br; I; —NO₂; straight chain or branched C₁˜C₆ alkyl; straight chain or branched C₁˜C₆ alkoxy; —CF₃; or —O-phenyl;

wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the following: F, Cl, Br, I, straight chain or branched C₁˜C₆ alkyl, or straight chain or branched C₁˜C₆ alkoxy;

wherein each R⁴⁵ is independently H; F; Cl; Br; I; —NO₂; straight chain or branched C₁˜C₆ alkyl; straight chain or branched C₁˜C₆ alkoxy; or —S(O)₂NH₂;

wherein each R⁴⁶ is independently H; F; Cl; Br; I; straight chain or branched C₁˜C₆ alkyl; straight chain or branched C₁˜C₆ alkoxy; or —C≡N;

wherein each R⁴⁷ is independently a phenyl group optionally substituted with one to three F, Cl, Br or I;

wherein each R⁴⁸ is independently H; F; Cl; Br; I; or straight chain or branched C₁˜C₆ alkyl;

wherein each R⁴⁹ is independently 1-naphthyl; 2-naphthyl; or phenyl, wherein said phenyl is optionally substituted with one or two groups, each independently selected from the following: F, Cl, Br, I, straight chain or branched C₁˜C₆ alkyl, straight chain or branched C₁˜C₆ alkoxy, or —CH₂CH═CH₂;

wherein each R⁵⁰ is independently H; F; Cl; Br; or I;

wherein each R⁵¹ is independently phenyl, wherein said phenyl is optionally substituted with one or two groups, each independently selected from the following: F, Cl, Br, I, or straight chain or branched C₁˜C₆ alkyl;

wherein each R⁵² is independently phenyl, wherein said phenyl is optionally substituted with one or two groups, each independently selected from the following: F, Cl, Br, or I;

wherein each R⁵³ is independently straight chain or branched C₁˜C₈ alkyl optionally substituted with C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; straight chain or branched C₃˜C₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl CC bond is located β, γ or δ with respect to the ether oxygen;

wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl;

wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle can be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups;

wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle can be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle can be aromatic or non-aromatic; methylenedioxy; —C≡N; —NO₂; —CF₃; —N(R⁵⁴)₂; —OR⁵⁴; —SH; ═O ; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; —C(═NH)N(R⁵⁴)OH; —C(═N—OR⁵⁴)NH₂; —S(O)₂N(R⁵⁴)₂; —NR⁵⁴S(O)₂R⁵⁴; —C(O)NHS(O)₂R⁵⁴; —S(O)₂R⁵⁴; —SO₃H; or —PO₃H₂;

wherein each R⁵⁷ is independently F; Cl; Br; I; straight chain or branched C₁˜C₆ alkyl; straight chain or branched C₁˜C₆ alkoxy; —C≡N; —CF₃; NO₂; —NH₂; or —NHC(O)R⁶⁶, wherein said R⁶⁶ is straight chain or branched C₂˜C₁₂ alkyl;

wherein each R⁵⁸ is independently straight chain or branched C₁˜C₂₀ alkyl; C₃˜C₈ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups;

wherein each R⁵⁹ is independently straight chain or branched C₁˜C₇ alkyl, alkenyl or alkynyl, said alkyl, alkenyl or alkynyl being substituted with one or more —R⁶⁰—R⁶¹;

wherein each R⁶⁰ is independently a covalent bond or —O—;

wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups;

wherein each R⁶² is independently a covalent bond; —CH₂—CH₂—; —CH═CH—; —O—; or —S—;

wherein each R⁶⁵ is independently straight chain or branched C₁˜C₁₂ acyclic hydrocarbon group, which can be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or CC bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;

wherein each R⁶⁷ is independently H; F; or straight chain or branched C₁˜C₆ alkyl, said alkyl group being optionally substituted by one or more R³⁰;

wherein each R⁶⁸ is independently R³⁶, R⁵⁴ or R⁶¹;

wherein each R⁶⁹ is independently H; or straight chain or branched C₁˜C₆ alkyl, said alkyl group being optionally substituted by one or more R³⁰;

wherein each R⁷⁰ is independently H; straight chain or branched C₁˜C₄ alkyl; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; or cyclopropyl;

wherein each R⁷¹ is independently R⁵⁶; straight chain or branched C₁˜C₄ halogenoalkyl exemplified by, but not limited to —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶;

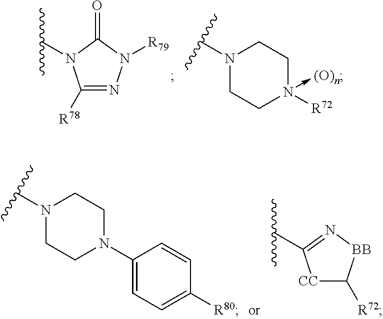

wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;

wherein each R⁷³ is independently H; straight chain or branched C₁˜C₄ alkyl;

C₃˜C₆ cycloalkyl; —C(O)R⁷²; or —C(O)CF₃;

wherein each R⁷⁴ is independently straight chain or branched C₁˜C₄ alkyl;

wherein each R⁷⁵ is independently H; —C(O)NH₂; —C(O)CH₃; —C≡N; —SO₂NHR⁷²; —SO₂R⁷²; —OR⁷²; —OC(O)R⁷²; or straight chain or branched —(C₁˜C₄ alkyl)-NH₂;

wherein each R⁷⁶ is independently phenyl optionally substituted with one or more R⁷⁷ groups;

wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²;

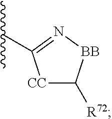

or phenyl, said phenyl being optionally substituted with one or more F, Cl, Br, I, —C≡N, —NO₂, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;

wherein each R⁷⁸ is independently H or —CH₃;

wherein each R⁷⁹ is independently H; isopropyl; cyclopentyl; cyclopropyl; 2-butyl; 3-pentyl; 3-hydroxy-2-butyl; or 2-hydroxy-3-pentyl;

wherein each R⁸⁰ is independently F; Cl; Br; I; —CN; —NO₂; —NH₂; straight chain or branched C₁˜C₄ halogenoalkyl; straight chain or branched C₁˜C₄ halogenoalkoxy; or

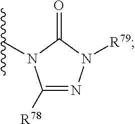

wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —C≡N, —NO₂, phenyl, phenylmethyl, benzoyl, halobenzoyl, —C(O)R⁴², —C(O)OR⁴², and —CF₃, with the proviso that when multiple substituents are present only 1 thereof can be selected from the group consisting of phenyl, phenylmethyl, benzoyl and halobenzoyl;

wherein each R⁸² is independently H or —OR⁸⁴;

wherein each R⁸³ is independently H; F; Cl; Br; R⁷⁴; —OR⁷⁴; —SR⁷⁴; —CH₂SC(═O)R⁷⁴; —COOH; or —C(═O)OCH₃;

wherein each R⁸⁴ is independently a group that is easily hydrolyzable under physiological conditions, preferably the acyl residue of an amino acid or a group represented by the formula —C(═O)R⁸⁵ or —P(═O)(OR⁸⁶)₂; wherein R⁸⁴ is exemplified by, but not limited to, formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like;

wherein each R⁸⁵ is independently H; —OR⁷⁴; or straight chain or branched C₁˜C₆ alkyl or alkenyl, wherein said alkyl or alkenyl can be optionally interrupted by a hydrolyzable functional group such as carboxamide or ester, wherein said alkyl or alkenyl can be optionally substituted with —COOH, —NH₂, —NHR⁷⁴, —N(R⁷⁴)₂, or R⁶¹, wherein R⁶¹ is exemplified by, but not limited to, phenyl, methoxyphenyl, pyridyl, pyrazinyl or furyl;

wherein each R⁸⁶ is independently H or R⁷⁴;

wherein each R⁸⁷ is independently straight chain or branched C₁˜C₅ alkyl; R⁶¹; pyridyl; pyrrolidinyl; or -DD—NH—R⁸⁹;

wherein each R⁸⁸ is independently H; F; Cl; Br; or —OR⁷⁴;

wherein each DD is independently straight chain or branched C₁˜C₄ alkylene; —CH₂NHC(═O)CH₂—; —CH(NH₂)CH₂CH₂CH₂—;

wherein each R⁸⁹ is independently H or straight chain or branched C₁˜C₅ alkyl;

wherein each BB is independently —CH₂— or —C(═O)—;

wherein each CC is independently NH or O;

wherein each EE is independently O, S or N—R⁹¹;

wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃˜C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched;

wherein each KK is independently O or S;

wherein each R⁹¹ is independently H or straight chain or branched C₁˜C₆ alkyl;

wherein each R⁹² is independently H; straight chain or branched C₁˜C₆ alkyl; C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—;

wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃;

wherein each GG is independently —C(═O)—; or —CH₂—, optionally substituted with up to two radicals selected from the group consisting of straight chain or branched C₁˜C₆ alkyl and straight chain or branched C₁˜C₆ alkoxy;

or FF and GG, taken together, form a bivalent radical of formula —N═CH—(FFGG′);

wherein HH has the same meaning as FF;

wherein JJ has the same meaning as GG;

or HH and JJ, taken together, form a bivalent radical of formula —N═CH—(FFGG′) or —CH═CH—(HHJJ′);

wherein the nitrogen atom in the bivalent radical FFGG′ is connected to NR⁹⁰; wherein one hydrogen in said radical FFGG′ and up to two hydrogens in radical HHJJ′ can be replaced by a straight chain or branched C₁˜C₆ alkyl radical;

wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group exemplified by, but not limited to —CH₂CH₂—, —CH₂CH₂CH₂—, —CH₂CH₂CH₂CH₂— and the like;

wherein each R⁹⁴ is independently H or C₁˜C₄ alkyl which is unsubstituted or substituted by 1-3 substituents each independently selected from the group consisting of halogen, hydroxyl, C₁˜C₄ alkoxy, and amino; or two R⁹⁴ groups attached to the same carbon atom together form ═S;

wherein each LL is independently a covalent bond; —C(═O)—; —C(═S)—; —SO₂—; or —N═N—;

wherein each R⁹⁵ is independently selected from the group consisting of

i) hydrogen,

ii) CN

iii) CHO

iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl,

v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido,

vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl,

vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and

viii) a group of the formula

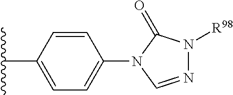

wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which can be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl;

wherein each MM is independently an ethylene group optionally substituted with R⁹¹; or MM is —NN═OO—, wherein NN and OO are the same or different and are each independently N or CR⁹¹;

wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶;

wherein each R¹⁰⁰ is independently —R⁵⁴ or —OR⁵⁴;

wherein each R¹⁰¹ is independently H; F; Cl; Br; or I;

wherein each R¹⁰² is independently F; Cl; Br; I; —SR¹⁰³; or —OR¹⁰⁴;

wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which can be the same or different;

wherein each R¹⁰⁴ is independently straight chain or branched C₁˜C₁₂ alkyl; or phenyl;

wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰;

wherein each R¹⁰⁶ is independently H, a pharmaceutically acceptable cation, or null (affording an azolium phosphate zwitterion);

wherein each chiral center independently may possess any relative or absolute stereoconfiguration or be any mixture thereof, unless specified otherwise herein; and

wherein each olefinic C═C bond that is capable of E/Z isomerism independently may possess either the E or Z stereoconfiguration or be any mixture thereof, unless specified otherwise herein.

In some embodiments, the at least one compound of Formula III is diastereomerically pure.

In some embodiments, the at least one compound of Formula III is a mixture of diastereomers in any ratio.

In some embodiments, the at least one compound of Formula III is enantiomerically pure.

In some embodiments, the at least one compound of Formula III is a mixture of enantiomers in any ratio, including a racemate.

In some embodiments, the at least one compound of Formula III is diastereomerically and enantiomerically pure.

In some embodiments, the at least one compound of Formula III is a mixture of diastereomers and enantiomers in any ratio.

In some embodiments, the at least one compound of Formula III has one or more hydrogen atoms replaced by deuterium.

In some embodiments, the at least one compound of Formula III is a pharmaceutically acceptable salt derived from the combination of a compound of Formula III with an acid or a base.

In some embodiments, the at least one compound of Formula III is an N-oxide form of an amine.

In some embodiments, the at least one compound of Formula III is a zwitterion.

In some embodiments, the at least one compound of Formula III is a tautomer of a structure described herein.

Examples of compounds of Formula III include, but are not limited to, the antifungal agents bifonazole, butoconazole, croconazole, econazole, enilconazole, clotrimazole, flutrimazole, isoconazole, fenticonazole, ketoconazole, sulconazole, tioconazole, oxiconazole, sertaconazole, miconazole, chlormidazole, omoconazole, posaconazole, elubiol, voriconazole, eberconazole, CAS RN 214543-30-3, democonazole, genaconazole, vibunazole, lombazole, pramiconazole, T 8581, fluconazole, albaconazole, tiabendazole, parconazole, ravuconazole, saperconazole, itraconazole, hydroxyitraconazole, TAK 456, terconazole, SYN 2869, neticonazole, NND-502, lanoconazole, fosfluconazole; and the thromboxane synthase inhibitors 7-(1-imidazolyl)heptanoic acid, ozagrel, and 1-benzyl imidazole.

Other compounds of interest not contained within Formula III include the antifungal chlordantoin; non-azole thromboxane synthase inhibitors, such as terbinafine, furegrelate, BM 567, and analogs of terbinafine, such as amorolfine, butenafine, naftifine; and Epoxygenase inhibitors, such as MS-PPOH, and PPOH.

According to some embodiments, an imidazole analog of the described invention is a compound of Formula IV, or a hydrate, solvate, salt, zwitterion, N-oxide, tautomer, prodrug or metabolite of Formula IV having the structure:

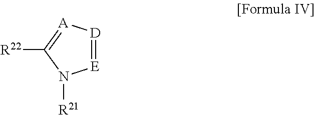

wherein each A is independently N or NR²⁰;

wherein each D is independently CR²³;

wherein each E is independently N or CR²⁴;

wherein R²³ is H;

wherein R²⁴ is H;

or wherein R²³ and R²⁴ together are —CH═CH—CH═CH—;

wherein R²⁰ is H and the compound of Formula IV is an imidazolium or triazolium salt with a pharmaceutically acceptable counter anion; or

wherein R²⁰ is a moiety that is readily cleaved in vivo, exemplified by, but not limited to, —CH₂OC(O)CH₃, and the compound of Formula IV is a prodrug and an imidazolium or triazolium salt with a pharmaceutically acceptable counter anion;

wherein R²² is H; —CH₃; or

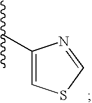

wherein R²¹ is H; —(CR²⁵R²⁶)m-CR²⁷R²⁸-Q-R²⁹;

wherein each m is independently 0 or 1;

wherein R²⁵ is H; —CH₃; or —C≡N;

wherein R²⁶ is H;

wherein R²⁷ is -T-U—V;

wherein R²⁸ is H; —OH; or

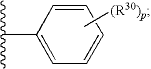

wherein each Q is independently a covalent bond or —S—;

wherein R²⁹ is

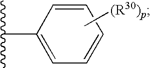

or straight chain or branched C₃˜C₁₀ alkyl optionally substituted with —CO₂H;

wherein each p is independently an integer from 0 to 3 inclusive;

wherein each T is independently a covalent bond; —CH₂CH₂—; —OCH₂—; —CH₂O—; —SCH₂—; —CH₂S—; —OCH₂CH₂O—; —CH(CH₃)—; —CH₂—; or —CF₂—;

wherein each U is independently a covalent bond;

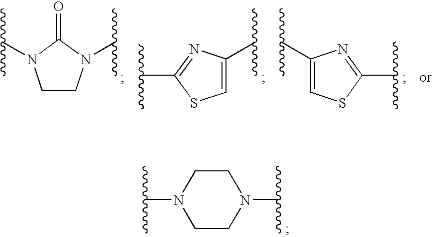

wherein each V is independently H; —S(O)₂CH₃; —C(O)NH₂; —CH₂C≡CH; —CH═CH₂;

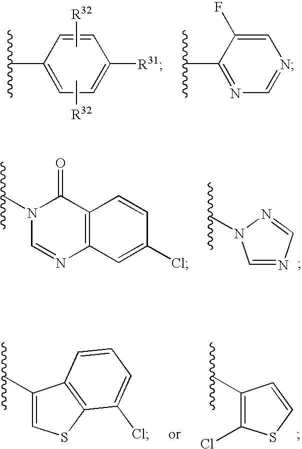

wherein each R³⁰ is independently F, Cl, —CH═CH—CO₂H;

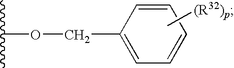

or straight chain or branched C₁˜C₁₀ alkoxy;

wherein each R³¹ is independently H; F; Cl; Br; I; —C≡N; N;

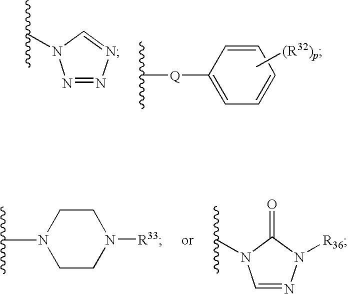

wherein each R³² is independently H; F; Cl; or —OCF₃;

wherein each R³³ is independently straight chain or branched C₁˜C₆ alkyl; —C(O)R³⁴; —C(O)OR³⁴;

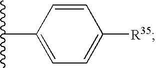

wherein each R³⁴ is independently straight chain or branched C₁˜C₆ alkyl;

wherein each R³⁵ is independently H;

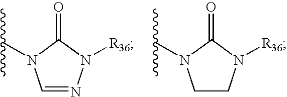

wherein each R³⁶ is independently straight chain or branched C₁˜C₆ alkyl optionally substituted with —OH; or

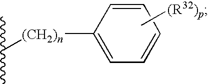

wherein each n is independently 0 or 1;

wherein R²⁶ and R²⁸ together with the two carbon atoms to which they attach can be C═C;

wherein R²⁷ and R²⁸ together can be

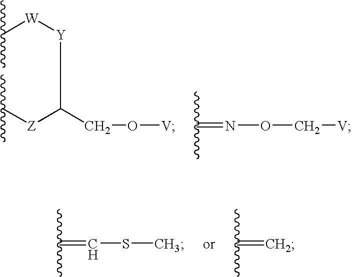

wherein W is —CH₂— and Y is —CH₂—; W is —O— and Y is —CH₂—; or W is —CH₂— and Y is —O—;

wherein Z is —CH₂— or —O—;

wherein R²⁷ together with -Q-R²⁹ can be

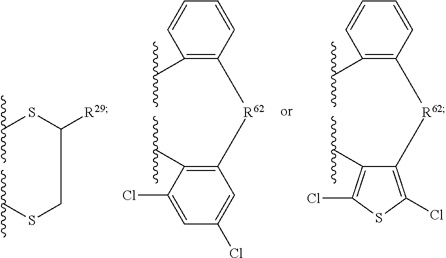

wherein each R⁶² is independently —CH₂—CH₂— or —CH═CH—.

In some embodiments, each chiral center independently possesses any relative or absolute stereoconfiguration or is any mixture thereof, unless specified otherwise herein.

In some embodiments, each olefinic C═C bond that is capable of E/Z isomerism independently can possess either the E or Z stereoconfiguration or be any mixture thereof, unless specified otherwise herein.

In some embodiments, the at least one compound of Formula IV is diastereomerically pure.

In some embodiments, the at least one compound of Formula IV is a mixture of diastereomers in any ratio.

In some embodiments, the at least one compound of Formula IV is enantiomerically pure.

In some embodiments, the at least one compound of Formula IV is a mixture of enantiomers in any ratio, including a racemate.

In some embodiments, the at least one compound of Formula IV is diastereomerically and enantiomerically pure.

In some embodiments, the at least one compound of Formula IV is a mixture of diastereomers and enantiomers in any ratio.

In some embodiments, the at least one compound of Formula IV has one or more hydrogen atoms replaced by deuterium.

In some embodiments, the at least one compound of Formula IV is a pharmaceutically acceptable salt derived from the combination of a compound of formula III with an acid or a base.

In some embodiments, the at least one compound of Formula IV is an N-oxide form of an amine described herein.

In some embodiments, the at least one compound of Formula IV is a zwitterion.

In some embodiments, the at least one compound of Formula IV is a tautomer of a structure described herein.

Examples of imidazole analogs according to Formula IV include, but are not limited to, histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole.

According to another embodiment, the compound of Formula IV, is miconazole or a solvate, a hydrate, a salt, a zwitterion, an N-oxide, a tautomer, a prodrug or a metabolite of miconazole.

According to another embodiment, the imidazole analog is a compound having a structure of Formula III or Formula IV, wherein the imidazole analog improves the efficacy of a composition comprising at least one prostaglandin analog of Formula I when delivered to a subject refractory to treatment with a composition containing the prostaglandin analog of Formula I alone.

Because hair loss due to chemotherapy can significantly alter the visual aspect of a patient, and adversely affect the patient's own self-perception of her or his appearance, in some embodiments, the described methods improve the appearance of the subject undergoing treatment with a chemotherapeutic agent.

### Carriers

According to another embodiment, the topical composition further comprises a carrier.

Some non-limiting representative examples of carriers include moisturizing agents or humectants, pH adjusting agents, hair conditioning agents, chelating agents, preservatives, emulsifiers, thickeners, solubilizing agents, penetration enhancers, anti-irritants, colorants and surfactants.

Representative examples of moisturizing or humectant agents that are usable in the described invention include, without limitation, guanidine, glycolic acid and glycolate salts (e.g. ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

As is widely recognized in the art, since the pH of the skin is 5.5, compositions for topical skin application (to avoid irritation) should have a pH value of between pH 4.0 and pH 7.0. In some embodiments, the pH is between pH 5.0 and pH 7.0. In some embodiments, the pH is about pH 5.5. Hence, a pH adjusting composition typically is added to bring the pH of the composition to the desired value. The compositions of the described invention therefore are formulated to have a pH value of about 7.0. Suitable pH adjusting agents include, for example, but are not limited to, one or more adipic acids, glycines, citric acids, calcium hydroxides, magnesium aluminometasilicates, buffers or any combinations thereof

Suitable hair conditioning agents that can be used in the context of the described invention include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof

Hair stimulating agents may be added to the compositions of the described invention. For example Procapil® (FR2 791 684 and WO0058347) promotes the visible appearance of thicker and fuller hair and prevents premature hair thinning and hair loss by boosting the synthesis of components at the epidermal junction where the hair anchors to the skin, which helps to anchor the hair follicles more firmly to the scalp. U.S. Pat. No. 6,861,077, incorporated by reference herein, describes methods to protect keratinous fibers from extrinsic damages comprising application of compositions comprising at least one plant extract. For example, a plant extract composed of purified glycoproteins obtained from white potatoes (Solanum tuberosum L. is commercially available from SEDERMA, Inc. (France) as Dermolectine® and Capilectine®. ANCRIN® (Sederma), a hydroglycolic solution containing octylbutyrate and glutamine peptide, reduces hair loss by supplying a vegetable substrate to transglutaminases, a group of enzymes known to increase protein reticulation in the scalp and help anchor the hair to the scalp. Capisome™ (Sederma) is a liposome that comprises homotaurine (3-aminopropane sulfonic acid), a bacterial filtrate of biotechnological origin from enterobacteria that contains high levels of peptides and the sulfur-containing amino acids methionine and cysteine; and marine sulfopolysaccharides. (See U.S. Pat. No. 6,376,557, incorporated herein by reference). Capigen™ (Sederma), is a complex that comprises homotaurine (3-aminopropane sulfonic acid), a bacterial filtrate obtained from a strain of microorganisms cultured in a medium comprising selected peptides, with the filtrate containing high levels of peptides, and a sulfomuycopolysaccharide of marine origin, which is a complex of sulfated polysaccharides that are soluble in water and are found in the connective tissue and synovial fluids. Follicusan® (Chemlishes Laboratorium Dr. Kurt Richter GmbH), is composed of a fraction derived from milk, ethyl pantenol, inositol and sulfur-containing amino acids (N-acetylcysteine and N-acetyl methionine in an aqueous alcoholic medium. Anageline® (Silab) contains an extract from white sweet lupine. Cprillisil (Exsymol S.A.M. of Monaco) is a 20% solution of dimethylsilanediol salicilate in butylenes glycol with triethanolamine. Mahanimba is an extract of the flowers and inflorescence of the neem tree (Melia azadirachta) and contains carotinoids, amino acids, phytosterols, mucins, polyacetylenes, and ses quiterpenes. Malkagni is an extract of the seeds, leaves and flowers of the intellect tree (Celastrus paniculata) and contains tannins, mineral salts, saponins, and iridic glycosides. Fitopur B is a complex available from Sederma, Inc., and comprises extracts of three plants: buchu (Buc hu barosma), henna (Lawsonia inermis), and venus hair (Adiatium capillus-veneris). The essential oil of buchu contains the terpenic oil diosphenol and sulfur compounds. The leaves of henna contain flavonic pigments, including luteoline and laxanthones, principally lawsone. Venus hair is a small firm native to the south of France; it has diuretic and emollient activity. Peptide-copper complexes containing dipeptides or tripeptides chelated to copper stimulate hair growth. (See U.S. Pat. No. 5,538,945 and U.S. Pat. No. 6,017,888, incorporated herein by reference). Hormone replacement therapy (HRT), including administration of micronized progesterone pills and creams and estrogen pills and creams, is used to treat androgenetic alopecia for women. Other such agents are known by persons of skill in the art.

Chelating agents optionally are added to the compositions of the described invention so as to enhance the preservative or preservative system. In some embodiments, the chelating agents are mild agents, such as, for example, ethylenediaminetetraacetic acid (EDTA), EDTA derivatives, or any combination thereof

Suitable preservatives for use in the compositions of the described composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or botanical agents, such as, but not limited to, leuconostocil or radish root ferment filtrate, or any combinations thereof

In another embodiment, the composition of the described invention, a carrier and other, optional ingredients can be dispersed in an emulsion.

In some embodiments, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

Suitable emulsifiers may be natural materials, finely divided solids, or synthetic materials. Natural emulsifying agents may be derived from either animal or vegetable sources. Those from animal sources include gelatin, egg yolk, casein, wool fat, or cholesterol. Those from vegetable sources include acacia, tragacanth, chondrus, or pectin. Vegetable sources specifically from cellulose derivatives include methyl cellulose and carboxymethyl cellulose to increase the viscosity. Finely divided emulsifiers include bentonite, magnesium hydroxide, aluminum hydroxide, or magnesium trisylicate. Synthetic agents include anionic, cationic or nonionic agents. Particularly useful are sodium lauryl sulfate, benzalkonium chloride or polyethylene glycol 400 monostearate, or any combinations thereof.

Suitable thickeners that can be used in the context of the described invention include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids, anionic polymers, and their alkali salts and mixtures thereof

Representative examples of solubilizing agents that are usable in the context of the described invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN® and spans, e.g., TWEEN 80®, which is polysorbate 80, and SPANS (for example, SPAN® 20) which is sorbitan monolaurate, available from Croda International PLC. Other solubilizers that are usable for the compositions of the described invention are, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

Suitable penetration enhancers usable in the described invention include, but are not limited to, a vegetable oil. Such oils include, for example, safflower oil, cottonseed oil and corn oil.

Additional thickeners, penetration enhancers and other adjuvants generally may be found in Remington's Pharmaceutical Sciences, 18th or 19th editions, published by the Mack Publishing Company of Easton, Pa., which is incorporated herein by reference.

Suitable anti-irritants that can be used in the context of the described invention include, for example, steroidal and non steroidal anti-inflammatory agents or other materials such as aloe vera, chamomile, alpha-bisabolol, cola nitida extract, green tea extract, tea tree oil, licorice extract, allantoin, caffeine or other xanthines, glycyrrhizic acid and its derivatives. Presently known anti-irritants can be divided into water-soluble anti-irritants and water-insoluble anti-irritants. Representative examples of such compositions are described, for example, in U.S. Pat. No. 5,482,710, which is incorporated herein by reference.

Colorants also may be used in the compositions of the invention. Preferred pigments include, but are not limited to, iron oxides, and titanium oxides. Suitable dyes include FD&C approved colorants, D&C approved colorants, and those approved for use in Europe and Japan. See Marmion, D. M., Handbook of US Colorants for Food, Drugs, Cosmetics, and Medical Devices, 3rd ed, 1991 herein incorporated by reference.

Suitable surfactants for use with the inventive compositions include, but are not limited to, sarcosinates, glutamates, sodium alkyl sulfates, ammonium alkyl sulfates, sodium alkyleth sulfates, ammonium alkyleth sulfates, ammonium laureth-n-sulfates, sodium laureth-n-sulfates, isothionates, glycerylether sulfonates, sulfosuccinates and combinations thereof. In some embodiments, the anionic surfactant is selected from the group consisting of sodium lauroyl sarcosinate, monosodium lauroyl glutamate, sodium alkyl sulfates, ammonium alkyl sulfates, sodium alkyleth sulfates, ammonium alkyleth sulfates, and combinations thereof

In some embodiments, a pharmaceutically acceptable carrier is included in the composition.

In another embodiment, the compositions of the described invention include a cosmetically acceptable carrier. It will be understood that cosmetically acceptable carriers and pharmaceutically acceptable carriers are similar, if not often identical, in nature.

Suitable pharmaceutically acceptable carriers include water, petroleum jelly (Vaseline™), petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. Also included are the carriers described hereinabove.

In another embodiment, the pharmaceutically acceptable carrier of the composition of the described invention includes a sustained release or delayed release carrier. The carrier can be any material capable of sustained or delayed release of the compound to provide a more efficient administration resulting in less frequent and/or decreased dosage of the compound, ease of handling, and extended or delayed effects on epithelial-related conditions. Non-limiting examples of such carriers include liposomes, microsponges, microspheres, or microcapsules of natural and synthetic polymers and the like. Liposomes which may enhance the localized delivery of the compounds of the inventive composition within skin layers, may be formed from a variety of phospholipids, such as cholesterol, stearylamines or phosphatidylcholines.

Suitable cosmetically acceptable carriers are described in the CTFA International Cosmetic Ingredient Dictionary and Handbook, 8th edition, edited by Wenninger and Canterbery, (The Cosmetic, Toiletry, and Fragrance Association, Inc., Washington, D.C., 2000), which is herein incorporated by reference. Also included are the carriers described hereinabove.

Mascara

According to some embodiments, the carrier is a mascara.

Mascara comes in three forms: liquid, cake and cream, is available in many formulas, tints and colors, and may be formulated as a water resistant mascara or a non-water resistant mascara.

Generally, the composition of a water resistant mascara is based on a volatile solvent (for example, isododecane—an isomer of dodecane), animal-derived waxes (for example, beeswax), vegetal based waxes (for example, carnauba wax, rice bran wax, candelila wax), mineral origin wax (for example, ozokerite, paraffin), pigments (for example, iron oxide, ultramarine) and filmifying polymers. Because these mascaras do not contain water-sensitive moieties, they offer an excellent resistance to water and only can be removed with a specific make-up remover that is able to dilute the dried mascara film.

Non-water-resistant mascaras are based on water, soft surfactants (for example, triethanolamine stearate), animal-derived waxes (for example, beeswax), vegetal based waxes (for example, carnauba wax, rice bran wax, candelilla wax), mineral origin waxes (for example, ozokerite, paraffin), pigments (for example, iron oxide, ultramarine), thickening polymers (for example, gum arabic, hydrophobically modified cellulose) and preservatives. They can run under the effect of tears, but are easily removed with some soap and water. Polymers in a water dispersed form (for example, latexes) can bring some level of water resistance to normally non-water resistant mascaras. Waterproof mascaras are similar to oil-based or solvent-based paints, while non water-resistant mascaras behave like water based paints. For intermediate water sensitivity, mascaras and latex-based paints (acrylates) contain polymer dispersions.

The choice of mascara depends on the type of eyelashes (short or long, stiff or curved, poor or bushy, fair or brown) and of the required effect (lengthened, curved and/or thicker eyelashes). Liquid mascaras are the most popular modern formulation, and they can be divided into water-based, solvent-based and water/solvent hybrid varieties.

Water-based mascaras are formulated from waxes (for example, beeswax, carnauba wax, and synthetic wax), water, pigments, which are often iron oxides, and resins dissolved in water. The water evaporates readily, creating a fast-drying product, which thickens and darkens the eyelashes. Some water-based mascaras, very rich in wax (about 30%), are labeled waterproof or water resistant. To color eyelashes, inorganic pigments are the most commonly used because the vast majority of mascaras are black. The formulae also contain antioxidizers to avoid the rancid smell of fatty substances and preservatives, which protect the eye from any risk of infection. Vitamins and hydrocarbon volatile or silicon solvents also can be used to improve the performance of the makeup.

Concerning the solvent-based mascaras, they are formulated with petroleum distillates to which pigments (for example, iron dioxides, ultramarine blue) and waxes (for example, candellila wax, ozokerite and hydrogenated castor oil) are added, making them waterproof.

If it is clear that the makeup effect depends on the formula, it also is important to consider the type of brush and the diameter of the aperture of the mascara tube. Indeed, it must automatically adjust the quantity of the product on the brush to avoid loads on the eyelashes during application. The packaging also must be totally airtight to avoid the degradation and oxidation of the formulation. Thus, to obtain a good application on the eyelashes, it is necessary to develop a compromise between the mascara formula viscosity and the brush type. A rheological approach can be made through the rheological characterization in situ of mascara pastes with the brushes. This procedure used to quantify the take up of mascara brush in the container allows to visualize the influences of the shaft, the bristle length, and the hardness and pattern on the take up, therefore to characterize the product transfer.

According to some embodiments, the topical compositions of the described invention may be formulated as a mascara. According to some such embodiments, the mascara is a water resistant mascara. According to some such embodiments, the mascara is a non-water-resistant mascara.

The mascara compositions of the described invention comprise at least one prostaglandin analog of formula I or formula II, and optionally an imidazole. In some embodiments, the imidazole is miconazole. Within the mascara compositions, agents may be utilized to convert a water dispersible colorant to a non-water dispersible colorant. Accordingly, the mascara compositions also may include at least one cosmetically acceptable hydrophobic colorant. For example, a hydrophobic agent may be coated onto at least one cosmetically acceptable colorant. Hydrophobic agents useful in providing this function include lecithin, dimethicone, dimethicone and mineral oil, polyethylene, isopropyl triisostearyl titanate, calcium stearate, isostearic acid and the combination of squalane, beeswax and lauric acid. All of these specific hydrophobic agents are reported in the CTFA Cosmetic Ingredient Dictionary, Third Edition or Third Edition, Supplement, published by the Cosmetic, Toiletry and Fragrance Association, Inc., Washington, D.C. These dictionary volumes, published 1982 and 1985, respectively, are incorporated in their entirety herein by reference.

The hydrophobically treated colorants suitable for such compositions include such cosmetically acceptable colorants as carmine, bismuth oxychloride, iron oxides (for example, iron oxide black, iron oxide yellow and iron oxide red), zinc oxide, kaolin, ultramarine blue, ultramarine green, ultramarine pink, ultramarine red, ultramarine violet, chromium hydroxide green, chromium oxide greens, silica and manganese violet.

In some embodiments, a cosmetically acceptable pigment treated with a hydrophobic agent, (i.e., a hydrophobic pigment), comprises between about 1% and about 12% by weight, based on the total weight of the mascara composition. In some embodiments, the hydrophobic pigment comprises between about 2% and about 10% by weight, based on the total weight of the mascara composition. In some embodiments, the hydrophobic pigment is present in a concentration in the range of between about 2% and about 8% by weight, based on the total weight of the mascara composition. In some embodiments, the hydrophobic pigment constitutes between about 2% and about 5% by weight, based on the total weight of the mascara composition.

The mascara composition may further comprise a film forming resin. Film forming resins include polyvinyl alcohol, polyvinyl acetate, PVP, ammonium acrylates copolymer, cellulose gum, carboxymethyl hydroxyethylcellulose, acrylate/ammonium methacrylate copolymer and acrylic/acrylate copolymer. These resins are fully defined in the CTFA Cosmetic Ingredient Dictionary, Third Edition and Third Edition, Supplement which are again incorporated herein by reference.

The film forming resin constituent of the cosmetic composition represents about 0.5% to about 4% by weight. In some embodiments, the film forming resin is present in the composition in a concentration in the range of between about 0.8% and about 2.5% by weight. In some embodiments, the film forming resin is present in the composition in a concentration in the range of between about 0.9% and about 1.5%, all the percentages being by weight, based on the total weight of the mascara composition. Those skilled in the art appreciate that the film forming polymers used in the mascara composition of the described invention do not produce sticky type products.

The mascara composition may further comprise a water dispersible thickening agent. In some embodiments, the water dispersible thickening agent is the combination of a base and a polymeric acid. Bases that may be employed to form the water dispersible thickening agent, include, but are not limited to, triethanolamine, isopropanolamine, diisopropanolamine, ethanolamine, sodium hydroxide and potassium hydroxide. Polymeric acids that may be used to form the water dispersible thickening agent are polymers of acrylic acid cross linked with polyfunctional agents such as Carbomer® 910, Carbomer® 934, Carbomer® 934P, Carbomer® 940 and Carbomer® 941.

In formulating such a mascara, the base and the polymeric acid are introduced separately into the composition, and react on contact to form the water dispersible thickening agent. For example, according to some embodiments, the water thickening agent is TEA-Carbomer® 940.

In addition to TEA-Carbomer® 940, other species which may be used as the water dispersible thickening agent of the mascara composition include magnesium aluminum silicate, TEA-Carbomer® 910, TEA-Carbomer® 934, TEA-Carbomer® 934P, TEA-Carbomer® 941, isopropylamine-Carbomer® 940, sodium hydroxide-Carbomer® 940, potassium hydroxide-Carbomer® 940, cellulose gum and xanthan gum. All of which are described in the CTFA Cosmetic Ingredient Dictionary, Third Edition and Third Edition, Supplement, which incorporated herein by reference.

The water dispersible thickening agent can represent between about 0.5% and about 5% by weight of the mascara composition. In some embodiments, the water dispersible thickening agent is present in a concentration in the range of between about 1% and about 4% by weight of the mascara composition. According to other embodiments, the water dispersible thickening agent represents between about 2% and about 3.5% by weight of the mascara composition.

The mascara composition may further comprise a humectant. Suitable humectants include, but are not limited to, glycerin, sorbitol, propylene glycol, glycol, glycol dibehenate, glycol dioctanoate, glycol distearate, glycol hydroxystearate, glycol oleate, glycol ricinoleate, glycol salicylate, glycol stearate, glycol stearate SE, sodium PCA and mixtures thereof. The humectant component of the mascara composition is present is between about 0.1% and about 3% by weight of the composition. According to some embodiments, the humectant comprises between about 0.2% and about 2% by weight of the mascara composition. According to another embodiment, the concentration of humectant represents between about 0.3% and about 1% by weight of the mascara composition.

The mascara composition may further comprise optional components such as a moisturizing agent, a preservative, a sequestering agent and a drying agent. Examples of moisturizing agents include hydrolyzed elastin, hydrolyzed keratin, hydrolyzed silk, hydrolyzed animal protein, hydrolyzed milk protein, hydrolyzed mucopolysaccharides, potassium coco-hydrolyzed animal protein, myristoyl hydrolyzed animal protein and mixtures thereof. In some embodiments, the moisturizing agent is present in a concentration in the range of between about 0.01% and about 1% by weight of the mascara composition.

Examples of preservatives include imidazolidinyl urea, diazolidinyl urea, Quaternium-15, methylparaben, ethylparaben, propylparaben, butylparaben, EDTA and mixtures thereof. In some embodiments, imidazolidinyl urea, methylparaben and mixtures thereof are used in the mascara composition. Preservatives used in the mascara composition may be present in the range of between about 0.01% and about 0.5% by weight.

According to some embodiments, a sequestering agent may be present in a concentration in the range of between about 0.01% and about 0.1% by weight of the mascara composition. In some embodiments, the sequestering agents include disodium EDTA and trisodium EDTA. In some embodiments, the sequestering agent is trisodium EDTA.

Drying agents may be present in the mascara composition in a concentration in the range of between about 1% and less than 5% by weight of the composition. In some embodiments, the drying agents include isopropyl alcohol and at least one alcohol designated as a SD alcohol, which is the designation given by the U.S. Bureau of Alcohol, Tobacco and Firearms to the denaturation method used. For example, SD-40 is ethanol denatured by adding tiny amounts of denatonium benzoate. In some embodiments, at least one of SD Alcohol 1 to SD Alcohol 46 may be used in the mascara composition of this invention.

Generally, water is present in a concentration in the range of between about 60% and about 95%, between about 75% and about 90%, and between about 85% and about 90% by weight of the mascara composition.

In some embodiments, a lecithin-treated cosmetically acceptable pigment, of from about 1.7% to about 4.2% by weight of the mascara composition may be used to produce green, brown, blue and black colored mascaras. In one embodiment wherein a green colored mascara composition is provided by using a mascara composition including between about 3.8% and about 4.2% of chromium hydroxide green by weight of the mascara composition. In another embodiment, brown colored mascara composition is provided by using a mascara composition including between about 0.5% to about 0.7% lecithin-treated iron oxide black by weight of the mascara composition; between about 0.6% to about 0.8% lecithin-treated iron oxide yellow by weight of the mascara composition; and between about 0.6% to about 0.8% lecithin treated iron oxide red by weight of the mascara composition. In another embodiment, a blue colored mascara is provided by using a mascara composition including between about 2.8% to about 3.2% of lecithin-treated ultramarine blue by weight of the mascara composition. In another embodiment, a black colored mascara composition is provided, by incorporating between about 2.3% and about 2.7% of lecithin-treated iron oxide black by weight of the mascara composition.

Topical compositions according to the described invention, which may further include one or more additional active ingredients, therefore can be further efficiently used, in addition to their use as a treatment for an epithelial-related condition, in the treatment of any medical, cosmetic and/or cosmeceutical condition in which applying the additional active ingredient is beneficial.

In another embodiment, the compositions of the described invention further include one or more additional compatible active ingredients, which are aimed at providing the composition with another pharmaceutical, cosmeceutical or cosmetic effect, in addition to that provided by a compound of the inventive composition.

Additional active ingredients included in the compositions according to the described invention used to prevent a hair-related side-effect to a subject associated with treatment of the subject with a chemotherapeutic agent, protect hair from a chemotherapy insult, or improve the appearance of a chemotherapy patient undergoing treatment with a chemotherapeutic agent, include, without limitation, one or more, in any combination, of a protective agent, an emollient, an astringent, an irritant, a keratolytic, a sun screening agent, a sun tanning agent, an antibiotic agent, a non-imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent.

According to another aspect of the described invention, there is provided a method of preparing the compositions described hereinabove. The process generally includes admixing the at least one compound of Formula I or II, optionally at least one compound of Formula III or Formula IV, as described hereinabove, and the pharmaceutically, cosmetically or cosmeceutically acceptable carrier. In cases where additional active ingredients, as detailed above, are present in the compositions, the process includes admixing these ingredients together with the active ingredients and the carrier. The mixing technique utilized in the process of the described invention can involve any one of the known techniques for formulating topical compositions. A variety of exemplary formulation techniques that are usable in the process of the described invention is described, for example, in Harry's Cosmeticology, Seventh Edition, Edited by J B Wilkinson and R J Moore, Longmann Scientific & Technical, 1982.

Protectives as described herein may take the form of dusting powders, adsorbents, mechanical protective agents, and plasters. Dusting powders are relatively inert and insoluble materials that are used to cover and protect epithelial surfaces, ulcers and wounds. Usually, these substances are finely subdivided powders that absorb moisture and can act as a dessicant. The absorption of skin moisture decreases friction and also discourages certain bacterial growth. Some of the materials used as protective adsorbents include bentonite, insoluble salts of bismuth, boric acid, calcium carbonate, (precipitated), cellulose, corn starch, magnesium stearate, talc, titanium dioxide, zinc oxide, and zinc stearate.

Protectives also can be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Mechanical protectives are generally either collodions or plasters. Examples include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

In some embodiments, protectives included in the topical composition of the invention are demulcents.

In some embodiments, the irritant is a rubefacient.

Representative examples of sun screening agents usable in context of the described invention include, without limitation, p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof

Representative examples of sunless tanning agents usable in the described invention include, without limitation, dihydroxyacetone, glyceraldehyde, indoles and their derivatives. The sunless tanning agents can be used in combination with the sunscreen agents.

In some embodiments, the topical composition further comprises vitaminized petides. In some embodiments, the composition further comprises vitaminized proteins.

In some embodiments, sclerosing agents or sclerosants may be also employed.

Cleansing agents which may be use in the described invention include surfactant based cleansing agents, examples of which have been listed hereinabove. Other non-surfactant-based cleansing agents known to those of skill in the art may also be employed.

The topical compositions of the described invention can be applied locally to the skin and may be in any form including solutions, oils, creams, ointments, gels, lotions, shampoos, milks, cleansers, moisturizers, sprays, skin patches and the like.

In another embodiment, a compound of the described invention, a carrier and, optionally, additional active ingredients are formed into a composition comprising a solution, emulsion or gel suspension.

In some embodiments, a compound of the described invention, a pharmaceutical or cosmetic carrier and, optionally, one or more additional active ingredients are in the form of a solution. A solution can be prepared by mixing a solute or dissolved substance (such as a compound of the invention and, optionally, one or more active ingredient(s)) uniformly throughout a solvent carrier such as water or organic solvents, such as the alcohols (e.g. ethanol or isopropanol, acetone).

Emulsifying agent carriers useful in the described invention are described hereinabove. When the composition of the described invention is an emulsion including a compound of the invention, non-lipid-based vehicles are preferred due to the lipophilic nature of the compounds.

In yet another embodiment, a topical composition containing at least one prostaglandin analog of the described invention can be mixed with a gel suspension, (a semi-solid carrier) or solid carrier to form a paste, powder, ointment, cream, lotion, hydrogel or the like.

For example, ointments may be prepared which are in gel-suspension form. These are semi-solid preparations intended for external application to the epithelium. Generally, ointment bases are categorized into hydrocarbon bases (oleaginous), which may use white petroleum as a base; adsorption bases (anhydrous), which might use hydrophilic petroleum or anhydrous lanolin; emulsion bases (water and oil type); emulsion bases (oil and water type); and water soluble bases, which often use polyethylene glycol as an ointment base.

Additional compositions of the described invention can be prepared readily using technology which is known in the art such as described in Remington's Pharmaceutical Sciences, 18th or 19th editions, published by the Mack Publishing Company of Easton, Pa.

In some embodiments, the compositions of the described invention include about 0.001% to about 10.0% w/w of at least one prostaglandin analog.

According to another aspect of the described invention, there is provided a method of treating a medical, cosmetic and/or cosmeceutical condition associated with epithelial tissue that comprises hair loss. The method is effected by topically applying, a pharmaceutically, cosmetically or cosmeceutically effective amount of the composition of the described invention as described above onto an epithelial or epithelial-related surface.

According to some embodiments of the described invention, the topical compositions of the described invention are applied topically as needed. According to some embodiments, the inventive topical compositions are topically applied between one and four times a day. According to some embodiments, the inventive topical compositions are topically applied twice a day (e.g., once in the morning and once in the evening). According to some embodiments, the topical application of the compositions of the described invention is carried out daily. Some conditions may require topical application for an indeterminate length of time.

In one embodiment, the inventive topical compositions are topically administered to an epithelial surface of a subject. Non limiting examples of epithelial surfaces onto which the compositions of the described invention can be applied topically include the lateral aspect of forearms, the lateral aspect of legs, elbows, feet, backhands, back, scalp, face, and any other skin surfaces, and any mucosal membrane described herein.

Alternatively, the compositions may be administered as a component of, for example, a bandage, adhesive, or transdermal patch. In these instances, the compositions may be an integral component of the bandage, adhesive, or transdermal patch and are thereby applied to the epithelial surface.

In some embodiments, the topical compositions of the described invention are administered to treat an epithelial-related condition that is already present, such as alopecia. In some embodiments, the compositions of the described invention are administered to treat an epithelial-related condition such as, for example, but not limited to, sparse hair growth, short hair growth, brittle hair growth, thin hair growth, and/or hair de-pigmenation.

The topical therapeutic compositions may be formulated as ophthalmic preparations to treat alopecia of the eyelashes and alopecia of the eyebrows. Ophthalmic preparations for topical administration can include (pharmaceutical) carriers such as sterile and non-sterile aqueous solutions, non-aqueous solutions in common solvents such as alcohols, or solutions comprising a compound of Formula I or Formula II in liquid or solid oil bases. Such preparations can be prepared readily using technology known in the art, for example, as described in “The Art, Science and Technology of Pharmaceutical Compounding,” Second Edition, edited by Loyd V. Allen, Jr., Ph.D., and published by the American Pharmaceutical Association of Washington D.C., and in “Remington's Pharmaceutical Sciences,” 18th or 19th Editions, published by the Mack Publishing Company of Easton, Pa. In preparing an ophthalmic product, a skilled artisan will take into consideration a number of general considerations, including sterility, buffer capacity and pH, tonicity, viscosity, stability, additives, particle size, packaging and preservatives.

Sterile isotonic solutions, properly preserved, are suitable for preparing ophthalmic solutions. In most cases, when the concentration of the active ingredient is low, i.e., less than about 2.5% to about 3.0%, the active ingredient can be dissolved directly in the isotonic vehicle. In some embodiments, compatible excipients, such as preservatives, antioxidants and viscosity enhancers, may be added. For comfort during administration, ophthalmic and nasal solution dosage forms must be “isotonic” with body fluids.

Ophthalmic suspensions are dispersions of finely divided, relatively insoluble ingredients in an aqueous vehicle containing suitable suspending and dispersing agents. Dosage uniformity often requires brisk shaking of the suspension to distribute the suspended substance. The size of particles in an ophthalmic suspension must be micronized so that the particles are small enough to not irritate the eye.

Ophthalmic ointments offer the advantage of longer contact time and greater total bioavailability. The amount of solid released in unit time is a function of concentration, solubility in the ointment base, and diffusivity of the substance in the base. Ophthalmic ointments are prepared so that they do not irritate the eye, do permit diffusion of the active ingredient, and do retain the activity of the active ingredient for a reasonable period of time when stored properly. White petrolatum is the base primarily used for ophthalmic ointments. Powders incorporated in the preparation must be micronized and sterilized to ensure that the final product is nongritty and thus nonirritating.

As used herein, the singular forms “a”, “an” and “the” include plural referents unless the context clearly dictates otherwise. For example, reference to a “polypeptide” means one or more polypeptides.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Use for Restoring Eyelashes

A composition prepared according to the described invention is formulated as a transparent gel. The formulation comprises water, at least one prostaglandin analog of Formula I or Formula II (concentration about 0.001% to about 0.07% w/w of the composition), optionally at least one imidazole analog of Formula III or Formula IV (concentration about 0.001% to about 1.5% w/w of the composition) (e.g., miconazole), a thickener, optionally a hair stimulating agent; a chelating agent, at least one penetrating agent, and a preservative, and the pH adjusted to 7.2.

The at least one prostaglandin analog is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-(R,E)-3-hydroxy-4-(3-(trifluoromethyl)phenoxy)but-1-enyl)-5-oxocyclopent-1-enyl)hept-5-enoate, (Z)—N-ethyl-7-(2-((R,E)-3-hydroxy-4-(3-(trifluoromethyl)phenoxy)but-1-enyl)-5-oxocyclopent-1-enyl)hept-5-enamide, (Z)—N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof

The gel is applied as a fine line at the skin-eyelash border every night on clean dry eyes. The gel is aqueous enough to spread to the hair follicles but thick enough not to drip.

### Example 2

### Use for Restoring Eyebrows

A composition prepared according to the described invention is formulated as a transparent gel. The formulation comprises water, at least one prostaglandin analog of Formula I or Formula II (concentration about 0.001% to about 0.07% w/w of the composition), optionally at least one imidazole analog of formula III or formula IV (concentration about 0.0001% to about 1.5% w/w of the composition), a thickener, optionally a hair stimulating agent; a chelating agent, at least one penetrating agent, and a preservative, and the pH adjusted to 7.2.

The at least one prostaglandin analog of Formula I or Formula II is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)—N-ethyl-7-(2-((R,E)-3-hydroxy-4-(3-(trifluoromethyl)phenoxy)but-1-enyl)-5-oxocyclopent-1-enyl)hept-5-enamide, (Z)—N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The imidazole analog of Formula III or Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof

The gel is applied by brush as a line at the browbone every night on clean, oil-free brows in the absence of makeup. The gel is aqueous enough to spread to the hair follicles but thick enough not to drip.

### Example 3

### Use for Restoring Scalp Hair

A composition prepared according to the described invention is formulated as an aerosol spray, a topical cream, ointment or a solution.

An aerosol containing approximately 0.005% to about 5.0% (w/w) of at least one prostaglandin analog of Formula I or Formula II, and approximately 0.001% to about 1.5% (w/w) of at least one imidazole analog of formula III or formula IV (e.g., miconazole) is prepared by dissolving the analog in absolute alcohol. The resulting solution is filtered and chilled to about minus 30 degrees C. A chilled mixture of dichlorodifluoromethane and dichlorotetrafluoroethane is added to the solution. Plastic-coated amber bottles are cold filled with the resulting solution and capped. About 1 cc of the formulation is sprayed on the scalp daily at night and rubbed into the scalp.

A topical cream is prepared as follows. Tegacid and spermaceti are melted together at a temperature of about 70 degrees C. to about 80 degree C. Methylparaben is dissolved in water and propylene glycol, polysorbate 80, and at least one prostaglandin analog of Formula I or Formula II (about 0.005% to about 5.0% (w/w)) and optionally at least one imidazole analog of Formula III or Formula IV (e.g., miconazole) (concentration about 0.001% to about 1.5% w/w of the composition) is added in turn, maintaining the temperature at about 75-80 degree C. The methylparaben mixture is added slowly to the tegacid and spermaceti melt with constant stirring for at least 30 minutes, with additional stirring until the temperature has dropped to 40-45 C. Finally, sufficient water is added to bring the final weight to 10000 gm and the preparation stirred to maintain homogeneity until cooled and congealed. The formulation is applied nightly.

A topical ointment containing at least one prostaglandin analog of Formula I or Formula II (about 0.001% to about 5.0% (w/w)) and optionally at least one imidazole analog of formula III or formula IV (concentration about 0.001% to about 1.5% w/w of the composition) (e.g. miconazole) is prepared as follows: White petrolatum and wool fat are melted, strained, and liquid petrolatum added. At least one prostaglandin analog of Formula I or Formula II, of the described invention and miconazole is added; optionally zinc oxide and calamine may be added as well. The mixture is milled until the powders are finely divided and uniformly dispersed. The mixture is stirred into the white petrolatum, melted and cooled with stirring until the ointment congeals. The formulation is applied nightly.

The at least one prostaglandin analog of Formula I or Formula II is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, and 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)—N-ethyl-7-(2-((R,E)-3-hydroxy-4-(3-(trifluoromethyl)phenoxy)but-1-enyl)-5-oxocyclopent-1-enyl)hept-5-enamide, (Z)—N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide;
16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

### Example 4

### Use for Restoring Hair Color

A composition prepared according to the described invention is formulated as an aerosol spray, a topical cream, an ointment or a solution. The total volume used is about 1 cc per application. The formulation is applied nightly. The concentration of the at least one prostaglandin analog of Formula I or Formula II in the composition is about 0.001% to about 5.0% (w/w); the concentration of an optionally added at least one imidazole analog of formula III or formula IV (e.g. miconazole) is about 0.001% to about 1.5% (w/w).

The at least one prostaglandin analog of Formula I or Formula II is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)—N-ethyl-7-(2-((R,E)-3-hydroxy-4-(3-(trifluoromethyl)phenoxy)but-1-enyl)-5-oxocyclopent-1-enyl)hept-5-enamide, (Z)—N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The imidazole analog of Formula III or Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

### Mascara Compositions

At least one prostaglandin analog of Formula I or Formula II, and an optionally added second component, wherein the second component is an imidazole analog of Formula III or Formula IV are ingredients of a black colored mascara composition according to Table 5.

The at least one prostaglandin analog Formula I or Formula II is selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

The prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof

The prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)—N-ethyl-7-(2-((R,E)-3-hydroxy-4-(3-(trifluoromethyl)phenoxy)but-1-enyl)-5-oxocyclopent-1-enyl)hept-5-enamide, (Z)—N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

The prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

The prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

The imidazole analog of Formula III or Formula IV is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof

TABLE 5
Non-limiting mascara formulations.

| Component | Black Mascara | Brown Mascara | Blue Mascara | Green Mascara |
|---|---|---|---|---|
| Water | up to 100 g | up to 100 g | up to 100 g | up to 100 g |
| SD Alcohol 40-B | 4.5 | 4.5 | 4.5 | 4.5 |
| TEA-Carbomer 940 | 3.275 | 3.3 | 3.3 | 2.94 |
| Polyvinyl alcohol | 1 | 1 | 1 | 1 |
| Glycerin | 0.5 | 0.5 | 0.5 | 0.5 |
| Methylparaben | 0.25 | 0.25 | 0.25 | 0.25 |
| Imidazolidinyl urea | 0.01 | 0.01 | 0.01 | 0.01 |
| Trisodium EDTA | 0.05 | 0.05 | 0.05 | 0.05 |
| Hydrolyzed animal | 0.05 | 0.05 | 0.05 | 0.05 |
| protein | | | | |
| lecithin-treated pigments | | | | |
| Chromium hydroxide | | | | 4 |
| green | | | | |
| Iron oxide black | 2.5 | 0.6 | | |
| Iron oxide yellow | | 0.7 | | |
| Iron oxide red | | 0.7 | | |
| Ultramarine blue | | | 3 | |
| Prostaglandin Analog | 0.001-5.0 | 0.001-5.0 | 0.001-5.0 | 0.001-5.0 |
| (Formula I) | | | | |
| Imidazole Analog | 0.001-1.5 | 0.001-1.5 | 0.001-1.5 | 0.001-1.5 |
| (Formula III or IV) | | | | |

### Example 5

### Preparation of a Black Colored Mascara Composition

A black colored mascara composition is prepared by charging about 16.9 parts (by weight) water into a steam jacketed first kettle provided with an agitator. Under agitation 1 part polyvinyl alcohol is added to the water in the first kettle, and mixed for about 15 minutes. An additional charge of water equal to that initially charged, about 16.9 parts, then is added to the agitated mass in the first kettle. After the second charge of water is added, the contents of the first kettle is heated to about 135o F for about 1 hour under agitation. With no visible solids present, 0.05 part of trisodium EDTA and 0.5 part glycerin is added to the contents of the first kettle. The newly added components are added under agitation for about 15 minutes.

After the contents of the first kettle are visibly completely free of solids, they are transferred to a second steam jacketed, agitator-equipped kettle. The contents during transfer are filtered through a fine mesh filter to insure the absence of solids.

In a separate high shear mixer, equipped with a variable speed propeller, are charged 8 parts water and 1 part of a 2.5% solution of Carbomer 940 in water. These components are agitated while 2.5 parts lecithin treated iron oxide black is added thereto. Agitation is continued until the pigment is completely dispersed. When dispersed, the contents of the high shear mixer are transferred into the second kettle.

A 2.5% solution of Carbomer 940 in water (43 parts) is introduced into a tank provided with an agitator, which is activated during introduction of the 2.5% solution. When the ingredients are smooth and lump-free, they are transferred from the tank to the second kettle. Prior to entering the second kettle, the contents of the tank, are strained through a fine mesh. The combined contents of the second kettle are agitated for about 30 minutes at a temperature of about 135° F. to make the contents smooth and uniform. The contents of the kettle are then cooled to about 90° F. under agitation.

In a premix container equipped with a propeller 1 part water, 0.05 part hydrolyzed animal protein and 0.01 part imidazolidinyl urea are mixed. These ingredients are dissolved into a liquid mass under agitation provided by the propeller and then transferred into the second kettle which is stirred after that addition for about 10 to 15 minutes.

In another premix container 4.5 parts of SD Alcohol 40-B and 0.2 part methylparaben are introduced and mixed to form a clear solution. This solution is added to the contents of the second kettle and mixing of the newly added contents continued for about 30 minutes.

In still another premix container 2.2 parts triethanolamine and 2.2 parts water are mixed until they were clear and uniform. When clear and uniform this mixture is added to the agitated contents of the second kettle. A prostaglandin analog of Formula I, Formula II, or Formula III and an optional second component then are added to the contents of the second kettle, such that the final concentration of the prostaglandin analog is from about 0.001% to about 5% w/w and the final concentration of the optional second component is from about 0.001% to about 5% w/w. The contents of the second kettle, the black colored mascara composition, are removed to a storage container.

### Example 6

### Preparation of a Brown Colored Mascara Composition

A brown colored mascara composition containing TEA-Carbomer® 940 and a lecithin-treated hydrophobic pigment is prepared in accordance with the procedure of Example 5.

### Example 7

### Preparation of a Blue Colored Mascara Composition

A blue colored mascara composition is prepared in accordance with the procedure of Example 5. The composition differs from that of Example 5 in the identity and concentration of the lecithin-treated pigments and the concentration of the TEA-Carbomer 940 and water components is summarized in the Table.

### Example 8

### Preparation of a Green Colored Mascara Composition

A green colored mascara composition is prepared in accordance with the procedure of Example 5. The composition differs from that of Example 5 only in the concentration of the TEA-Carbomer 940 and water components and the identity and concentration of the lecithin-treated hydrophobic pigments The composition of the green colored mascara composition of this example is summarized in the Table.

### Example 9

### Prevention of Hair Loss from Chemotherapy Insults

Patient YZ began to apply a composition according to Example 1 containing 0.03% of one of 16-phenoxy-17,18,19,20-tetranor PGF_{2α} N-cyclopropylamide, 16-phenoxy-17,18,19,20-tetranor PGF_{2α} N-cyclopropylmethylamide, 17-phenyl-18,19,20-trinor PGF_{2α} N-cyclopropylamide, 17-phenyl-18,19,20-trinor PGF_{2α} N-cyclopropylmethylamide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, chloprostenol N-methylamide, 17 phenyl-18,19,20-trinor prostaglandin E₂ (N-(1,3-dihydroxypropan-2-yl))amide, 17 phenyl-18,19,20-trinor prostaglandin F_{2α} (N-(1,3-dihydroxypropan-2-yl))amide, and 16 phenoxy, 17, 18,19,20-tetranor prostaglandin F_{2α} (N-(1,3-dihydroxypropan-2-yl))amide to as a fine line at the skin-eyelash border of each eyelid once a day several weeks prior to the initiation of an ACT chemotherapy regimen (doxorubicin, cyclophosphamide, and paclitaxel) for treatment of breast cancer. Patient YZ continued applying the composition throughout and after cessation of the ACT chemotherapy regimen to the eyelid of both upper and lower lashes.

Shortly after initiation of the ACT chemotherapy regimen (about 10-14 days), Patient YZ experienced total hair loss with the exception of her eyelashes which continued to grow and become fuller. Patient YZ reported that she felt “very happy” that at least some of her hair had remained. A few weeks after completion of the ACT chemotherapy, Patient AB stopped applying the composition to her lower eyelashes. Patient YZ's lower eyelashes fell out within 2-3 days. Patient YZ continued to apply the composition as a fine line at the skin-eyelash border of the upper eyelid which have remained long, dark, thick, curled and full.

### Example 10

### Protection from Hair Loss from Chemotherapy Insults

Patient WX began to apply a topical composition comprising at least one of 16-phenoxy-17,18,19,20-tetranor PGF_{2α} N-cyclopropylamide, 16-phenoxy-17,18,19,20-tetranor PGF_{2α} N-cyclopropylmethylamide, 17-phenyl-18,19,20-trinor PGF_{2α} N-cyclopropylamide, 17-phenyl-18,19,20-trinor PGF_{2α} N-cyclopropylmethylamide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, chloprostenol N-methylamide, 17 phenyl-18,19,20-trinor prostaglandin E₂ (N-(1,3-dihydroxypropan-2-yl))amide, 17 phenyl-18,19,20-trinor prostaglandin F_{2α} (N-(1,3-dihydroxypropan-2-yl))amide, and 16 phenoxy, 17, 18,19,20-tetranor prostaglandin F_{2α} (N-(1,3-dihydroxypropan-2-yl))amide to her scalp once a day (at night) two weeks prior to the initiation of a FOLFOX® chemotherapy regimen (infusional 5-fluoruracil, leucovorin and oxaliplatin) for treatment of colorectal cancer.

Shortly after initiation of the FOLFOX chemotherapy regimen, Patient WX experienced complete hair loss with the exception of the hair of her scalp, which remained throughout treatment.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for preventing or reducing a hair-related side effect caused by treatment of a subject in need thereof with at least one chemotherapeutic agent, wherein the side effect is selected from the group consisting of sparse hair growth, brittle hair growth, short hair growth, thin hair growth, alopecia and hair depigmentation, the method comprising the steps:
(a) providing a topical composition, the topical composition comprising a first component and an optional second component, the first component comprising:
(i) at least one compound of Formula I or a pharmaceutically acceptable salt, hydrate, solvate, prodrug or metabolite thereof, 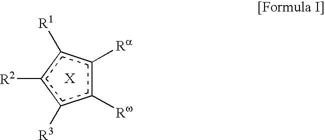
wherein ring X is selected from 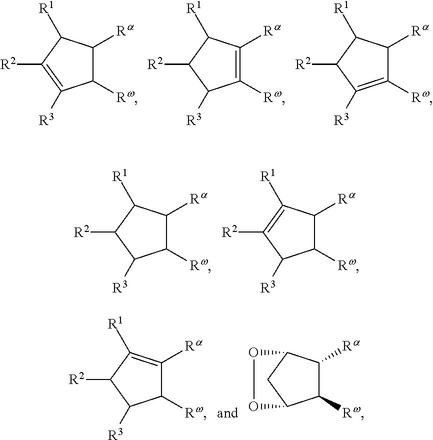
wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each,
wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1˜25 or a pharmaceutically acceptable salt thereof; —P(O)(OH)₂ or a pharmaceutically acceptable salt thereof,
wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,
wherein R^{α} is 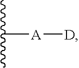
wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one 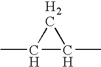
in either cis or trans configuration; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof,
wherein each R⁷ is independently H, F, or straight chain or branched C₁˜C₅ alkyl,
wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,
wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; NO₂; —SR¹⁰; —CH═NOR¹⁰; —C(═O)NR¹¹OR¹²; —S(O)₂NR⁹₂; —NR⁹S(O)₂R¹³; —OS(O)₂NR⁹₂; —C(O)NHS(O)₂R¹³; —S(O)₂R¹³; —SO₃H; —PO₃H₂; 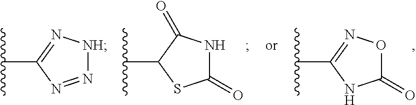
wherein R^{ω} is 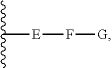
wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety can independently be E or Z and each allenic moiety or chiral center can independently possess any relative or absolute stereoconfiguration or any mixture thereof,
wherein F is —CH₂—, —O—; —S—; —S(O)—; —S(O₂)—; —C(O)—; —C(O)O—; —C(O)S—; —C(O)NR⁹—; —NR⁹—; or a covalent bond,
wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,
wherein each R⁸ is independently selected from the group consisting of: H; a pharmaceutically acceptable cation optionally selected from the group consisting of sodium, potassium, magnesium, calcium or an organic cation optionally selected from the group consisting of an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester optionally selected from the group consisting of a lower alkyl ester, a lower acyloxy-alkyl ester optionally selected from the group consisting of acetoxymethyl, acetoxyethyl, aminocarbonyloxymethyl, pivaloyloxymethyl or pivaloyloxyethyl ester, a lactonyl ester optionally selected from the group consisting of a phthalidyl or thiophthalidyl ester, a lower alkoxyacyloxyalkyl ester optionally selected from the group consisting of a methoxycarbonyloxymethyl, ethoxycarbonyloxyethyl or isopropoxycarbonyloxyethyl ester, an alkoxyalkyl ester, choline ester or acylamino alkyl ester optionally selected from the group consisting of an acetamidomethyl ester; or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,
wherein each R⁹ is independently selected from the following: H; a C₁˜C₂₀ straight chain or branched acyclic hydrocarbon group containing zero to four C═C or C≡C bonds wherein each C═C bond independently can be of E or Z configuration; a C₁˜C₂₀ straight chain or branched acyl group containing zero to four C═C or C≡C bonds wherein each C═C bond independently can be of E or Z configuration; —(CH₂)_{q}OH, —(CH₂)_{q}OR¹⁴, —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl optionally selected from the group consisting of acetoxymethyl, acetoxyethyl, aminocarbonyloxymethyl, pivaloyloxymethyl or pivaloyloxyethyl, lactonyl optionally selected from the group consisting of a phthalidyl or thiophthalidyl, lower alkoxyacyloxyalkyl optionally selected from the group consisting of a methoxycarbonyloxymethyl, ethoxycarbonyloxyethyl or isopropoxycarbonyloxyethyl, or acylamino alkyl optionally selected from the group consisting of acetamidomethyl; or -J-K; or —NR⁹₂ can be a cycloamido radical optionally selected from the group consisting of 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like,
wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl
wherein each R¹¹ is independently H or —C(O)R¹⁶,
wherein each R¹² is independently R¹⁶ or —C(O)R¹⁶,
wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen),
wherein each R¹⁴ is independently straight chain or branched C₁˜C₆ alkyl or —CH₂OCH₃,
wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂; 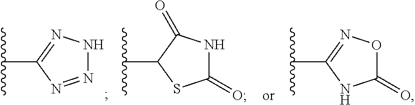
wherein each R¹⁶ is independently H or straight chain or branched C₁˜C₆ alkyl, phenyl or —CH₂OCH₃,
wherein each R¹⁷ is independently straight chain or branched C₁˜C₆ alkyl; —C(O)OR¹⁶; phenyl; F; Cl; Br; I; CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; NO₂; —S(O)₂N(R¹⁶)₂; —NR¹⁶S(O)₂R¹³, with the proviso that, if R¹⁷ is —NR¹⁶S(O)₂R¹³, R¹, R², R³, R^{α} and R^{ω} are selected such that the molecular weight of the compound of Formula I does not exceed about 2000 atomic mass units, and
wherein not more than four of R⁷ are other than H or F and not more than four of R⁷ are F; and
wherein each chiral center or allenic moiety independently possesses any relative or absolute stereoconfiguration or comprises any mixture thereof; and
(ii) a carrier;
(b) topically administering a hair-protective amount of the topical composition onto an epithelial-related surface of the subject concurrent with administration of at least one chemotherapeutic agent; and
(c) stimulating hair growth on the epithelial-related surface to which the topical composition has been applied.

2. The method according to claim 1, wherein the at least one compound of Formula I is diastereomerically pure.

3. The method according to claim 1, wherein the at least one compound of Formula I is a mixture of diastereomers in any ratio.

4. The method according to claim 1, wherein the at least one compound of Formula I is enantiomerically pure.

5. The method according to claim 1, wherein the at least one compound of Formula I is a mixture of enantiomers in any ratio, including a racemate.

6. The method according to claim 1, wherein the at least one compound of Formula I is diastereomerically and enantiomerically pure.

7. The method according to claim 1, wherein the at least one compound of Formula I is a mixture of diastereomers and enantiomers in any ratio.

8. The method according to claim 1, wherein the at least one compound of Formula I has one or more hydrogen atoms replaced by deuterium.

9. The method according to claim 1, wherein the optional second component is an imidazole analog according to Formula III, or a hydrate, solvate, salt, zwitterion, N-oxide, tautomer, prodrug, or metabolite thereof: 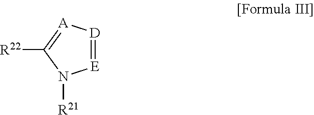
wherein A is N; NR²⁰; NR⁶³ or CH;
wherein D is N or CR²³;
wherein E is N; NR⁶³ or CR²⁴;
with the proviso that, if A is CH, then at least one among D and E is a nitrogen-containing moiety;
with the further proviso that A and E are not simultaneously both NR⁶³;
wherein R²³ is H; F, Cl, Br; I; —NO₂; —N(R⁴²)₂; straight chain or branched C₁˜C₆ alkyl, alkenyl or alkoxy; or phenyl;
wherein R²⁴ is H; F, Cl, Br; I; —NO₂; —N(R⁴₂)₂; straight chain or branched C₁˜C₆ alkyl, alkenyl or alkoxy; or phenyl;
or wherein R²³ and R²⁴ together are —CR⁶²═CR⁶²—CR⁶²═CR⁶²—;
wherein R²⁰ is selected from the group consisting of: H; straight chain or branched C₁˜C₁₂ alkyl; 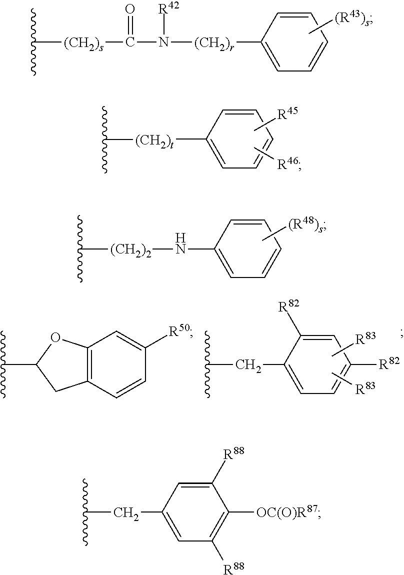
—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula III is an imidazolium or triazolium salt with a pharmaceutically acceptable counter anion or an internal salt (zwitterion); or
wherein R⁶³ is a moiety that is readily cleaved in vivo, wherein R⁶³ optionally is selected from the group consisting of —CHR⁴²OC(═O)(O)ₙR⁵⁵ or —C(R⁴²)₂OP(═O)(OR¹⁰⁶)₂ and the compound of Formula III is a prodrug and an imidazolium or triazolium salt with a pharmaceutically acceptable counter ion or an internal salt (zwitterion);
wherein s is 1 or 2;
wherein t is an integer from 1 to 4 inclusive;
wherein u is 2 or 3;
wherein R²² is H; F, Cl, Br; I; —NO₂; —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical containing one S and one or two N atoms, said heterocyclic radical optionally selected from the group consisting of thiazolyl or thiazol-4-yl;
wherein R²¹ is H; or —(CR²⁵R²⁶)ₘ—CR²⁷R²⁸-Q-R²⁹;
wherein each m is independently an integer from 0 to 4 inclusive;
wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;
wherein R²⁶ is H; or straight chain or branched C₁˜C₆ alkyl;
wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(═NH)N(R⁶⁸)OR⁶⁸; —CF(R⁶⁷)C(═NH)N(R⁶⁸)OH; or —CF(R⁶⁷)C(═N—OR⁶⁹)NH₂;
wherein each r is independently an integer from 0 to 2 inclusive;
wherein each L is independently α-tetralyl; or phenyl, said phenyl being optionally substituted with up to three groups selected from the group consisting of: straight chain or branched C₁˜C₆ alkyl, straight chain or branched C₁˜C₆ alkoxy, —C≡N, —NO₂, or —NH₂;
wherein R²⁸ is H; F; Cl; Br; —OR⁴²; —OC(═O)R¹⁰⁰; 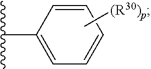
straight chain or branched C₁˜C₆ alkyl; R61; or —OP(═O)(OH)₂ or a pharmaceutically acceptable salt or zwitterion form thereof;
wherein each Q is independently a covalent bond; —O—; —S—; —S(O)—; or —S(O)₂—;
wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; straight chain or branched C₁˜C₆ alkyl optionally substituted with —CO₂H; or 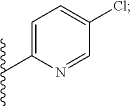
wherein each p is independently an integer from 0 to 3 inclusive;
wherein each T is independently a covalent bond; —CH₂CH₂—; —CH═CH—; —(O)ₙC(R⁴²)₂—; —CH₂O—; —SCH₂—; —CH₂S—; —OCH₂CH₂O—; —CH(CH₃)—; —CH₂—; —CF₂—; —C(R⁹³)₂—; or 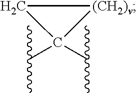
wherein each v is independently an integer from 1 to 3 inclusive;
wherein each U is independently a covalent bond; 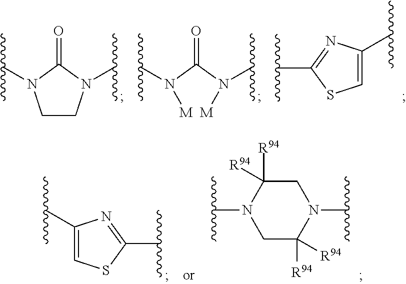
wherein each V is independently H; —S(O)₂CH₃; —C(O)NH₂; —CH₂C≡CH; —CH═CH₂; —S(O)ᵣR⁵⁵; 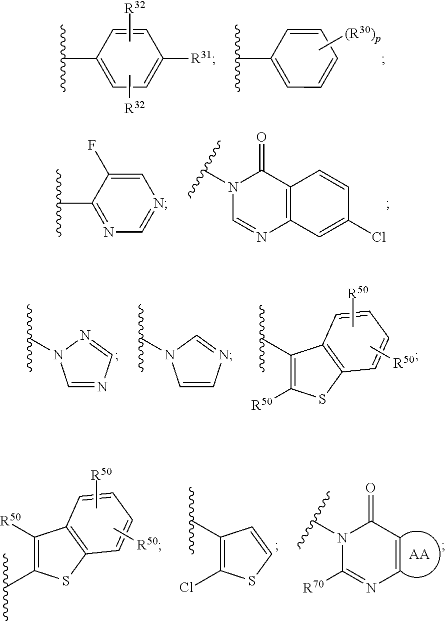
R⁵⁹; —R⁶¹; or -LL-R⁹⁵;
wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings;
wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴; —OC(O)N(R⁵⁴)₂; —SH; —SR⁴¹; —S(O)ᵣR⁵⁸; —CH═CH—CO₂H; 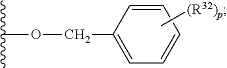
straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to —CH₂C≡N, —CH₂CH₂C≡N, —CH₂CH₂CH₂C≡N, and —CH₂CH(CH₃)C≡N; straight chain or branched C₁˜C₆ halogenoalkyl optionally selected from the group consisting of —CF₃, —CF₂CF₃, —CH₂CF₃, —CHF₂, —CH₂F, —CH₂Cl, and —CH₂Br; straight chain or branched C₁˜C₅ halogenoalkoxy optionally selected from the group consisting of —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₆ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy;
wherein each R³¹ is independently H; F; Cl; Br; I; —C≡N; —NO₂; —CF₃; —N═C═S; —N(R⁴²)₂; —NH—C(═O)-(M)ₙ-R⁵⁴; —NH—C(═S)—NH—R⁵⁴; straight chain or branched C₁˜C₆ alkoxy or alkylthio; straight chain or branched C₁˜C₁₂ alkyl; —(CH₂)ᵣ—R⁹⁹; 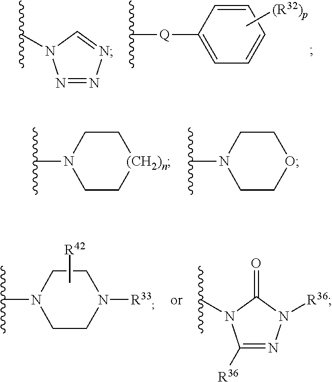
wherein each M is independently O or NH, with the proviso that when M is O and n is 1, R⁵⁴ is other than H;
wherein each R³² is independently H; F; Cl; Br; I; —C≡N; —NO₂; straight chain or branched C₁˜C₁₂ alkyl; straight chain or branched C₁˜C₄ alkoxy; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; straight chain or branched C₁˜C₃ halogenoalkoxy optionally selected from the group consisting of —OCF₃, —OCF₂CF₃, —OCH₂CF₃, —OCHF₂, —OCH₂F; straight chain or branched C₁˜C₄ alkylthio or haloalkylthio; straight chain or branched C₁˜C₄ alkylthionyl or haloalkylthionyl; or straight chain or branched C₁˜C₄ alkylsulfonyl or haloalkylsulfonyl;
wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)—C(O)OR³⁴; —C(O)O-phenyl; —(CH₂)—C(O)N(R⁴²)₂; —CH₂C(S)N(R⁴²)₂; —CH₂C(S)SR³⁴; —(CH₂)n-phenyl; benzoyl, wherein said benzoyl is optionally substituted with one or two R⁴⁶ groups; or 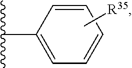
or, preferably, 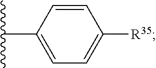
wherein each R³⁴ is independently straight chain or branched C₁˜C₆ alkyl;
wherein each R³⁵ is independently H; 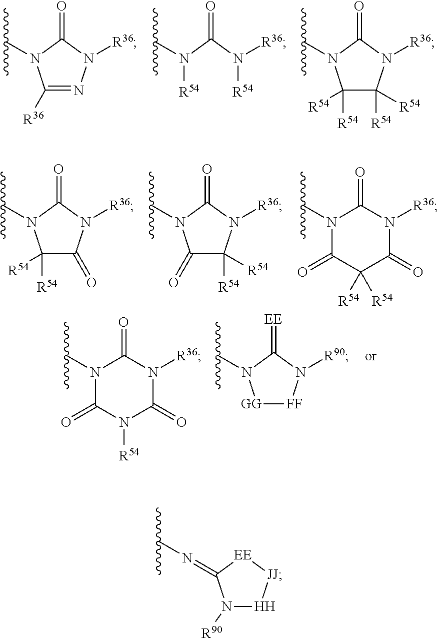
wherein each R³⁶ is independently H; straight chain or branched C₃˜C₇ alkenyl, with the proviso that the olefinic double bond is not located α to N; straight chain or branched C₃˜C₇ alkynyl, with the proviso that the alkynyl C≡C bond is not located α to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or 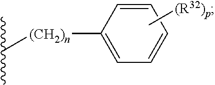
wherein each n is independently 0 or 1;
wherein R²⁶ and R²⁸ together with the two carbon atoms to which they attach can be C═C; wherein R²⁷ and R²⁸ together can be 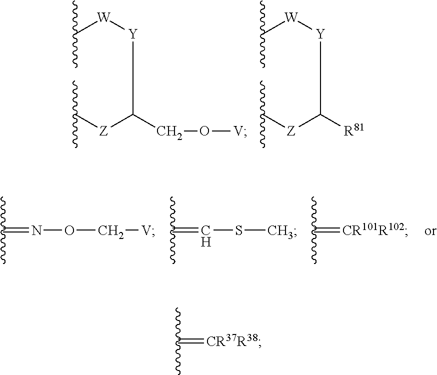
wherein W is —CH₂— and Y is —CH₂—; W is —O— and Y is —CH₂—; or W is —CH₂— and Y is —O—;
wherein Z is —CH₂— or —O—;
wherein R²⁷ together with -Q-R²⁹ can be 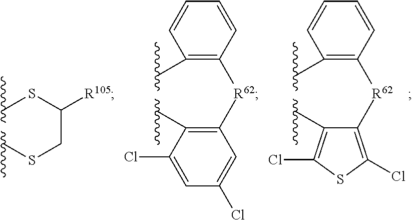
or, when R²⁷ and R²⁹ each independently represents a cycloalkyl, aryl or heterocyclic ring and Q is a covalent bond, R²⁷ together with -Q-R²⁹ can be 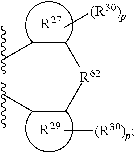
wherein each R³⁷ is independently H; C₁˜C₂ alkyl or phenyl;
wherein each R³⁸ is independently H or C₁˜C₂ alkyl;
wherein each R³⁹ is independently H; straight chain or branched C₁˜C₅ alkyl; straight chain or branched C₁˜C₅ alkanoyl; or straight chain or branched C₁˜C₅ haloalkanoyl optionally selected from the group consisting of —C(O)CF₃, —C(O)CF₂CF₃, —C(O)CH₂CF₃, —C(O)CHF₂, and —C(O)CH₂F;
wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;
wherein each R⁴¹ is independently straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;
wherein each q is independently an integer from 0 to 4 inclusive;
wherein each G¹ is independently a covalent bond; —O—; or —S—;
wherein each J is independently phenyl, 2-thienyl or 3-thienyl wherein said phenyl, 2-thienyl or 3-thienyl is optionally substituted with one to three groups selected from the group consisting of: F; Cl; Br; I; —NO₂; straight chain or branched C₁˜C₅ alkyl; straight chain or branched C₁˜C₅ alkoxy;
wherein each R⁴² is independently H; or straight chain or branched C₁˜C₆ alkyl;
wherein each R⁴³ is independently H; F; Cl; Br; I; —NO₂; straight chain or branched C₁˜C₆ alkyl; straight chain or branched C₁˜C₆ alkoxy; —CF₃; or —O— phenyl;
wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the group consisting of: F, Cl, Br, I, straight chain or branched C₁˜C₆ alkyl, or straight chain or branched C₁˜C₆ alkoxy;
wherein each R⁴⁵ is independently H; F; Cl; Br; I; —NO₂; straight chain or branched C₁˜C₆ alkyl; straight chain or branched C₁˜C₆ alkoxy; or —S(O)₂NH₂;
wherein each R⁴⁶ is independently H; F; Cl; Br; I; straight chain or branched C₁˜C₆ alkyl; straight chain or branched C₁˜C₆ alkoxy; or —C≡N;
wherein each R⁴⁷ is independently a phenyl group optionally substituted with one to three F, Cl, Br or I;
wherein each R⁴⁸ is independently H; F; Cl; Br; I; or straight chain or branched C₁˜C₆ alkyl;
wherein each R⁴⁹ is independently 1-naphthyl; 2-naphthyl; or phenyl, wherein said phenyl is optionally substituted with one or two groups, each independently selected from the group consisting of: F, Cl, Br, I, straight chain or branched C₁˜C₆ alkyl, straight chain or branched C₁˜C₆ alkoxy, or —CH₂CH═CH₂;
wherein each R⁵⁰ is independently H; F; Cl; Br; or I;
wherein each R⁵¹ is independently phenyl, wherein said phenyl is optionally substituted with one or two groups, each independently selected from the group consisting of: F, Cl, Br, I, or straight chain or branched C₁˜C₆ alkyl;
wherein each R⁵² is independently phenyl, wherein said phenyl is optionally substituted with one or two groups, each independently selected from the group consisting of: F, Cl, Br, or I;
wherein each R⁵³ is independently straight chain or branched C₁˜C₈ alkyl optionally substituted with C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; straight chain or branched C₃˜C₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl C≡C bond is located β, γ or δ with respect to the ether oxygen;
wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl;
wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle can be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups;
wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle can be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle can be aromatic or non-aromatic; methylenedioxy; —C≡N; —NO₂; —CF₃; —N(R⁵⁴)₂; —OR⁵⁴; —SH; ═O; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; —C(═NH)N(R⁵⁴)OH; —C(═N—OR⁵⁴)NH₂; —S(O)₂N(R⁵⁴)₂; —NR⁵⁴S(O)₂R⁵⁴; —C(O)NHS(O)₂R⁵⁴; —S(O)₂R⁵⁴; —SO₃H; Or —PO₃H₂;
wherein each R⁵⁷ is independently F; Cl; Br; I; straight chain or branched C₁˜C₆ alkyl; straight chain or branched C₁˜C₆ alkoxy; —C≡N; —CF₃; —NO₂; —NH₂; or —NHC(O)R⁶⁶, wherein said R⁶⁶ is straight chain or branched C₂˜C₁₂ alkyl;
wherein each R⁵⁸ is independently straight chain or branched C₁˜C₂₀ alkyl; C₃˜C₈ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups;
wherein each R⁵⁹ is independently straight chain or branched C₁˜C₇ alkyl, alkenyl or alkynyl, said alkyl, alkenyl or alkynyl being substituted with one or more —R⁶⁰—R⁶¹;
wherein each R⁶⁰ is independently a covalent bond or —O—;
wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups;
wherein each R⁶² is independently a covalent bond; —CH₂—CH₂—; —CH═CH—; —O—; or —S—;
wherein each R⁶⁵ is independently straight chain or branched C₁˜C₁₂ acyclic hydrocarbon group, which can be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or C≡C bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;
wherein each R⁶⁷ is independently H; F; or straight chain or branched C₁˜C₆ alkyl, said alkyl group being optionally substituted by one or more R³⁰;
wherein each R⁶⁸ is independently R³⁶, R⁵⁴ or R⁶¹;
wherein each R⁶⁹ is independently H; or straight chain or branched C₁˜C₆ alkyl, said alkyl group being optionally substituted by one or more R³⁰;
wherein each R⁷⁰ is independently H; straight chain or branched C₁˜C₄ alkyl; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; or cyclopropyl;
wherein each R⁷¹ is independently R⁵⁶; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)rR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶; 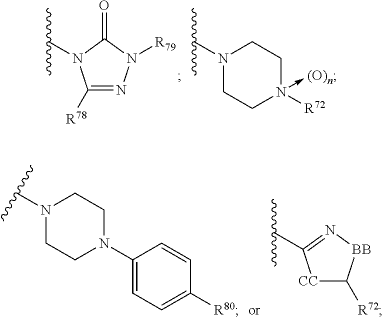
wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;
wherein each R⁷³ is independently H; straight chain or branched C₁˜C₄ alkyl; C₃˜C₆ cycloalkyl; —C(O)R⁷²; or —C(O)CF₃;
wherein each R⁷⁴ is independently straight chain or branched C₁˜C₄ alkyl;
wherein each R⁷⁵ is independently H; —C(O)NH₂; —C(O)CH₃; —C≡N; —SO₂NHR⁷²; —SO₂R⁷²; —OR⁷²; —OC(O)R⁷²; or straight chain or branched —(C₁˜C₄ alkyl)-NH₂;
wherein each R⁷⁶ is independently phenyl optionally substituted with one or more R⁷⁷ groups;
wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂, —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²; 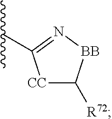
or phenyl, said phenyl being optionally substituted with one or more F, Cl, Br, I, —C≡N, —NO₂, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;
wherein each R⁷⁸ is independently H or —CH₃;
wherein each R⁷⁹ is independently H; isopropyl; cyclopentyl; cyclopropyl; 2-butyl; 3-pentyl; 3-hydroxy-2-butyl; or 2-hydroxy-3-pentyl;
wherein each R⁸⁰ is independently F; Cl; Br; I; —C≡N; —NO₂; —NH₂; straight chain or branched C₁˜C₄ halogenoalkyl; straight chain or branched C₁˜C₄ halogenoalkoxy; or 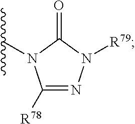
wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —C≡N, —NO₂, phenyl, phenylmethyl, benzoyl, halobenzoyl, —C(O)R⁴², —C(O)OR⁴², and —CF₃, with the proviso that when multiple substituents are present only 1 thereof can be selected from the group consisting of phenyl, phenylmethyl, benzoyl and halobenzoyl;
wherein each R⁸² is independently H or —OR⁸⁴;
wherein each R⁸³ is independently H; F; Cl; Br; R⁷⁴; —OR⁷⁴; —SR⁷⁴; —CH₂SC(═O)R⁷⁴; —COOH; or —C(═O)OCH₃;
wherein each R⁸⁴ is independently a group that is easily hydrolyzable under physiological conditions, optionally at the acyl residue of an amino acid or a group represented by the formula —C(═O)R⁸⁵ or —P(═O)(OR⁸⁶)₂; wherein R⁸⁴ is optionally selected from the group consisting of formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like;
wherein each R⁸⁵ is independently H; —OR⁷⁴; or straight chain or branched C₁˜C₆ alkyl or alkenyl, wherein said alkyl or alkenyl can be optionally interrupted by a hydrolyzable functional group such as carboxamide or ester, wherein said alkyl or alkenyl can be optionally substituted with —COOH, —NH₂, —NHR⁷⁴, —N(R⁷⁴)₂, or R⁶¹, wherein R⁶¹ is optionally selected from the group consisting of phenyl, methoxyphenyl, pyridyl, pyrazinyl or furyl;
wherein each R⁸⁶ is independently H or R⁷⁴;
wherein each R⁸⁷ is independently straight chain or branched C₁˜C₅ alkyl; R⁶¹; pyridyl; pyrrolidinyl; or -DD-NH—R⁸⁹;
wherein each R⁸⁸ is independently H; F; Cl; Br; or —OR⁷⁴;
wherein each DD is independently straight chain or branched C₁˜C₄ alkylene; —CH₂NHC(═O)CH₂—; —CH(NH₂)CH₂CH₂CH₂—;
wherein each R⁸⁹ is independently H or straight chain or branched C₁˜C₅ alkyl;
wherein each BB is independently —CH₂— or —C(═O)—;
wherein each CC is independently NH or O;
wherein each EE is independently O, S or N—R⁹¹;
wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃˜C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched;
wherein each KK is independently O or S;
wherein each R⁹¹ is independently H or straight chain or branched C₁˜C₆ alkyl;
wherein each R⁹² is independently H; straight chain or branched C₁˜C₆ alkyl; C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—;
wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃;
wherein each GG is independently —C(═O)—; or —CH₂—, optionally substituted with up to two radicals selected from the group consisting of straight chain or branched C₁˜C₆ alkyl and straight chain or branched C₁˜C₆ alkoxy;
or FF and GG, taken together, form a bivalent radical of formula —N═CH—(FFGG′);
wherein HH has the same meaning as FF;
wherein JJ has the same meaning as GG;
or HH and JJ, taken together, form a bivalent radical of formula —N═CH—(FFGG′) or —CH═CH—(HHJJ′);
wherein the nitrogen atom in the bivalent radical FFGG′ is connected to NR⁹⁰; wherein one hydrogen in said radical FFGG′ and up to two hydrogens in radical HHJJ′ can be replaced by a straight chain or branched C₁˜C₆ alkyl radical;
wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group optionally selected from the group consisting of —CH₂CH₂—, —CH₂CH₂CH₂—, —CH₂CH₂CH₂CH₂— and the like;
wherein each R⁹⁴ is independently H or C₁˜C₄ alkyl which is unsubstituted or substituted by 1-3 substituents each independently selected from the group consisting of halogen, hydroxyl, C₁˜C₄ alkoxy, and amino; or two R⁹⁴ groups attached to the same carbon atom together form ═S;
wherein each LL is independently a covalent bond; —C(═O)—; —C(═S)—; —SO₂—; or —N═N—;
wherein each R⁹⁵ is independently selected from the group consisting of
i) hydrogen,
ii) CN
iii) CHO
iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl,
v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido,
vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl,
vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and
viii) a group of the formula 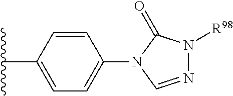
wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (O) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which can be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl;
wherein each MM is independently an ethylene group optionally substituted with R91; or MM is —NN═OO—, wherein NN and OO are the same or different and are each independently N or CR⁹¹;
wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶;
wherein each R¹⁰⁰ is independently —R⁵⁴ or —OR⁵⁴;
wherein each R¹⁰¹ is independently H; F; Cl; Br; or I;
wherein each R¹⁰² is independently F; Cl; Br; I; —SR¹⁰³; or —OR¹⁰⁴;
wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which can be the same or different;
wherein each R¹⁰⁴ is independently straight chain or branched C₁˜C₁₂ alkyl; or phenyl;
wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰;
wherein each R¹⁰⁶ is independently H, a pharmaceutically acceptable cation, or null (affording an azolium phosphate zwitterion);
wherein each chiral center independently may possess any relative or absolute stereoconfiguration or be any mixture thereof, unless specified otherwise herein; and
wherein each olefinic C═C bond that is capable of E/Z isomerism independently may possess either the E or Z stereoconfiguration or be any mixture thereof, unless specified otherwise;
wherein the imidazole analog improves efficacy of the at least one prostaglandin analog of Formula I when the composition is delivered to a subject refractory to treatment by a composition containing the prostaglandin analog of Formula I alone.

10. The method according to claim 9, wherein the imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

11. The method according to claim 9, wherein the imidazole analog is miconazole or ketoconazole or a pharmaceutically acceptable salt, hydrate, solvate, prodrug or metabolite thereof.

12. The method according to claim 1, wherein the at least one compound of formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

13. The method according to claim 12, wherein the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-(R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

14. The method according to claim 12, wherein the prostaglandin B analog is selected from the group consisting of 16-phenoxy-17,18,19,20-tetranor PGB₂ N-cyclopropylamide, 16-phenoxy-17,18,19,20-tetranor PGB₂ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (R,Z)-isopropyl 7-(2-(3-hydroxy-5-phenylpentyl)-5-oxocyclopent-1-enyl)hept-5-enoate, (Z)-isopropyl 7-(2-((R,E)-3-hydroxy-4-(3-(trifluoromethyl)phenoxy)but-1-enyl)-5-oxocyclopent-1-enyl)hept-5-enoate, (Z)—N-ethyl-7-(2-((R,E)-3-hydroxy-4-(3-(trifluoromethyl)phenoxy)but-1-enyl)-5-oxocyclopent-1-enyl)hept-5-enamide, (Z)—N-ethyl-7-(2-((S,E)-3-hydroxy-5-phenylpent-1-enyl)-5-oxocyclopent-1-enyl)hept-5-enamide, (Z)-7-(2-((R,E)-3-hydroxy-4-(3-(trifluoromethyl)phenoxy)but-1-enyl)-5-oxocyclopent-1-enyl)hept-5-enoic acid, (Z)-7-(2-((R,E)-3-hydroxy-4-(3-(trifluoromethyl)phenoxy)but-1-enyl)-5-oxocyclopent-1-enyl)-N-methylhept-5-enamide, (Z)-7-(2-((R,E)-4-(3-chlorophenoxy)-3-hydroxybut-1-enyl)-5-oxocyclopent-1-enyl)hept-5-enoic acid, (Z)-isopropyl 7-(2-((R,E)-4-(3-chlorophenoxy)-3-hydroxybut-1-enyl)-5-oxocyclopent-1-enyl)hept-5-enoate, (Z)-7-(2-((R,E)-4-(3-chlorophenoxy)-3-hydroxybut-1-enyl)-5-oxocyclopent-1-enyl)-N-methylhept-5-enamide or a pharmaceutically acceptable salt, hydrate, solvate, prodrug or metabolite thereof.

15. The method according to claim 12, wherein the prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

16. The method according to claim 12, wherein the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

17. The method according to claim 12, wherein the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

18. The method according to claim 12, wherein the prostaglandin F analog is selected from the group consisting of 16-phenoxy-17,18,19,20-tetranor PGF_{2α} N-cyclopropylamide, 16-phenoxy-17,18,19,20-tetranor PGF_{2α} N-cyclopropylmethylamide, 16-phenoxy-17,18,19,20-PGF_{2α} N-(1,3-dihydroxypropan-2-yl))amide, -phenyl-18,19,20-trinor PGF_{2α} N-cyclopropylamide, 17-phenyl-18,19,20-trinor PGF_{2α} N-cyclopropylmethylamide, 17-phenyl-18,19,20-trinor PGF_{2α} N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α} N-cyclopropylmethylamide, 6-(3-chlorophenyl)-17,18,19,20-tetranor PGF_{2α} N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

19. The method according to claim 12, wherein the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

20. The method according to claim 1, wherein the optional second component of the composition comprises an additional active agent selected from the group consisting of a protective agent, an emollient, an astringent, an irritant, a keratolytic, a sun screening agent, a sun tanning agent, an antibiotic agent, a non-imidazole analog antifungal agent, an imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant, a chemotherapeutic agent, an anti-histamine, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent, a hypo-pigmenting agent, or a combination thereof.

21. The method according to claim 1, wherein the at least one chemotherapeutic agent is at least one agent selected from the group consisting of an alkylating agent, an antimetabolite, a natural product, a biologic agent, a hormone or hormone-related agent, and a miscellaneous agent.

22. The method according to claim 1, wherein the at least one chemotherapeutic agent is an alkylating agent, and wherein the side-effect of treatment with the alkylating agent is alopecia.

23. The method according to claim 22, wherein the alkylating agent is selected from the group consisting of temozolomide, busulfan, ifosamide, melphalan hydrochloride, carmustine, lomustine and cyclophosphamide.

24. The method according to claim 1, wherein the at least one chemotherapeutic agent is an antimetabolite, and wherein the side-effect of treatment with the antimetabolite is alopecia.

25. The method according to claim 24, wherein the antimetabolite is selected from the group consisting of 5-fluorouracil, capecitabine, gemcitabine, floxuridine, decitabine, mercaptopurine, pemetrexed disodium, methotrexate and dacarbazine.

26. The method according to claim 1, wherein the at least one chemotherapeutic agent is a natural product, and wherein the side-effect of treatment with the natural product is alopecia.

27. The method according to claim 26, wherein the natural product is selected from the group consisting of vincristine, vinblastine, vinorelbine tartrate, paclitaxel, docetaxel, ixabepilone, daunorubicin, epirubicin, doxorubicin, idarubicin, mitoxantrone, mitomycin, dactinomycin, irinotecan, topotecan, etoposide, teniposide, etoposide phosphate, and bleomycin sulfate.

28. The method according to claim 1, wherein the at least one chemotherapeutic is a biologic agent, and wherein the side-effect of treatment with the biologic agent is alopecia.

29. The method according to claim 28, wherein the biologic agent is selected from the group consisting of filgrastim, pegfilgrastim, bevacizumab, sargramostim and panitumumab.

30. The method according to claim 1, wherein the at least one chemotherapeutic agent is a hormone or a hormone-related agent, and wherein the side-effect of treatment with the hormone or the hormone-related agent is alopecia.

31. The method according to claim 30, wherein the hormone or the hormone-related agent is selected from the group consisting of megestrol acetate, fluoxymesterone, leuprolide, octreotide acetate, tamoxifen citrate and fluxymesterone.

32. The method according to claim 1, wherein the at least one chemotherapeutic agent is a miscellaneous agent, and wherein the side-effect of treatment with the miscellaneous agent is alopecia.

33. The method according to claim 1, wherein the miscellaneous agent is selected from the group consisting of sorafenib, erlotinib, oxaliplatin, dexrazoxane, anagrelide, isotretinoin, bexarotene and vorinostat.

34. The method according to claim 1, wherein the topical composition is administered concurrently with a chemotherapy regimen for treating breast cancer.

35. The method according to claim 34, wherein the chemotherapy regimen is an Adriamycin-Cytoxan-Taxol regimen.

36. The method according to claim 1, wherein the topical composition is administered concurrently with a chemotherapy regimen for treating colorectal cancer.

37. The method according to claim 36, wherein the chemotherapy regimen comprises a combination of infusional 5-fluoruracil, leucovorin and oxaliplatin (FOLFOX®).

38. The method according to claim 36, wherein the chemotherapeutic regimen for colorectal cancer comprises FOLFOX® with bevacizumab.

39. The method according to claim 36, wherein the chemotherapy regimen comprises infusional 5-fluoruracil, leucovorin, and irinotecan (FOLFIRI®) with bevacizumab.

40. The method according to claim 1, wherein the composition is formulated as a mascara.

41. A method for preventing or reducing hair loss due to a chemotherapy insult in a subject in need thereof, the method comprising the steps
(a) providing a topical composition, the topical composition comprising a first component and an optional second component, the first component comprising:
(i) at least one compound of Formula I or a pharmaceutically acceptable salt, hydrate, solvate, prodrug or metabolite thereof, 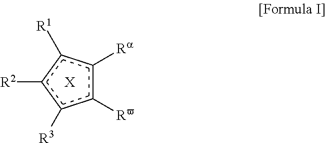
wherein ring X is selected from 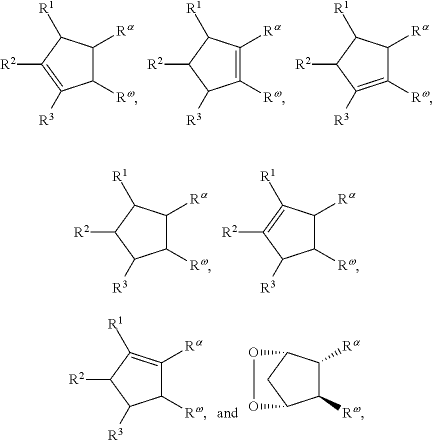
wherein R¹, R² and R³ are independently H, —OR⁴, ═O, or —OC(O)R⁵, where the carbon atoms to which R¹, R² and R³ attach bear the appropriate number of additional H atoms so as to have exactly 4 bonds each,
wherein each R⁴ is independently H; C₁˜C₁₀ straight chain or branched alkyl; an alkyl radical having from two to six carbon atoms interrupted by one or two —O— or —S—, where no two heteroatoms are adjacent; a monosaccharide, oligosaccharide or polysaccharide attached via an anomeric carbon atom; —(PO₂OH)ₛH where s is 1˜25 or a pharmaceutically acceptable salt thereof; —P(O)(OH)₂ or a pharmaceutically acceptable salt thereof,
wherein each R⁵ is independently H; saturated or unsaturated, straight chain or branched C₁˜C₂₀ acyclic hydrocarbon or —(CH₂)ₘR⁶ wherein m is an integer from 0˜10 and R⁶ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,
wherein R^{α} is 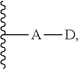
wherein A is a divalent hydrocarbon radical having from two to ten carbon atoms, which can be interrupted by one or more —O— or —S—, zero or one 1,2-phenylene, 1,3-phenylene, or 1,4-phenylene radical, and zero or one 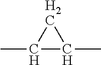
in either cis or trans configuration; said hydrocarbon radical containing zero to four C═C or C≡C bonds and zero to one C═C═C moiety;
said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵, R⁷ or M groups; wherein each olefinic moiety may independently be E or Z and each allenic moiety or chiral center may independently possess any relative or absolute stereoconfiguration or any mixture thereof,
wherein each R⁷ is independently H, F, or straight chain or branched C₁˜C₅ alkyl,
wherein M is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁷ groups,
wherein D is —C(O)OR⁸; —OC(O)OR⁸; —C(O)NR⁹₂; —OC(O)NR⁹₂; —C(O)NR⁹NR⁹₂; —OC(O)NR⁹NR⁹₂; —C(O)NR⁹C(O)R⁵; —NR⁹₂; —NR⁹₃⁺; —NR⁹C(═NR⁹)NR⁹₂; —N(R⁹)C(O)OR⁸; —N(R⁹)C(O)NR⁹₂; —N(R⁹)C(O)R⁵; —C(O)R¹⁰; —OC(O)R¹⁰; —OR¹⁰; H; —C≡N; —N₃; F; Cl; —CF₃; —CF₂CH₂OH; NO₂; —SR¹⁰; —CH═NOR¹⁰; —C(═O)NR¹¹OR¹²; —S(O)₂NR⁹₂; —NR⁹S(O)₂R¹³; —OS(O)₂NR⁹₂; —C(O)NHS(O)₂R¹³; —S(O)₂R¹³; —SO₃H; —PO₃H₂; 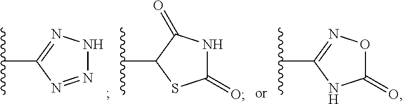
wherein R^{ω} is 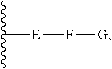
wherein E is a divalent hydrocarbon radical having from two to ten carbon atoms, which can be interrupted by one or more —O— or —S— and zero or one 1,2-phenylene, 1,3-phenylene, or 1,4-phenylene radical; said hydrocarbon radical containing zero to four C═C or C≡C bonds and zero to one C═C═C moiety; said hydrocarbon radical having no two heteroatoms adjacent and no heteroatom adjacent to a non-aromatic C—C multiple bond; said hydrocarbon radical being optionally substituted by one or more —OR⁵, ═O, ═S, —O(CO)R⁵ or R⁷ groups; wherein each olefinic moiety may independently be E or Z and each allenic moiety or chiral center may independently possess any relative or absolute stereoconfiguration or any mixture thereof,
wherein F is —CH₂—, —O—; —S—; —S(O)—; —S(O₂)—; —C(O)—; —C(O)O—; —C(O)S—; —C(O)NR⁹—; —NR⁹—; or a covalent bond,
wherein G is H; cycloalkyl; aryl; heterocycle; —CR⁷═N-aryl; —CR⁷═N-heterocycle; wherein cycloalkyl is C₃˜C₁₀ cycloalkyl containing from one to four rings, aryl is C₆˜C₁₀ aryl containing one or two rings, and heterocycle is 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,
wherein each R⁸ is independently selected from the group consisting of: H; a pharmaceutically acceptable cation optionally selected from the group consisting of sodium, potassium, magnesium, calcium or an organic cation optionally selected from the group consisting of an ammonium ion; a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁵ groups; —(CH₂)_{q}OH, —(CH₂)_{q}OR¹⁴ or —(CH₂)_{q}OC(O)R¹⁴ where q is an integer from 1 to 6 inclusive; —CH₂CH(OH)CH₂OH; —CH(CH₂OH)₂; —CH₂CH(CH₂OH)₂; a biohydrolyzable ester optionally selected from the group consisting of a lower alkyl ester, a lower acyloxy-alkyl ester optionally selected from the group consisting of acetoxymethyl, acetoxyethyl, aminocarbonyloxymethyl, pivaloyloxymethyl or pivaloyloxyethyl ester, a lactonyl ester optionally selected from the group consisting of a phthalidyl or thiophthalidyl ester, a lower alkoxyacyloxyalkyl ester optionally selected from the group consisting of a methoxycarbonyloxymethyl, ethoxycarbonyloxyethyl or isopropoxycarbonyloxyethyl ester, an alkoxyalkyl ester, choline ester or acylamino alkyl ester optionally selected from the group consisting of an acetamidomethyl ester; or -J-K, wherein J is a covalent bond or a C₁˜C₁₀ straight chain or branched alkyl and K is C₃˜C₁₀ cycloalkyl containing from one to four rings, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R¹⁵ groups,
wherein each R⁹ is independently selected from the group consisting of: H; a C₁˜C₂₀ straight chain or branched acyclic hydrocarbon group containing zero to four C═C or C≡C bonds wherein each C═C bond independently can be of E or Z configuration; a C₁˜C₂₀ straight chain or branched acyl group containing zero to four C═C or C≡C bonds wherein each C═C bond independently can be of E or Z configuration; —(CH₂)_{q}OH, —(CH₂)_{q}OR¹⁴, —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁵ groups; —CH₂CH(OH)CH₂OH; —CH(CH₂OH)₂; —CH₂CH(CH₂OH)₂; lower acyloxy-alkyl optionally selected from the group consisting of acetoxymethyl, acetoxyethyl, aminocarbonyloxymethyl, pivaloyloxymethyl or pivaloyloxyethyl, lactonyl optionally selected from the group consisting of a phthalidyl or thiophthalidyl, lower alkoxyacyloxyalkyl optionally selected from the group consisting of a methoxycarbonyloxymethyl, ethoxycarbonyloxyethyl or isopropoxycarbonyloxyethyl, or acylamino alkyl optionally selected from the group consisting of acetamidomethyl; or -J-K; or —NR⁹₂ can be a cycloamido radical optionally selected from the group consisting of 1-pyrrolidinyl, 1-piperidinyl, 4-morpholinyl, hexahydro-1H-azepin-1-yl, 3-pyrrolin-1-yl, 3,6-dihydro-1(2H)-pyridinyl substituted by one or two R⁹ groups which can be alike or different, or 1-piperazinyl substituted at the 4-position by R⁹, and the like,
wherein each R¹⁰ is independently H; a C₁˜C₂₀ straight chain or branched acyclic hydrocarbon group containing zero to four C═C or C≡C bonds wherein each C═C bond independently can be of E or Z configuration; —(CH₂)_{q}OH, —(CH₂)_{q}OR¹⁴ or —(CH₂)_{q}OC(O)R¹⁴, —(CH₂)_{q}CN, —(CH₂)_{q}CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; or -L-M, wherein L is a covalent bond or a C₁˜C₁₀ straight chain or branched alkyl
wherein each R¹¹ is independently H or —C(O)R¹⁶,
wherein each R¹² is independently R¹⁶ or —C(O)R¹⁶,
wherein each R¹³ is independently a C₁˜C₂₀ straight chain or branched acyclic hydrocarbon group, which can be interrupted by one or more —O— or —S—, said hydrocarbon group containing zero to four C═C or C≡C bonds wherein each C═C bond independently can be of E or Z configuration, said hydrocarbon group having no two heteroatoms adjacent and no heteroatom adjacent to a non-aromatic C═C or C≡C bond, said hydrocarbon group being substituted with zero to four R¹⁷ groups; —(CH₂)_{q}OH, —(CH₂)_{q}OR¹⁴ or —(CH₂)_{q}OC(O)R¹⁴, —(CH₂)_{q}CN, —(CH₂)—CO₂H, —(CH₂)_{q}OC(O)NH₂, or —(CH₂)_{q}C(O)phenyl, where q is an integer from 1 to 6 inclusive and said phenyl is optionally substituted with one to three R¹⁷ groups; -L-M; or -L-O-M (“O” being oxygen),
wherein each R¹⁴ is independently straight chain or branched C₁˜C₆ alkyl or —CH₂OCH₃,
wherein each R¹⁵ is independently straight chain or branched C₁˜C₆ alkyl; straight chain or branched fluoro-substituted C₁˜C₆ alkyl; straight chain or branched fluoro-substituted C₁˜C₆ alkoxy; straight chain or branched C₁˜C₄ alkyl substituted with one, two or three hydroxyl groups; —C(O)OR¹⁶; phenyl; phenyl substituted with one to three R¹⁷; F; Cl; Br; I; CF₃; —C(O)N(R¹⁶)₂; —OR¹⁰; —N(R¹⁶)C(O)OR¹⁶; —N(R¹⁶)C(O)N(R¹⁶)₂; —OC(O)N(R¹⁶)₂; —N(R¹⁶)C(O)R⁵; —N(R¹⁶)₂; —C(O)R⁵; —OC(O)R⁵; —OC(O)OR¹⁶; —C≡N; —N₃; —CF₂OH; —NO₂; —SR¹⁰; —CH═NOR¹⁰; —CH═N—NH—C(O)—NH₂; —C(═O)NR¹¹OR¹²; —S(O)₂N(R¹⁶)₂; —NR¹⁶S(O)₂R¹³; —C(O)NHS(O)₂R¹³; —S(O)₂R¹³; —SO₃H; —PO₃H₂; 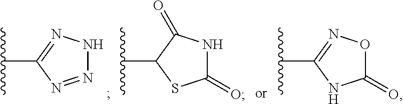
wherein each R¹⁶ is independently H or straight chain or branched C₁˜C₆ alkyl, phenyl or —CH₂OCH₃,
wherein each R¹⁷ is independently straight chain or branched C₁˜C₆ alkyl; —C(O)OR¹⁶; phenyl; F; Cl; Br; I; CF₃; —C(O)N(R¹⁶)₂; —OR¹⁶; —N(R¹⁶)C(O)R¹⁶; —N(R¹⁶)₂; —C(O)R¹⁶; —OC(O)R¹⁶; —C≡N; NO₂; —S(O)₂N(R¹⁶)₂; —NR¹⁶S(O)₂R¹³, with the proviso that, if R¹⁷ is —NR¹⁶S(O)₂R¹³, R¹, R², R³, R^{α} and R^{ω} are selected such that the molecular weight of the compound of Formula I does not exceed about 2000 atomic mass units, and
wherein not more than four of R⁷ are other than H or F and not more than four of R⁷ are F; and
wherein each chiral center or allenic moiety independently possesses any relative or absolute stereoconfiguration or comprises any mixture thereof; and
(ii) a carrier;
(b) topically administering a hair-protective amount of the topical composition onto an epithelial-related surface of a subject in need thereof concurrent with administration of at least one chemotherapeutic agent to the subject;
(c) stimulating hair growth on the epithelial-related surface to which the topical composition has been applied; and
(d) preventing an epithelial-related disorder from occurring on the epithelial surface of step (b),
wherein the epithelial-related disorder is selected from the group consisting of sparse hair growth, short hair growth, thin hair growth, brittle hair growth, alopecia, and hair depigmentation.

42. The method according to claim 41, wherein the at least one compound of Formula I is diastereomerically pure.

43. The method according to claim 41, wherein the at least one compound of Formula I is a mixture of diastereomers in any ratio.

44. The method according to claim 41, wherein the at least one compound of Formula I is enantiomerically pure.

45. The method according to claim 41, wherein the at least one compound of Formula I is a mixture of enantiomers in any ratio, including a racemate.

46. The method according to claim 41, wherein the at least one compound of Formula I is diastereomerically and enantiomerically pure.

47. The method according to claim 41, wherein the at least one compound of Formula I is a mixture of diastereomers and enantiomers in any ratio.

48. The method according to claim 41, wherein the at least one compound of Formula I has one or more hydrogen atoms replaced by deuterium.

49. The method according to claim 41, wherein the optional second component is an imidazole analog, according to Formula III, or a hydrate, solvate, salt, zwitterion, N-oxide, tautomer, prodrug, or metabolite thereof: 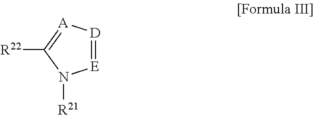
wherein A is N; NR²⁰; NR⁶³ or CH;
wherein D is N or CR²³;
wherein E is N; NR⁶³ or CR²⁴;
with the proviso that, if A is CH, then at least one among D and E is a nitrogen-containing moiety;
with the further proviso that A and E are not simultaneously both NR⁶³;
wherein R²³ is H; F, Cl, Br; I; —NO₂; —N(R⁴²)₂; straight chain or branched C₁˜C₆ alkyl, alkenyl or alkoxy; or phenyl;
wherein R²⁴ is H; F, Cl, Br; I; —NO₂; —N(R⁴₂)₂; straight chain or branched C₁˜C₆ alkyl, alkenyl or alkoxy; or phenyl;
or wherein R²³ and R²⁴ together are —CR⁶²═CR⁶²—CR⁶²═CR⁶²—;
wherein R²⁰ is selected from the group consisting of: H; straight chain or branched C₁˜C₁₂ alkyl; 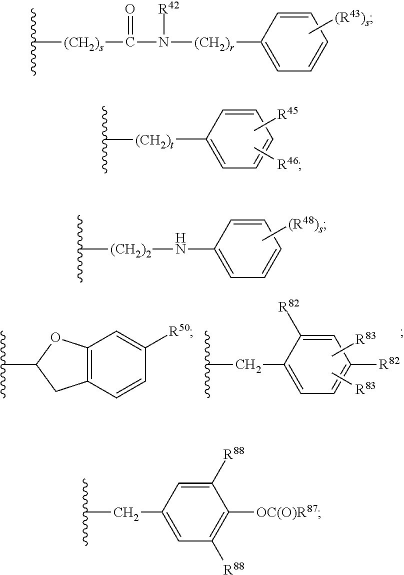
—(CH₂)ₜC(O)R⁴⁴; —CH(R⁴⁷)₂; —(CH₂)ᵤOR⁴⁹; —(CH²)ᵤSR⁴⁹; —CH₂CH(OH)R⁵¹; —CH₂CH═CHR⁵²; or —CH₂C(O)CH₂OR⁵²; and the compound of Formula III is an imidazolium or triazolium salt with a pharmaceutically acceptable counter anion or an internal salt (zwitterion); or
wherein R⁶³ is a moiety that is readily cleaved in vivo, wherein R⁶³ optionally is selected from the group consisting of —CHR⁴²OC(═O)(O)ₙR⁵⁵ or —C(R⁴²)₂OP(═O)(OR¹⁰⁶)₂ and the compound of Formula III is a prodrug and an imidazolium or triazolium salt with a pharmaceutically acceptable counter ion or an internal salt (zwitterion);
wherein s is 1 or 2;
wherein t is an integer from 1 to 4 inclusive;
wherein u is 2 or 3;
wherein R²² is H; F, Cl, Br; I; —NO₂; —N(R⁴²)₂; straight chain or branched C₁˜C₆ alkyl, alkoxy, alkenyl or hydroxyalkyl; phenyl; or a 5˜6-membered aromatic heterocyclic radical containing one or more N, O or S atoms, said heterocyclic radical preferably containing one S and one or two N atoms, said heterocyclic radical optionally selected from the group consisting of thiazolyl or thiazol-4-yl;
wherein R²¹ is H; or —(CR²⁵R²⁶)ₘ—CR²⁷R²⁸-Q-R²⁹;
wherein each m is independently an integer from 0 to 4 inclusive;
wherein R²⁵ is H; —C≡N; straight chain or branched C₁˜C₁₂ acyclic hydrocarbon group, said hydrocarbon group optionally containing one or more C═C or C≡C bonds; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;
wherein R²⁶ is H; or straight chain or branched C₁˜C₆ alkyl;
wherein R²⁷ is -T-U—V; —O—(CH₂)ᵣ-L; —NH—(CH₂)ᵣ-L; —S(O)ᵣ—R⁵⁵; —OR⁵³; —OR⁶⁵; —CF(R⁶⁷)R⁵⁶; —CF(R⁶⁷)C(O)R⁶⁸; —CF(R⁶⁷)C(O)N(R⁶⁸)₂; —CF(R⁶⁷)C(O)N(R⁶⁸)N(R⁶⁸)₂; —CF(R⁶⁷)C(O)N(R⁶⁸)OR⁶⁸; —CF(R⁶⁷)C(O)N(R⁶⁸)OH; —CF(R⁶⁷)C(═NH)R⁶⁸; —CF(R⁶⁷)C(═NH)N(R⁶⁸)₂; —CF(R⁶⁷)C(═NH)N(R⁶⁸)N(R⁶⁸)₂; —CF(R⁶⁷)C(═NH)N(R⁶⁸)OR⁶⁸; —CF(R⁶⁷)C(═NH)N(R⁶⁸)OH; or —CF(R⁶⁷)C(═N—OR⁶⁹)NH₂;
wherein each r is independently an integer from 0 to 2 inclusive;
wherein each L is independently α-tetralyl; or phenyl, said phenyl being optionally substituted with up to three groups selected from the group consisting of: straight chain or branched C₁˜C₆ alkyl, straight chain or branched C₁˜C₆ alkoxy, —C≡N, —NO₂, or —NH₂; wherein R²⁸ is H; F; Cl; Br; —OR⁴²; —OC(═O)R¹⁰⁰; 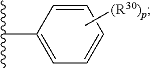
straight chain or branched C₁˜C₆ alkyl; R61; or —OP(═O)(OH)₂ or a pharmaceutically acceptable salt or zwitterion form thereof;
wherein each Q is independently a covalent bond; —O—; —S—; —S(O)—; or —S(O)₂—;
wherein R²⁹ is C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups; straight chain or branched C₁˜C₆ alkyl optionally substituted with —CO₂H; or 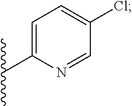
wherein each p is independently an integer from 0 to 3 inclusive;
wherein each T is independently a covalent bond; —CH₂CH₂—; —CH═CH—; —(O)ₙC(R⁴²)₂—; —CH₂O—; —SCH₂—; —CH₂S—; —OCH₂CH₂O—; —CH(CH₃)—; —CH₂—; —CF₂—; —C(R⁹³)₂—; or 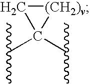
wherein each v is independently an integer from 1 to 3 inclusive;
wherein each U is independently a covalent bond; 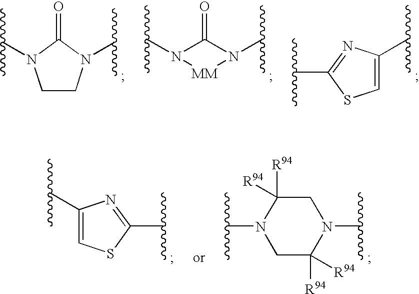
wherein each V is independently H; —S(O)₂CH₃; —C(O)NH₂; —CH₂C≡CH; —CH═CH₂; —S(O)ᵣ—R⁵⁵; 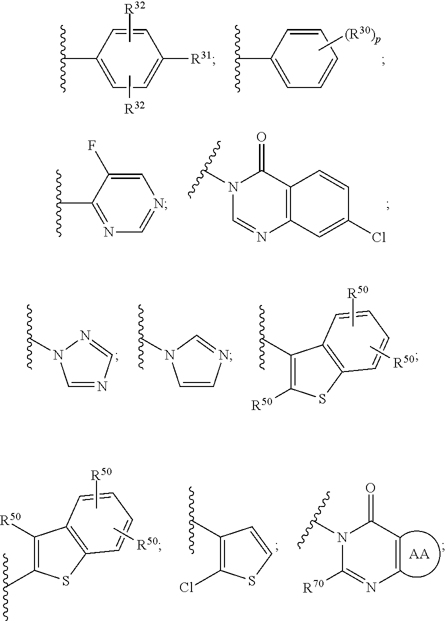
—R⁵⁹; —R⁶¹; or -LL-R⁹⁵;
wherein AA represents a benzene ring or a 5- or 6-membered heterocyclic ring wherein one or more of the ring atoms are selected from the group consisting of N, O and S, which rings can be optionally fused to a benzene ring or to a 5- or 6-membered heterocyclic ring containing one or more heteroatoms selected from N, O and S and wherein AA can be unsubstituted or have 1, 2, 3 or 4 substituents R⁷¹ in any of the rings;
wherein each R³⁰ is independently F; Cl; Br; I; —NO₂; —C≡N; —S—C≡N; —NR³⁹R⁴⁰; —OR⁴¹; —OR⁵⁴; —OH; —OC(O)R⁵⁴; —C(O)R⁵⁴; —C(O)OR⁵⁴; —C(O)OH; —N(R⁵⁴)C(O)OR⁵⁴; —OC(O)N(R⁵⁴)₂; —SH; —SR⁴¹; —S(O)ᵣR⁵⁸; —CH═CH—CO₂H; 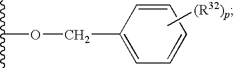
straight chain or branched C₁˜C₁₂ alkyl; straight chain or branched C₁˜C₁₀ alkoxy; methylenedioxy; straight chain or branched C₁˜C₆ cyanoalkyl exemplified by, but not limited to —CH₂C≡N, —CH₂CH₂C≡N, —CH₂CH₂CH₂C≡N, and —CH₂CH(CH₃)C≡N; straight chain or branched C₁˜C₆ halogenoalkyl optionally selected from the group consisting of —CF₃, —CF₂CF₃, —CH₂CF₃, —CHF₂, —CH₂F, —CH₂Cl, and —CH₂Br; straight chain or branched C₁˜C₅ halogenoalkoxy optionally selected from the group consisting of —OCF₃, —OCF₂CF₃, —OCH₂CF₃, —OCHF₂, and —OCH₂F; straight chain or branched C₁˜C₆ alkylthio or haloalkylthio; straight chain or branched C₁˜C₆ alkylthionyl or haloalkylthionyl; or straight chain or branched C₁˜C₆ alkylsulfonyl or haloalkylsulfonyl; C₃˜C₇ cycloalkyl, C₆˜C₁₄ aryl containing one, two or three rings, or C₆˜C₁₄ aryloxy containing one, two or three rings, said cycloalkyl, aryl or aryloxy being optionally substituted with one to three moieties independently selected from F, Cl, Br, I, —NO₂, or straight chain or branched C₁˜C₆ alkyl, halogenoalkyl or alkoxy; straight chain or branched C₁˜C₄ alkyl substituted with C₃˜C₆ cycloalkyl or C₆˜C₁₄ aryl containing one, two or three rings, said cycloalkylalkyl or arylalkyl being optionally substituted with one to three moieties independently selected from F, Cl, Br, I, —NO₂, or straight chain or branched C₁˜C₆ alkyl, halogenoalkyl or alkoxy;
wherein each R³¹ is independently H; F; Cl; Br; I; —C≡N; —NO₂; —CF₃; —N═C═S; —N(R⁴²)₂; —NH—C(═O)-(M)ₙ-R⁵⁴; —NH—C(═S)—NH—R⁵⁴; straight chain or branched C₁˜C₆ alkoxy or alkylthio; straight chain or branched C₁˜C₁₂ alkyl; —(CH₂)ᵣ—R⁹⁹; 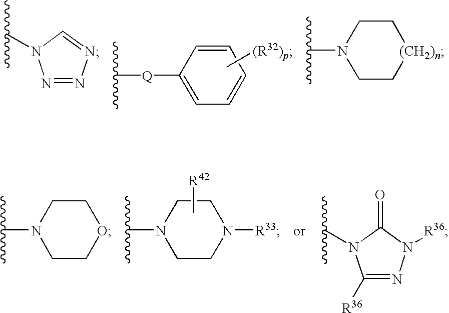
wherein each M is independently O or NH, with the proviso that when M is O and n is 1, R⁵⁴ is other than H;
wherein each R³² is independently H; F; Cl; Br; I; —C≡N; —NO₂; straight chain or branched C₁˜C₁₂ alkyl; straight chain or branched C₁˜C₄ alkoxy; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; straight chain or branched C₁˜C₃ halogenoalkoxy optionally selected from the group consisting of —OCF₃, —OCF₂CF₃, —OCH₂CF₃, —OCHF₂, —OCH₂F; straight chain or branched C₁˜C₄ alkylthio or haloalkylthio; straight chain or branched C₁˜C₄ alkylthionyl or haloalkylthionyl; or straight chain or branched C₁˜C₄ alkylsulfonyl or haloalkylsulfonyl;
wherein each R³³ is independently H; straight chain or branched C₁˜C₆ alkyl, wherein said alkyl is optionally substituted with —OH or —OR³⁴; —S(O)₂R³⁴; —S(O)₂—CH₂-phenyl; —C(O)R⁴²; —(CH₂)—C(O)OR³⁴; —C(O)O-phenyl; —(CH₂)ₙC(O)N(R⁴²)₂; —CH₂C(S)N(R⁴²)₂; —CH₂C(S)SR³⁴; —(CH₂)n-phenyl; benzoyl, wherein said benzoyl is optionally substituted with one or two R⁴⁶ groups; or 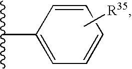
or, preferably, 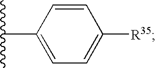
wherein each R³⁴ is independently straight chain or branched C₁˜C₆ alkyl;
wherein each R³⁵ is independently H; 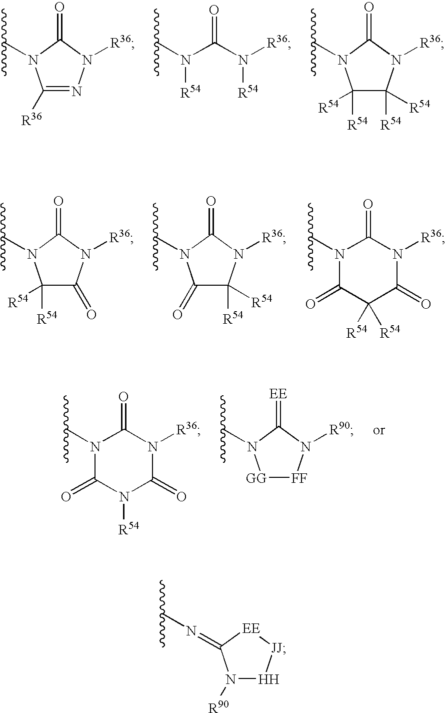
wherein each R³⁶ is independently H; straight chain or branched C₃˜C₇ alkenyl, with the proviso that the olefinic double bond is not located a to N; straight chain or branched C₃˜C₇ alkynyl, with the proviso that the alkynyl C≡C bond is not located α to N; C₃˜C₁₀ cycloalkyl, said cycloalkyl being optionally substituted with one to three R³⁰ groups; C₆˜C₁₄ aryl containing one, two or three rings, said aryl being optionally substituted with one to three R³⁰ groups; straight chain or branched C₁˜C₈ alkyl, said alkyl being optionally substituted with one to three R³⁰ groups; or 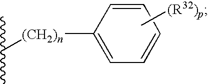
wherein each n is independently 0 or 1;
wherein R²⁶ and R²⁸ together with the two carbon atoms to which they attach can be C═C; wherein R²⁷ and R²⁸ together can be 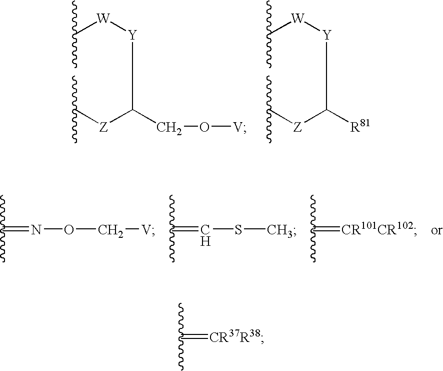
wherein W is —CH₂— and Y is —CH₂—; W is —O— and Y is —CH₂—; or W is —CH₂— and Y is —O—;
wherein Z is —CH₂— or —O—;
wherein R²⁷ together with -Q-R²⁹ can be 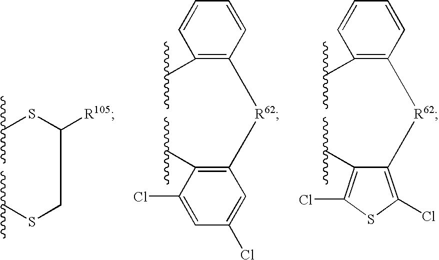
or, when R²⁷ and R²⁹ each independently represents a cycloalkyl, aryl or heterocyclic ring and Q is a covalent bond, R²⁷ together with -Q-R²⁹ can be 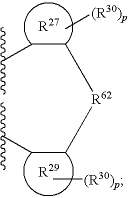
wherein each R³⁷ is independently H; C₁˜C₂ alkyl or phenyl;
wherein each R³⁸ is independently H or C₁˜C₂ alkyl;
wherein each R³⁹ is independently H; straight chain or branched C₁˜C₅ alkyl; straight chain or branched C₁˜C₅ alkanoyl; or straight chain or branched C₁˜C₅ haloalkanoyl optionally selected from the group consisting of —C(O)CF₃, —C(O)CF₂CF₃, —C(O)CH₂CF₃, —C(O)CHF₂, and —C(O)CH₂F;
wherein each R⁴⁰ is independently H; straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;
wherein each R⁴¹ is independently straight chain or branched C₁˜C₅ alkyl; C₃˜C₁₀ dialkylaminoalkyl; C₁₅˜C₂₀ dibenzylaminoalkyl; 1-pyrrolidinyl; 2-imidazolin-2-yl; or —(CH₂)q-G-J;
wherein each q is independently an integer from 0 to 4 inclusive;
wherein each G¹ is independently a covalent bond; —O—; or —S—;
wherein each J is independently phenyl, 2-thienyl or 3-thienyl wherein said phenyl, 2-thienyl or 3-thienyl is optionally substituted with one to three groups selected from the group consisting of:
F; Cl; Br; I; —NO₂; straight chain or branched C₁˜C₅ alkyl; straight chain or branched C₁˜C₅ alkoxy;
wherein each R⁴² is independently H; or straight chain or branched C₁˜C₆ alkyl;
wherein each R⁴³ is independently H; F; Cl; Br; I; —NO₂; straight chain or branched C₁˜C₆ alkyl;
straight chain or branched C₁˜C₆ alkoxy; —CF₃; or —O— phenyl;
wherein each R⁴⁴ is independently 2-thienyl or 3-thienyl, wherein said 2-thienyl or 3-thienyl is optionally substituted with F, Cl, Br, or I; or phenyl, wherein said phenyl is optionally substituted with one to two identical or different groups selected from the group consisting of: F, Cl, Br, I, straight chain or branched C₁˜C₆ alkyl, or straight chain or branched C₁˜C₆ alkoxy;
wherein each R⁴⁵ is independently H; F; Cl; Br; I; —NO₂; straight chain or branched C₁˜C₆ alkyl; straight chain or branched C₁˜C₆ alkoxy; or —S(O)₂NH₂;
wherein each R⁴⁶ is independently H; F; Cl; Br; I; straight chain or branched C₁˜C₆ alkyl; straight chain or branched C₁˜C₆ alkoxy; or —C≡N;
wherein each R⁴⁷ is independently a phenyl group optionally substituted with one to three F, Cl, Br or I;
wherein each R⁴⁸ is independently H; F; Cl; Br; I; or straight chain or branched C₁˜C₆ alkyl;
wherein each R⁴⁹ is independently 1-naphthyl; 2-naphthyl; or phenyl, wherein said phenyl is optionally substituted with one or two groups, each independently selected from the group consisting of: F, Cl, Br, I, straight chain or branched C₁˜C₆ alkyl, straight chain or branched C₁˜C₆ alkoxy, or —CH₂CH═CH₂;
wherein each R⁵⁰ is independently H; F; Cl; Br; or I;
wherein each R⁵¹ is independently phenyl, wherein said phenyl is optionally substituted with one or two groups, each independently selected from the group consisting of: F, Cl, Br, I, or straight chain or branched C₁˜C₆ alkyl;
wherein each R⁵² is independently phenyl, wherein said phenyl is optionally substituted with one or two groups, each independently selected from the group consisting of: F, Cl, Br, or I;
wherein each R⁵³ is independently straight chain or branched C₁˜C₈ alkyl optionally substituted with C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; straight chain or branched C₃—O₅ alkenyl optionally substituted with a phenyl bearing one or more halogen substituents, wherein the olefinic double bond is located β, γ or δ with respect to the ether oxygen; straight chain or branched C₃˜C₅ alkynyl, wherein the alkynyl C≡C bond is located β, γ or δ with respect to the ether oxygen;
wherein each R⁵⁴ is independently H; straight chain or branched C₁˜C₆ alkyl, said alkyl being optionally substituted with one or two moieties selected from the group consisting of F, Cl, Br, and I; C₆˜C₁₄ aryl, said aryl being optionally substituted with one or two moieties independently selected from the group consisting of F, Cl, Br, I, straight chain or branched C₁˜C₆ alkyl, and straight chain or branched C₁˜C₆ alkoxy; C₇˜C₁₉ aralkyl; C₃˜C₁₀ cycloalkyl; or —CH₂R⁶⁴, wherein said R⁶⁴ is straight chain or branched C₂˜C₄ alkenyl or alkynyl;
wherein each R⁵⁵ is independently C₃˜C₁₀ cycloalkyl, said cycloalkyl being optionally substituted with one to three R⁵⁶ groups; C₆˜C₁₄ aryl containing one, two or three rings, said aryl being optionally substituted with one to three R⁵⁶ groups; 3˜10-membered heterocycle containing one or two rings and one or more N, O or S atoms, wherein such heterocycle can be aromatic or non-aromatic, said heterocycle being optionally substituted with one to three R⁵⁶ groups; straight chain or branched C₁˜C₂₀ alkyl; straight chain or branched C₂˜C₁₂ alkenyl; straight chain or branched C₂˜C₄ alkenyl substituted with phenyl or 1- or 2-naphthyl wherein said phenyl or naphthyl is optionally substituted with one to three R⁵⁶ groups; straight chain or branched C₂˜C₁₂ alkynyl; straight chain or branched C₁˜C₄ alkyl substituted with C₃˜C₈ cycloalkyl, C₆˜C₁₄ aryl containing one, two or three rings or 3˜10-membered heterocycle containing one or two rings and one or more N, O or S atoms, wherein such heterocycle can be aromatic or non-aromatic, said cycloalkyl, aryl or heterocycle being optionally substituted with one to three R⁵⁶ groups; phenyl or 1- or 2-naphthyl wherein said phenyl or naphthyl is optionally substituted with one to three R⁵⁷ groups;
wherein each R⁵⁶ is independently F; Cl; Br; I; straight chain or branched C₁˜C₆ alkyl; straight chain or branched C₁˜C₆ alkoxy; straight chain or branched C₁˜C₆ alkylthio, alkylthionyl or alkylsulfonyl; C₃˜C₁₀ cycloalkyl; C₃˜C₁₀ cycloalkyloxy; C₃˜C₁₀ cycloalkylamino; C₃˜C₁₀ cycloalkylthio, cycloalkylthionyl or cycloalkylsulfonyl; C₆˜C₁₄ aryl; C₆˜C₁₄ aryloxy; C₆˜C₁₄ arylamino; C₆˜C₁₄ arylthio, arylthionyl or arylsulfonyl; C₇˜C₁₉ aralkyl; C₇˜C₁₉ aralkyloxy; C₇˜C₁₉ aralkylamino; C₇˜C₁₉ aralkylthio, aralkylthionyl or aralkylsulfonyl; 3˜10-membered heterocycle containing one or two rings and one or more N, O or S atoms, wherein such heterocycle can be aromatic or non-aromatic; 3˜10-membered heterocycle-oxy, heterocycle-amino, heterocycle-thio, heterocycle-thionyl, or heterocycle-sulfonyl, wherein said heterocycle contains one or two rings and one or more N, O or S atoms, wherein said heterocycle can be aromatic or non-aromatic; methylenedioxy; —C≡N; —NO₂; —CF₃; —N(R⁵⁴)₂; —OR⁵⁴; —SH; ═O; —C(O)R⁵⁴; —C(O)OR⁵⁴; —OC(O)R⁵⁴; —C(O)N(R⁵⁴)₂; —N(R⁵⁴)C(O)R⁵⁴; —N(R⁵⁴)C(O)OR⁵⁴; —OC(O)N(R⁵⁴)₂; —N(R⁵⁴)C(O)N(R⁵⁴)₂; —OC(O)OR⁵⁴; —C(O)N(R⁵⁴)N(R⁵⁴)₂; —C(O)N(R⁵⁴)OR⁵⁴; —C(O)N(R⁵⁴)OH; ═S; —C(S)R⁵⁴; —C(S)OR⁵⁴; —OC(S)R⁵⁴; —C(S)N(R⁵⁴)₂; —N(R⁵⁴)C(S)R⁵⁴; —N(R⁵⁴)C(S)OR⁵⁴; —OC(S)N(R⁵⁴)₂; —N(R⁵⁴)C(S)N(R⁵⁴)₂; —C(═NH)R⁵⁴; —C(═NH)N(R⁵⁴)₂; —C(═NH)N(R⁵⁴)N(R⁵⁴)₂; —C(═NH)N(R⁵⁴)OR⁵⁴; —C(═NH)N(R⁵⁴)OH; —C(═N—OR⁵⁴)NH₂; —S(O)₂N(R⁵⁴)₂; —NR⁵⁴S(O)₂R⁵⁴; —C(O)NHS(O)₂R⁵⁴; —S(O)₂R⁵⁴; —SO₃H; or —PO₃H₂;
wherein each R⁵⁷ is independently F; Cl; Br; I; straight chain or branched C₁˜C₆ alkyl; straight chain or branched C₁˜C₆ alkoxy; —C≡N; —CF₃; NO₂; —NH₂; or —NHC(O)R⁶⁶, wherein said R⁶⁶ is straight chain or branched C₂˜C₁₂ alkyl;
wherein each R⁵⁸ is independently straight chain or branched C₁˜C₂₀ alkyl; C₃˜C₈ cycloalkyl; straight chain or branched C₁˜C₄ alkyl substituted with phenyl or 1- or 2-naphthyl wherein said phenyl or naphthyl is optionally substituted with one to three R⁵⁶ groups; or phenyl or 1- or 2-naphthyl wherein said phenyl or naphthyl is optionally substituted with one to three R⁵⁷ groups;
wherein each R⁵⁹ is independently straight chain or branched C₁˜C₇ alkyl, alkenyl or alkynyl, said alkyl, alkenyl or alkynyl being substituted with one or more —R⁶⁰— R⁶¹;
wherein each R⁶⁰ is independently a covalent bond or —O—;
wherein each R⁶¹ is independently C₃˜C₇ cycloalkyl, C₆˜C₁₀ aryl containing one or two rings or 3˜10-membered heterocycle containing one or two rings and one or more N, O or S atoms, wherein such heterocycle can be aromatic or non-aromatic, said cycloalkyl, aryl or heterocycle being optionally substituted with one to three R³⁰ groups;
wherein each R⁶² is independently a covalent bond; —CH₂—CH₂—; —CH═CH—; —O—; or —S—;
wherein each R⁶⁵ is independently straight chain or branched C₁˜C₁₂ acyclic hydrocarbon group, which can be interrupted by one or more —O—, —S—, —S(O)—, —S(O)₂—, said hydrocarbon group optionally containing one or more C═C or C≡C bonds, said hydrocarbon group having no two heteroatoms adjacent and no heteroatom adjacent to a non-aromatic C═C or C≡C bond; C₃˜C₁₀ cycloalkyl or cycloalkenyl; C₄˜C₁₂ cycloalkylalkyl; C₆˜C₁₂ cycloalkenylalkyl; C₆˜C₁₄ aryl containing one, two or three rings, said aryl being optionally substituted with one to three R³⁰ groups; or straight chain or branched C₁˜C₆ alkyl substituted with C₆˜C₁₄ aryl containing one, two or three rings, said aryl being optionally substituted with one to three R³⁰ groups;
wherein each R⁶⁷ is independently H; F; or straight chain or branched C₁˜C₆ alkyl, said alkyl group being optionally substituted by one or more R³⁰;
wherein each R⁶⁸ is independently R³⁶, R⁵⁴ or R⁶¹;
wherein each R⁶⁹ is independently H; or straight chain or branched C₁˜C₆ alkyl, said alkyl group being optionally substituted by one or more R³⁰;
wherein each R⁷⁰ is independently H; straight chain or branched C₁˜C₄ alkyl; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; or cyclopropyl;
wherein each R⁷¹ is independently R⁵⁶; straight chain or branched C₁˜C₄ halogenoalkyl optionally selected from the group consisting of —CF₃, —CF₂CF₃, —CH₂CF₃, —CHF₂, —CH₂F, —CH₂Cl, —CH₂Br; hydroxymethyl; —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²; and additionally one of the R⁷¹ groups can also represent 1-pyrrolyl; 1-imidazolyl; 1H-1,2,4-triazol-1-yl; 5-tetrazolyl (optionally substituted with straight chain or branched C₁˜C₄ alkyl); 1-pyrrolidinyl; 4-morpholinyl; 4-morpholinyl-N-oxide; —OR⁷⁶; —S(O)ᵣR⁷⁶; —N(R⁷²)R⁷⁶; —C(O)R⁷⁶; 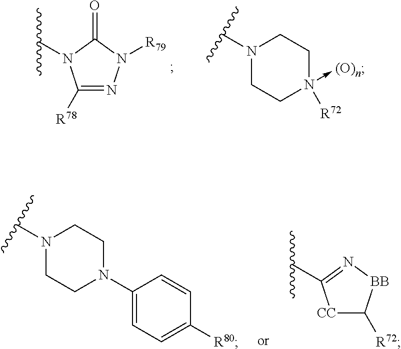
wherein each R⁷² is independently H; straight chain or branched C₁˜C₄ alkyl; C₃˜C₆ cycloalkyl; or straight chain or branched C₁˜C₄ alkyl substituted with phenyl, said phenyl being optionally substituted with one or more halogen, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;
wherein each R⁷³ is independently H; straight chain or branched C₁˜C₄ alkyl; C₃˜C₆ cycloalkyl; —C(O)R⁷²; or —C(O)CF₃;
wherein each R⁷⁴ is independently straight chain or branched C₁˜C₄ alkyl;
wherein each R⁷⁵ is independently H; —C(O)NH₂; —C(O)CH₃; —C≡N; —SO₂NHR⁷²; —SO₂R⁷²; —OR⁷²; —OC(O)R⁷²; or straight chain or branched —(C₁˜C₄ alkyl)-NH₂;
wherein each R⁷⁶ is independently phenyl optionally substituted with one or more R⁷⁷ groups;
wherein each R⁷⁷ is independently straight chain or branched C₁˜C₄ alkyl; C₃˜C₆ cycloalkyl; straight chain or branched C₁˜C₄ halogenoalkyl; straight chain or branched C₁˜C₄ alkoxy; straight chain or branched C₁˜C₄ halogenoalkoxy; F; Cl; Br; I; —C≡N; —NO₂, —NR⁷²R⁷³; —C(O)NR⁷²R⁷³; —CH₂OC(O)R⁷²; —C(O)R⁷²; —C(O)OR⁷²; —S(O)ᵣR⁷⁴; —C(═NR⁷²)NHR⁷⁵; —C(═NR⁷⁵)OR⁷²; 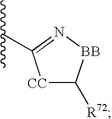
or phenyl, said phenyl being optionally substituted with one or more F, Cl, Br, I, —C≡N, —NO₂, straight chain or branched C₁˜C₄ alkyl, straight chain or branched C₁˜C₄ halogenoalkyl, straight chain or branched C₁˜C₄ alkoxy, or straight chain or branched C₁˜C₄ halogenoalkoxy;
wherein each R⁷⁸ is independently H or —CH₃;
wherein each R⁷⁹ is independently H; isopropyl; cyclopentyl; cyclopropyl; 2-butyl; 3-pentyl; 3-hydroxy-2-butyl; or 2-hydroxy-3-pentyl;
wherein each R⁸⁰ is independently F; Cl; Br; I; —C≡N; —NO₂; —NH₂; straight chain or branched C₁˜C₄ halogenoalkyl; straight chain or branched C₁˜C₄ halogenoalkoxy; or 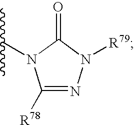
wherein each R⁸¹ is independently alkyloxymethyl wherein said alkyl group is straight chain or branched and has from 1 to 10 carbon atoms; alkenyl or alkenyloxymethyl wherein said alkenyl group is straight chain or branched and has from 2 to 10 carbon atoms; hydroxymethyl; 2-propynyloxymethyl; halomethyl; arylmethyl and aryloxymethyl wherein said aryl is phenyl, substituted phenyl, naphthalenyl or mono- or di-halo-naphthalenyl and wherein said substituted phenyl is phenyl having from 1 to 3 substituents independently selected from the group consisting of halogen, straight chain or branched C₁˜C₆ alkyl, straight chain or branched C₁˜C₆ alkoxy, —C≡N, —NO₂, phenyl, phenylmethyl, benzoyl, halobenzoyl, —C(O)R⁴², —C(O)OR⁴², and —CF₃, with the proviso that when multiple substituents are present only 1 thereof can be selected from the group consisting of phenyl, phenylmethyl, benzoyl and halobenzoyl;
wherein each R⁸² is independently H or —OR⁸⁴;
wherein each R⁸³ is independently H; F; Cl; Br; R⁷⁴; —OR⁷⁴; —SR⁷⁴; —CH₂SC(═O)R⁷⁴; —COOH; or —C(═O)OCH₃;
wherein each R⁸⁴ is independently a group that is easily hydrolyzable under physiological conditions, optionally at the acyl residue of an amino acid or a group represented by the formula —C(═O)R⁸⁵ or —P(═O)(OR⁸⁶)₂; wherein R⁸⁴ is optionally selected from the group consisting of formyl, acetyl, propionyl, isobutyryl, pivaloyl, succinoyl, benzoyl, nicotinyl, phosphoryl, dimethylphosphoryl, aminoacetyl, 3-aminopropionyl, 4-aminobutyryl, (2-amino-acetylamino)-acetyl, (S)-2,5-diaminopentoyl, (S)-2-aminopropionyl, (S)-pyrrolidine-2-carbonyl, (methylamino)acetyl, (propylamino)acetyl, (S)-2-(methylamino)propionyl, 3-(methylamino)propionyl, (S)-2-amino-3-methylbutanoyl, (isopropylamino)acetyl, (2S)-2-(ethylamino)propionyl, (ethylamino)acetyl and the like;
wherein each R⁸⁵ is independently H; —OR⁷⁴; or straight chain or branched C₁˜C₆ alkyl or alkenyl, wherein said alkyl or alkenyl can be optionally interrupted by a hydrolyzable functional group such as carboxamide or ester, wherein said alkyl or alkenyl can be optionally substituted with —COOH, —NH₂, —NHR⁷⁴, —N(R⁷⁴)₂, or R⁶¹, wherein R⁶¹ is optionally selected from the group consisting of phenyl, methoxyphenyl, pyridyl, pyrazinyl or furyl;
wherein each R⁸⁶ is independently H or R⁷⁴;
wherein each R⁸⁷ is independently straight chain or branched C₁˜C₅ alkyl; R⁶¹; pyridyl; pyrrolidinyl; or -DD-NH—R⁸⁹;
wherein each R⁸⁸ is independently H; F; Cl; Br; or —OR⁷⁴;
wherein each DD is independently straight chain or branched C₁˜C₄ alkylene; —CH₂NHC(═O)CH₂—; —CH(NH₂)CH₂CH₂CH₂—;
wherein each R⁸⁹ is independently H or straight chain or branched C₁˜C₅ alkyl;
wherein each BB is independently —CH₂— or —C(═O)—;
wherein each CC is independently NH or O;
wherein each EE is independently O, S or N—R⁹¹;
wherein each R⁹⁰ is independently tetrahydrofuranyl-(C₁˜C₆ alkyl); or C₁˜C₆ alkyl, C₃˜C₆ cycloalkyl, phenyl-(C₁˜C₆ alkyl) or (C₃˜C₆ cycloalkyl)-(C₁˜C₆ alkyl) all substituted on the C₁˜C₆ alkyl and/or C₃˜C₆ cycloalkyl moiety with ═O, ═S or with one or two radicals of formula KK—R⁹²; wherein said tetrahydrofuranyl is tetrahydrofuran-2-yl or tetrahydrofuran-3-yl; wherein said phenyl is optionally substituted with one to three substituents each independently selected from the group consisting of F, Cl, Br, I, —C≡N, —NO₂, —NH₂, C₁˜C₆ alkyl, C₁˜C₆ alkoxy, and —CF₃; and wherein all said C₁˜C₆ alkyl moieties are straight chain or branched;
wherein each KK is independently O or S;
wherein each R⁹¹ is independently H or straight chain or branched C₁˜C₆ alkyl;
wherein each R⁹² is independently H; straight chain or branched C₁˜C₆ alkyl; C₃˜C₆ cycloalkyl; tetrahydro-2H-pyran-2-yl; phenyl, wherein said phenyl is optionally substituted with one to three substituents each independently selected from the group consisting of F, Cl, Br, I, —C≡N, —NO₂, —NH₂, straight chain or branched C₁˜C₆ alkyl, straight chain or branched C₁˜C₆ alkoxy, and —CF₃; or where R⁹⁰ is substituted with two KK—R⁹² radicals, two R⁹² radicals, taken together, may form a bivalent radical of formula —CH₂—, —CH(CH₃)—, —C(CH³)₂—, —CH₂CH₂—, —CH(CH₃)CH₂—, —CH₂CH₂CH₂—;
wherein each FF is independently —C(═O)—; NR⁹¹; or —CH₂—, optionally substituted with up to two radicals selected from the group consisting of straight chain or branched C₁˜C₆ alkyl and phenyl, said phenyl being optionally substituted with one to three substituents each independently selected from the group consisting of F, Cl, Br, I, —C≡N, —NO₂, —NH₂, C₁˜C₆ alkyl, C₁˜C₆ alkoxy, and —CF₃;
wherein each GG is independently —C(═O)—; or —CH₂—, optionally substituted with up to two radicals selected from the group consisting of straight chain or branched C₁˜C₆ alkyl and straight chain or branched C₁˜C₆ alkoxy;
or FF and GG, taken together, form a bivalent radical of formula —N═CH—(FFGG′);
wherein HH has the same meaning as FF;
wherein JJ has the same meaning as GG;
or HH and JJ, taken together, form a bivalent radical of formula —N═CH—(FFGG′) or —CH═CH—(HHJJ′);
wherein the nitrogen atom in the bivalent radical FFGG′ is connected to NR⁹⁰; wherein one hydrogen in said radical FFGG′ and up to two hydrogens in radical HHJJ′ can be replaced by a straight chain or branched C₁˜C₆ alkyl radical;
wherein each R⁹³ is independently H or C₁˜C₄ alkyl which is unsubstituted or substituted by 1-3 substituents each independently selected from the group consisting of halogen, hydroxyl, C₁˜C₄ alkoxy, and amino; or two R⁹³ groups attached to the same carbon atom together form a lower alkylene group optionally selected from the group consisting of —CH₂CH₂—, —CH₂CH₂CH₂—, —CH₂CH₂CH₂CH₂— and the like;
wherein each R⁹⁴ is independently H or C₁˜C₄ alkyl which is unsubstituted or substituted by 1-3 substituents each independently selected from the group consisting of halogen, hydroxyl, C₁˜C₄ alkoxy, and amino; or two R⁹⁴ groups attached to the same carbon atom together form ═S;
wherein each LL is independently a covalent bond; —C(═O)—; —C(═S)—; —SO₂—; or —N═N—;
wherein each R⁹⁵ is independently selected from the group consisting of
i) hydrogen,
ii) CN
iii) CHO iv) phenyl which is unsubstituted or substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) C₁˜C₄ alkoxy, (3) halogen, (4) formyl, (5) carboxyl, (6) C₁˜C₄ acyloxy, (7) C₁˜C₄ alkoxycarbonylamino, (8) phenyl- or naphthyl-oxycarbonylamino, (9) semicarbazido, (10) formamido, (11) thioformamido, (12) hydroxy, (13) nitro, (14) amino, (15) furyl, (16) triazolyl, (17) thienyl, (18) oxazolyl, (19) imidazolyl and (20) triazolone-yl,
v) a 5- or 6-membered monocyclic or 8- to 10-membered bicyclic heterocycle having 1-4 heteroatoms each independently selected from the group consisting of N, O and S, which heterocycle is unsubstituted or ring-substituted with 1-3 substituents each independently selected from the group consisting of (1) C₁˜C₄ alkyl which is unsubstituted or substituted with 1-3 substituents each independently selected from the group consisting of halogen, hydroxy, C₁˜C₄ alkoxy and amino, (2) benzyl which is unsubstituted or substituted with 1-3 substituents selected from the group consisting of C₁˜C₄ alkyl, CF₃, halogen and OCF₃, (3) halogen, (4) hydroxy, (5) nitro, (6) amino, (7) C₁˜C₄ acylamino, (8) formyl, (9) formamido, (10) thioformamido, (11) C₁˜C₄ alkoxycarbonylamino, (12) phenyl- or naphthyl-oxycarbonylamino and (13) semicarbazido,
vi) NHR⁹⁶ wherein R⁹⁶ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl, and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of hydroxy, halogen, amino and carboxyl,
vii) OR⁹⁷ wherein R⁹⁷ is selected from the group consisting of (1) C₁˜C₄ alkyl which is unsubstituted or substituted with 1-3 substituents each independently selected from the group consisting of halogen, hydroxy, C₁˜C₄ alkoxy and amino, (2) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl, (e) carboxyl, (f) C₁˜C₄ acyloxy, (g) C₁˜C₄ alkoxycarbonylamino, (h) phenyl- or naphthyloxycarbonylamino, (i) semicarbazido, (j) formamido, (k) thioformamido, (l) hydroxy, (m) nitro, (n) amino, (o) furyl, (p) triazolyl, (q) thienyl, (r) oxazolyl, (s) imidazolyl and (t) triazolone-yl and (3) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8 to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, and
viii) a group of the formula 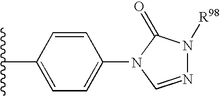
wherein R⁹⁸ is selected from the group consisting of (1) hydrogen, (2) C₁˜C₁₀ alkyl which is unsubstituted or substituted by 1-5 substituents each independently selected from the group consisting of halogen, hydroxy, C₁˜C₄ alkoxy and amino, (3) phenyl which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) C₁˜C₄ alkoxy, (c) halogen, (d) formyl. (e) carboxyl, (f) C₁˜C₄ acyloxy, (g) C₁˜C₄ alkoxycarbonylamino, (h) phenyl- or naphthyl-oxycarbonylamino, (i) semicarbazido, (j) formamido, (k) thioformamido, (l) hydroxy, (m) nitro, (n) amino, (O) furyl, (p) triazolyl, (q) thienyl, (r) oxazolyl, (s) imidazolyl, (t) triazolone-yl, (u) CF₃ and (v) OCF₃, (4) a 5- or 6-membered monocyclic or 8- to 10-membered bicyclic heterocycle having 1-3 heteroatoms each independently selected from the group consisting of N, O and S, which is unsubstituted or substituted with 1-3 substituents each independently selected from the group consisting of (a) C₁˜C₄ alkyl which is unsubstituted or substituted with 1-3 substituents each independently selected from the group consisting of halogen, hydroxy, C₁˜C₄ alkoxy and amino, (b) phenyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyloxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (c) naphthyl which is unsubstituted or substituted with 1-3 substituents each independently selected from the group consisting of (A) C₁˜C₄ alkyl which is unsubstituted or substituted with 1-3 substituents each independently selected from the group consisting of halogen, hydroxy, C₁˜C₄ alkoxy and amino, (B) C₁˜C₄ alkoxy, (C) halogen, (D) formyl, (E) carboxyl, (F) C₁˜C₄ acyloxy, (G) C₁˜C₄ alkoxycarbonylamino, (H) phenyl- or naphthyl-oxycarbonylamino, (I) semicarbazido, (J) formamido, (K) thioformamido, (L) hydroxy, (M) nitro, (N) amino, (O) furyl, (P) triazolyl, (Q) thienyl, (R) oxazolyl, (S) imidazolyl and (T) triazolone-yl, (d) a 5- or 6-membered monocyclic or 8- to 10-membered bicyclic heterocycle having 1-3 heteroatoms each independently selected from the group consisting of N, O and S, (e) (C₁˜C₄ alkyl)phenyl, (f) (C₁˜C₄ alkyl)naphthyl, (g) hydroxy, (h) halogen, (i) amino and (j) carboxyl, (5) phenyl(C₁˜C₄ alkyl) which is unsubstituted or ring-substituted with 1-3 substituents each independently selected from the group consisting of (a) C₁˜C₅ alkyl which is unsubstituted or substituted with 1-3 substituents each independently selected from the group consisting of halogen, hydroxy, C₁˜C₄ alkoxy and amino, (b) halogen, (c) halo(C₁˜C₄ alkyl), (d) C₁˜C₄ alkoxy, (e) hydroxy, (f) amino, (g) carboxyl, (h) trifluormethoxyl, (i) trifluoromethyl, (j) tetrafluoroethyl, (k) tetrafluoroethoxyl, (l) tetrafluoropropyl and (m) tetrafluoropropoxyl, (6) naphthyl(C₁˜C₄ alkyl) which can be substituted with 1-6 substituents selected from (a) C₁˜C₄ alkyl which is unsubstituted or substituted with 1-3 substituents each independently selected from the group consisting of halogen, hydroxy, C₁˜C₄ alkoxy and amino, (b) halogen, (c) (C₁˜C₄ alkyl)halo, (d) C₁˜C₄ alkoxy, (e) hydroxy, (f) amino, (g) carboxyl, (h) trifluoromethoxyl, (i) trifluoromethyl, (j) tetrafluoroethyl, (k) tetrafluoroethoxyl, (l) tetrafluoropropyl and (m) tetrafluoropropoxyl, (7) methoxyl, (8) trifluoromethoxyl, (9) trifluoromethyl, (10) trifluoroethyl, (11) tetrafluoroethyl, (12) tetrafluoroethoxyl, (13) tetrafluoropropyl and (14) tetrafluoropropoxyl;
wherein each MM is independently an ethylene group optionally substituted with R91; or MM is —NN═OO—, wherein NN and OO are the same or different and are each independently N or CR⁹¹;
wherein each R⁹⁹ is independently a five-membered aromatic heterocyclic group containing one to four nitrogen atoms as ring-constituent atoms, which may further contain a heteroatom selected from sulfur or oxygen as a ring-constituent atom, said heterocyclic group being optionally substituted on the ring-constituent carbon and/or nitrogen atoms with one to three substituents selected from the group consisting of R³⁰ and R⁵⁶;
wherein each R¹⁰⁰ is independently —R⁵⁴ or —OR⁵⁴;
wherein each R¹⁰¹ is independently H; F; Cl; Br; or I;
wherein each R¹⁰² is independently F; Cl; Br; I; —SR¹⁰³; or —OR¹⁰⁴;
wherein each R¹⁰³ is independently straight chain or branched C₁˜C₁₂ alkyl, phenyl, furfuryl, —CH₂R⁶⁴, or benzyl, wherein said benzyl is optionally substituted by one to three R⁵⁶ substituents which can be the same or different;
wherein each R¹⁰⁴ is independently straight chain or branched C₁˜C₁₂ alkyl; or phenyl;
wherein each R¹⁰⁵ is independently H; R²⁹; straight chain or branched C₁˜C₈ alkyl; ═CH₂; straight chain or branched C₁˜C₆ alkenyl; straight chain or branched C₁˜C₆ alkyl substituted with one or two groups selected from the group consisting of F, Cl, Br, I, —C≡N, straight chain or branched C₁˜C₆ alkoxy, straight chain or branched C₁˜C₆ alkylthio, —C(═O)NH₂, —C(═O)—CH₂R⁶⁴, —C(═O)R⁴²; benzyl; methylenedioxybenzyl; C₇˜C₁₀ phenoxyalkyl optionally substituted with one to three halogen atoms; naphthyl; pyridyl optionally substituted with one to three substituents independently selected from the group consisting of halogen and R³⁰;
wherein each R¹⁰⁶ is independently H, a pharmaceutically acceptable cation, or null (affording an azolium phosphate zwitterion);
wherein each chiral center independently may possess any relative or absolute stereoconfiguration or be any mixture thereof, unless specified otherwise herein; and
wherein each olefinic C═C bond that is capable of E/Z isomerism independently may possess either the E or Z stereoconfiguration or be any mixture thereof, unless specified otherwise;
wherein the imidazole analog improves efficacy of the at least one prostaglandin analog of Formula I when the composition is delivered to a subject refractory to treatment by a composition containing the prostaglandin analog of Formula I alone.

50. The method according to claim 49, wherein the imidazole analog is at least one selected from the group consisting of histidine, bifonazole, butoconazole, chordentoin, chlorimidazole, cloconazole, clotrimazole, econazole, enilconazole, fenticonazole, flutrimazole, isocanazole, ketoconazole, lanoconazole, miconazole, omoconazole, oxiconazole, sertaconazole, sulconazole, and ioconazole or a pharmaceutically acceptable salt, hydrate, solvate, prodrug or metabolite thereof.

51. The method according to claim 49, wherein the imidazole analog is miconazole or ketoconazole or a pharmaceutically acceptable salt, hydrate, solvate, prodrug or metabolite thereof.

52. The method according to claim 41, wherein the at least one compound of formula I is at least one compound selected from the group consisting of a prostaglandin A analog, a prostaglandin B analog, a prostaglandin C analog, a prostaglandin D analog, a prostaglandin E analog, a prostaglandin F analog, a prostaglandin I analog and a prostaglandin J analog.

53. The method according to claim 52, wherein the prostaglandin A analog is selected from the group consisting of 16-phenoxy-17,18,19,20-tetranor PGA₂ N-cyclopropylamide, 16-phenoxy-17,18,19,20-tetranor PGA₁ N-cyclopropylmethylamide, 16-phenoxy-17,18,19,20-PGA1N-(1,3-dihydroxypropan-2-yl))amide, 17-phenyl-18,19,20-trinor PGA₂ N-cyclopropylamide, 17-phenyl-18,19,20-trinor PGA₁ N-cyclopropylmethylamide, 17-phenyl-18,19,20-trinor PGA2 N-(1,3-dihydroxypropan-2-yl))amide; 16-(3-chlorophenyl)-17,18,19,20-tetranor PGA₂ N-cyclopropylamide, 6-(3-chlorophenyl)-17,18,19,20-tetranor PGA₁ N-cyclopropylmethylamide, 6-(3-chlorophenyl)-17,18,19,20-tetranor PGA₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

54. The method according to claim 52, wherein the prostaglandin B analog is selected from the group consisting of 16-phenoxy-17,18,19,20-tetranor PGB₂ N-cyclopropylamide, 16-phenoxy-17,18,19,20-tetranor PGB₁ N-cyclopropylmethylamide, 16-phenoxy-17,18,19,20-PGB₁ N-(1,3-dihydroxypropan-2-yl))amide; 17-phenyl-18,19,20-trinor PGB₂ N-cyclopropylamide, 17-phenyl-18,19,20-trinor PGB₁ N-cyclopropylmethylamide, 17-phenyl-18,19,20-trinor PGB₂ N-(1,3-dihydroxypropan-2-yl))amide; 16-(3-chlorophenyl)-17,18,19,20-tetranor PGB₂ N-cyclopropylamide, 16-(3-chlorophenyl)-17,18,19,20-tetranor PGB₁ N-cyclopropylmethylamide, 6-(3-chlorophenyl)-17,18,19,20-tetranor PGB₁ N-(1,3-dihydroxypropan-2-yl))amide, (Z)-isopropyl 7-((1R,2S)-2-((R)-3-hydroxy-5-phenylpentyl)-5-oxocyclopent-3-enyl)hept-5-enoate, (Z)-isopropyl 7-((1R,2S)-2-((R,E)-3-hydroxy-4-(3-(trifluoromethyl)phenoxy)but-1-enyl)-5-oxocyclopent-3-enyl)hept-5-enoate, (Z)—N-ethyl-7-((1R,2S)-2-((R,E)-3-hydroxy-4-(3-(trifluoromethyl)phenoxy)but-1-enyl)-5-oxocyclopent-3-enyl)hept-5-enamide, (Z)—N-ethyl-7-((1R,2S)-2-((S,E)-3-hydroxy-5-phenylpent-1-enyl)-5-oxocyclopent-3-enyl)hept-5-enamide, (Z)-7-((1R,2S)-2-((R,E)-3-hydroxy-4-(3-(trifluoromethyl)phenoxy)but-1-enyl)-5-oxocyclopent-3-enyl)hept-5-enoic acid (Z)-7-((1R,2S)-2-((R,E)-3-hydroxy-4-(3-(trifluoromethyl)phenoxy)but-1-enyl)-5-oxocyclopent-3-enyl)-N-methylhept-5-enamide (Z)-7-((1R,2S)-2-((R,E)-4-(3-chlorophenoxy)-3-hydroxybut-1-enyl)-5-oxocyclopent-3-enyl)hept-5-enoic acid, (Z)-isopropyl 7-((1R,2S)-2-((R,E)-4-(3-chlorophenoxy)-3-hydroxybut-1-enyl)-5-oxocyclopent-3-enyl)hept-5-enoate, and (Z)-7-((1R,2S)-2-((R,E)-4-(3-chlorophenoxy)-3-hydroxybut-1-enyl)-5-oxocyclopent-3-enyl)-N-methylhept-5-enamide or a pharmaceutically acceptable salt, hydrate, solvate, prodrug or metabolite thereof.

55. The method according to claim 52, wherein the prostaglandin C analog is selected from the group consisting of 16-phenoxy-17,18,19,20-tetranor PGC2 N-cyclopropylamide, 16-phenoxy-17,18,19,20-tetranor PGC₁ N-cyclopropylmethylamide, 16-phenoxy-17,18,19,20-PGC₁ N-(1,3-dihydroxypropan-2-yl))amide; 17-phenyl-18,19,20-trinor PGC₂ N-cyclopropylamide, 17-phenyl-18,19,20-trinor PGC₁ N-cyclopropylmethylamide, 17-phenyl-18,19,20-trinor PGC₂ N-(1,3-dihydroxypropan-2-yl))amide; 16-(3-chlorophenyl)-17,18,19,20-tetranor PGC₂ N-cyclopropylamide, 16-(3-chlorophenyl)-17,18,19,20-tetranor PGC₁ N-cyclopropylmethylamide, 6-(3-chlorophenyl)-17,18,19,20-tetranor PGC₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

56. The method according to claim 52, wherein the prostaglandin D analog is selected from the group consisting of 16-phenoxy-17,18,19,20-tetranor PGD₂ N-cyclopropylamide, 16-phenoxy-17,18,19,20-tetranor PGD₁ N-cyclopropylmethylamide, 16-phenoxy-17,18,19,20-PGD₁ N-(1,3-dihydroxypropan-2-yl))amide; 17-phenyl-18,19,20-trinor PGD₂ N-cyclopropylamide, 17-phenyl-18,19,20-trinor PGD₁ N-cyclopropylmethylamide, 17-phenyl-18,19,20-trinor PGD₂N-(1,3-dihydroxypropan-2-yl))amide; 16-(3-chlorophenyl)-17,18,19,20-tetranor PGD₂ N-cyclopropylamide, 16-(3-chlorophenyl)-17,18,19,20-tetranor PGD₁ N-cyclopropylmethylamide, 6-(3-chlorophenyl)-17,18,19,20-tetranor PGD₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

57. The method according to claim 52, wherein the prostaglandin E analog is selected from the group consisting of 16-phenoxy-17,18,19,20-tetranor PGE₂ N-cyclopropylamide, 16-phenoxy-17,18,19,20-tetranor PGE₁ N-cyclopropylmethylamide, 16-phenoxy-17,18,19,20-PGE₁N-(1,3-dihydroxypropan-2-yl))amide; 17-phenyl-18,19,20-trinor PGE₂ N-cyclopropylamide, 17-phenyl-18,19,20-trinor PGE₁ N-cyclopropylmethylamide, 17-phenyl-18,19,20-trinor PGE₂ N-(1,3-dihydroxypropan-2-yl))amide; 16-(3-chlorophenyl)-17,18,19,20-tetranor PGE₂ N-cyclopropylamide, 16-(3-chlorophenyl)-17,18,19,20-tetranor PGE₁ N-cyclopropylmethylamide, 6-(3-chlorophenyl)-17,18,19,20-tetranor PGE₁N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

58. The method according to claim 52, wherein the prostaglandin F analog is selected from the group consisting of 16-phenoxy-17,18,19,20-tetranor PGF_{2α}, N-cyclopropylamide, 16-phenoxy-17,18,19,20-tetranor PGF_{2α}, N-cyclopropylmethylamide, 16-phenoxy-17,18,19,20-PGF_{2α}, N-(1,3-dihydroxypropan-2-yl))amide, -phenyl-18,19,20-trinor PGF_{2α}, N-cyclopropylamide, 17-phenyl-18,19,20-trinor PGF_{2α}, N-cyclopropylmethylamide, 17-phenyl-18,19,20-trinor PGF_{2α}, N-(1,3-dihydroxypropan-2-yl))amide, 16-(3-chlorophenyl)-17,18,19,20-tetranor PGF_{2α} N-cyclopropylamide, 16-(3-chlorophenyl)-17,18,19,20-tetranor PGF_{2α}, N-cyclopropylmethylamide, 6-(3-chlorophenyl)-17,18,19,20-tetranor PGF_{2α}, N-(1,3-dihydroxypropan-2-yl))amide, latanoprost, travoprost, travoprost N-ethylamide, bimatoprost, fluprostenol, fluprostenol isopropyl ester, fluprostenol N-methylamide, 9-keto fluprostenol isopropyl ester, cloprostenol, cloprostenol isopropyl ester, and chloprostenol N-methylamide or a pharmaceutically acceptable salt, hydrate, solvate, prodrug or metabolite thereof.

59. The method according to claim 52, wherein the prostaglandin J analog is selected from the group consisting of 16-phenoxy-17,18,19,20-tetranor PGJ₂ N-cyclopropylamide, 16-phenoxy-17,18,19,20-tetranor PGJ₁ N-cyclopropylmethylamide, 16-phenoxy-17,18,19,20-PGJ₁ N-(1,3-dihydroxypropan-2-yl))amide; 17-phenyl-18,19,20-trinor PGJ₂ N-cyclopropylamide, 17-phenyl-18,19,20-trinor PGJ₁ N-cyclopropylmethylamide, 17-phenyl-18,19,20-trinor PGJ₂N-(1,3-dihydroxypropan-2-yl))amide; 16-(3-chlorophenyl)-17,18,19,20-tetranor PGJ₂ N-cyclopropylamide, 16-(3-chlorophenyl)-17,18,19,20-tetranor PGJ₁ N-cyclopropylmethylamide, 6-(3-chlorophenyl)-17,18,19,20-tetranor PGJ₁ N-(1,3-dihydroxypropan-2-yl))amide, (Z)-isopropyl 7-((R)-2-((R)-3-hydroxy-5-phenylpentyl)-5-oxocyclopent-2-enyl)hept-5-enoate, (Z)-isopropyl 7-((R)-2-((R,E)-3-hydroxy-4-(3-(trifluoromethyl)phenoxy)but-1-enyl)-5-oxocyclopent-2-enyl)hept-5-enoate, (Z)—N-ethyl-7-((R)-2-((R,E)-3-hydroxy-4-(3-(trifluoromethyl)phenoxy)but-1-enyl)-5-oxocyclopent-2-enyl)hept-5-enamide, (Z)—N-ethyl-7-((R)-2-((S,E)-3-hydroxy-5-phenylpent-1-enyl)-5-oxocyclopent-2-enyl)hept-5-enamide, (Z)-7-((R)-2-((R,E)-3-hydroxy-4-(3-(trifluoromethyl)phenoxy)but-1-enyl)-5-oxocyclopent-2-enyl)hept-5-enoic acid, (Z)-7-((R)-2-((R,E)-3-hydroxy-4-(3-(trifluoromethyl)phenoxy)but-1-enyl)-5-oxocyclopent-2-enyl)-N-methylhept-5-enamide, (Z)-7-((R)-2-((R,E)-4-(3-chlorophenoxy)-3-hydroxybut-1-enyl)-5-oxocyclopent-2-enyl)hept-5-enoic acid, (Z)-isopropyl 7-((R)-2-((R,E)-4-(3-chlorophenoxy)-3-hydroxybut-1-enyl)-5-oxocyclopent-2-enyl)hept-5-enoate, (Z)-7-((R)-2-((R,E)-4-(3-chlorophenoxy)-3-hydroxybut-1-enyl)-5-oxocyclopent-2-enyl)-N-methylhept-5-enamide or a pharmaceutically acceptable salt, hydrate, solvate, prodrug or metabolite thereof.

60. The method according to claim 41, wherein the optional second component of the composition comprises an additional active agent selected from the group consisting of a protective agent, an emollient, an astringent, an irritant, a keratolytic, a sun screening agent, a sun tanning agent, an antibiotic agent, a non-imidazole analog antifungal agent, an imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant, a chemotherapeutic agent, an anti-histamine, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent, a hypo-pigmenting agent, or a combination thereof.

61. The method according to claim 41, wherein the at least one chemotherapeutic agent is at least one agent selected from the group consisting of an alkylating agent, an antimetabolite, a natural product, a biologic agent, a hormone or hormone-related agent, and a miscellaneous agent.

62. The method according to claim 41, wherein the at least one chemotherapeutic agent is an alkylating agent, and wherein the side-effect of treatment with the alkylating agent is alopecia.

63. The method according to claim 62, wherein the alkylating agent is selected from the group consisting of temozolomide, busulfan, ifosamide, melphalan hydrochloride, carmustine, lomustine and cyclophosphamide.

64. The method according to claim 41, wherein the at least one chemotherapeutic agent is an antimetabolite, and wherein the side-effect of treatment with the antimetabolite is alopecia.

65. The method according to claim 64, wherein the antimetabolite is selected from the group consisting of 5-fluorouracil, capecitabine, gemcitabine, floxuridine, decitabine, mercaptopurine, pemetrexed disodium, methotrexate and dacarbazine.

66. The method according to claim 41, wherein the at least one chemotherapeutic agent is a natural product, and wherein the side-effect of treatment with the natural product is alopecia.

67. The method according to claim 66, wherein the natural product is selected from the group consisting of vincristine, vinblastine, vinorelbine tartrate, paclitaxel, docetaxel, ixabepilone, daunorubicin, epirubicin, doxorubicin, idarubicin, mitoxantrone, mitomycin, dactinomycin, irinotecan, topotecan, etoposide, teniposide, etoposide phosphate, and bleomycin sulfate.

68. The method according to claim 41, wherein the at least one chemotherapeutic is a biologic agent, and wherein the side-effect of treatment with the biologic agent is alopecia.

69. The method according to claim 68, wherein the biologic agent is selected from the group consisting of filgrastim, pegfilgrastim, bevacizumab, sargramostim and panitumumab.

70. The method according to claim 41, wherein the at least one chemotherapeutic agent is a hormone or a hormone-related agent, and wherein the side-effect of treatment with the hormone or the hormone-related agent is alopecia.

71. The method according to claim 70, wherein the hormone or the hormone-related agent is selected from the group consisting of megestrol acetate, fluoxymesterone, leuprolide, octreotide acetate, tamoxifen citrate and fluxymesterone.

72. The method according to claim 41, wherein the at least one chemotherapeutic agent is a miscellaneous agent, and wherein the side-effect of treatment with the miscellaneous agent is alopecia.

73. The method according to claim 41, wherein the miscellaneous agent is selected from the group consisting of sorafenib, erlotinib, oxaliplatin, dexrazoxane, anagrelide, isotretinoin, bexarotene and vorinostat.

74. The method according to claim 41, wherein the topical composition is administered concurrently with a chemotherapy regimen for treating breast cancer.

75. The method according to claim 74, wherein the chemotherapy regimen is an Adriamycin-Cytoxan-Taxol regimen.

76. The method according to claim 41, wherein the topical composition is administered concurrently with a chemotherapy regimen for treating colorectal cancer.

77. The method according to claim 76, wherein the chemotherapy regimen comprises a combination of infusional 5-fluoruracil, leucovorin and oxaliplatin (FOLFOX®).

78. The method according to claim 76, wherein the chemotherapeutic regimen for colorectal cancer comprises FOLFOX® with bevacizumab.

79. The method according to claim 76, wherein the chemotherapy regimen comprises infusional 5-fluoruracil, leucovorin, and irinotecan (FOLFIRI®) with bevacizumab.

80. The method according to claim 41, wherein the composition is formulated as a mascara.
